(19) United States
(12) Patent Application Publication
(10) Pub. No.: US 2019/0284136 A1
(43) Pub. Date: Sep. 19, 2019

(54) INDOLINE DERIVATIVES

(71) Applicant: ViiV HEALTHCARE UK LIMITED, Brentford, Middlesex (GB)

(72) Inventors: Emile Johann VELTHUISEN, Research Triangle Park, NC (US); Jason Gordon WEATHERHEAD, Research Triangle Park, NC (US)

(21) Appl. No.: 16/317,047

(22) PCT Filed: Jul. 17, 2017
(86) PCT No.: PCT/IB2017/054308

§ 371 Date: Jan. 11, 2019

Related U.S. Application Data
(60) Provisional application No. 62/366,190, filed on Jul. 25, 2016.

(51) Int. Cl. C07D 209/12; C07D 405/10; C07D 401/06; C07D 417/06; C07D 401/12; C07D 405/14

(52) U.S. Cl. CPC C07D 209/12; C07D 405/10; C07D 405/14; C07D 417/06; C07D 401/12; C07D 401/06

(57) ABSTRACT

Compounds of Formula I are disclosed and methods of treating viral infections with compositions comprising such compounds.

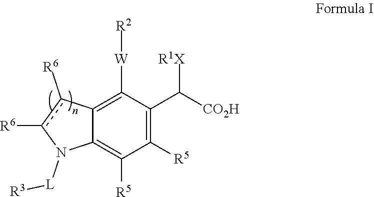

### FIELD OF THE INVENTION

The present invention relates to substituted indoline compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors.

However, resistance to all three new drug classes has already been reported both in the lab and in patients. Sustained successful treatment of HIV-1-infected patients with antiretroviral drugs will therefore require the continued development of new and improved drugs with new targets and mechanisms of action.

For example, over the last decade HIV inhibitors have been reported to target the protein-protein interaction between HIV-1 integrase and Lens Epithelium Derived Growth Factor/p75 (“LEDGF”). LEDGF is a cellular transcriptional cofactor of HIV-1 integrase that promotes viral integration of reverse transcribed viral cDNA into the host cell's genome by tethering the preintegration complex to the chromatin. Because of its crucial role in the early steps of HIV replication, the interaction between LEDGF and integrase represents another attractive target for HIV drug therapy.

The following patent applications disclose certain compounds useful for treating HIV: WO 2013/012649; WO 2012/102985; WO 2013/043553; WO 2014/009794; WO 2016/005878; WO 2016/012913; WO 2016/012930; U.S. Ser. No. 62/219,687; U.S. Ser. No. 62/262,935; 62/262,937; U.S. Ser. No. 62/262,938; and U.S. Ser. No. 62/282,934.

### SUMMARY OF THE INVENTION

Briefly, in one aspect, the present invention discloses compounds of Formula I:

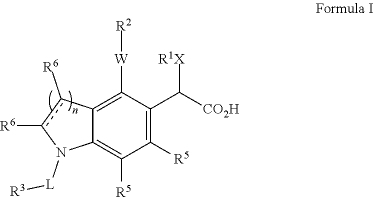

wherein:

The dashed line between the carbons to which the R⁶ groups are bonded is meant to indicate that the bond can be either a single bond or a double bond;
n is 1 or 2 with the proviso that when n is 2 the dashed line must be a single bond;
X is O or CH₂;
R¹ is C₁₋₆alkyl wherein said alkyl may contain cycloalkyl portions;
W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂(CO)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆heteroalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring; L is a bond, —CH₂(CO)—, C₁₋₃alkylene, —SO₂—, —SO₂NH—, —C(O)—, —C(O)NH—, —C(O)NHC₁₋₂alkyl-, —C(O)OCH₂—, —C(O)O—, —C(O)C(O)—, or —C(O)C₁₋₂alkyl-; R³ is H, CN, C₁₋₆alkyl, C₅₋₁₄aryl, C₅₋₁₄aryl, C₃₋₇cycloalkyl, C₃₋₇cycloalkyl, C₃₋₇spirocycloalkyl, C₃₋₇cycloalkenyl, C₃₋₉heterocycle, C₅₋₉heteroaryl, or tetrahydronaphthyl, and wherein R³ is optionally substituted by one to four substituents selected from halo, oxo, C₁₋₆alkyl, C₃₋₇cycloalkyl, C₁₋₃fluoroalkyl, —OC₁₋₆alkyl, —C(O)C₁₋₃alkyl, —C(O)N(H)C₁₋₃alkyl, —NHC(O)C₁₋₃alkyl, —C(O)NHR⁴, C₅₋₁₄aryl, C₁₋₆heteroalkyl, —B(OH)₂, C₃₋₉-heterocycle, C₅₋₉-heteroaryl, —C(O)OC₁₋₆alkyl, or the following divalent substituents may be bonded to adjacent atoms of R³ to form a fused ring, —C₂₋₅alkylene-, —OC₁₋₃alkyleneO—, —OC₁₋₄alkylene-, or —N═C(CH₃)O—;
each R⁵ is independently H, C₁₋₃alkyl, C₃₋₆cycloalkyl, CH₂F, CHF₂, or CF₃;
each R⁶ is independently H, C₁₋₃alkyl, C₅₋₁₄aryl, C₃₋₉-heterocycle, C₅₋₉-heteroaryl, —C(O)NR⁴, or —C(O)NHR⁴, or an R⁶ may represent a gem dimethyl, or two R⁶ groups may together comprise 2-4 carbon atoms and join together to form a fused ring system wherein the ring formed by the two R⁶ groups can be cycloalkyl, or heterocycle, aryl, or heteroaryl; and wherein each heterocycle, heteroaryl, heteroalkyl, and heteroalkylene comprises one to three heteroatoms selected from S, N, B, or O.

In another aspect the present invention discloses pharmaceutically acceptable salts of the compounds of Formula I.

In another aspect, the present invention discloses pharmaceutical compositions comprising a compound of Formula I or a pharmaceutically acceptable salt thereof.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

In accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

### DETAILED DESCRIPTION OF THE INVENTION

Preferably the dashed line represents a single bond.

Preferably n is 1.

Preferably W is a bond.

Preferably R¹ is C₁₋₆alkyl. Most preferably, R¹ is t-butyl.

Preferably X is O.

Preferably R² is phenyl optionally substituted by one to four substituents selected from halo, C₁₋₆alkyl, C₁₋₆heteroalkyl, or C₁₋₆alkylene or C₁₋₆hetereoalklylene wherein said C₁₋₆alkylene or C₁₋₆hetereoalklylene is bonded to adjacent carbon atoms on said phenyl to form a fused ring and wherein each heteroalkyl and heteroalkylene comprises one to two heteroatoms selected from S, N, or O. Most preferably, R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

Preferably L is CH₂, —C(O)—, a bond, —C(O)C(O)—, —C(O)NH—, —C(O)O—, —C(O)CH₂—, SO₂, —C(O)CH₂CH₂—, —CH₂C(O)—, or —C(O)CH₂—. Most preferably L is —C(O)—.

Preferably R³ is C₂₋₆alkyl, C₅₋₆cycloalkenyl, C₅₋₆aryl, C₃₋₆cycloalkyl, C₅₋₆heterocycle containing 1 oxygen atom or 1 nitrogen atom, C₅₋₆heteroaryl containing 1-3 heteroatoms selected from N, S, and O, wherein R³ is optionally substituted by one to three substituents selected from F, Cl, C₁₋₃alkyl, OC₁₋₃alkyl, C₁₋₃fluoroalkyl, NHC(O)C₁₋₃alkyl, C(O)NHC₁₋₃alkyl, C(O)OC₁₋₃alkyl, or the following divalent substituents may be bonded to adjacent atoms of R³ to form a fused ring, —C₂₋₅alkylene-, —OC₁₋₃alkyleneO—, —OC₁₋₄alkylene-, or —N═C(CH₃)O—.

Most preferably R³ is phenyl optionally substituted by one to three substituents selected from F, Cl, C₁₋₃alkyl, OC₁₋₃alkyl, C₁₋₃fluoroalkyl, NHC(O)C₁₋₃alkyl, C(O)NHC₁₋₃alkyl, C(O)OC₁₋₃alkyl, or the following divalent substituents may be bonded to adjacent atoms of R³ to form a fused ring, —C₂₋₅alkylene-, —OC₁₋₃alkyleneO—, —OC₁₋₄alkylene-, or —N═C(CH₃)O—.

Preferably one R⁵ is methyl and the other is H.

Preferably each R⁶ is H.

Preferably the stereochemistry on the carbon to which XR¹ is bound is as depicted below.

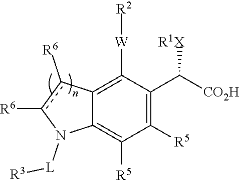

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

### EXAMPLES

The compounds of this invention may be made by a variety of methods, including well-known standard synthetic methods. Illustrative general synthetic methods are set out below and then specific compounds of the invention are prepared in the working examples.

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.
Ar=aryl
aq.=aqueous
μL=microliters
μM=micromolar
NMR=nuclear magnetic resonance
boc=tert-butoxycarbonyl
BPin=pinacoladoboronic ester
(Bpin)₂=bispinacoladodiborane
br=broad
Cbz=benzyloxycarbonyl
d=doublet
δ=chemical shift
° C.=degrees Celsius
DCM=dichloromethane
dd=doublet of doublets
DEAD=diethylazidodicarboxylate
DMEM=Dulbeco's Modified Eagle's Medium
DMF=N,N-dimethylformamide
DMSO=dimethylsulfoxide
EtOAc=ethyl acetate
g=gram
h or hr=hours
HCV=hepatitis C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H+
mg=milligram
min=minutes
mL=milliliter
mM=millimolar
mmol=millimole
MS=mass spectrum
nm=nanomolar
NMP=N-methylpyrolidinone
ppm=parts per million
q.s.=sufficient amount
s=singlet
RT=room temperature
sat.=saturated
t=triplet
TES=triethylsilyl
TFA=trifluoroacetic acid
Tf=triflyl
TMS=trimethylsilyl
Z=benzyloxycarbonyl

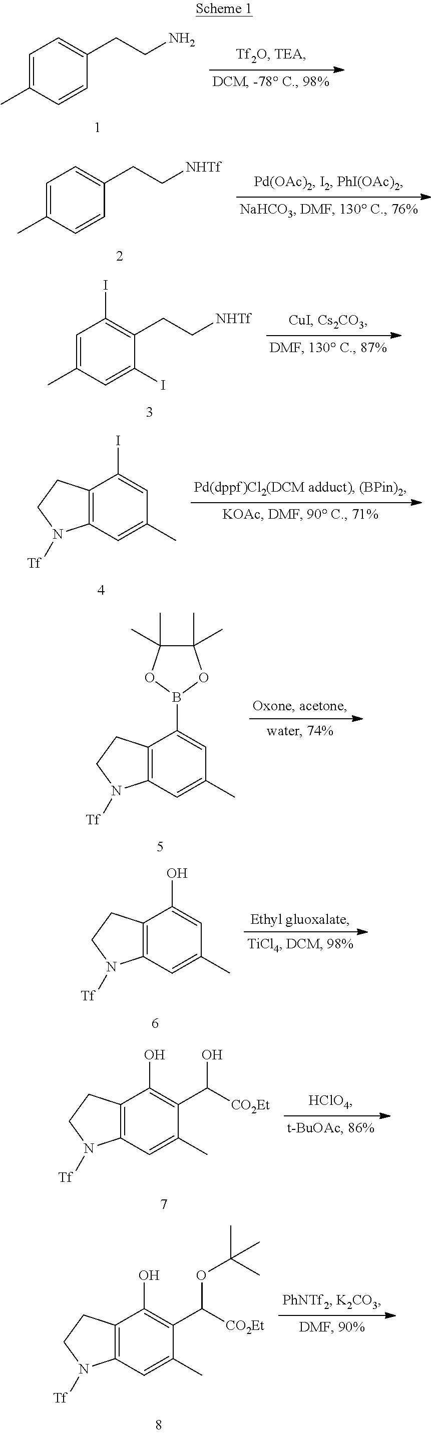

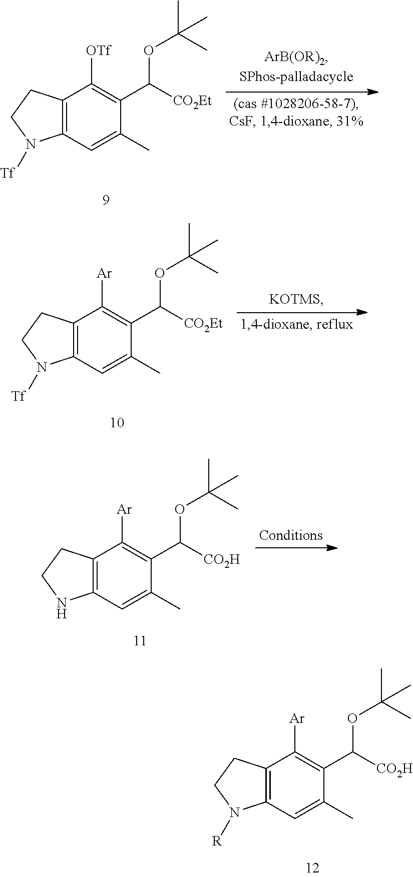

### Example 1: (2-(tert-Butoxy)-2-(1-(3,4-difluorobenzyl)-6-methyl-4-(p-tolyl)indolin-5-yl)acetic acid

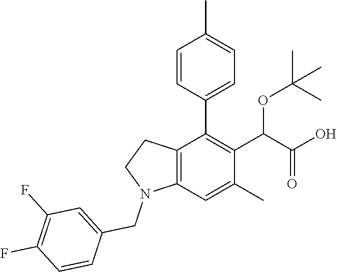 [SMILES: Cc1ccc(-c2c3c(cc(C)c2C(OC(C)(C)C)C(=O)O)N(Cc2ccc(F)c(F)c2)CC3)cc1]

### Step 1: 1,1,1-Trifluoro-N-(4-methylphenethyl)methanesulfonamide

2-(p-Tolyl)ethanamine (631 mg, 4.67 mmol) was dissolved in DCM (10 mL) and cooled to −78° C. TEA (0.72 mL), 5.13 mmol) was added, followed by the dropwise addition of triflic anhydride (0.87 mL, 5.13 mmol) The mixture was stirred for 20 minutes at this temperature. TLC (5% MeOH/DCM and 8/2 hexanes/EtOAc) confirmed complete formation of nonpolar product. The reaction was poured into ice water and extracted with DCM. The organic layer was washed with brine, dried org over sodium sulfate, concentrated in vacuo, and purified by silica gel chromatography (0-100% EtOAc/hexanes) to give the desired product as a clear oil (1.13 g, 91%). LCMS (ES+) (m/z): 266.2 (M−1).

### Step 2: N-(2,6-Diiodo-4-methylphenethyl)-1,1,1-trifluoromethanesulfonamide

In a flask open to the air, the product from Step 1 (1.12 g, 4.2 mmol) was dissolved in DMF (20 mL) and palladium (II) acetate (0.1 eq, 0.42 mmol, 94 mg), PhI(OAc)₂ (2 eq, 8.4 mmol, 2.71 g), iodine (2 eq, 8.4 mmol, 2.14 g), and NaHCO₃ (1 eq, 4.2 mmol, 353 mg) were added and mixture was heated at 130° C. for −12 hours until complete by TLC (9:1 hexanes:EtOAc) The mixture was concentrated in vacuo. and purified by silica gel chromatography (0-100% EtOAc/hexanes) to give the desired product as a white solid (1.57 g, 72%). LCMS (ES+) (m/z): 542.1 (M+1).

### Step 3: 4-Iodo-6-methyl-1-((trifluoromethyl)sulfonyl)indoline

The product from Step 2 (1.57 g, 3.02 mmol) was combined with CuI (0.5 eq, 1.51 mmol, 288 mg) and cesium carbonate (1 eq, 3.02 mmol, 984 mg) in DMF (15 mL) and the mixture was immersed in a 130° C. oil bath. After 1 hour at this temperature the reaction was cooled to room temperature and diluted with water (exotherm) and then filtered through Celite. The Celite was washed with diethyl ether and then the filtrate was extracted with diethyl ether. The organics were dried over sodium sulfate, concentrated in vacuo. and purified by silica gel chromatography (0-100% EtOAc/hexanes) to give the desired product as a clear oil (929 mg, 79%). 7.32 (s, 1H), 7.21 (s, 1H), 4.21 (t, J=8 Hz, 2H), 3.11 (t, J=8 Hz, 2H), 2.31 (s, 3H).

### Step 4: 6-Methyl-4-(4,4,5,5-tetramethyl-1,3,2-dioxaborolan-2-yl)-1-((trifluoromethyl)sulfonyl)indoline

The product from Step 3 (100 mg, 0.256 mmol) was dissolved in DMF (3 mL) and the mixture was degassed while (BPin)₂ (97 mg, 0.383 mmol), KOAc (75 mg, 0.767 mmol), and Pd(dppf)Cl₂ (DCM adduct) (21 mg, 0.026 mmol) was added. The mixture was immersed in a 90° C. bath for 2 hours and then cooled to room temperature and poured over ice water. The mixture was filtered through Celite, the filter washed with diethyl ether, and then the filtrate was extracted with diethyl ether. The organics were dried over sodium sulfate, concentrated in vacuo. and purified by silica gel chromatography (0-100% EtOAc/hexanes) to give the desired product as a white solid (72 mg, 72%). ¹H NMR (400 MHz, CHLOROFORM-d) δ ppm 7.36 (m, 2H), 4.18 (m, 2H), 3.36 (m, 2H), 2.34 (s, 3H), 1.33 (s, 12H).

### Step 5: 6-Methyl-1-((trifluoromethyl)sulfonyl)indolin-4-ol

The product from Step 4 (31 mg, 0.08 mmol) was suspended in acetone (2 mL) and the mixture was cooled to 0° C. Oxone (1.33 eq, 0.107mmo, 33 mgl) in water (1 mL) was added dropwise, then the reaction was warmed to room temperature and stirred 1 hour. The reaction was cooled to 0° C. and additional Oxone was added to push the reaction to completion (37 mg in two portions). The mixture was poured over ice water and diluted with NaHCO₃ solution, and extracted with EtOAc. The organics were dried over sodium sulfate, concentrated in vacuo, and purified by silica gel chromatography (0-100% EtOAc/hexanes) to give the desired product as a white solid (15 mg, 68%). LCMS (ES+) (m/z): 282.2 (M+1).

### Step 6: Ethyl 2-hydroxy-2-(4-hydroxy-6-methyl-1-((trifluoromethyl)sulfonyl)indolin-5-yl)acetate

The product from Step 5 (221 mg, 0.79 mmol) was dissolved in DCM (8 mL) and cooled to 0° C. TiCl4 (1.16 mL, 1.16 mmol, 1 M in DCM) was added dropwise and the mixture was stirred 10 minutes to give a red suspension. Ethyl Glyoxalate (0.19 mL, 0.98 mmol) was added and the reaction was quickly complete by TLC (6/4 hexanes/EtOAc). The mixture was poured onto 1 N HCl and ice water and extracted with DCM. The organics were washed with brine, dried over sodium sulfate and concentrated in vacuo, and purified by silica gel chromatography (0-100% EtOAc/hexanes) to give the desired product as a tan solid (270 mg, 89%). LCMS (ES+) (m/z): 384.3 (M+1).

### Step 7: Ethyl 2-(tert-butoxy)-2-(4-hydroxy-6-methyl-1-((trifluoromethyl)sulfonyl)indolin-5-yl)acetate

The product from Step 6 (206 mg, 0.54 mmol) was dissolved in tert-butyl acetate (15 mL) and HClO₄ (1.6 ml) was added. The mixture was stirred 10 minutes at room temperature. The reaction was cooled to 0° C., made basic with a combination of 1 N and 50% NaOH solutions, and then extracted with EtOAc. The organics were washed with brine, dried over sodium sulfate concentrated in vacuo, and purified by silica gel chromatography (0-100% EtOAc/hexanes) to give the desired product as a clear oil (153 mg, 65%). LCMS (ES+) (m/z): 462.3 (M+23).

### Step 8: Ethyl 2-(tert-butoxy)-2-(6-methyl-1-((trifluoromethyl)sulfonyl)-4-(((trifluoromethyl)sulfonyl)oxy)indolin-5-yl)acetate

The product from Step 7 (125 mg, 0.28 mmol) was dissolved in DMF (2 mL) and K₂CO₃ (78 mg, 0.57 mmol) and PhNTf₂ (110 mg, 0.038 mmol) were added. The reaction was stirred at room temperature for 20 minutes and then poured into and ice water and NaHCO₃ solution. The mixture was extracted with diethyl ether, and the organics were dried over sodium sulfate, concentrated in vacuo, and purified by silica gel chromatography (0-100% EtOAc/hexanes) to give the desired product (149 mg, 94%). LCMS (ES+) (m/z): 594.3 (M+23).

### Step 9: Ethyl 2-(tert-butoxy)-2-(6-methyl-4-(p-tolyl)-1-((trifluoromethyl)sulfonyl)indolin-5-yl)acetate

The product from Step 8 (24 mg, 0.042 mmol) was dissolved in 1,4-dioxane (2 mL) and degassed while adding p-tolylboronic acid 14.27 mg, 0.105 mmol), S-Phos palladacycle (9.58 mg, 0.013 mmol) (CAS #1028206-58-7), and CsF (25.5 mg, 0.168 mmol). The mixture was irradiated in a microwave at 130° C. for 40 minutes. The reaction was filtered through Celite and the filtrated was extracted with EtOAc, the organics dried over sodium sulfate, concentrated in vacuo, and purified by silica gel chromatography (0-100% EtOAc/hexanes) to give the desired product (18 mg, 83%). LCMS (ES+) (m/z): 336.4 (M+23).

### Step 10: 2-(tert-Butoxy)-2-(6-methyl-4-(p-tolyl)indolin-5-yl)acetic acid

The product from Step 9 (18 mg, 0.035 mmol) was dissolved in 1,4-dioxane (3 mL) and KOTMS (18 mg, 0.141 mmol) was added. The mixture was heated to 100° C. and formed a yellow suspension. After 20 minutes, the reaction was cooled to room temperature and poured into a 1 N HCl-ice water mixture and extracted with EtOAc and a solution of CHCl3:IPA (3:1). The organics were dried over sodium sulfate, concentrated in vacuo, and purified by reverse-phase HPLC to give the desired product (8 mg, 65%). LCMS (ES+) (m/z): 354.4 (M+1).

### Step 11: 2-(tert-Butoxy)-2-(1-(3,4-difluorobenzyl)-6-methyl-4-(p-tolyl)indolin-5-yl)acetic acid

The amino acid from Step 10 (8 mg, 0.023 mmol) was dissolved in 1,2-dichloroethane (1.3 mL) and 3,4-difluorobenzaldehyde (4.5 mg, 0.032 mmol) was added and the reaction was stirred for 10 minutes at room temperature. NaBH(OAc)₃ (7.2 mg, 0.034 mmol) was added and the reaction was stirred for 1 hour. The mixture was poured onto ice water and extracted with DCM. The organics were dried over sodium sulfate, concentrated in vacuo, and purified by reverse-phase HPLC to give the title compound as a yellow oil (4 mg, 34%). ¹H NMR (400 MHz, CHLOROFORM-d) δ ppm 7.47 (m, 1H), 7.05-7.26 (m, 6H), 6.30 (s, 1H), 5.21 (s, 1H), 4.02-4.35 (m, 2H), 3.08-3.49 (m, 2H), 2.60-2.97 (m, 1H), 2.23-2.57 (m, 8H), 0.96 (s, 9H). LCMS (ES+) (m/z): 480.4 (M+1).

### Example 2: 2-(tert-Butoxy)-2-(1-(3,4-difluorobenzyl)-4-(8-fluoro-5-methylchroman-6-yl)-6-methylindolin-5-yl)acetic acid

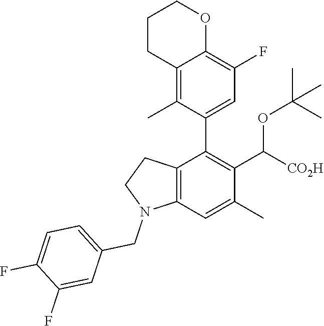 [SMILES: Cc1cc2c(c(-c3cc(F)c4c(c3C)CCCO4)c1C(OC(C)(C)C)C(=O)O)CCN2Cc1ccc(F)c(F)c1]

In a manner similar to that described in Example 1, from 2-(tert-Butoxy)-2-(6-methyl-4-(p-tolyl)indolin-5-yl)acetic acid, the title compound was prepared as a yellow oil (8 mg, 23%). ¹H NMR (400 MHz, CHLOROFORM-d) δ ppm 7.04-7.24 (m, 3H), 6.73 (d, J=12 Hz, 1H), 6.30 (s, 1H), 4.99 (s, 1H), 4.18-4.30 (m, 4H), 3.65 (br. m, 2H), 3.23 (m, 2H), 2.33-2.75 (m, 5H), 2.11 (m, 2H), 1.98 (s, 3H), 1.13 (s, 9H). LCMS (ES+) (m/z): 554.4 (M+1).

### Example 3: 2-(tert-Butoxy)-2-(1-(3,4-difluorobenzoyl)-6-methyl-4-(p-tolyl)indolin-5-yl)acetic acid

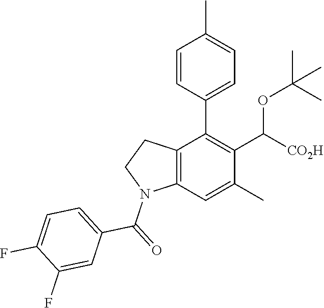 [SMILES: Cc1ccc(-c2c3c(cc(C)c2C(OC(C)(C)C)C(=O)O)N(C(=O)c2ccc(F)c(F)c2)CC3)cc1]

2-(tert-Butoxy)-2-(6-methyl-4-(p-tolyl)indolin-5-yl)acetic acid (25 mg, 0.071 mmol) was dissolved in EtOAc (2 mL) and TEA (0.03 mL, 0.212 mmol) and T3P (0.105 mL, 0.177 mmol, 50 wt. % in EtOAc) were added. The reaction was stirred 30 minutes and then diluted with NaHCO₃solution and extracted with EtOAc. The organics were dried over sodium sulfate, concentrated in vacuo, and purified by reverse-phase HPLC to give the title compound as an oil (4 mg, 12%). ¹H NMR (400 MHz, CHLOROFORM-d) δ ppm 7.30-7.51 (m, 4H), 7.06-7.25 (m, 4H), 5.23 (s, 1H), 3.97 (m, 2H), 2.60 (m, 2H), 2.41 (br. s, 6H), 0.97 (s, 9H). LCMS (ES+) (m/z): 494.3 (M+1).

### Example 4: 2-(tert-Butoxy)-2-(1-cyclohexyl-6-methyl-4-(p-tolyl)indolin-5-yl)acetic acid

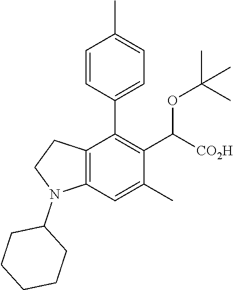 [SMILES: Cc1ccc(-c2c3c(cc(C)c2C(OC(C)(C)C)C(=O)O)N(C2CCCCC2)CC3)cc1]

In a manner similar to that described in Example 1, from 2-(tert-Butoxy)-2-(6-methyl-4-(p-tolyl)indolin-5-yl)acetic acid the title compound was prepared as a yellow oil (8 mg, 23%). ¹H NMR (400 MHz, CHLOROFORM-d) δ ppm 7.45 (m, 1H), 7.13-7.24 (m, 3H), 6.95 (s, 1H), 5.24 (s, 1H), 3.50-3.84 (m, 2H), 2.61 (m, 1H), 2.34-2.46 (m, 8H), 1.68-1.93 (m, 3H), 1.21-1.47 (m, 8H), 0.96 (s, 9H), LCMS (ES+) (m/z): 436.5 (M+1).

### Example 5: 2-(tert-Butoxy)-2-(6-methyl-1-(2-oxo-2-(piperidin-1-yl)acetyl)-4-(p-tolyl)indolin-5-yl)acetic acid

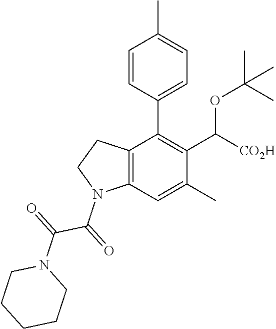 [SMILES: Cc1ccc(-c2c3c(cc(C)c2C(OC(C)(C)C)C(=O)O)N(C(=O)C(=O)N2CCCCC2)CC3)cc1]

In a manner similar to that described in Example 3, from 2-(tert-Butoxy)-2-(6-methyl-4-(p-tolyl)indolin-5-yl)acetic acid the title compound was prepared as a brown oil (14 mg, 30%). ¹H NMR (400 MHz, CHLOROFORM-d) 5 ppm (mixture of rotamers) 8.06 (s, 1H), 7.44 (m, 1H), 7.21-7.26 (m, 2H), 7.13 (1H), 6.83 (s, 1H), 5.22 (m, 1H), 3.86-4.26 (m, 2H), 3.74 (m, 1H), 3.62 (m, 2H), 3.34-3.49 (m, 2H), 2.67 (m, 1H), 2.33-2.46 (m, 6H), 1.55-1.76 (m, 6H), 0.97 (m, 9H). LCMS (ES+) (m/z): 515.4 (M+23).

### Example 6: 2-(tert-Butoxy)-2-(6-methyl-1-(piperidine-1-carbonyl)-4-(p-tolyl)indolin-5-yl)acetic acid

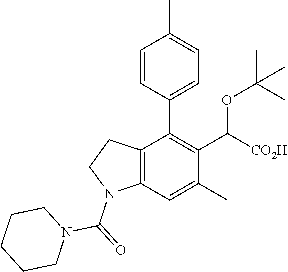 [SMILES: Cc1ccc(-c2c3c(cc(C)c2C(OC(C)(C)C)C(=O)O)N(C(=O)N2CCCCC2)CC3)cc1]

2-(tert-Butoxy)-2-(6-methyl-4-(p-tolyl)indolin-5-yl)acetic acid (22 mg, 0.062 mmol) was dissolved in DCM and cooled to 0° C. Piperidine-1-carbonyl chloride (0.04 mL, 5.14 mmol) was added followed by pyridine (0.01 mL, 0.124 mmol) and a small amount of DMAP. An additional amount of piperidine-1-carbonyl chloride (0.05 mL) and DMAP were added to complete the reaction. Poured over ice-1 N HCl mixture, extracted with DCM. The organics were dried over sodium sulfate, concentrated in vacuo, and purified by reverse-phase HPLC to give the title compound as an oil (4 mg, 14%). ¹H NMR (400 MHz, CHLOROFORM-d) δ ppm 7.45 (m, 1H, 7.10-7.26 (m, 4H), 6.88 (s, 1H), 5.20 (s, 1H), 3.84 (m, 2H), 3.35 (br. s, 4H), 2.32-2.57 (m, 8H), 1.66 (br. s, 6H), 0.97 (s, 9H). LCMS (ES+) (m/z): 465.4 (M+23).

### Example 7: 2-(tert-Butoxy)-2-(1-((4-fluorophenyl)carbamoyl)-6-methyl-4-(p-tolyl)indolin-5-yl)acetic acid

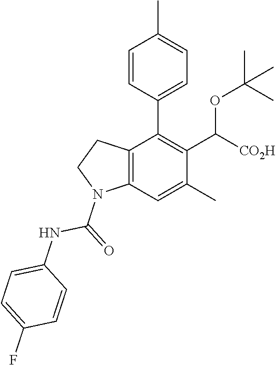 [SMILES: Cc1ccc(-c2c3c(cc(C)c2C(OC(C)(C)C)C(=O)O)N(C(=O)Nc2ccc(F)cc2)CC3)cc1]

2-(tert-Butoxy)-2-(6-methyl-4-(p-tolyl)indolin-5-yl)acetic acid (15 mg, 0.042 mmol) was dissolved in 1,2-dichloroethane (1 mL) and TEA (0.018 mL, 0.127 mmol) was added followed by 1-fluoro-4-isocyanatobenzene (0.01 mL, 0.088 mmol). The reaction was stirred at room temperature for 30 minutes, then diluted with 1 N HCl. The mixture was extracted with DCM, and the organics were dried over sodium sulfate, concentrated in vacuo, and purified by reverse-phase HPLC to give the title compound as a tan solid (5 mg, 24%). ¹H NMR (400 MHz, CHLOROFORM-d) δ ppm 7.78 (s, 1H), 7.36-7.49 (m, 3H), 7.21-7.26 (m, 1H), 6.99-7.16 (m, 3H), 6.40 (s, 1H), 5.20 (s, 1H), 3.91-4.09 (m, 2H), 3.68 (s, 1H), 2.71 (m, 1H), 2.37-2.45 (m, 6H), 0.98 (s, 9H). LCMS (ES+) (m/z): 491.4 (M+23).

### Example 8: 2-(tert-Butoxy)-2-(1-(cyclohexanecarbonyl)-6-methyl-4-(p-tolyl)indolin-5-yl)acetic acid

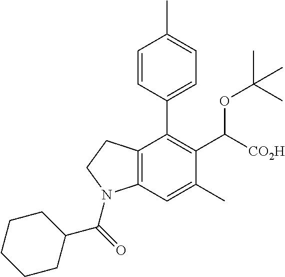 [SMILES: Cc1ccc(-c2c3c(cc(C)c2C(OC(C)(C)C)C(=O)O)N(C(=O)C2CCCCC2)CC3)cc1]

In a manner similar to that described in Example 3, from 2-(tert-butoxy)-2-(6-methyl-4-(p-tolyl)indolin-5-yl)acetic acid (14 mg, 0.040 mmol) and cyclohexanecarboxylic acid (10.3 mg, 0.080 mmol), the title compound was prepared as a yellow oil (1.1 mg, 6%). ¹H NMR (400 MHz, CHLOROFORM-d) δ ppm 8.15 (s, 1H) 7.46 (m, 1H), 7.24 (m, 1H), 7.12 (m, 1H), 5.20 (s, 1H), 3.94-4.14 (m, 2H), 2.65 (m, 1H), 2.39-2.45 (m, 7H), 1.43-2.01 (m, 11H), 0.96 (s, 9H). LCMS (ES+) (m/z): 464.4 (M+23).

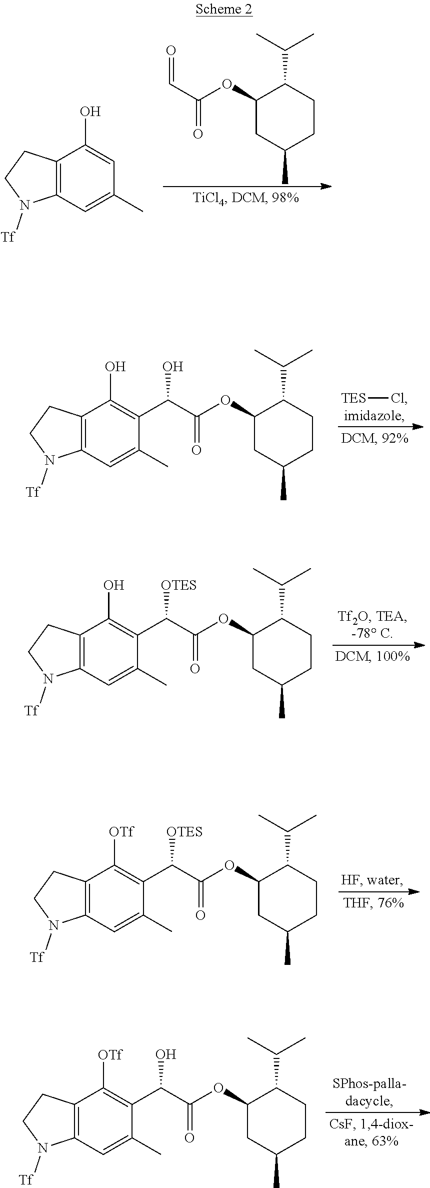

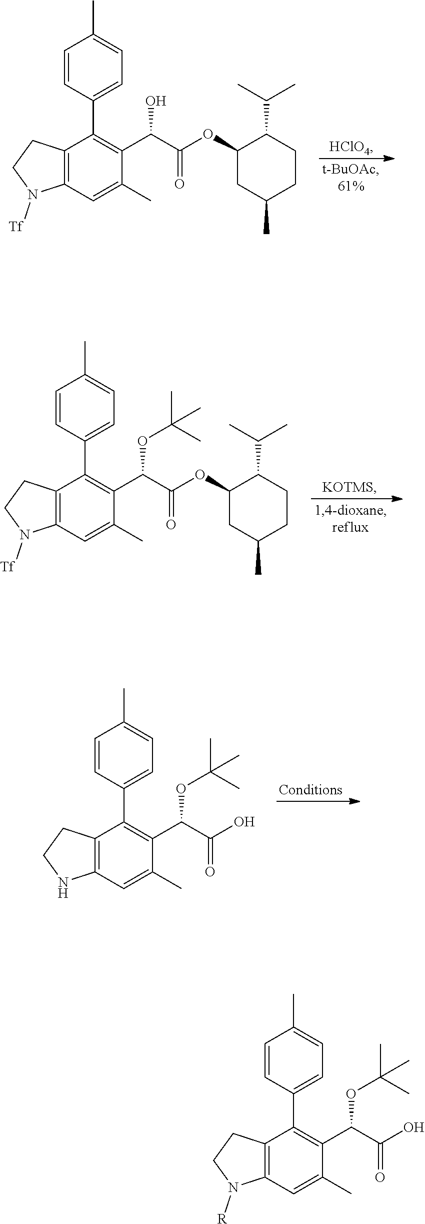

### Example 9: (S)-2-(tert-Butoxy)-2-(1-(2,4-dimethylbenzoyl)-6-methyl-4-(p-tolyl)indolin-5-yl)acetic acid

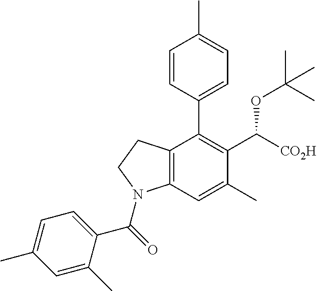 [SMILES: Cc1ccc(-c2c3c(cc(C)c2[C@H](OC(C)(C)C)C(=O)O)N(C(=O)c2ccc(C)cc2C)CC3)cc1]

### Step 1: (S)-(1R,2S,5R)-2-Isopropyl-5-methylcyclohexyl 2-hydroxy-2-(4-hydroxy-6-methyl-1-((trifluoromethyl)sulfonyl)indolin-5-yl)acetate

6-Methyl-1-((trifluoromethyl)sulfonyl)indolin-4-ol (309 mg, 1.1 mmol) was dissolved in DCM (15 mL) and cooled to 0° C. TiCl₄ (1.4 eq., 1.54 mmol, 1.54 mL, 1 M in DCM) was added dropwise and the reaction was stirred 5 minutes. (1R,2S,5R)-2-Isopropyl-5-methylcyclohexyl 2-oxoacetate was added (1.82 eq., 2 mmol) as a solution in DCM dropwise. The reaction was stirred at room temperature for 15 minutes, and then the reaction was poured into 1 N HCl and extracted with DCM. The organics dried over sodium sulfate, concentrated in vacuo, and purified by silica gel chromatography (0-100% EtOAc/hexanes) to give the desired product as ˜26:1 d.r. by ¹H NMR (methine proton). ¹H NMR (400 MHz, CHLOROFORM-d) δ ppm 7.94 (s, 1H), 6.85 (s, 1H), 5.53 (s, 1H), 4.76 (m, 1H), 4.21 (m, 2H), 3.49 (s, 1H), 3.12 (m, 2H), 2.37 (s, 3H), 1.80 (m, 2H), 1.66 (m, 2H), 1.58 (s, 1H), 1.30-1.50 (m, 2H), 1.04 (m, 1H), 0.91 (m, 3H), 0.85 (m, 3H), 0.74-0.82 (m, 4H). LCMS (ES−)(m/z): 492.5 (M−1).

### Step 2: (S)-(1R,2S,5R)-2-Isopropyl-5-methylcyclohexyl 2-(4-hydroxy-6-methyl-1-((trifluoromethyl)sulfonyl)indolin-5-yl)-2-((triethylsilyl)oxy)acetate

The product from Step 1 (657 mg, 1.1 mmol) and imidazole (75 mg) were dissolved in DCM and cooled to 0° C. TES-Cl (0.19 ml) was then added and the mixture was warmed to room temperature and stirred for 1 hour. The mixture was poured into ice water, extracted with DCM, and the organics were washed with 1 N HCl, dried over sodium sulfate and purified by silica gel chromatography (0-100% EtOAc/hexanes) to give the desired product as a colorless oil. (ES−)(m/z): 606.6 (M−1).

### Step 3: (S)-(1R,2S,5R)-2-Isopropyl-5-methylcyclohexyl 2-(6-methyl-1-((trifluoromethyl)sulfonyl)-4-(((trifluoromethyl)sulfon y)oxy)indolin-5-yl)-2-((triethylsilyl)oxy)acetate

The compound from Step 2 above (597 mg, 0.982 mmol) was dissolved in DCM (8 mL) and cooled to −78° C. TEA (2.4 eq, 2.36 mmol) was added, followed by triflic anhydride (1.1 eq, 1.08 mmol). The reaction was stirred 5 minutes and poured into water, extracted with DCM, the organics were washed with 1 N HCl, dried over sodium sulfate and concentrated in vacuo. to give a yellow oil (687 mg, 95%) that was used in the next step without purification. LCMS (ES+) (m/z): 762.6 (M+23).

### Step 4: (S)-(1R,2S,5R)-2-Isopropyl-5-methylcyclohexyl 2-hydroxy-2-(6-methyl-1-((trifluoromethyl)sulfonyl)-4-(((trifluoromethyl)sulfonyl)oxy)indolin-5-yl)acetate

The product from Step 3 above (54 mg, 0.073 mmol) was dissolved in THF and HF (48% in water, ˜27.6M) (35 eq, 2.6 mmol, 0.1 ml) was added and the reaction was stirred overnight at room temperature. Solid sodium bicarbonate was added and the mixture was diluted with EtOAc and a saturated sodium bicarbonate solution. The reaction was extracted with EtOAc, and the organics were dried over sodium sulfate and concentrated in vacuo. Purification by silica gel chromatography (0-100% EtOAc/hexanes) gave the desired product as a residue (31 mg, 67%). LCMS (ES+) (m/z): 648.5 (M+23).

### Step 5: (S)-(1R,2S,5R)-2-Isopropyl-5-methylcyclohexyl 2-hydroxy-2-(6-methyl-4-(p-tolyl)-1-((trifluoromethyl)sulfonyl)indolin-5-yl)acetate

The product from Step 4 above (513 mg, 0.819 mmol) was run in 100 mg batches in 5 separate runs in the microwave and then combined and purified to give 474 mg desired product (slightly impure). A representative procedure follows:

The product from Step 4 above (102 mg, 1 eq.) in 1,4-dioxane (2 mL) in a microwave vial (0.5-2 mL) set to Low absorption was treated with CsF (99 mg, 4 eq.) and p-tolylboronic acid (62 mg, 2.5 eq.) while degassing with N2 (g), SPhos palladacycle (37 mg, 0.3 eq.) (CAS #1028206-58-7) was added and the reaction was sealed and heated in the microwave at 130° C. for 30 minutes. This was repeated for 5 batches in total. All the batches were combined and filtered through Celite, the filter rinsed with DCM, and the filtrate concentrated in vacuo. and purified by silica gel chromatography (0-100% EtOAc/hexanes) to give the desired product (474 mg, impure). LCMS (ES+) (m/z): 590.5 (M+23).

### Step 6: (S)-(1R,2S,5R)-2-Isopropyl-5-methylcyclohexyl 2-(tert-butoxy)-2-(6-methyl-4-(p-tolyl)-1-((trifluoromethyl)sulfonyl)indolin-5-yl)acetate

The product from Step 5 (724 mg, 1 eq.) in t-BuOAc (18 mL, 120 eq., 0.07 M) was treated with perchloric acid (0.45 mL, 4 eq., 70% solution) at room temperature, and the reaction was stirred until complete by TLC and LCMS. The reaction was poured over ice/NaOH (50%, aq., pH˜14), and extracted with EtOAc (×3). The organics were washed with brine, dried over sodium sulfate, and concentrated in vacuo. and purified by silica gel chromatography (0-100% EtOAc/hexanes) to give the desired product (410 mg, 52%). LCMS (ES+) (m/z): 646.7 (M+23).

### Step 7: (S)-2-(tert-Butoxy)-2-(6-methyl-4-(p-tolyl)indolin-5-yl)acetic acid

The product from Step 6 (269 mg, 1 eq.) in 1,4-Dioxane (43 mL) was treated with KOTMS (442 mg, 8 eq.) and heated to reflux. The reaction was monitored by LCMS of an aliquot treated with 1 N HCl and extracted with EtOAc. After completion, the reaction was poured rover ice/HCl (1M, pH˜1), and extracted with EtOAc (×3) and 3:1 CHCl₃:IPA (×3). The combined organics were dried over sodium sulfate, filtered, and concentrated in vacuo. to give the desired product (276 mg) as a orange-red solid. The product was carried forward without purification. LCMS (ES+) (m/z): 354.5 (M+1).

### Step 8: (S)-2-(tert-Butoxy)-2-(1-(2,4-dimethylbenzoyl)-6-methyl-4-(p-tolyl)indolin-5-yl)acetic acid

In a manner similar to that described in Example 3, from (S)-2-(tert-butoxy)-2-(6-methyl-4-(p-tolyl)indolin-5-yl)acetic acid (21 mg, 0.059 mmol) and 2,4-dimethylbenzoic acid (16 mg, 0.107 mmol, the title compound was prepared as a white solid (4 mg, 14%). 8.20 (s, 1H), 6.67-7.60 (m, 8H), under CHCl₃), 5.23 (s, 1H), 3.54 (m, 2H), 3.05 (m, 1H), 1.80-2.54 (m, 12H), 0.98 (s, 9H). LCMS (ES+) (m/z): 486.1 (M+1).

### Example 10: (S)-2-(tert-Butoxy)-2-(1-(4-methoxy-2-methylbenzoyl)-6-methyl-4-(p-tolyl)indolin-5-yl)acetic acid

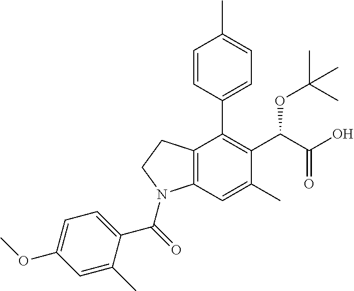 [SMILES: COc1ccc(C(=O)N2CCc3c2cc(C)c([C@H](OC(C)(C)C)C(=O)O)c3-c2ccc(C)cc2)c(C)c1]

In a manner similar to that described in Example 9, from (S)-2-(tert-butoxy)-2-(6-methyl-4-(p-tolyl)indolin-5-yl)acetic acid (17 mg, 0.048 mmol) and 4-methoxy-2-methylbenzoic acid (16 mg, 0.096 mmol, the title compound was prepared as a white solid (3.9 mg, 13%). ¹H NMR (400 MHz, CHLOROFORM-d) δ ppm 8.20 (br.s., 1H), 7.45 (m, 1H), 7.05-7.35 (m, 5H), 7.30 (m, 1H), 5.10 (br.s, 1H), 3.50-4.00 (4H), 3.25-2.80 (m, 1H), 2.00-2.70 (m, 10H), 0.97 (s, 9H). LCMS (ES+) (m/z): 502.10 (M+1).

### Example 11: (S)-2-(tert-Butoxy)-2-(1-(3-fluoro-4-methoxybenzoyl)-6-methyl-4-(p-tolyl)indolin-5-yl)acetic acid

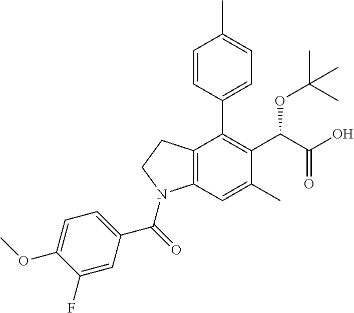 [SMILES: COc1ccc(C(=O)N2CCc3c2cc(C)c([C@H](OC(C)(C)C)C(=O)O)c3-c2ccc(C)cc2)cc1F]

In a manner similar to that described in Example 9, from (S)-2-(tert-butoxy)-2-(6-methyl-4-(p-tolyl)indolin-5-yl)acetic acid (17 mg, 0.048 mmol) and 3-fluoro-4-methoxybenzoic acid (16 mg, 0.094 mmol, the title compound was prepared as a white solid (6.6 mg, 22%). ¹H NMR (400 MHz, CHLOROFORM-d) δ ppm 6.95-7.55 (m, 8H) (under CHCl₃), 5.23 (s, 1H), 3.85-4.15 (m, 5H), 3.05 (m, 1H), 2.25-2.65 (m, 8H), 0.97 (s, 9H). LCMS (ES+) (m/z): 506.05 (M+1).

### Example 12: (S)-2-(tert-Butoxy)-2-(1-(3,4-difluoro-5-methoxybenzoyl)-6-methyl-4-(p-tolyl)indolin-5-yl)acetic acid

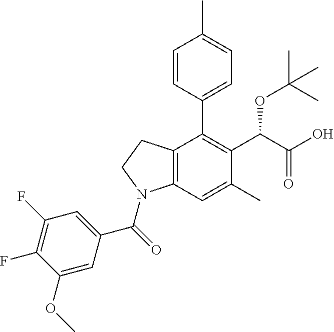 [SMILES: COc1cc(C(=O)N2CCc3c2cc(C)c([C@H](OC(C)(C)C)C(=O)O)c3-c2ccc(C)cc2)cc(F)c1F]

In a manner similar to that described in Example 9, from (S)-2-(tert-butoxy)-2-(6-methyl-4-(p-tolyl)indolin-5-yl)acetic acid (25 mg, 0.071 mmol) and 3,4-difluoro-5-methoxybenzoic acid (23 mg, 0.122 mmol, the title compound was prepared as a white solid (7.7 mg, 21%). ¹H NMR (400 MHz, CHLOROFORM-d) δ ppm 7.44-7.49 (m, 1H), 7.19-7.30/(m, 4H) (under CHCl₃), 6.95-7.14 (m, 3H), 5.23 (s, 1H), 3.85-4.06 (m, 5H), 3.07 (m, 1H), 2.51-2.67 (m, 1H), 2.34-2.48 (m, 6H), 0.97 (s, 9H). LCMS (ES+) (m/z): 524.09 (M+1).

### Example 13: (S)-2-(tert-Butoxy)-2-(1-(4-methoxybenzoyl)-6-methyl-4-(p-tolyl)indolin-5-yl)acetic acid

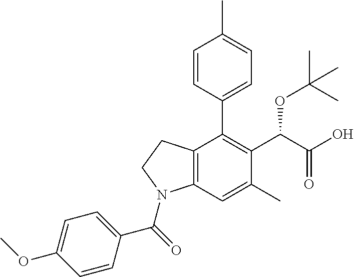 [SMILES: COc1ccc(C(=O)N2CCc3c2cc(C)c([C@H](OC(C)(C)C)C(=O)O)c3-c2ccc(C)cc2)cc1]

In a manner similar to that described in Example 9, from (S)-2-(tert-butoxy)-2-(6-methyl-4-(p-tolyl)indolin-5-yl)acetic acid (25 mg, 0.074 mmol) and 4-methoxybenzoic acid (29 mg, 0.184 mmol, the title compound was prepared as a white solid (8.5 mg, 25%). ¹H NMR (400 MHz, CHLOROFORM-d) δ ppm 7.43-7.60 (m, 3H), 7.08-7.29 (m, 4H) (under CHCl₃), 6.95 (m, 2H), 5.22 (s, 1H), 4.02 (m, 2H), 3.87 (s, 3H), 3.01 (m, 1H), 2.57 (m, 2H), 2.29-2.45 (m, 6H), 0.97 (s, 9H). LCMS (ES+) (m/z): 488.10 (M+1).

### Example 14: (S)-2-(tert-Butoxy)-2-(1-(4-fluorobenzoyl)-6-methyl-4-(p-tolyl)indolin-5-yl)acetic acid

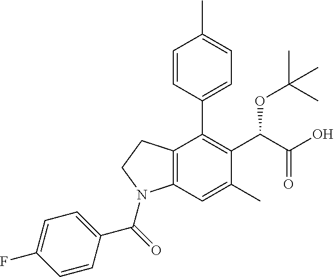 [SMILES: Cc1ccc(-c2c3c(cc(C)c2[C@H](OC(C)(C)C)C(=O)O)N(C(=O)c2ccc(F)cc2)CC3)cc1]

In a manner similar to that described in Example 9, from (S)-2-(tert-butoxy)-2-(6-methyl-4-(p-tolyl)indolin-5-yl)acetic acid (20 mg, 0.057 mmol) and 4-fluorobenzoic acid (10 mg, 0.071 mmol, the title compound was prepared as a white solid (7 mg, 35%). ¹H NMR (400 MHz, CHLOROFORM-d) δ ppm 7.43-7.61 (m, 3H), 7.08-7.31 (m, 6H) (under CHCl₃), 5.22 (s, 1H), 3.98 (m, 2H), 3.02 (m, 2H), 2.58 (m, 1H), 2.33-2.45 (m, 6H), 0.97 (s, 9H). LCMS (ES+) (m/z): 476.05 (M+1).

### Example 15: (S)-2-(tert-Butoxy)-2-(1-(3,6-difluoro-2-methoxybenzoyl)-6-methyl-4-(p-tolyl)indolin-5-yl)acetic acid

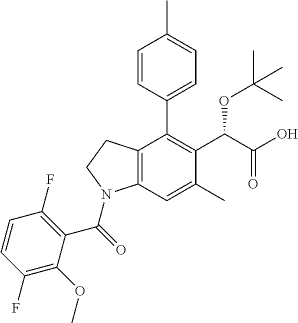 [SMILES: COc1c(F)ccc(F)c1C(=O)N1CCc2c1cc(C)c([C@H](OC(C)(C)C)C(=O)O)c2-c1ccc(C)cc1]

In a manner similar to that described in Example 9, from (S)-2-(tert-butoxy)-2-(6-methyl-4-(p-tolyl)indolin-5-yl)acetic acid (27 mg, 0.076 mmol) and 3,6-difluoro-2-methoxybenzoic acid (21 mg, 0.114 mmol), the title compound was prepared as a white solid (1.7 mg, 6%) as a mixture of rotamers. ¹H NMR (400 MHz, CHLOROFORM-d) δ ppm 8.12-8.26 (m, 1H), 7.42-7.55 (m, 1H), 7.05-7.30 (m, 4H), 6.81 (m, 1H), 5.24 (s, 1H), 3.68-4.07 (m, 4H), 3.03 (m, 1H), 2.61 (m, 2H), 2.35-2.50 (m, 6H), 1.00 (m, 9H). LCMS (ES+) (m/z): 524.08 (M+1).

### Example 16: (S)-2-(tert-Butoxy)-2-(1-(3-methoxybenzoyl)-6-methyl-4-(p-tolyl)indolin-5-yl)acetic acid

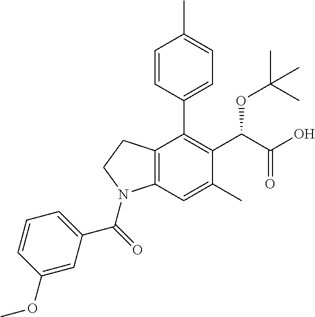 [SMILES: COc1cccc(C(=O)N2CCc3c2cc(C)c([C@H](OC(C)(C)C)C(=O)O)c3-c2ccc(C)cc2)c1]

In a manner similar to that described in Example 9, from (S)-2-(tert-butoxy)-2-(6-methyl-4-(p-tolyl)indolin-5-yl)acetic acid (27 mg, 0.076 mmol) and 3-methoxybenzoic acid (17 mg, 0.115 mmol), the title compound was prepared as a white solid (15 mg, 40%). ¹H NMR (400 MHz, CHLOROFORM-d) δ ppm 7.46 (m, 1H), 7.36 (m, 1H), 7.19-7.29 (m, 3H), 6.99-7.15 (m, 4H), 5.23 (s, 1H), 3.98 (m, 2H), 3.84 (s, 3H), 3.00 (m, 1H), 2.58 (m, 1H), 2.30-2.47 (m, 6H), 0.98 (s, 9H). LCMS (ES+) (m/z): 486.02 (M+1).

### Example 17: (S)-2-(tert-Butoxy)-2-(1-(2-methoxybenzoyl)-6-methyl-4-(p-tolyl)indolin-5-yl)acetic acid

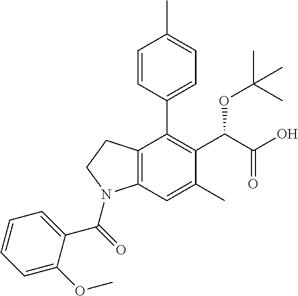 [SMILES: COc1ccccc1C(=O)N1CCc2c1cc(C)c([C@H](OC(C)(C)C)C(=O)O)c2-c1ccc(C)cc1]

In a manner similar to that described in Example 9, from (S)-2-(tert-butoxy)-2-(6-methyl-4-(p-tolyl)indolin-5-yl)acetic acid (27 mg, 0.076 mmol) and 2-methoxybenzoic acid (17 mg, 0.115 mmol), the title compound was prepared as a white solid (5 mg, 13%). ¹H NMR (400 MHz, CHLOROFORM-d) δ ppm 8.23 (s, 1H), 6.90-7.55 (m, 8H), 5.24 (s, 1H), 3.66-3.91 (m, 5H), 2.99 (m, 1H), 2.56 (m, 1H), 2.34-2.50 (m, 6H), 2.03 (m, 1H), 0.98 (s, 9H). LCMS (ES+) (m/z): 488.07 (M+1).

### Example 18: (S)-2-(tert-Butoxy)-2-(1-(2-fluorobenzoyl)-6-methyl-4-(p-tolyl)indolin-5-yl)acetic acid

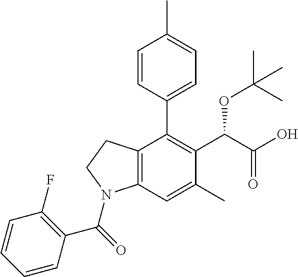 [SMILES: Cc1ccc(-c2c3c(cc(C)c2[C@H](OC(C)(C)C)C(=O)O)N(C(=O)c2ccccc2F)CC3)cc1]

In a manner similar to that described in Example 9, from (S)-2-(tert-butoxy)-2-(6-methyl-4-(p-tolyl)indolin-5-yl)acetic acid (27 mg, 0.076 mmol) and 2-fluorobenzoic acid (16 mg, 0.115 mmol), the title compound was prepared as a white solid (2 mg, 6%). ¹H NMR (400 MHz, CHLOROFORM-d) δ ppm 8.18 (s, 1H), 7.39-7.57 (m, 3H), 7.09-7.29 (m, 5H), 5.24 (s, 1H), 3.85 (m, 2H), 3.04 (m, 1H), 2.60 (m, 1H), 2.36-2.53 (m, 6H), 0.99 (s, 9H). LCMS (ES+) (m/z): 476.07 (M+1).

### Example 19: (S)-2-(tert-Butoxy)-2-(1-(2-chloro-4-fluorobenzoyl)-6-methyl-4-(p-tolyl)indolin-5-yl)acetic acid

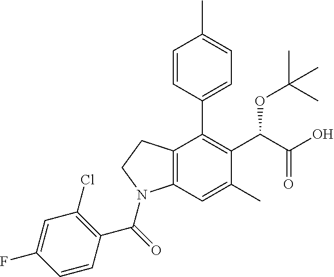 [SMILES: Cc1ccc(-c2c3c(cc(C)c2[C@H](OC(C)(C)C)C(=O)O)N(C(=O)c2ccc(F)cc2Cl)CC3)cc1]

In a manner similar to that described in Example 9, from (S)-2-(tert-butoxy)-2-(6-methyl-4-(p-tolyl)indolin-5-yl)acetic acid (27 mg, 0.076 mmol) 2-chloro-4-fluorobenzoic acid (20 mg, 0.115 mmol), the title compound was prepared as a white solid (8 mg, 21%). ¹H NMR (400 MHz, CHLOROFORM-d) 5 ppm (mixture of rotamers) 8.20 (s, 1H), 7.03-7.53 (m, 7H), 5.25 (s, 1H), 4.16 (m, 1H), 3.74 (m, 2H), 3.03 (m, 1H), 2.62 (m, 1H), 2.36-2.53 (m, 6H), 0.98 (m, 9H). LCMS (ES+) (m/z): 510.02 (M+1).

### Example 20: (S)-2-(tert-Butoxy)-2-(1-(2-fluoro-3-methoxybenzoyl)-6-methyl-4-(p-tolyl)indolin-5-yl)acetic acid

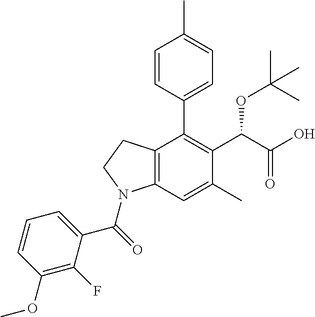 [SMILES: COc1cccc(C(=O)N2CCc3c2cc(C)c([C@H](OC(C)(C)C)C(=O)O)c3-c2ccc(C)cc2)c1F]

In a manner similar to that described in Example 9, from (S)-2-(tert-butoxy)-2-(6-methyl-4-(p-tolyl)indolin-5-yl)acetic acid (27 mg, 0.076 mmol) 2-fluoro-3-methoxybenzoic acid (19 mg, 0.115 mmol), the title compound was prepared as a white solid (7 mg, 18%).

¹H NMR (400 MHz, CHLOROFORM-d) δ ppm 8.18 (s, 1H), 7.47 (m, 1H), 6.94-7.30 (m, 6H), 5.24 (s, 1H), 3.76-4.00 (m, 5H), 3.00 (m, 2H), 2.60 (m, 1H), 2.35-2.51 (m, 6H), 0.99 (s, 9H). LCMS (ES+) (m/z): 506.08 (M+1).

### Example 21: (S)-2-(tert-Butoxy)-2-(1-(4-fluoro-2-methoxybenzoyl)-6-methyl-4-(p-tolyl)indolin-5-yl)acetic acid

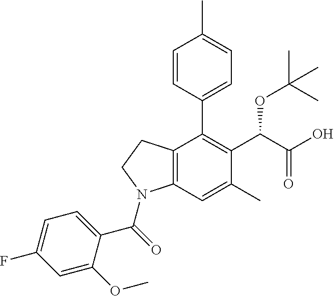 [SMILES: COc1cc(F)ccc1C(=O)N1CCc2c1cc(C)c([C@H](OC(C)(C)C)C(=O)O)c2-c1ccc(C)cc1]

In a manner similar to that described in Example 9, from (S)-2-(tert-butoxy)-2-(6-methyl-4-(p-tolyl)indolin-5-yl)acetic acid (27 mg, 0.076 mmol) 4-fluoro-2-methoxybenzoic acid (19 mg, 0.115 mmol), the title compound was prepared as a white solid (8 mg, 21%).

¹H NMR (400 MHz, CHLOROFORM-d) δ ppm 8.19 (s, 1H), 7.46 (m, 1H), 7.06-7.35 (m, 4H), 6.71 (m, 2H), 5.24 (s, 1H), 3.60-3.91 (m, 5H), 3.00 (m, 2H), 2.57 (m, 1H), 2.34-2.50 (m, 6H), 0.98 (s, 9H). LCMS (ES+) (m/z): 506.06 (M+1).

### Example 22: (S)-2-(tert-Butoxy)-2-(1-(4-chlorobenzoyl)-6-methyl-4-(p-tolyl)indolin-5-yl)acetic acid

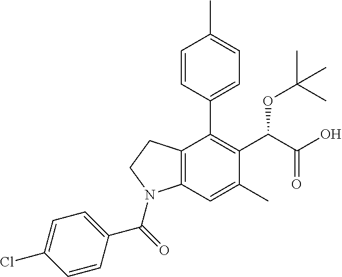 [SMILES: Cc1ccc(-c2c3c(cc(C)c2[C@H](OC(C)(C)C)C(=O)O)N(C(=O)c2ccc(Cl)cc2)CC3)cc1]

In a manner similar to that described in Example 9, from (S)-2-(tert-butoxy)-2-(6-methyl-4-(p-tolyl)indolin-5-yl)acetic acid (27 mg, 0.076 mmol) 4-chlorobenzoic acid (18 mg, 0.115 mmol), the title compound was prepared as a white solid (5 mg, 13%). ¹H NMR (400 MHz, CHLOROFORM-d) δ ppm 7.40-7.54 (m, 5H), 7.08-7.30 (m, 4H), 5.23 (s, 1H), 3.97 (m, 2H), 3.01 (m, 1H), 2.58 (m, 1H), 2.29-2.49 (m, 6H), 0.98 (s, 9H). LCMS (ES+) (m/z): 492.02 (M+1).

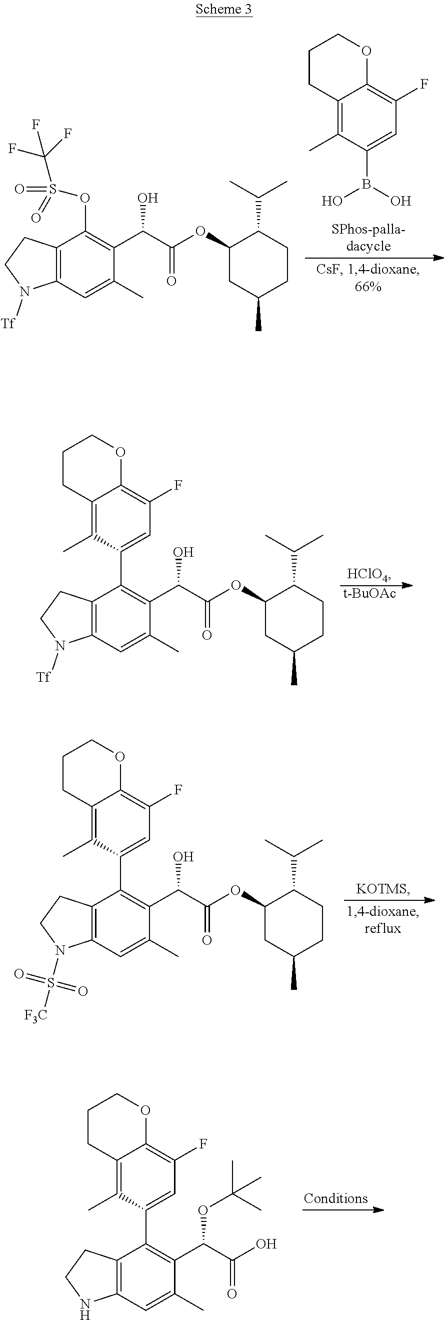

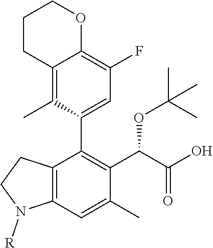

### Example 23: (S)-2-(tert-Butoxy)-2-((P)-1-(3-fluoro-2-methoxybenzoyl)-4-(8-fluoro-5-methylchroman-6-yl)-6-methylindolin-5-yl)acetic acid

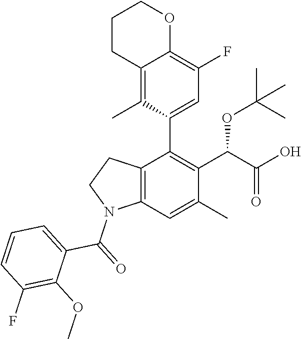

### Step 1: (S)-(1R,2S,5R)-2-Isopropyl-5-methylcyclohexyl 2-((P)-4-(8-fluoro-5-methylchroman-6-yl)-6-methyl-1-((trifluoromethyl)sulfonyl)indolin-5-yl)-2-hydroxyacetate

(S)-(1R,2S,5R)-2-Isopropyl-5-methylcyclohexyl 2-hydroxy-2-(6-methyl-1-((trifluoromethyl)sulfonyl)-4-(((trifluoromethyl)sulfonyl)oxy)indolin-5-yl)acetate (103 mg, 0.165 mmol) and (8-fluoro-5-methylchroman-6-yl)boronic acid (2.55 eq, 0.419 mmol, 88 mg) was suspended in dioxane (2 mL) and degassed while adding CsF (4 eq, 100 mg, 0.659 mmol) and SPhos palladacycle (cas 1028206-58-7) (0.3 eq., 0.049 mmol, 38 mg). The reaction was heated in a 0.5-2 mL mw vial with absorption set to low at 130° C. for 30 minutes, then filtered through Celite and the filtrate was concentrated in vacuo. The crude isolate was purified by silica gel chromatography (0-100% EtOAc/hexanes) to give the desired product (70 mg, 66%) as a brown oil with ˜3:1 mixture of diastereomers. LCMS (ES−)(m/z): 640.60 (M−1).

### Step 2: (S)-(1R,2S,5R)-2-Isopropyl-5-methylcyclohexyl 2-(tert-butoxy)-2-((P)-4-(8-fluoro-5-methylchroman-6-yl)-6-methyl-1-((trifluoromethyl)sulfonyl)indolin-5-yl)acetate

In a manner similar to that described in Example 9, Step 6, from (S)-(1R,2S,5R)-2-Isopropyl-5-methylcyclohexyl 2-((P)-4-(8-fluoro-5-methylchroman-6-yl)-6-methyl-1-((trifluoromethyl)sulfonyl)indolin-5-yl)-2-hydroxyacetate (70 mg, 0.109 mmol), tert-butyl acetate (6 mL), and 70% perchloric acid (0.3 mL), the desired product was obtained after purification using silica gel chromatography (0-100% EtOAc/hexanes and 0-100% MTBE/hexanes) (26 mg, 34%). LCMS (ES+) (m/z): 698.35 (M+1).

### Step 3: (S)-2-(tert-butoxy)-2-((P)-4-(8-fluoro-5-methylchroman-6-yl)-6-methylindolin-5-yl)acetic acid

In a manner similar to that described in Example 9, Step 7, from (S)-(1R,2S,5R)-2-Isopropyl-5-methylcyclohexyl 2-(tert-butoxy)-2-((P)-4-(8-fluoro-5-methylchroman-6-yl)-6-methyl-1-((trifluoromethyl)sulfonyl)indolin-5-yl)acetate (26 mg, 0.037 mmol) and KOTMS (8 eq., 0.296 mmol, 38 mg), the desired product was obtained (33 mg) and used without further purification. LCMS (ES+) (m/z): 427.99 (M+1).

### Step 4: (S)-2-(tert-Butoxy)-2-((P)-1-(3-fluoro-2-methoxybenzoyl)-4-(8-fluoro-5-methylchroman-6-yl)-6-methylindolin-5-yl)acetic acid

In a manner similar to that described in Example 3, from (S)-2-(tert-butoxy)-2-((P)-4-(8-fluoro-5-methylchroman-6-yl)-6-methylindolin-5-yl)acetic acid (33 mg, 0.077 mmol) and 3-fluoro-2-methoxybenzoic acid (19.7 mg, 0.116 mmol), the title compound was obtained as a residue (2.6 mg, 6%). ¹H NMR (400 MHz, CHLOROFORM-d) δ ppm (mixture of rotamers) 8.20 (s, 1H), (6.97-7.22, m, 5H), 5.22 (s, 1H), 4.26 (m, 2H), 3.99 (s, 3H), 3.40-3.92 (m, 3H), 2.48-2.73 (m, 8H), 2.10 (m, 3H). LCMS (ES+) (m/z): 580.13 (M+1); 602.2 (M+23).

### Example 24: (S)-2-(tert-Butoxy)-2-(1-(tert-butoxycarbonyl)-6-methyl-4-(p-tolyl)indolin-5-yl)acetic acid

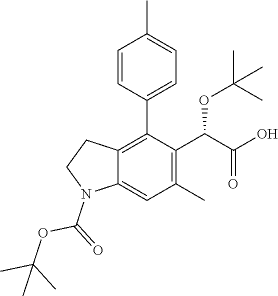 [SMILES: Cc1ccc(-c2c3c(cc(C)c2[C@H](OC(C)(C)C)C(=O)O)N(C(=O)OC(C)(C)C)CC3)cc1]

To a solution of (S)-2-(tert-butoxy)-2-(6-methyl-4-(p-tolyl)indolin-5-yl)acetic acid (10 mg, 0.028 mmol) in 2 mL MeOH was added TEA (0.012 mL, 0.085 mmol) followed by di-tert-butyl dicarbonate (12.35 mg, 0.057 mmol). The solution was stirred for 1 hour, then diluted with EtOAc and 1M HCl, extracted with EtOAc, washed with brine, dried over Na₂SO₄ and the solvent was removed in vacuo. The resulting clear colorless oil was purified by HPLC to yield the title compound as white solid (4.4 mg, 9.70 μmol, 34.3% yield). ¹H NMR (400 MHz, CDCl₃) δ 7.44 (m, 1H), 7.23 (m, 3H), 7.12 (m, 1H), 5.19 (s, 1H), 3.90 (m, 2H), 2.98 (m, 1H), 2.57 (m, 1H), 2.40 (m, 6H), 1.57 (m, 9H), 0.97 (m, 9H).

LCMS (ES+) (m/z): 476.55 (M+23), 930.07 (2M+23).

### Example 25: (S)-2-(tert-Butoxy)-2-(1-(2-cyclohexylacetyl)-6-methyl-4-(p-tolyl)indolin-5-yl)acetic acid

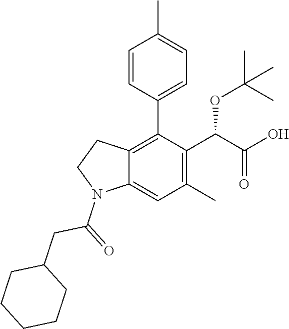 [SMILES: Cc1ccc(-c2c3c(cc(C)c2[C@H](OC(C)(C)C)C(=O)O)N(C(=O)CC2CCCCC2)CC3)cc1]

### Step 1: (S)-2-(tert-Butoxy)-2-(1-(2-cyclohexylacetyl)-6-methyl-4-(p-tolyl)indolin-5-yl)acetic 2-cyclohexylacetic anhydride

To a solution of (S)-2-(tert-butoxy)-2-(6-methyl-4-(p-tolyl)indolin-5-yl)acetic acid (10 mg, 0.028 mmol) in 2 mL DCM was added pyridine (2.75 μl, 0.034 mmol) followed by 2-cyclohexylacetyl chloride (0.1 mL, 0.033 mmol) dropwise. The solution was stirred for 1 hour, then diluted with DCM and 1M HCl, extracted with DCM, washed with brine, dried over Na₂SO₄ and the solvent was removed in vacuo. The title compound was carried on to the next step without further purification as a brown oil (17.03 mg, 0.028 mmol, 100% yield). LCMS (ES+) (m/z): 624.67 (M+23), 1204.43 (2M+1).

### Step 2: (S)-2-(tert-Butoxy)-2-(1-(2-cyclohexylacetyl)-6-methyl-4-(p-tolyl)indolin-5-yl)acetic acid

To a solution of (S)-2-(tert-butoxy)-2-(1-(2-cyclohexylacetyl)-6-methyl-4-(p-tolyl)indolin-5-yl)acetic 2-cyclohexylacetic anhydride (17 mg, 0.028 mmol) in 2 mL dioxane was added LiOH (0.424 mL, 0.424 mmol). The solution was stirred at room temperature for 30 minutes, diluted with EtOAc and 1M HCl, washed with brine, dried over Na₂SO₄, and the solvent was removed in vacuo. The resulting clear oil was purified by reverse phase HPLC to yield the title compound as a white solid (2.9 mg, 6.07 μmol, 21.49% yield). ¹H NMR (400 MHz, CDCl₃) δ 8.14 (s, 1H), 7.46 (m, 1H), 7.24 (m, 2H), 7.12 (m, 1H), 5.21 (s, 1H), 4-11-3.87 (m, 2H), 3.08 (m, 1H), 2.65 (m, 1H), 2.40 (m, 6H), 2.29 (m, 2H), 2.06-1.23 (m, 11H), 0.97 (m, 9H). LCMS (ES+) (m/z): 478.59 (M+1), 956.12 (2M+1).

### Example 26: (S)-2-(tert-Butoxy)-2-(1-(2,5-dimethylthiazole-4-carbonyl)-6-methyl-4-(p-tolyl)indolin-5-yl)acetic acid

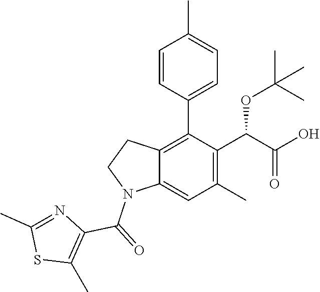 [SMILES: Cc1ccc(-c2c3c(cc(C)c2[C@H](OC(C)(C)C)C(=O)O)N(C(=O)c2nc(C)sc2C)CC3)cc1]

In a manner similar to that described in Example 9, Step 8, the title compound was isolated as a white solid after reverse phase hplc. ¹H NMR (400 MHz, CDCl₃) δ 8.11 (m, 1H), 7.47 (m, 1H), 7.24 (m, 2H), 7.12 (m, 1H), 5.22 (s, 1H), 4.30-3.99 (m, 2H), 3.03 (m, 1H), 2.71-2.50 (m, 7H), 2.41 (m, 6H), 0.96 (m, 9H). LCMS (ES+) (m/z): 493.49 (M+1), 985.90 (2M+1).

### Example 27: (S)-2-(tert-Butoxy)-2-(1-(3-fluoro-2-methoxybenzoyl)-6-methyl-4-(p-tolyl)indolin-5-yl)acetic acid

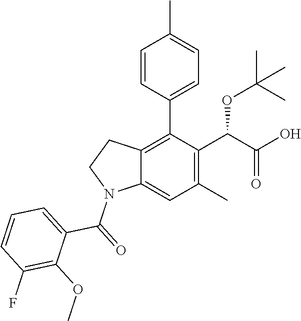 [SMILES: COc1c(F)cccc1C(=O)N1CCc2c1cc(C)c([C@H](OC(C)(C)C)C(=O)O)c2-c1ccc(C)cc1]

In a manner similar to that described in Example 9, Step 8, the title compound was Isolated as a white solid after reverse phase hplc. ¹H NMR (400 MHz, CDCl₃) δ 8.19 (m, 1H), 7.54-7.03 (m, 7H), 5.24 (s, 1H), 3.98 (s, 3H), 3.75 (m, 2H), 3.00 (m, 1H), 2.58 (m, 1H), 2.44 (m, 6H), 0.98 (m, 9H). LCMS (ES+) (m/z): 506.53 (M+1), 1011.95 (2M+1).

### Example 28: (S)-2-(tert-Butoxy)-2-(1-(cyclohexylsulfonyl)-6-methyl-4-(p-tolyl)indolin-5-yl)acetic acid

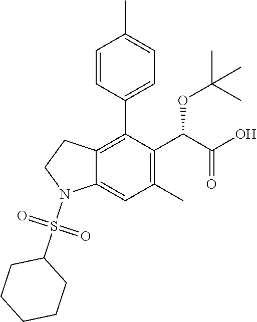 [SMILES: Cc1ccc(-c2c3c(cc(C)c2[C@H](OC(C)(C)C)C(=O)O)N(S(=O)(=O)C2CCCCC2)CC3)cc1]

To a solution of (S)-2-(tert-butoxy)-2-(6-methyl-4-(p-tolyl)indolin-5-yl)acetic acid (10 mg, 0.028 mmol) was added pyridine (2.75 μl, 0.034 mmol) followed by cyclohexanesulfonyl chloride (4.10 μl, 0.028 mmol). The solution was stirred overnight, diluted with DCM and 1M HCl, extracted with DCM, washed with brine, dried over sodium sulfate and solvent was concentrate in vacuo. The Pink oil was purified by reverse-phase HPLC to yield a white solid (S)-2-(tert-butoxy)-2-(1-(cyclohexylsulfonyl)-6-methyl-4-(p-tolyl)indolin-5-yl)acetic acid (0.8 mg, 1.601 μmol, 5.66% yield). ¹H NMR (400 MHz, CDCl₃) δ 7.44 (m, 1H), 7.24 (m, 1H), 7.12 (m, 2H), 5.20 (s, 1H), 4.17-3.85 (m, 2H), 3.23-2.94 (m, 2H), 2.59 (m, 1H), 2.39 (m, 6H), 2.25-1.15 (m, 10H), 0.96 (m, 9H). LCMS (ES+) (m/z): 522.47 (M+23), 1022.12 (2M+23).

### Example 29: (S)-2-(tert-Butoxy)-2-(1-(3-fluorobenzoyl)-6-methyl-4-(p-tolyl)indolin-5-yl)acetic acid

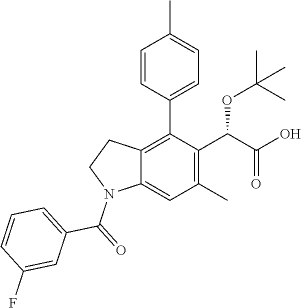 [SMILES: Cc1ccc(-c2c3c(cc(C)c2[C@H](OC(C)(C)C)C(=O)O)N(C(=O)c2cccc(F)c2)CC3)cc1]

In a manner similar to that described in Example 9, Step 8, the title compound was isolated as a white solid after reverse phase hplc. ¹H NMR (400 MHz, CDCl₃) δ 7.52-7.07 (m, 8H), 5.22 (s, 1H), 3.96 (m, 2H), 3.03 (m, 1H), 2.60 (m, 1H), 2.40 (m, 6H), 0.98 (m, 9H).

LCMS (ES+) (m/z): 476.40 (M+1), 951.94 (2M+1).

### Example 30: (S)-2-(tert-Butoxy)-2-(1-(3-fluoro-2-methylbenzo v)-6-methyl-4-(p-tolyl)indolin-5-yl)acetic acid

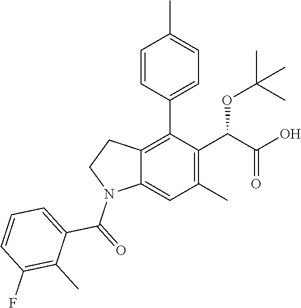 [SMILES: Cc1ccc(-c2c3c(cc(C)c2[C@H](OC(C)(C)C)C(=O)O)N(C(=O)c2cccc(F)c2C)CC3)cc1]

In a manner similar to that described in Example 9, Step 8, the title compound was isolated as a white solid after reverse phase hplc. ¹H NMR (400 MHz, CDCl₃) δ 8.21 (m, 1H), 7.52-7.00 (m, 7H), 5.24 (s, 1H), 3.66 (m, 2H), 3.01 (m, 1H), 2.59 (m, 1H), 2.51-2.36 (m, 6H), 2.30 (m, 3H), 1.00 (m, 9H). LCMS (ES+) (m/z): 490.36 (M+1), 980.58 (2M+1).

### Example 31: (S)-2-(tert-Butoxy)-2-(1-(3-fluoro-4-methylbenzo v)-6-methyl-4-(p-tolyl)indolin-5-yl)acetic acid

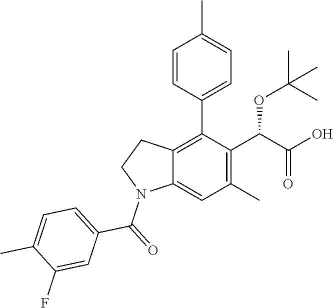 [SMILES: Cc1ccc(-c2c3c(cc(C)c2[C@H](OC(C)(C)C)C(=O)O)N(C(=O)c2ccc(C)c(F)c2)CC3)cc1]

In a manner similar to that described in Example 9, Step 8, the title compound was isolated as a white solid after reverse phase hplc. ¹H NMR (400 MHz, CDCl₃) δ 7.47 (m, 1H), 7.24 (m, 6H), 7.12 (m, 1H), 5.23 (s, 1H), 3.98 (m, 2H), 3.01 (m, 1H), 2.57 (m, 1H), 2.48-2.27 (m, 9H), 0.97 (m, 9H). LCMS (ES+) (m/z): 490.41 (M+1), 979.95 (2M+1).

### Example 32: (S)-2-(tert-Butoxy)-2-(1-(3-fluoro-5-methylbenzo v)-6-methyl-4-(p-tolyl)indolin-5-yl)acetic acid

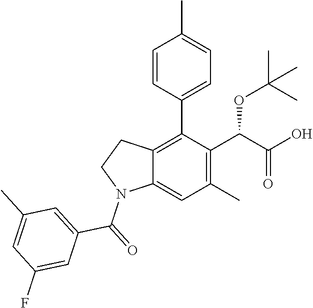 [SMILES: Cc1ccc(-c2c3c(cc(C)c2[C@H](OC(C)(C)C)C(=O)O)N(C(=O)c2cc(C)cc(F)c2)CC3)cc1]

In a manner similar to that described in Example 9, Step 8, the title compound was isolated as a white solid after reverse phase hplc. ¹H NMR (400 MHz, CDCl₃) δ 7.47 (m, 1H), 7.30-6.95 (m, 7H), 5.22 (s, 1H), 3.94 (m, 2H), 3.02 (m, 1H), 2.58 (m, 1H), 2.40 (m, 9H), 0.96 (m, 9H). LCMS (ES+) (m/z): 490.52 (M+1), 979.94 (2M+1).

### Example 33: (S)-2-(tert-Butoxy)-2-(1-(5-fluoro-2-methylbenzoyl)-6-methyl-4-(p-tolyl)indolin-5-yl)acetic acid)

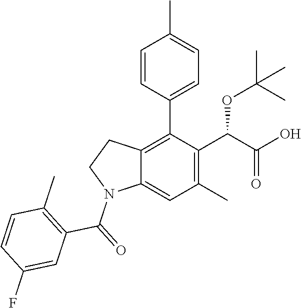 [SMILES: Cc1ccc(-c2c3c(cc(C)c2[C@H](OC(C)(C)C)C(=O)O)N(C(=O)c2cc(F)ccc2C)CC3)cc1]

In a manner similar to that described in Example 9, Step 8, the title compound was isolated as a white solid after reverse phase hplc. ¹H NMR (400 MHz, CDCl₃) δ 8.20 (m, 1H), 7.46 (m, 1H), 7.22 (m, 3H), 7.13 (m, 1H), 7.01 (m, 2H), 5.22 (m, 1H), 3.68 (m, 2H), 3.03 (m, 1H), 2.59 (m, 1H), 2.52-2.29 (m, 9H), 0.99 (m, 9H). LCMS (ES+) (m/z): 490.53 (M+1), 979.97 (2M+1).

### Example 34: (S)-2-(tert-Butoxy)-2-(1-(2-(3,4-difluorophenyl)acetyl)-6-methyl-4-(p-tolyl)indolin-5-yl)acetic acid

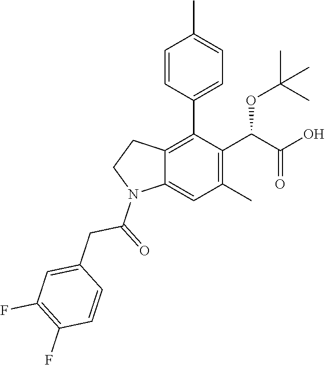 [SMILES: Cc1ccc(-c2c3c(cc(C)c2[C@H](OC(C)(C)C)C(=O)O)N(C(=O)Cc2ccc(F)c(F)c2)CC3)cc1]

In a manner similar to that described in Example 9, Step 8, the title compound was isolated as a white solid after reverse phase hplc ¹H NMR (400 MHz, CDCl₃) δ 8.11 (s, 1H), 7.45 (m, 1H), 7.23 (m, 2H), 7.14 (m, 2H), 7.02 (m, 1H), 5.21 (s, 1H), 4.02 (m, 2H), 3.74 (s, 2H), 3.08 (m, 1H), 2.68 (m, 1H), 2.40 (m, 6H), 0.96 (m, 9H). LCMS (ES+) (m/z): 508.41 (M+1), 1015.87 (2M+1).

### Example 35: (S)-2-(tert-Butoxy)-2-(1-((3,4-difluorophenyl)sulfonyl)-6-methyl-4-(p-tolyl)indolin-5-yl)acetic acid

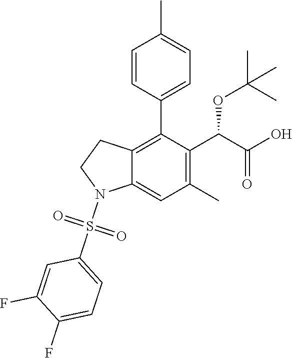 [SMILES: Cc1ccc(-c2c3c(cc(C)c2[C@H](OC(C)(C)C)C(=O)O)N(S(=O)(=O)c2ccc(F)c(F)c2)CC3)cc1]

In a manner similar to that described in Example 28, the title compound was isolated as a white solid after reverse phase hplc. ¹H NMR (400 MHz, CDCl₃) δ 7.73-7.60 (m, 2H), 7.41 (s, 1H), 7.28 (m, 1H), 7.19-7.10 (m, 2H), 6.90 (m, 1H), 6.68 (m, 1H), 5.10 (s, 1H), 3.80 (m, 2H), 2.77-2.24 (m, 8H), 0.87 (s, 9H). LCMS (ES+) (m/z): 1058.85 (2M+1).

### Example 36: (S)-2-(tert-Butoxy)-2-(6-methyl-1-(piperidin-1-ylsulfonyl)-4-(p-tolyl)indolin-5-yl)acetic acid

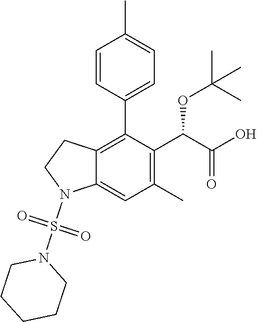 [SMILES: Cc1ccc(-c2c3c(cc(C)c2[C@H](OC(C)(C)C)C(=O)O)N(S(=O)(=O)N2CCCCC2)CC3)cc1]

In a manner similar to that described in Example 28, the title compound was isolated as a white solid after reverse phase hplc. ¹H NMR (400 MHz, CDCl₃) δ 7.44 (m, 1H), 7.24 (m, 2H), 7.13 (m, 2H), 5.20 (s, 1H), 4.07-3.78 (m, 2H), 3.30 (m, 4H), 3.03 (m, 1H), 2.59 (m, 1H), 2.39 (m, 6H), 1.70-1.50 (m, 6H), 0.96 (m, 9H). LCMS (ES+) (m/z): 501.39 (M+1), 1023.82 (2M+1).

### Example 37: (S)-2-(tert-Butoxy)-2-(1-(cyclohexylcarbamoyl)-6-methyl-4-(p-tolyl)indolin-5-yl)acetic acid

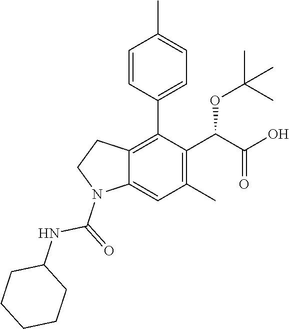 [SMILES: Cc1ccc(-c2c3c(cc(C)c2[C@H](OC(C)(C)C)C(=O)O)N(C(=O)NC2CCCCC2)CC3)cc1]

In a manner similar to that described in Example 7, from (S)-2-(tert-butoxy)-2-(6-methyl-4-(p-tolyl)indolin-5-yl)acetic acid and isocyanatocyclohexane, the title compound was isolated as a white solid after reverse phase hplc. ¹H NMR (400 MHz, CDCl₃) δ 7.78 (m, 1H), 7.45 (m, 1H), 7.23 (m, 2H), 7.12 (m, 1H), 5.18 (s, 1H), 4.39 (m, 1H), 3.78 (m, 3H), 3.06 (m, 1H), 2.65 (m, 1H), 2.38 (m, 6H), 2.02 (m, 2H), 1.81-1.58 (m, 3H), 1.41 (m, 2H), 1.17 (m, 3H), 0.96 (m, 9H). LCMS (ES+) (m/z): 479.46 (M+1), 957.99 (2M+1).

### Example 38: (S)-2-(tert-Butoxy)-2-(6-methyl-1-picolinoyl-4-(p-tolyl)indolin-5-yl)acetic acid

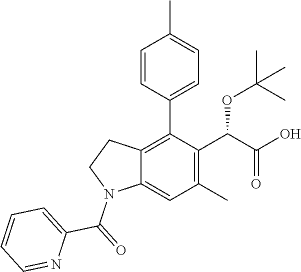 [SMILES: Cc1ccc(-c2c3c(cc(C)c2[C@H](OC(C)(C)C)C(=O)O)N(C(=O)c2ccccn2)CC3)cc1]

In a manner similar to that described in Example 9, Step 8, the title compound was isolated as a white solid after reverse phase hplc. ¹H NMR (400 MHz, CDCl₃) δ 8.64 (m, 1H), 8.19 (m, 1H), 7.86 (m, 2H), 7.45 (m, 2H), 7.23 (m, 2H), 7.13 (m, 1H), 5.25 (s, 1H), 4.38-4.08 (m, 2H), 3.05 (m, 1H), 2.61 (m, 1H), 2.44 (m, 6H), 0.98 (m, 9H).

LCMS (ES+) (m/z): 459.40 (M+1), 917.77 (2M+1).

### Example 39: (S)-2-(tert-Butoxy)-2-(6-methyl-1-nicotinoyl-4-(p-tolyl)indolin-5-yl)acetic acid

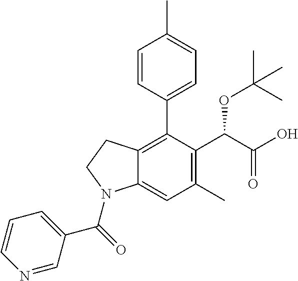 [SMILES: Cc1ccc(-c2c3c(cc(C)c2[C@H](OC(C)(C)C)C(=O)O)N(C(=O)c2cccnc2)CC3)cc1]

In a manner similar to that described in Example 9, Step 8, the title compound was isolated as a white solid after reverse phase hplc. ¹H NMR (400 MHz, CDCl₃) δ 8.90 (m, 1H), 8.78 (m, 1H), 8.08 (m, 1H), 7.59 (m, 1H), 7.46 (m, 1H), 7.25 (m, 3H), 7.13 (m, 1H), 5.23 (s, 1H), 4.00 (m, 2H), 3.08 (m, 1H), 2.63 (m, 1H), 2.41 (m, 6H), 0.97 (m, 9H).

LCMS (ES+) (m/z): 459.52 (M+1), 917.93 (2M+1).

### Example 40: (S)-2-(tert-Butoxy)-2-(1-isonicotinoyl-6-methyl-4-(p-tolyl)indolin-5-yl)acetic acid

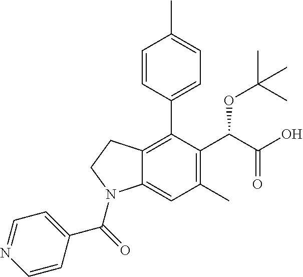 [SMILES: Cc1ccc(-c2c3c(cc(C)c2[C@H](OC(C)(C)C)C(=O)O)N(C(=O)c2ccncc2)CC3)cc1]

In a manner similar to that described in Example 9, Step 8, the title compound was isolated as a white solid after reverse phase hplc. ¹H NMR (400 MHz, CDCl₃) δ 8.83 (m, 2H), 8.10 (m, 1H), 7.59 (m, 2H), 7.46 (m, 1H), 7.25 (m, 2H), 7.11 (m, 1H), 5.24 (s, 1H), 3.87 (m, 2H), 3.07 (m, 1H), 2.64 (m, 1H), 2.44 (m, 6H), 0.98 (m, 9H). LCMS (ES+) (m/z): 459.53 (M+1).

### Example 41: (S)-2-(tert-Butoxy)-2-(1-(cyclohexanecarbonyl)-6-methyl-4-(p-tolyl)indolin-5-yl)acetic acid

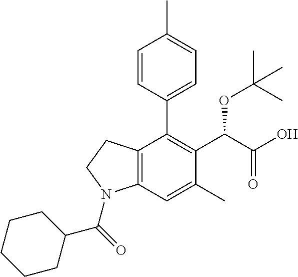 [SMILES: Cc1ccc(-c2c3c(cc(C)c2[C@H](OC(C)(C)C)C(=O)O)N(C(=O)C2CCCCC2)CC3)cc1]

In a manner similar to that described in Example 9, Step 8, the title compound was isolated as a tan solid after reverse phase hplc (13 mg, 45%). ¹H NMR (400 MHz, CDCl₃) δ 8.15 (s, 1H), 7.46 (m, 1H), 7.27-7.22 (m, 2H), 7.12 (m, 1H), 5.20 (s, 1H), 4.16-3.96 (m, 2H), 3.08 (s, 1H), 2.66 (s, 1H), 2.47-2.40 (m, 7H), 1.86-1.58 (m, 8H), 1.30-1.27 (m, 2H), 0.97 (s, 9H). LCMS (ES+) (m/z): 464.56 (M+1); LCMS (ES−)(m/z): 462.48 (M−1).

### Example 42: (S)-2-(tert-Butoxy)-2-(1-(3,4-difluorobenzoyl)-6-methyl-4-(p-tolyl)indolin-5-yl)acetic acid

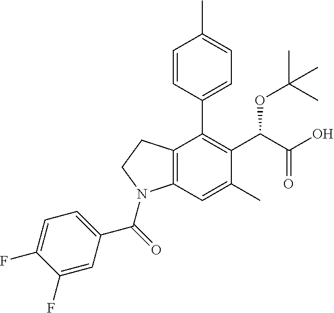 [SMILES: Cc1ccc(-c2c3c(cc(C)c2[C@H](OC(C)(C)C)C(=O)O)N(C(=O)c2ccc(F)c(F)c2)CC3)cc1]

In a manner similar to that described in Example 9, Step 8, the title compound was isolated as a tan solid after reverse phase hplc (7 mg, 25%). ¹H NMR (400 MHz, CDCl₃) δ 7.48-7.39 (m, 2H), 7.32 (m, 1H), 7.27-7.22 (m, 4H), 7.12 (m, 1H), 5.23 (s, 1H), 3.99-3.97 (m, 2H), 3.04 (m, 1H), 2.60 (m, 1H), 2.42-2.39 (m, 6H), 0.98 (s, 9H). LCMS (ES+) (m/z): 494.51 (M+1), 516.53 (M+23); LCMS (ES−)(m/z): 492.48 (M−1).

### Example 43: (S)-2-(tert-Butoxy)-2-(1-(2,3-difluorobenzoyl)-6-methyl-4-(p-tolyl)indolin-5-yl)acetic acid

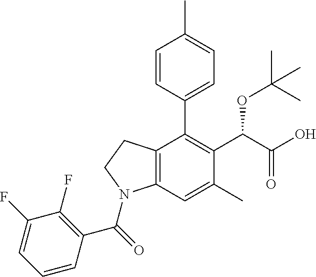 [SMILES: Cc1ccc(-c2c3c(cc(C)c2[C@H](OC(C)(C)C)C(=O)O)N(C(=O)c2cccc(F)c2F)CC3)cc1]

In a manner similar to that described in Example 9, Step 8, the title compound was isolated as a tan solid after reverse phase hplc (13 mg, 39%). ¹H NMR (400 MHz, CDCl₃) δ 8.17 (s, 1H), 7.48 (m, 1H), 7.26-7.21 (m, 5H), 7.13 (m, 1H), 5.24 (s, 1H), 3.91-3.80 (m, 2H), 3.05 (m, 1H), 2.62 (m, 1H), 2.47 (s, 3H), 2.41 (s, 3H), 0.99 (s, 9H). LCMS (ES+) (m/z): 494.53 (M+1), 516.46 (M+23); LCMS (ES−)(m/z): 492.45 (M−1).

### Example 44: (S)-2-(tert-Butoxy)-2-(1-(4-fluoro-2-methylbenzoyl)-6-methyl-4-(p-tolyl)indolin-5-yl)acetic acid

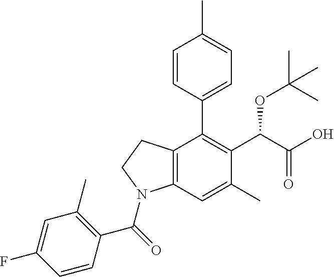 [SMILES: Cc1ccc(-c2c3c(cc(C)c2[C@H](OC(C)(C)C)C(=O)O)N(C(=O)c2ccc(F)cc2C)CC3)cc1]

In a manner similar to that described in Example 9, Step 8, the title compound was isolated as a tan solid after reverse phase hplc (14 mg, 44%). ¹H NMR (400 MHz, CDCl₃) δ 8.20 (s, 1H), 7.46 (s, 1H), 7.27-7.22 (m, 3H), 7.13 (m, 1H), 6.99-6.97 (m, 2H), 5.24-5.16 (m, 1H), 3.65 (s, 1H), 3.01 (s, 1H), 2.59 (s, 1H), 2.46-2.32 (m, 9H), 2.06 (m, 1H), 0.99 (s, 9H). LCMS (ES+) (m/z): 490.54 (M+1); LCMS (ES−)(m/z): 488.53 (M−1).

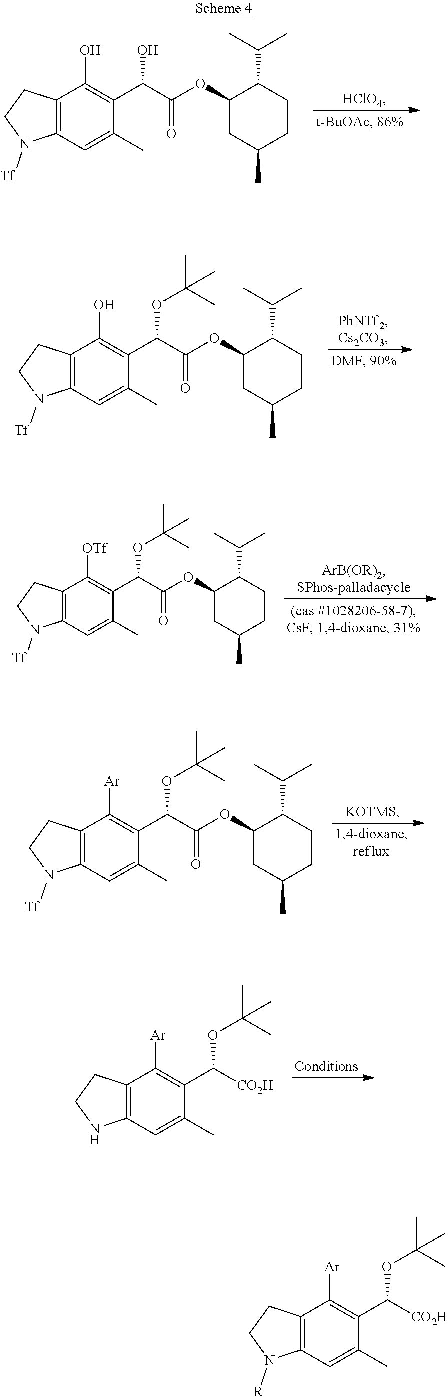

### Example 45: (S)-2-(tert-Butoxy)-2-((R)-1-(3-fluoro-2-methoxybenzoyl)-4-(8-fluoro-5-methylchroman-6-yl)-6-methylindolin-5-yl)acetic acid

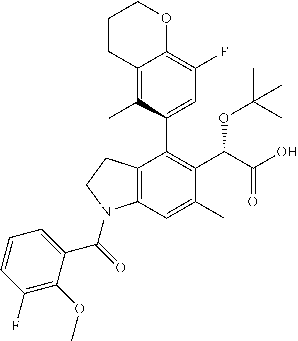

### Step 1: (S)-(1R,2S,5R)-2-Isopropyl-5-methylcyclohexyl 2-(tert-butoxy)-2-(4-hydroxy-6-methyl-1-((trifluoromethyl)sulfonyl)indolin-5-yl)acetate

To a solution of (1R,2S,5R)-2-isopropyl-5-methylcyclohexyl (S)-2-hydroxy-2-(4-hydroxy-6-methyl-1-((trifluoromethyl)sulfonyl)indolin-5-yl)acetate (4.0 g, 8.11 mmol) in t-BuOAc (80 mL) was added HClO₄ (70%, 2.33 g, 16.2 mmol). After stirring at room temperature for 30 minutes, the resulting mixture was poured into ice cold sat. NaHCO₃ aq. solution and extracted with EtOAc. The organic layer was washed with brine, dried over sodium sulfate, filtered, and concentrated under reduced pressure to give the crude product which was purified by column chromatography (silica gel, 0-30% EtOAc in hexanes) to afford the desired product as a yellow solid (3.5 g, 79% yield). LC-MS (ESI): m/z (M+1)=550.0.

### Step 2: (S)-(1R,2S,5R)-2-Isopropyl-5-methylcyclohexyl 2-(tert-butoxy)-2-(6-methyl-1-((trifluoromethyl)sulfonyl)-4-(((trifluoromethyl)sulfonyl)oxy)indolin-5-yl)acetate

A mixture of (1R,2S,5R)-2-isopropyl-5-methylcyclohexyl (S)-2-(tert-butoxy)-2-(4-hydroxy-6-methyl-1-((trifluoromethyl)sulfonyl)indolin-5-yl)acetate (3.5 g, 6.38 mmol), PhNTf₂ (2.74 g, 7.67 mmol) and Cs₂CO₃ (4.16 g, 12.7 mmol) in DMF (40 mL) was stirred at room temperature for 1 hour. The resulting mixture was partitioned between EtOAc and H₂O. The organic layer was washed with brine, dried over sodium sulfate, filtered, and concentrated under reduced pressure to give the crude product which was purified by column chromatography (silica gel, 0-20% EtOAc in hexanes) to afford the desired product as a yellow oil (3.9 g, 90% yield). LC-MS (ESI): m/z (M+23)=704.0.

### Step 3: (S)-(1R,2S,5R)-2-Isopropyl-5-methylcyclohexyl 2-(tert-butoxy)-2-((M)-4-(8-fluoro-5-methylchroman-6-yl)-6-methyl-1-((trifluoromethyl)sulfonyl)indolin-5-yl)acetate

A mixture of the product from Step 2 (300 mg, 0.44 mmol), (8-fluoro-5-methylchroman-6-yl)boronic acid (231 mg, 1.1 mmol, 2.5 eq.), S-Phos Palladacycle (100 mg, 0.13 mmol, 0.3 eq.) and CsF (268 mg, 1.75 mmol, 4 eq.) in anhydrous 1,4-dioxane (6 mL) was thoroughly degassed and purged with N₂. The reaction was irritated in microwave apparatus (low absorption, 20 mL microwave vial) at 130° C. for 30 minutes. After cooling to room temperature, the resulting mixture was diluted with EtOAc and filtered through a pad of Celite. The filtrated was concentrated under reduced pressure to give the crude product which was purified by chromatography (12 g silica gel column, 0-10% PE in EtOAc) to afford the desired product (155 mg, 50% yield) as a light yellow oil. ¹H NMR (400 MHz, CDCl₃) δ 7.22 (s, 1H), 6.71 (d, J=11.1 Hz, 1H), 4.88 (s, 1H), 4.63 (td, J=10.9, 4.3 Hz, 1H), 4.32-4.23 (m, 2H), 4.12 (ddd, J=28.1, 18.8, 9.5 Hz, 2H), 2.67 (dt, J=15.7, 7.6 Hz, 4H), 2.51 (s, 3H), 2.13 (dd, J=6.1, 4.0 Hz, 2H), 1.84 (s, 3H), 1.77 (d, J=11.4 Hz, 1H), 1.64 (ddd, J=13.5, 6.9, 2.8 Hz, 3H), 1.48-1.28 (m, 3H), 1.09 (s, 9H), 1.03-0.97 (m, 1H), 0.85 (t, J=6.2 Hz, 6H), 0.77 (d, J=11.7 Hz, 1H), 0.64 (d, J=6.9 Hz, 3H). LC-MS (ESI): m/z (M+Na)=720.1.

### Step 4: (S)-2-(tert-Butoxy)-2-((M)-4-(8-fluoro-5-methylchroman-6-yl)-6-methylindolin-5-yl)acetic acid

To a solution of the product from Step 3 (540 mg, 0.774 mmol) in 1,4-dioxane was added KOTMS (993 mg, 7.74 mmol) and the resulting mixture was stirred at 110° C. overnight under N₂. After cooling to 0° C., the reaction mixture was acidified with 1 N HCl to pH ˜2, and then extracted with a DCM/IPA solution (85/15) (×3), and the combined organic layer was washed with brine, dried over sodium sulfate, filtered, and concentrated in vacuo. to give the desired product which was used without further purification. LCMS (ES+) (m/z): 428.4 (M+1).

### Step 5: (S)-2-(tert-Butoxy)-2-((M)-1-(3-fluoro-2-methoxybenzoyl)-4-(8-fluoro-5-methylchroman-6-yl)-6-methylindolin-5-yl)acetic acid

In a manner similar to that described in Example 3, from (S)-2-(tert-butoxy)-2-((M)-4-(8-fluoro-5-methylchroman-6-yl)-6-methylindolin-5-yl)acetic acid and 3-fluoro-2-methoxybenzoic acid, the title compound was prepared after purification by reverse-phase HPLC. ¹H NMR (400 MHz, CDCl₃) δ 9.49 (br, 1H), 7.24-7.00 (m, 4H), 6.71 (d, J=11.2 Hz, 1H), 5.07 (s, 1H), 4.29-4.23 (m, 2H), 3.99 (d, J=1.6 Hz, 3H), 3.85-3.70 (m, 2H), 2.72-2.46 (m, 7H), 2.14-1.94 (m, 5H), 1.12 (d, J=19.2 Hz, 9H). LCMS (ES+) (m/z): 580.1 (M+1).

### Example 46: (S)-2-(tert-Butoxy)-2-((R)-4-(8-fluoro-5-methylchroman-6-yl)-6-methyl-1-(4-(trifluoromethyl)benzoyl)indolin-5-yl)acetic acid

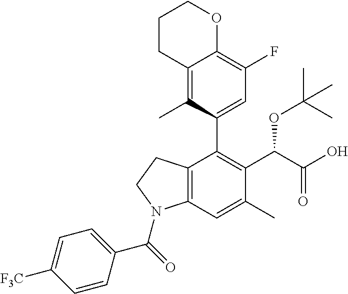

In a manner similar to that described in Example 45, the title compound was prepared after purification by reverse-phase HPLC. ¹H NMR (400 MHz, CDCl₃) δ 9.62 (br, 1H), 8.10 (s, 1H), 7.74 (d, J=8.2 Hz, 2H), 7.66 (d, J=8.1 Hz, 2H), 6.71 (d, J=11.3 Hz, 1H), 5.05 (s, 1H), 4.31-4.21 (m, 2H), 4.04-3.84 (m, 2H), 2.77-2.32 (m, 7H), 2.17-2.08 (m, 2H), 1.94 (s, 3H), 1.13 (s, 9H). LCMS (ES+) (m/z): 600.0 (M+1).

### Example 47: (S)-2-(tert-Butoxy)-2-((R)-1-(4-chloro-2-methylbenzoyl)-4-(8-fluoro-5-methylchroman-6-yl)-6-methylindolin-5-yl)acetic acid

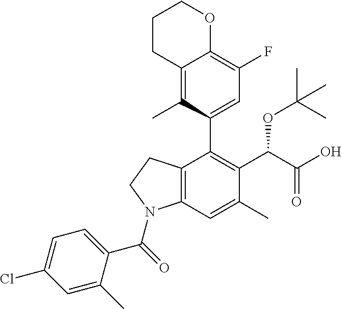

In a manner similar to that described in Example 45, the title compound was prepared after purification by reverse-phase HPLC. ¹H NMR (400 MHz, CDCl₃) δ 9.59 (br, 1H), 8.19 (s, 1H), 7.75-7.53 (m, 1H), 7.26-7.18 (m, 2H), 6.71 (d, J=11.4 Hz, 1H), 5.06 (s, 1H), 4.29-4.22 (m, 2H), 3.65 (d, J=6.4 Hz, 2H), 2.75-2.25 (m, 10H), 2.14-2.07 (m, 2H), 1.94 (s, 3H), 1.14 (s, 9H). LCMS (ES+) (m/z): 580.1/582.4 (M/M+2).

### Example 48: (S)-2-(tert-Butoxy)-2-((R)-1-(4-fluoro-3-methylbenzoyl)-4-(8-fluoro-5-methylchroman-6-yl)-6-methylindolin-5-yl)acetic acid

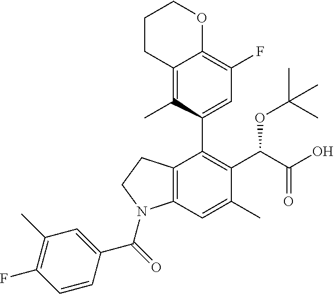

In a manner similar to that described in Example 45, the title compound was prepared after purification by reverse-phase HPLC. ¹H NMR (400 MHz, CDCl₃) δ 9.56 (br, 1H), 7.44-7.39 (m, 1H), 7.39-7.34 (m, 1H), 7.06 (t, J=8.9 Hz, 2H), 6.71 (d, J=11.2 Hz, 1H), 5.05 (s, 1H), 4.28-4.23 (m, 2H), 3.97 (t, J=7.9 Hz, 2H), 2.71-2.39 (m, 7H), 2.32 (d, J=1.4 Hz, 3H), 2.14-2.07 (m, 2H), 1.94 (s, 3H), 1.13 (s, 9H). LCMS (ES+) (m/z): 564.2 (M+1).

### Example 49: (S)-2-(tert-Butoxy)-2-((R)-1-(2-chloro-3-fluorobenzoyl)-4-(8-fluoro-5-methylchroman-6-yl)-6-methylindolin-5-yl)acetic acid

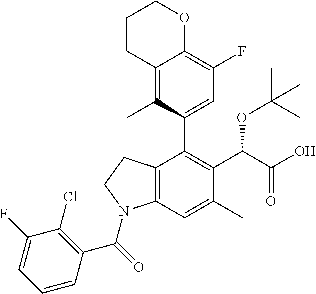

In a manner similar to that described in Example 45, the title compound was prepared after purification by reverse-phase HPLC. ¹H NMR (400 MHz, CDCl₃) δ 9.57 (br, 1H), 7.45-7.30 (m, 2H), 7.24-7.15 (m, 2H), 6.72 (d, J=10.2 Hz, 1H), 5.07 (s, 1H), 4.28-4.23 (m, 2H), 3.81-3.65 (m, 2H), 2.74-2.49 (m, 7H), 2.14-2.09 (m, 2H), 1.95 (s, 3H), 1.14 (s, 9H). LCMS (ES+) (m/z): 584.1/586.3 (M/M+2).

### Example 50: (S)-2-(tert-Butoxy)-2-((R)-1-(5-chloro-2-methoxybenzoyl)-4-(8-fluoro-5-methylchroman-6-yl)-6-methylindolin-5-yl)acetic acid

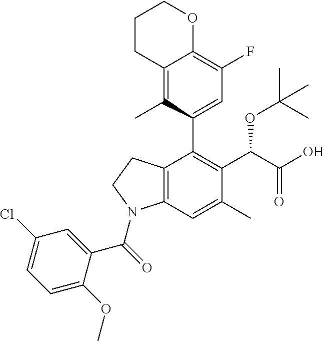

In a manner similar to that described in Example 45, the title compound was prepared after purification by reverse-phase HPLC. ¹H NMR (400 MHz, CDCl₃) δ 9.59 (br, 1H), 8.19 (s, 1H), 7.36-7.28 (m, 2H), 6.89 (d, J=8.8 Hz, 1H), 6.71 (d, J=11.2 Hz, 1H), 5.06 (s, 1H), 4.25 (t, J=5.1 Hz, 2H), 3.91-3.64 (m, 5H), 2.78-2.44 (m, 7H), 2.15-2.08 (m, 2H), 1.95 (s, 3H), 1.14 (s, 9H). LCMS (ES+) (m/z): 596.0/598.3 (M/M+2).

### Example 51: (S)-2-(tert-Butoxy)-2-((R)-1-(4-chloronicotinoyl)-4-(8-fluoro-5-methylchroman-6-yl)-6-methylindolin-5-yl)acetic acid

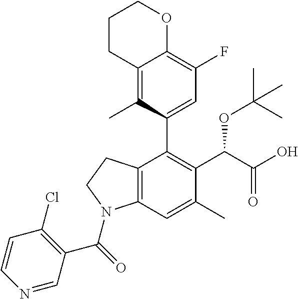

In a manner similar to that described in Example 45, the title compound was prepared after purification by reverse-phase HPLC. ¹H NMR (400 MHz, CDCl₃) δ 9.62 (br, 1H), 8.63 (s, 1H), 8.58 (d, J=5.4 Hz, 1H), 8.21 (s, 1H), 7.43 (d, J=5.4 Hz, 1H), 6.74-6.67 (m, 1H), 5.08 (s, 1H), 4.28-4.23 (m, 2H), 3.76 (t, J=9.5 Hz, 2H), 2.72-2.49 (m, 7H), 2.13-2.08 (m, 2H), 1.95 (s, 3H), 1.15 (s, 9H). LCMS (ES+) (m/z): 567.1/569.3 (M/M+2).

### Example 52: (S)-2-(tert-Butoxy)-2-((R)-1-(3-chlorobenzoyl)-4-(8-fluoro-5-methylchroman-6-yl)-6-methylindolin-5-yl)acetic acid

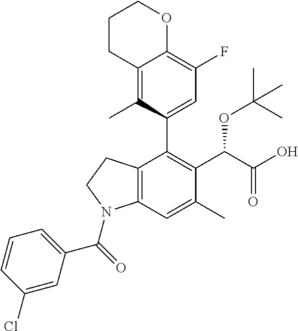

In a manner similar to that described in Example 45, the title compound was prepared after purification by reverse-phase HPLC. ¹H NMR (400 MHz, DMSO) δ 12.26 (s, 1H), 7.91 (s, 1H), 7.64 (s, 1H), 7.62-7.49 (m, 3H), 6.65 (d, J=11.4 Hz, 1H), 4.81 (s, 1H), 4.18 (t, J=5.0 Hz, 2H), 3.88 (t, J=7.8 Hz, 2H), 2.66 (t, J=5.6 Hz, 2H), 2.49-2.39 (m, 5H), 2.07-1.97 (m, 2H), 1.82 (s, 3H), 1.02 (s, 9H). LCMS (ES+) (m/z): 566.0/568.1 (M/M+2).

### Example 53: (S)-2-(tert-Butoxy)-2-((R)-4-(8-fluoro-5-methylchroman-6-yl)-6-methyl-1-(2-methylbenzoyl)indolin-5-yl)acetic acid

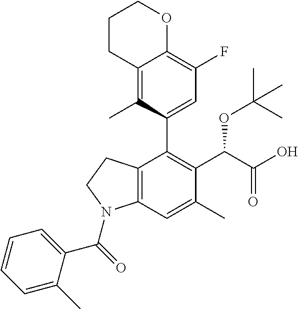

In a manner similar to that described in Example 45, the title compound was prepared after purification by reverse-phase HPLC. ¹H NMR (400 MHz, DMSO) δ 12.19 (s, 1H), 8.02 (s, 1H), 7.58-7.19 (m, 4H), 6.67 (d, J=11.7 Hz, 1H), 4.82 (s, 1H), 4.18 (t, J=4.2 Hz, 2H), 3.74-3.45 (m, 2H), 2.71-2.61 (m, 2H), 2.49-2.40 (m, 5H), 2.27 (s, 3H), 2.06-1.95 (m, 2H), 1.83 (s, 3H), 1.02 (s, 9H). LCMS (ES+) (m/z): 546.0 (M+1).

### Example 54: (S)-2-(tert-Butoxy)-2-((R)-1-(cyclopentanecarbonyl)-4-(8-fluoro-5-methylchroman-6-yl)-6-methylindolin-5-yl)acetic acid

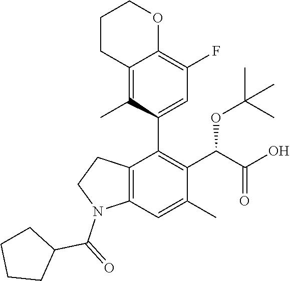

In a manner similar to that described in Example 45, the title compound was prepared after purification by reverse-phase HPLC. ¹H NMR (400 MHz, DMSO) δ 12.13 (s, 1H), 7.93 (s, 1H), 6.65 (d, J=11.5 Hz, 1H), 4.78 (s, 1H), 4.19 (t, J=5.0 Hz, 2H), 4.11-4.01 (m, 2H), 3.02-2.92 (m, 1H), 2.66 (t, J=5.8 Hz, 2H), 2.52 (s, 2H), 2.43-2.34 (m, 3H), 2.06-1.97 (m, 2H), 1.90-1.78 (m, 5H), 1.76-1.51 (m, 6H), 0.94 (s, 9H). LCMS (ES+) (m/z): 524.0 (M+1).

### Example 55: (S)-2-(tert-Butoxy)-2-((R)-1-(2-fluoro-5-methylbenzoyl)-4-(8-fluoro-5-methylchroman-6-yl)-6-methylindolin-5-yl)acetic acid

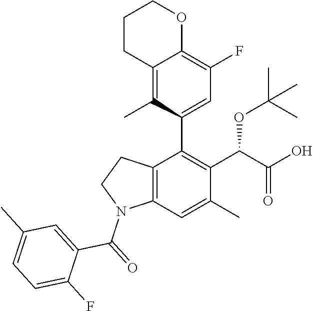

In a manner similar to that described in Example 45, the title compound was prepared after purification by reverse-phase HPLC. ¹H NMR (400 MHz, CDCl₃) δ 9.59 (br, 1H), 8.17 (s, 1H), 7.25-7.17 (m, 2H), 7.06-6.98 (m, 1H), 6.72 (d, J=11.2 Hz, 1H), 5.06 (s, 1H), 4.32-4.21 (m, 2H), 3.85 (t, J=7.8 Hz, 2H), 2.78-2.43 (m, 7H), 2.35 (s, 3H), 2.15-2.08 (m, 2H), 1.94 (s, 3H), 1.14 (s, 9H). LCMS (ES+) (m/z): 564.0 (M+1).

### Example 56: (S)-2-(tert-Butoxy)-2-((R)-1-(4-chloro-3-fluorobenzoyl)-4-(8-fluoro-5-methylchroman-6-yl)-6-methylindolin-5-yl)acetic acid

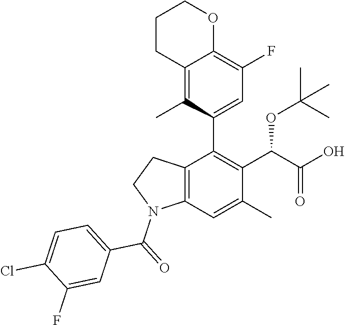

In a manner similar to that described in Example 45, the title compound was prepared after purification by reverse-phase HPLC. ¹H NMR (400 MHz, DMSO) δ 12.27 (br, 1H), 7.90 (s, 1H), 7.78-7.63 (m, 2H), 7.45 (d, J=8.3 Hz, 1H), 6.65 (d, J=11.5 Hz, 1H), 4.82 (s, 1H), 4.18 (t, J=5.0 Hz, 2H), 3.89 (t, J=8.0 Hz, 2H), 2.66 (t, J=5.6 Hz, 2H), 2.49-2.38 (m, 5H), 2.08-1.93 (m, 2H), 1.82 (s, 3H), 1.02 (s, 9H). LCMS (ES+) (m/z): 584.0/586.3 (M/M+2).

### Example 57: (S)-2-(tert-Butoxy)-2-((R)-1-(3,5-difluorobenzoyl)-4-(8-fluoro-5-methylchroman-6-yl)-6-methylindolin-5-yl)acetic acid

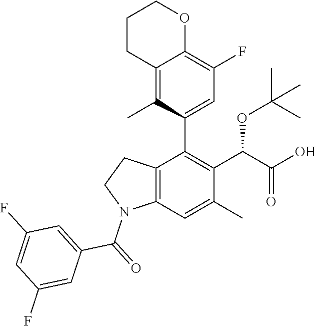

In a manner similar to that described in Example 45, the title compound was prepared after purification by reverse-phase HPLC. ¹H NMR (400 MHz, CDCl₃) δ 9.60 (br, 1H), 8.04 (s, 1H), 7.14-7.02 (m, 2H), 6.93 (t, J=8.9 Hz, 1H), 6.71 (d, J=11.2 Hz, 1H), 5.06 (s, 1H), 4.31-4.20 (m, 2H), 4.04-3.85 (m, 2H), 2.79-2.35 (m, 7H), 2.17-2.07 (m, 2H), 1.94 (s, 3H), 1.13 (s, 9H). LCMS (ES+) (m/z): 568.0 (M+1).

### Example 58: (S)-2-(tert-Butoxy)-2-((R)-4-(8-fluoro-5-methylchroman-6-yl)-6-methyl-1-(3-phen yl/propano yl)indolin-5-yl)acetic acid

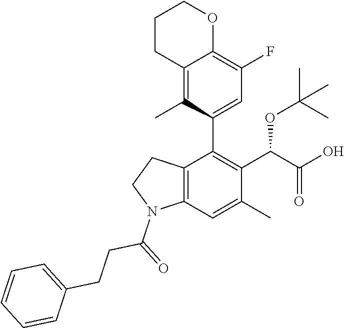

In a manner similar to that described in Example 45, the title compound was prepared after purification by reverse-phase HPLC. ¹H NMR (400 MHz, CDCl₃) δ 9.64 (br, 1H), 8.13 (s, 1H), 7.33-7.26 (m, 3H), 7.25-7.18 (m, 2H), 6.70 (d, J=11.3 Hz, 1H), 5.03 (s, 1H), 4.30-4.23 (m, 2H), 3.90 (t, J=8.5 Hz, 2H), 3.07 (t, J=7.7 Hz, 2H), 2.75-2.50 (m, 6H), 2.43 (s, 3H), 2.15-2.06 (m, 2H), 1.91 (s, 3H), 1.12 (s, 9H). LCMS (ES+) (m/z): 560.0 (M+1).

### Example 59: (S)-2-(tert-Butoxy)-2-((R)-4-(8-fluoro-5-methylchroman-6-yl)-6-methyl-1-(5,6,7,8-tetrahydronaphthalene-1-carbonyl)indolin-5-yl)acetic acid

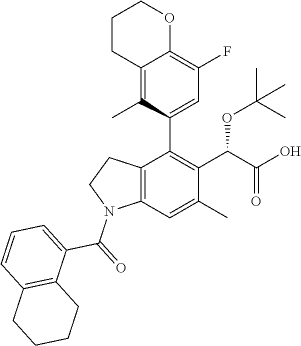

In a manner similar to that described in Example 45, the title compound was prepared after purification by reverse-phase HPLC. ¹H NMR (400 MHz, CDCl₃) δ 9.61 (br, 1H), 8.22 (s, 1H), 7.23-7.05 (m, 3H), 6.71 (d, J=11.4 Hz, 1H), 5.01 (d, J=39.0 Hz, 1H), 4.27-4.21 (m, 2H), 3.81-3.43 (m, 2H), 2.88-2.80 (m, 2H), 2.75-2.46 (m, 7H), 2.14-2.08 (m, 2H), 2.03-1.69 (m, 9H), 1.11 (d, J=18.3 Hz, 9H). LCMS (ES+) (m/z): 586.2 (M+1).

### Example 60: (S)-2-(tert-Butoxy)-2-((R)-1-(2,4-difluorobenzoyl)-4-(8-fluoro-5-methylchroman-6-yl)-6-methylindolin-5-yl)acetic acid

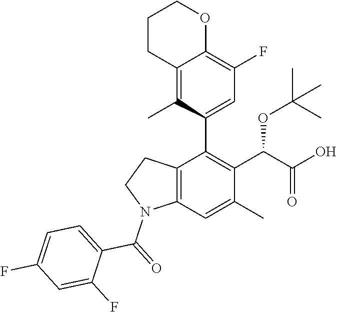

In a manner similar to that described in Example 45, the title compound was prepared after purification by reverse-phase HPLC. ¹H NMR (400 MHz, CDCl₃) δ 9.54 (br, 1H), 8.15 (s, 1H), 7.51-7.44 (m, 1H), 7.02-6.87 (m, 2H), 6.71 (d, J=11.2 Hz, 1H), 5.06 (s, 1H), 4.31-4.21 (m, 2H), 3.94-3.78 (m, 2H), 2.76-2.43 (m, 7H), 2.15-2.07 (m, 2H), 1.94 (s, 3H), 1.14 (s, 9H). LCMS (ES+) (m/z): 568.0 (M+1).

### Example 61: (S)-2-(tert-Butoxy)-2-((R)-1-(3,4-dichlorobenzoyl)-4-(8-fluoro-5-methylchroman-6-yl)-6-methylindolin-5-yl)acetic acid

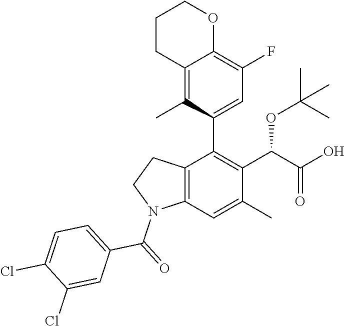

In a manner similar to that described in Example 45, the title compound was prepared after purification by reverse-phase HPLC. ¹H NMR (400 MHz, CDCl₃) δ 9.69 (br, 1H), 7.98 (s, 1H), 7.66 (d, J=1.8 Hz, 1H), 7.54 (d, J=8.2 Hz, 1H), 7.39 (dd, J=8.2, 1.9 Hz, 1H), 6.71 (d, J=11.3 Hz, 1H), 5.05 (s, 1H), 4.28-4.23 (m, 2H), 4.01-3.90 (m, 2H), 2.70-2.41 (m, 7H), 2.15-2.08 (m, 2H), 1.94 (s, 3H), 1.13 (s, 9H). LCMS (ES+) (m/z): 600.0/602.0 (M/M+2).

### Example 62: (S)-2-(tert-Butoxy)-2-((R)-1-(4-fluoro-2-methylbenzoyl)-4-(8-fluoro-5-methylchroman-6-yl)-6-methylindolin-5-yl)acetic acid

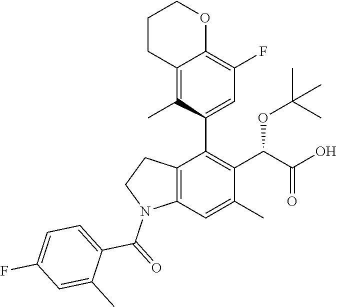

In a manner similar to that described in Example 45, the title compound was prepared after purification by reverse-phase HPLC. ¹H NMR (400 MHz, CDCl₃) δ 9.59 (br, 1H), 8.20 (s, 1H), 7.15-6.83 (m, 3H), 6.71 (d, J=11.3 Hz, 1H), 5.06 (s, 1H), 4.30-4.20 (m, 2H), 3.77-3.53 (m, 2H), 2.77-2.16 (m, 10H), 2.14-2.07 (m, 2H), 1.94 (s, 3H), 1.13 (s, 9H). LCMS (ES+) (m/z): 564.0 (M+1).

### Example 63: (S)-2-(tert-Butoxy)-2-((R)-1-(3,5-dimethoxybenzoyl)-4-(8-fluoro-5-methylchroman-6-yl)-6-methylindolin-5-yl)acetic acid

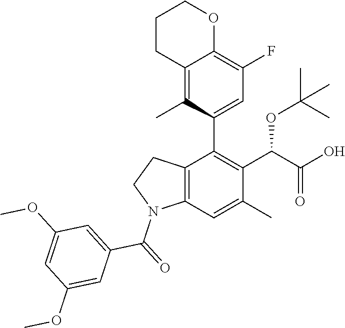

In a manner similar to that described in Example 45, the title compound was prepared after purification by reverse-phase HPLC. ¹H NMR (400 MHz, CDCl₃) δ 9.65 (br, 1H), 8.03 (s, 1H), 6.71 (d, J=11.3 Hz, 1H), 6.65 (d, J=2.3 Hz, 2H), 6.55 (s, 1H), 5.05 (s, 1H), 4.31-4.21 (m, 2H), 4.04-3.90 (m, 2H), 3.80 (d, J=9.7 Hz, 6H), 2.73-2.35 (m, 7H), 2.14-2.06 (m, 2H), 1.94 (s, 3H), 1.13 (s, 9H). LCMS (ES+) (m/z): 592.1 (M+1).

### Example 64: (S)-2-(tert-Butoxy)-2-((R)-1-(4-fluoro-2-(trifluoromethyl)benzoyl)-4-(8-fluoro-5-methylchroman-6-yl)-6-methylindolin-5-yl)acetic acid

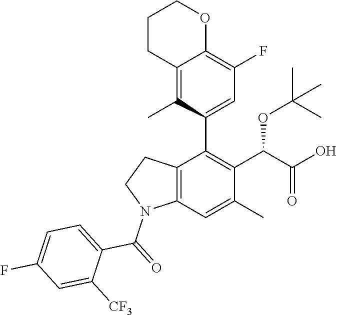

In a manner similar to that described in Example 45, the title compound was prepared after purification by reverse-phase HPLC. ¹H NMR (400 MHz, CDCl₃) δ 9.78 (br, 1H), 8.17 (s, 1H), 7.52-7.30 (m, 3H), 6.72 (d, J=9.1 Hz, 1H), 5.07 (s, 1H), 4.30-4.21 (m, 2H), 3.71-3.55 (m, 2H), 2.77-2.39 (m, 7H), 2.12-1.93 (m, 5H), 1.14 (s, 9H). LCMS (ES+) (m/z): 618.1 (M+1).

### Example 65: (S)-2-(tert-Butoxy)-2-((R)-4-(8-fluoro-5-methylchroman-6-yl)-6-methyl-1-(tetrahydro-2H-pyran-4-carbonyl)indolin-5-yl)acetic acid

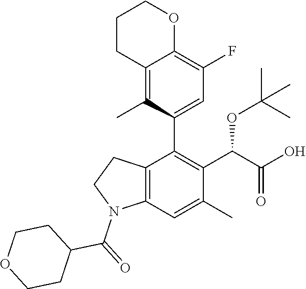

In a manner similar to that described in Example 45, the title compound was prepared after purification by reverse-phase HPLC. ¹H NMR (400 MHz, CDCl₃) δ 9.60 (br, 1H), 8.15 (s, 1H), 6.71 (d, J=11.3 Hz, 1H), 5.03 (s, 1H), 4.26 (t, J=5.2 Hz, 2H), 4.14-3.97 (m, 4H), 3.48 (t, J=11.4 Hz, 2H), 2.79-2.54 (m, 5H), 2.43 (s, 3H), 2.14-1.98 (m, 4H), 1.93 (s, 3H), 1.78-1.68 (m, 2H), 1.12 (s, 9H). LCMS (ES+) (m/z): 540.2 (M+1).

### Example 66: (S)-2-(tert-Butoxy)-2-((R)-1-(3-chloro-2-methylbenzoyl)-4-(8-fluoro-5-methylchroman-6-yl)-6-methylindolin-5-yl)acetic acid

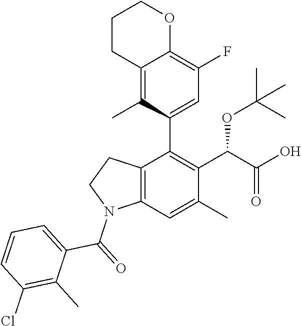

In a manner similar to that described in Example 45, the title compound was prepared after purification by reverse-phase HPLC. ¹H NMR (400 MHz, CDCl₃) δ 9.63 (br, 1H), 8.22 (s, 1H), 7.41 (d, J=7.4 Hz, 1H), 7.24-7.15 (m, 2H), 6.72 (d, J=11.2 Hz, 1H), 5.07 (s, 1H), 4.29-4.23 (m, 2H), 3.71-3.55 (m, 2H), 2.74-2.46 (m, 7H), 2.40 (s, 3H), 2.14-2.08 (m, 2H), 1.94 (d, J=9.4 Hz, 3H), 1.12 (d, J=17.6 Hz, 9H). LCMS (ES+) (m/z): 580.1/582.2 (M/M+2).

### Example 67: (S)-2-(tert-Butoxy)-2-((R)-4-(8-fluoro-5-methylchroman-6-yl)-1-(3-methoxy-2-methylbenzoyl)-6-methylindolin-5-yl)acetic acid

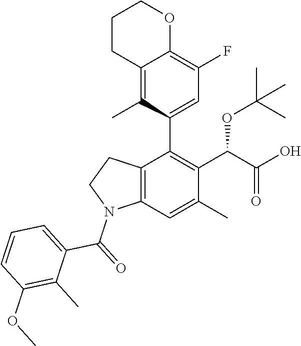

In a manner similar to that described in Example 45, the title compound was prepared after purification by reverse-phase HPLC. ¹H NMR (400 MHz, CDCl₃) δ 9.57 (br, 1H), 8.23 (s, 1H), 7.22 (d, J=7.9 Hz, 1H), 6.94-6.82 (m, 2H), 6.72 (d, J=11.3 Hz, 1H), 5.06 (s, 1H), 4.27-4.21 (m, 2H), 3.92-3.81 (m, 3H), 3.73-3.53 (m, 2H), 2.78-2.42 (m, 7H), 2.22 (s, 3H), 2.14-2.08 (m, 2H), 1.94 (d, J=9.9 Hz, 3H), 1.12 (d, J=19.4 Hz, 9H). LCMS (ES+) (m/z): 576.0 (M+1).

### Example 68: (S)-2-(tert-Butoxy)-2-((R)-1-(2,5-dichlorobenzoyl)-4-(8-fluoro-5-methylchroman-6-yl)-6-methylindolin-5-yl)acetic acid

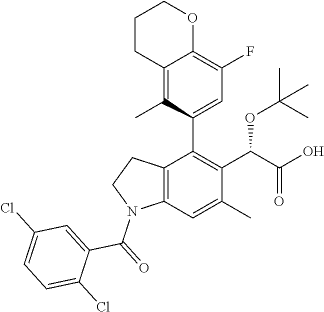

In a manner similar to that described in Example 45, the title compound was prepared after purification by reverse-phase HPLC. ¹H NMR (400 MHz, CDCl₃) δ 9.58 (br, 1H), 8.19 (s, 1H), 7.45-7.32 (m, 3H), 6.72 (d, J=10.9 Hz, 1H), 5.07 (s, 1H), 4.30-4.22 (m, 2H), 3.86-3.67 (m, 2H), 2.75-2.45 (m, 7H), 2.15-2.09 (m, 2H), 1.95 (s, 3H), 1.14 (s, 9H). LCMS (ES+) (m/z): 600.0/602.0 (M/M+2).

### Example 69: (S)-2-(tert-Butoxy)-2-((R)-1-(2,5-dimethylbenzoyl)-4-(8-fluoro-5-methylchroman-6-yl)-6-methylindolin-5-yl)acetic acid

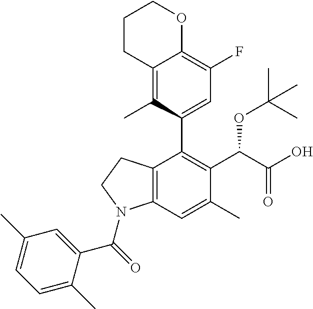

In a manner similar to that described in Example 45, the title compound was prepared after purification by reverse-phase HPLC. ¹H NMR (400 MHz, CDCl₃) δ 9.55 (br, 1H), 8.22 (s, 1H), 7.19-7.06 (m, 3H), 6.72 (d, J=11.4 Hz, 1H), 5.06 (s, 1H), 4.28-4.22 (m, 2H), 3.73-3.57 (m, 2H), 2.72-2.46 (m, 7H), 2.33 (s, 6H), 2.13-2.07 (m, 2H), 1.94 (s, 3H), 1.14 (s, 9H). LCMS (ES+) (m/z): 560.1 (M+1).

### Example 70: (S)-2-(tert-Butoxy)-2-((R)-1-(2,4-dichlorobenzoyl)-4-(8-fluoro-5-methylchroman-6-yl)-6-methylindolin-5-yl)acetic acid

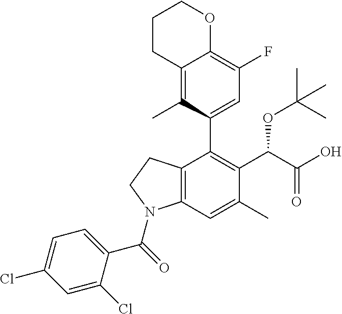

In a manner similar to that described in Example 45, the title compound was prepared after purification by reverse-phase HPLC. ¹H NMR (400 MHz, CDCl₃) δ 9.56 (br, 1H), 8.19 (s, 1H), 7.47 (d, J=1.6 Hz, 1H), 7.41-7.29 (m, 2H), 6.72 (d, J=11.0 Hz, 1H), 5.07 (s, 1H), 4.25 (t, J=5.1 Hz, 2H), 3.83-3.61 (m, 2H), 2.77-2.46 (m, 7H), 2.13-2.08 (m, 2H), 1.94 (s, 3H), 1.12 (d, J=14.9 Hz, 9H). LCMS (ES+) (m/z): 600.1/602.1 (M/M+2).

### Example 71: (S)-2-(tert-Butoxy)-2-((R)-4-(8-fluoro-5-methylchroman-6-yl)-1-(3-methoxy-4-methylbenzoyl)-6-methylindolin-5-yl)acetic acid

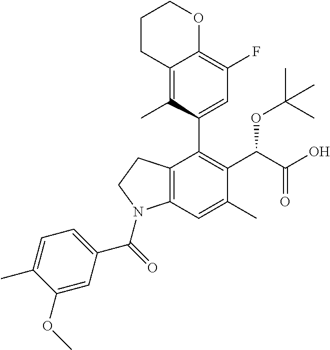

In a manner similar to that described in Example 45, the title compound was prepared after purification by reverse-phase HPLC. ¹H NMR (400 MHz, CDCl₃) δ 9.53 (br, 1H), 7.90 (s, 1H), 7.18 (d, J=7.9 Hz, 1H), 7.05-7.01 (m, 2H), 6.71 (d, J=11.3 Hz, 1H), 5.05 (s, 1H), 4.25 (t, J=5.1 Hz, 2H), 4.00 (t, J=8.1 Hz, 2H), 3.85 (s, 3H), 2.72-2.38 (m, 7H), 2.26 (s, 3H), 2.14-2.06 (m, 2H), 1.94 (s, 3H), 1.13 (s, 9H). LCMS (ES+) (m/z): 578.0 (M+1).

### Example 72: (S)-2-(tert-Butoxy)-2-((R)-1-(4-chloro-2-methoxybenzoyl)-4-(8-fluoro-5-methylchroman-6-yl)-6-methylindolin-5-yl)acetic acid

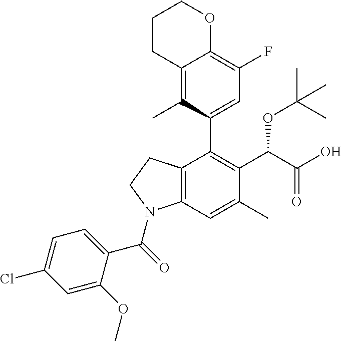

In a manner similar to that described in Example 45, the title compound was prepared after purification by reverse-phase HPLC. ¹H NMR (400 MHz, CDCl₃) δ 9.58 (br, 1H), 8.20 (s, 1H), 7.27-7.24 (m, 1H), 7.05-6.94 (m, 2H), 6.72 (d, J=11.3 Hz, 1H), 5.07 (s, 1H), 4.30-4.23 (m, 2H), 3.86 (s, 3H), 3.80-3.69 (m, 2H), 2.72-2.46 (m, 7H), 2.16-2.10 (m, 2H), 1.95 (s, 3H), 1.14 (s, 9H). LCMS (ES+) (m/z): 596.1/598.1 (M/M+2).

### Example 73: (S)-2-(tert-Butoxy)-2-((R)-1-(3-fluoro-4-methylbenzoyl)-4-(8-fluoro-5-methylchroman-6-yl)-6-methylindolin-5-yl)acetic acid

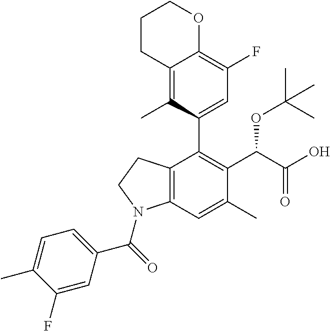

In a manner similar to that described in Example 45, the title compound was prepared after purification by reverse-phase HPLC. ¹H NMR (400 MHz, CDCl₃) δ 9.54 (br, 1H), 7.95 (s, 1H), 7.26-7.19 (m, 3H), 6.71 (d, J=11.3 Hz, 1H), 5.05 (s, 1H), 4.32-4.18 (m, 2H), 3.97 (t, J=7.7 Hz, 2H), 2.80-2.21 (m, 1 OH), 2.16-2.05 (m, 2H), 1.94 (s, 3H), 1.13 (s, 9H). LCMS (ES+) (m/z): 564.1 (M+1).

### Example 74: (S)-2-(tert-butoxy)-2-((R)-1-(3-fluoro-4-methoxybenzoyl)-4-(8-fluoro-5-methylchroman-6-yl)-6-methylindolin-5-yl)acetic acid

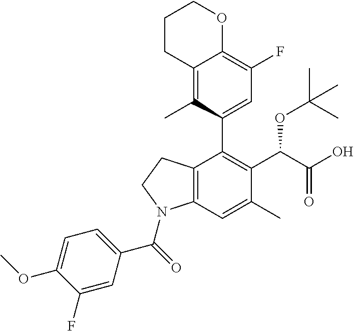

In a manner similar to that described in Example 45, the title compound was prepared after purification by reverse-phase HPLC. ¹H NMR (400 MHz, CDCl₃) δ 9.45 (br, 1H), 7.82 (s, 1H), 7.39-7.28 (m, 2H), 7.01 (t, J=8.5 Hz, 1H), 6.71 (d, J=11.3 Hz, 1H), 5.05 (s, 1H), 4.33-4.19 (m, 2H), 4.01 (t, J=8.2 Hz, 2H), 3.95 (s, 3H), 2.76-2.33 (m, 7H), 2.16-2.06 (m, 2H), 1.94 (s, 3H), 1.13 (s, 9H). LCMS (ES+) (m/z): 580.0 (M+1).

### Example 75: (S)-2-(tert-Butoxy)-2-((R)-1-(3-chloro-2-methoxybenzoyl)-4-(8-fluoro-5-methylchroman-6-yl)-6-methylindolin-5-yl)acetic acid

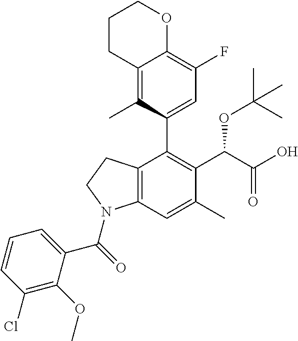

In a manner similar to that described in Example 45, the title compound was prepared after purification by reverse-phase HPLC. ¹H NMR (400 MHz, CDCl₃) (9.57 (br, 1H), 8.20 (s, 1H), 7.45 (dd, J=8.0, 1.5 Hz, 1H), 7.24 (d, J=1.5 Hz, 1H), 7.14 (t, J=7.8 Hz, 1H), 6.71 (d, J=11.5 Hz, 1H), 5.07 (s, 1H), 4.31-4.17 (m, 2H), 3.93 (s, 3H), 3.84-3.52 (m, 2H), 2.85-2.29 (m, 7H), 2.16-2.04 (m, 2H), 1.94 (s, 3H), 1.14 (s, 9H). LCMS (ES+) (m/z): 596.1/598.2 (M/M+2).

### Example 76: (S)-2-(tert-Butoxy)-2-((R)-4-(8-fluoro-5-methylchroman-6-yl)-1-(5-fluoronicotinoyl)-6-methylindolin-5-yl)acetic acid

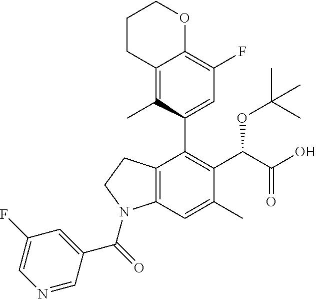

At 0° C., to a solution of 5-fluoronicotinic acid (26 mg, 0.19 mmol) and DMF (1 drop) in DCM (1.5 mL) was added (COCl)₂ (0.031 mL, 0.38 mmol). After stirred at r.t. for 1 hr, the resulting mixture was concentrated under reduced pressure to give the crude acyl chloride intermediate which was diluted with DCM (1 mL) and treated with (S)-2-(tert-butoxy)-2-((R)-4-(8-fluoro-5-methylchroman-6-yl)-6-methylindolin-5-yl)acetic acid (40 mg, the crude product from the previous step) and pyridine (0.5 mL). After stirred at r.t. for 1 hr, the reaction mixture was quenched with sat. NaHCO₃ aq. solution and extracted with DCM.

The organic layer was washed with brine, dried over Na₂SO₄, filtered and concentrated under reduced pressure to give the crude product which was purified by HPLC (C18, 60-100% MeCN in H₂O with 0.1% formic acid) to afford the title compound (6.4 mg, 24% yield) as a white powder. ¹H NMR (400 MHz, CDCl₃) δ 9.59 (br, 1H), 8.74-8.47 (m, 2H), 8.09 (s, 1H), 7.66-7.56 (m, 1H), 6.71 (d, J=11.2 Hz, 1H), 5.06 (s, 1H), 4.33-4.17 (m, 2H), 4.10-3.88 (m, 2H), 2.83-2.27 (m, 7H), 2.17-2.04 (m, 2H), 1.94 (s, 3H), 1.14 (s, 9H). LCMS (ES+) (m/z): 551.3 (M+1).

### Example 77: (S)-2-(tert-Butoxy)-2-((R)-1-(3,5-dimethylbenzoyl)-4-(8-fluoro-5-methylchroman-6-yl)-6-methylindolin-5-yl)acetic acid

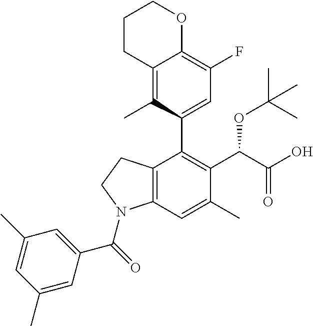

In a manner similar to that described in Example 45, the title compound was prepared after purification by reverse-phase HPLC. ¹H NMR (400 MHz, CDCl₃) δ 9.62 (br, 1H), 7.99 (s, 1H), 7.16-7.05 (m, 3H), 6.71 (d, J=11.4 Hz, 1H), 5.04 (s, 1H), 4.31-4.20 (m, 2H), 4.02-3.88 (m, 2H), 2.75-2.50 (m, 4H), 2.51-2.27 (m, 9H), 2.15-2.06 (m, 2H), 1.94 (s, 3H), 1.13 (s, 9H). LCMS (ES+) (m/z): 560.4 (M+1).

### Example 78: (S)-2-((R)-1-(3-Acetamidobenzoyl)-4-(8-fluoro-5-methylchroman-6-yl)-6-methylindolin-5-yl)-2-(tert-butoxy)acetic acid

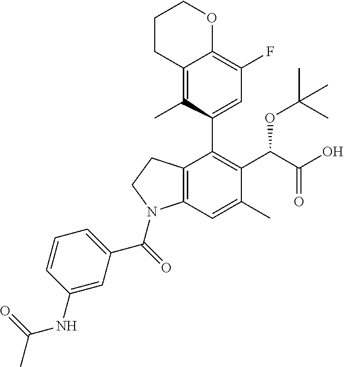

In a manner similar to that described in Example 45, the title compound was prepared after purification by reverse-phase HPLC. ¹H NMR (400 MHz, CDCl₃) δ 9.52 (br, 1H), 8.04 (br, 1H), 7.81-7.31 (m, 5H), 6.71 (d, J=11.3 Hz, 1H), 5.05 (s, 1H), 4.31-4.20 (m, 2H), 4.05-3.91 (m, 2H), 2.73-2.32 (m, 7H), 2.19 (s, 3H), 2.14-2.06 (m, 2H), 1.93 (s, 3H), 1.13 (s, 9H). LCMS (ES+) (m/z): 589.3 (M+1).

### Example 79: (S)-2-(tert-Butoxy)-2-((R)-1-(2,6-dimethoxybenzoyl)-4-(8-fluoro-5-methylchroman-6-yl)-6-methylindolin-5-yl)acetic acid

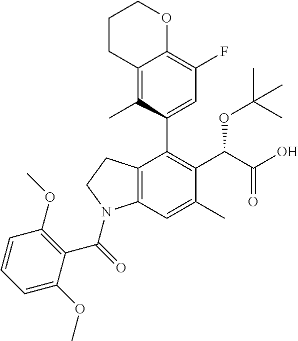

In a manner similar to that described in Example 45, the title compound was prepared after purification by reverse-phase HPLC. ¹H NMR (400 MHz, CDCl₃) δ 9.54 (br, 1H), 8.29 (s, 1H), 7.32-7.27 (m, 1H), 6.72 (d, J=11.3 Hz, 1H), 6.59 (d, J=8.4 Hz, 2H), 5.05 (s, 1H), 4.27-4.23 (m, 2H), 3.83-3.69 (m, 8H), 2.69-2.46 (m, 7H), 2.14-2.08 (m, 2H), 1.96 (s, 3H), 1.13 (s, 9H). LCMS (ES+) (m/z): 592.3 (M+1).

### Example 80: (S)-2-(tert-Butoxy)-2-((R)-1-(5-fluoro-2-methylbenzoyl)-4-(8-fluoro-5-methylchroman-6-yl)-6-methylindolin-5-yl)acetic acid

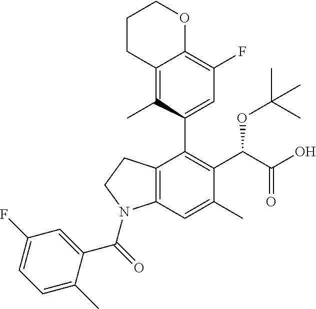

In a manner similar to that described in Example 45, the title compound was prepared after purification by reverse-phase HPLC. ¹H NMR (400 MHz, CDCl₃) δ 9.52 (br, 1H), 8.20 (s, 1H), 7.21-6.95 (m, 3H), 6.71 (d, J=11.3 Hz, 1H), 5.07 (s, 1H), 4.25 (t, J=5.1 Hz, 2H), 3.75-3.55 (m, 2H), 2.71-2.48 (m, 7H), 2.34 (s, 3H), 2.13-2.09 (m, 2H), 1.94 (s, 3H), 1.14 (s, 9H). LCMS (ES+) (m/z): 564.3 (M+1).

### Example 81: (S)-2-(tert-Butoxy)-2-((R)-4-(8-fluoro-5-methylchroman-6-yl)-6-methyl-1-(5-methylnicotinoyl)indolin-5-yl)acetic acid

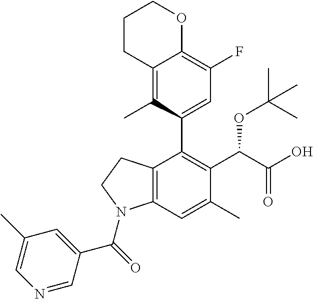

In a manner similar to that described Example 76, the title compound was prepared after purification by reverse-phase HPLC. ¹H NMR (400 MHz, CDCl₃) δ 9.63 (br, 1H), 8.58 (d, J=19.9 Hz, 2H), 8.08 (s, 1H), 7.69 (s, 1H), 6.71 (d, J=11.3 Hz, 1H), 5.05 (s, 1H), 4.26 (t, J=5.2 Hz, 2H), 4.07-3.90 (m, 2H), 2.74-2.36 (m, 10H), 2.15-2.08 (m, 2H), 1.94 (s, 3H), 1.13 (s, 9H). LCMS (ES+) (m/z): 547.4 (M+1).

### Example 82: (S)-2-(tert-Butoxy)-2-((R)-1-(3,5-dichlorobenzoyl)-4-(8-fluoro-5-methylchroman-6-yl)-6-methylindolin-5-yl)acetic acid

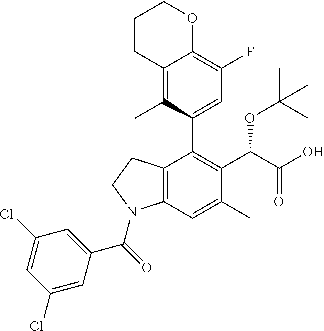

In a manner similar to that described in Example 45, the title compound was prepared after purification by reverse-phase HPLC. ¹H NMR (400 MHz, CDCl₃) δ 9.63 (br, 1H), 8.04 (s, 1H), 7.48 (s, 1H), 7.42 (d, J=1.9 Hz, 2H), 6.72 (d, J=11.3 Hz, 1H), 5.06 (s, 1H), 4.31-4.23 (m, 2H), 4.01-3.86 (m, 2H), 2.73-2.40 (m, 7H), 2.16-2.09 (m, 2H), 1.94 (s, 3H), 1.14 (s, 9H). LCMS (ES+) (m/z): 600.2/602.2 (M/M+2).

### Example 83: (S)-2-(tert-Butoxy)-2-((R)-1-(2,3-dihydrobenzo[b][1,4]dioxine-5-carbonyl)-4-(8-fluoro-5-methylchroman-6-yl)-6-methylindolin-5-yl)acetic acid

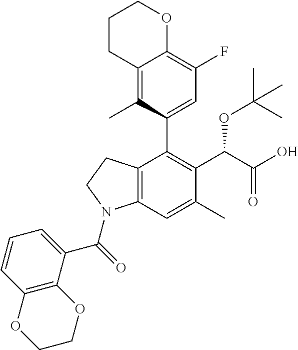

In a manner similar to that described in Example 45, the title compound was prepared after purification by reverse-phase HPLC. ¹H NMR (400 MHz, CDCl₃) δ 9.53 (br, 1H), 8.21 (s, 1H), 6.98-6.84 (m, 3H), 6.71 (d, J=11.2 Hz, 1H), 5.06 (s, 1H), 4.35-4.18 (m, 6H), 3.90-3.75 (m, 2H), 2.75-2.43 (m, 7H), 2.14-2.09 (m, 2H), 1.95 (s, 3H), 1.13 (s, 9H). LCMS (ES+) (m/z): 590.2 (M+1).

### Example 84: (S)-2-(tert-Butoxy)-2-((R)-1-(2,3-dimethoxybenzoyl)-4-(8-fluoro-5-methylchroman-6-yl)-6-methylindolin-5-yl)acetic acid

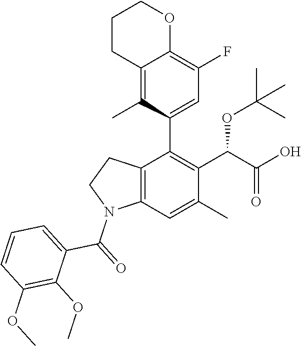

In a manner similar to that described in Example 45, the title compound was prepared after purification by reverse-phase HPLC. ¹H NMR (400 MHz, CDCl₃) δ 9.46 (br, 1H), 8.21 (s, 1H), 7.13 (t, J=7.9 Hz, 1H), 6.97 (d, J=8.3 Hz, 1H), 6.91 (dd, J=7.6, 1.3 Hz, 1H), 6.71 (d, J=11.7 Hz, 1H), 5.06 (s, 1H), 4.27-4.22 (m, 2H), 3.97-3.76 (m, 8H), 2.69-2.47 (m, 7H), 2.13-2.08 (m, 2H), 1.94 (s, 3H), 1.14 (s, 9H). LCMS (ES+) (m/z): 592.4 (M+1).

### Example 85: (S)-2-(tert-Butoxy)-2-((R)-1-(2-fluoro-4-methoxybenzoyl)-4-(8-fluoro-5-methylchroman-6-yl)-6-methylindolin-5-yl)acetic acid

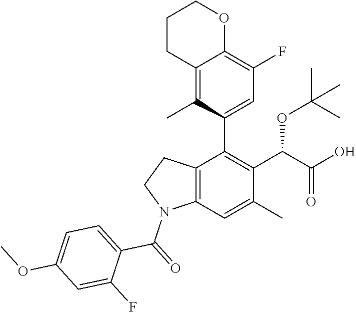

In a manner similar to that described in Example 45, the title compound was prepared after purification by reverse-phase HPLC. ¹H NMR (400 MHz, CDCl₃) δ 9.58 (br, 1H), 8.14 (s, 1H), 7.41 (t, J=8.3 Hz, 1H), 6.82-6.59 (m, 3H), 5.05 (s, 1H), 4.31-4.20 (m, 2H), 4.05-3.68 (m, 5H), 2.79-2.30 (m, 7H), 2.15-2.05 (m, 2H), 1.94 (s, 3H), 1.13 (s, 9H). LCMS (ES+) (m/z): 580.2 (M+1).

### Example 86: (S)-2-(tert-Butoxy)-2-((R)-1-(3,4-dimethoxybenzoyl)-4-(8-fluoro-5-methylchroman-6-yl)-6-methylindolin-5-yl)acetic acid

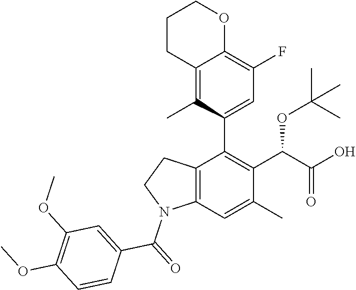

In a manner similar to that described in Example 45, the title compound was prepared after purification by reverse-phase HPLC. ¹H NMR (400 MHz, CDCl₃) δ 9.53 (br, 1H), 7.66 (s, 1H), 7.20-7.11 (m, 2H), 6.90 (d, J=8.2 Hz, 1H), 6.72 (d, J=11.2 Hz, 1H), 5.05 (s, 1H), 4.32-4.22 (m, 2H), 4.04 (t, J=8.3 Hz, 2H), 3.92 (d, J=12.9 Hz, 6H), 2.72-2.37 (m, 7H), 2.16-2.07 (m, 2H), 1.94 (s, 3H), 1.13 (s, 9H). LCMS (ES+) (m/z): 592.0 (M+1).

### Example 87: (S)-2-(tert-Butoxy)-2-((R)-4-(8-fluoro-5-methylchroman-6-yl)-1-(2-methoxy-3-methylbenzoyl)-6-methylindolin-5-yl)acetic acid

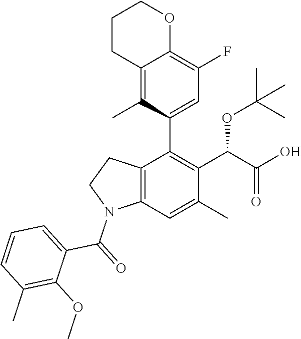

In a manner similar to that described in Example 45, the title compound was prepared after purification by reverse-phase HPLC. ¹H NMR (400 MHz, CDCl₃) δ 9.60 (br, 1H), 8.21 (s, 1H), 7.25-7.03 (m, 3H), 6.71 (d, J=11.1 Hz, 1H), 5.06 (s, 1H), 4.28-4.20 (m, 2H), 3.89-3.58 (m, 5H), 2.77-2.39 (m, 7H), 2.31 (s, 3H), 2.15-2.06 (m, 2H), 1.94 (s, 3H), 1.14 (s, 9H). LCMS (ES+) (m/z): 576.3 (M+1).

### Example 88: (S)-2-(tert-Butoxy)-2-((R)-1-(2,4-dimethoxybenzoyl)-4-(8-fluoro-5-methylchroman-6-yl)-6-methylindolin-5-yl)acetic acid

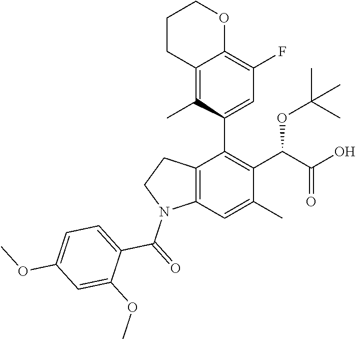

In a manner similar to that described in Example 45, the title compound was prepared after purification by reverse-phase HPLC. ¹H NMR (400 MHz, CDCl₃) δ 9.61 (br, 1H), 8.19 (s, 1H), 7.32-7.27 (m, 1H), 6.71 (d, J=11.3 Hz, 1H), 6.59-6.47 (m, 2H), 5.05 (s, 1H), 4.29-4.21 (m, 2H), 3.93-3.55 (m, 8H), 2.77-2.41 (m, 7H), 2.14-2.07 (m, 2H), 1.94 (s, 3H), 1.13 (s, 9H). LCMS (ES+) (m/z): 592.3 (M+1).

### Example 89: (S)-2-(tert-Butoxy)-2-((R)-4-(8-fluoro-5-methylchroman-6-yl)-1-(3-methoxy-5-methylbenzoyl)-6-methylindolin-5-yl)acetic acid

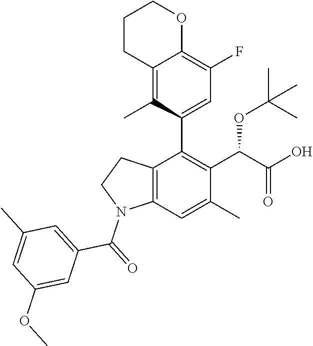

In a manner similar to that described in Example 45, the title compound was prepared after purification by reverse-phase HPLC. ¹H NMR (400 MHz, CDCl₃) δ 9.58 (br, 1H), 7.95 (s, 1H), 6.92 (s, 1H), 6.84 (d, J=11.7 Hz, 2H), 6.71 (d, J=11.3 Hz, 1H), 5.05 (s, 1H), 4.30-4.20 (m, 2H), 4.02-3.88 (m, 2H), 3.81 (s, 3H), 2.75-2.32 (m, 10H), 2.15-2.06 (m, 2H), 1.94 (s, 3H), 1.13 (s, 9H). LCMS (ES+) (m/z): 576.3 (M+1).

### Example 90: (S)-2-(tert-Butoxy)-2-((R)-4-(8-fluoro-5-methylchroman-6-yl)-1-(2-methoxynicotinoyl)-6-methylindolin-5-yl)acetic acid

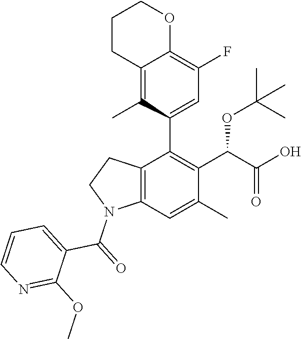

In a manner similar to that described in Example 76, the title compound was prepared after purification by reverse-phase HPLC. ¹H NMR (400 MHz, CDCl₃) δ 9.63 (br, 1H), 8.25 (d, J=3.5 Hz, 1H), 8.19 (s, 1H), 7.67 (d, J=5.7 Hz, 1H), 7.02-6.93 (m, 1H), 6.72 (d, J=11.3 Hz, 1H), 5.07 (s, 1H), 4.25 (t, J=5.1 Hz, 2H), 3.99 (s, 3H), 3.87-3.73 (m, 2H), 2.81-2.37 (m, 7H), 2.14-2.07 (m, 2H), 1.95 (s, 3H), 1.14 (s, 9H). LCMS (ES+) (m/z): 563.3 (M+1).

### Example 91: (S)-2-(tert-Butoxy)-2-((R)-4-(8-fluoro-5-methylchroman-6-yl)-1-(3-fluoroisonicotinoyl)-6-methylindolin-5-yl)acetic acid

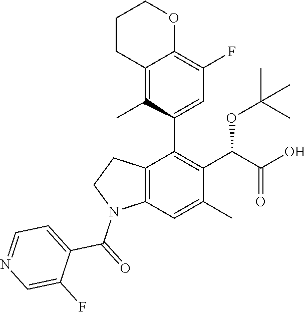

In a manner similar to that described in Example 76, the title compound was prepared after purification by reverse-phase HPLC. ¹H NMR (400 MHz, CDCl₃) δ 9.60 (br, 1H), 8.66-8.49 (m, 2H), 8.16 (s, 1H), 7.40 (t, J=5.1 Hz, 1H), 6.71 (d, J=11.3 Hz, 1H), 5.07 (s, 1H), 4.26 (t, J=5.2 Hz, 2H), 3.92-3.69 (m, 2H), 2.89-2.30 (m, 7H), 2.16-2.06 (m, 2H), 1.94 (s, 3H), 1.14 (s, 9H). LCMS (ES+) (m/z): 551.4 (M+1).

### Example 92: (S)-2-(tert-Butoxy)-2-((R)-1-(2,3-dichlorobenzoyl)-4-(8-fluoro-5-methylchroman-6-yl)-6-methylindolin-5-yl)acetic acid

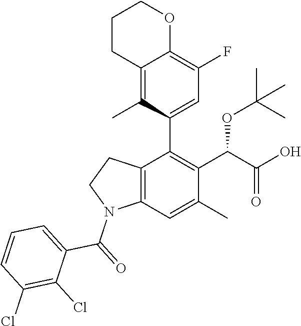

In a manner similar to that described in Example 45, the title compound was prepared after purification by reverse-phase HPLC. ¹H NMR (400 MHz, CDCl₃) δ 9.56 (br, 1H), 8.21 (s, 1H), 7.53 (dd, J=7.5, 2.0 Hz, 1H), 7.33-7.27 (m, 2H), 6.77-6.67 (m, 1H), 5.07 (s, 1H), 4.28-4.22 (m, 2H), 3.84-3.63 (m, 2H), 2.75-2.45 (m, 7H), 2.14-2.08 (m, 2H), 1.93 (d, J=9.8 Hz, 3H), 1.14 (s, 9H). LCMS (ES+) (m/z): 600.2/602.2 (M/M+2).

### Example 93: (S)-2-(tert-Butoxy)-2-((R)-4-(8-fluoro-5-methylchroman-6-yl)-6-methyl-1-(3-(trifluoromethyl)benzoyl)indolin-5-yl)acetic acid

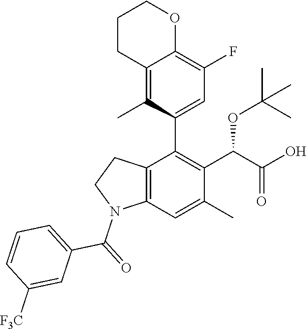

In a manner similar to that described in Example 45, the title compound was prepared after purification by reverse-phase HPLC. ¹H NMR (400 MHz, CDCl₃) δ 9.54 (br, 1H), 8.06 (s, 1H), 7.82 (s, 1H), 7.75 (d, J=7.5 Hz, 2H), 7.61 (t, J=7.7 Hz, 1H), 6.71 (d, J=11.3 Hz, 1H), 5.05 (s, 1H), 4.31-4.18 (m, 2H), 4.06-3.87 (m, 2H), 2.76-2.30 (m, 7H), 2.15-2.05 (m, 2H), 1.94 (s, 3H), 1.13 (s, 9H). LCMS (ES+) (m/z): 600.2 (M+1).

### Example 94: (S)-2-(tert-Butoxy)-2-((R)-1-(3,4-dimethylbenzoyl)-4-(8-fluoro-5-methylchroman-6-yl)-6-methylindolin-5-yl)acetic acid

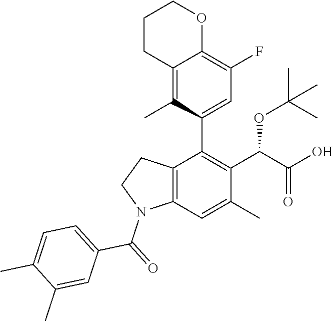

In a manner similar to that described in Example 45, the title compound was prepared after purification by reverse-phase HPLC. ¹H NMR (400 MHz, CDCl₃) δ 9.60 (br, 1H), 7.85 (s, 1H), 7.33 (s, 1H), 7.28-7.26 (m, 1H), 7.19 (d, J=7.8 Hz, 1H), 6.71 (d, J=11.4 Hz, 1H), 5.04 (s, 1H), 4.30-4.21 (m, 2H), 3.98 (t, J=8.2 Hz, 2H), 2.72-2.37 (m, 7H), 2.31 (d, J=4.1 Hz, 6H), 2.14-2.06 (m, 2H), 1.94 (s, 3H), 1.13 (s, 9H). LCMS (ES+) (m/z): 560.2 (M+1).

### Example 95; (S)-2-(tert-Butoxy)-2-((R)-4-(8-fluoro-5-methylchroman-6-yl)-6-methyl-1-(4-(methylcarbamoyl)benzoyl)indolin-5-yl)acetic acid

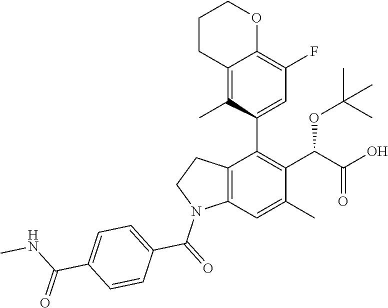

In a manner similar to that described in Example 45, the title compound was prepared after purification by reverse-phase HPLC. ¹H NMR (400 MHz, CDCl₃) δ 9.50 (br, 1H), 8.08 (s, 1H), 7.84 (d, J=8.1 Hz, 2H), 7.60 (d, J=8.3 Hz, 2H), 6.71 (d, J=11.3 Hz, 1H), 6.19 (s, 1H), 5.05 (s, 1H), 4.32-4.18 (m, 2H), 4.04-3.79 (m, 2H), 3.05 (d, J=4.8 Hz, 3H), 2.83-2.26 (m, 7H), 2.17-2.04 (m, 2H), 1.94 (s, 3H), 1.13 (s, 9H). LCMS (ES+) (m/z): 589.3 (M+1).

### Example 96: (S)-2-(tert-Butoxy)-2-((R)-1-(3-fluoro-2-methylbenzoyl)-4-(8-fluoro-5-methylchroman-6-yl)-6-methylindolin-5-yl)acetic acid

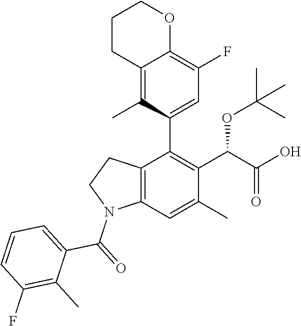

In a manner similar to that described in Example 45, the title compound was prepared after purification by reverse-phase HPLC. ¹H NMR (400 MHz, CDCl₃) δ 9.66 (br, 1H), 8.21 (s, 1H), 7.22 (d, J=8.1 Hz, 1H), 7.07 (t, J=7.5 Hz, 2H), 6.72 (d, J=11.3 Hz, 1H), 5.07 (s, 1H), 4.28-4.24 (m, 2H), 3.71-3.59 (m, 2H), 2.70-2.48 (m, 7H), 2.29 (s, 3H), 2.14-2.09 (m, 2H), 1.95 (s, 3H), 1.14 (s, 9H). LCMS (ES+) (m/z): 564.3 (M+1).

### Example 97: (S)-2-(tert-Butoxy)-2-((R)-1-(3-fluoro-5-methylbenzoyl)-4-(8-fluoro-5-methylchroman-6-yl)-6-methylindolin-5-yl)acetic acid

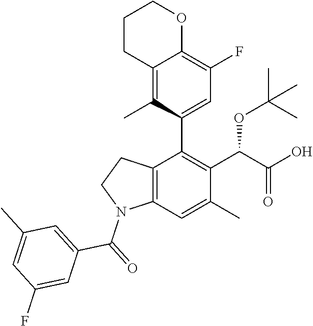

In a manner similar to that described in Example 45, the title compound was prepared after purification by reverse-phase HPLC. ¹H NMR (400 MHz, CDCl₃) δ 9.62 (br, 1H), 8.03 (s, 1H), 7.13 (s, 1H), 7.01 (t, J=10.6 Hz, 2H), 6.71 (d, J=11.3 Hz, 1H), 5.05 (s, 1H), 4.30-4.20 (m, 2H), 4.02-3.87 (m, 2H), 2.75-2.31 (m, 10H), 2.15-2.07 (m, 2H), 1.94 (s, 3H), 1.13 (s, 9H). LCMS (ES+) (m/z): 564.3 (M+1).

### Example 98: (S)-2-(tert-Butoxy)-2-((R)-1-(2-chlorobenzoyl)-4-(8-fluoro-5-methylchroman-6-yl)-6-methylindolin-5-yl)acetic acid

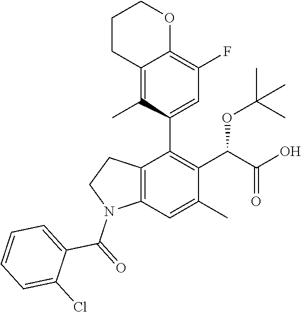

In a manner similar to that described in Example 45, the title compound was prepared after purification by reverse-phase HPLC. ¹H NMR (400 MHz, CDCl₃) δ 9.56 (br, 1H), 8.22 (s, 1H), 7.49-7.34 (m, 4H), 6.72 (d, J=11.8 Hz, 1H), 5.07 (s, 1H), 4.28-4.23 (m, 2H), 3.81-3.63 (m, 2H), 2.72-2.47 (m, 7H), 2.14-2.07 (m, 2H), 1.95 (s, 3H), 1.14 (s, 9H). LCMS (ES+) (m/z): 566.2/568.2 (M/M+2).

### Example 99: (S)-2-(tert-Butoxy)-2-((R)-4-(8-fluoro-5-methylchroman-6-yl)-1-(2-methoxy-5-methylbenzoyl)-6-methylindolin-5-yl)acetic acid

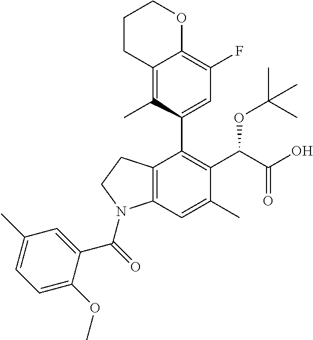

In a manner similar to that described in Example 45, the title compound was prepared after purification by reverse-phase HPLC. ¹H NMR (400 MHz, CDCl₃) δ 9.63 (br, 1H), 8.22 (s, 1H), 7.21-7.06 (m, 2H), 6.84 (d, J=8.4 Hz, 1H), 6.71 (d, J=11.2 Hz, 1H), 5.06 (s, 1H), 4.25 (t, J=5.1 Hz, 2H), 3.92-3.54 (m, 5H), 2.77-2.39 (m, 7H), 2.30 (s, 3H), 2.16-2.07 (m, 2H), 1.95 (s, 3H), 1.14 (s, 9H). LCMS (ES+) (m/z): 576.3 (M+1).

### Example 100: (S)-2-(tert-Butoxy)-2-((R)-1-(2,3-dihydrobenzofuran-7-carbonyl)-4-(8-fluoro-5-methylchroman-6-yl)-6-methylindolin-5-yl)acetic acid

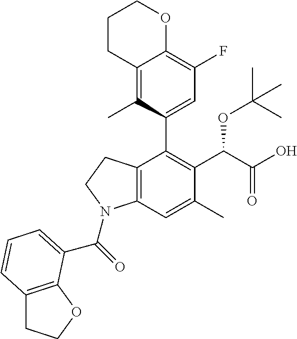

In a manner similar to that described in Example 45, the title compound was prepared after purification by reverse-phase HPLC. ¹H NMR (400 MHz, CDCl₃) δ 9.61 (br, 1H), 8.16 (s, 1H), 7.26-7.23 (m, 2H), 6.94-6.89 (m, 1H), 6.72 (d, J=11.3 Hz, 1H), 5.04 (s, 1H), 4.66-4.53 (m, 2H), 4.30-4.20 (m, 2H), 4.04-3.86 (m, 2H), 3.24 (t, J=8.7 Hz, 2H), 2.79-2.28 (m, 7H), 2.17-2.06 (m, 2H), 1.94 (s, 3H), 1.13 (s, 9H). LCMS (ES+) (m/z): 574.3 (M+1).

### Example 101: (S)-2-(tert-Butoxy)-2-((R)-1-(5-fluoro-2-methoxybenzoyl)-4-(8-fluoro-5-methylchroman-6-yl)-6-methylindolin-5-yl)acetic acid

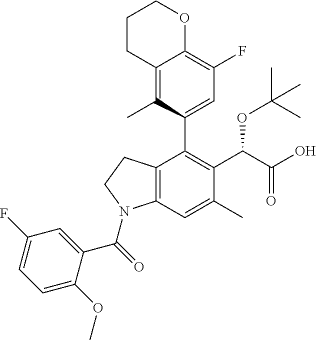

In a manner similar to that described in Example 45, the title compound was prepared after purification by reverse-phase HPLC. ¹H NMR (400 MHz, CDCl₃) δ 9.59 (br, 1H), 8.20 (s, 1H), 7.15-6.99 (m, 2H), 6.89 (dd, J=8.9, 4.1 Hz, 1H), 6.71 (d, J=11.3 Hz, 1H), 5.06 (s, 1H), 4.25 (t, J=5.1 Hz, 2H), 3.93-3.54 (m, 5H), 2.80-2.32 (m, 7H), 2.17-2.07 (m, 2H), 1.95 (s, 3H), 1.14 (s, 9H). LCMS (ES+) (m/z): 580.3 (M+1).

### Example 102: (S)-2-(tert-Butoxy)-2-((R)-4-(8-fluoro-5-methylchroman-6-yl)-1-(4-methoxy-3-methylbenzoyl)-6-methylindolin-5-yl)acetic acid

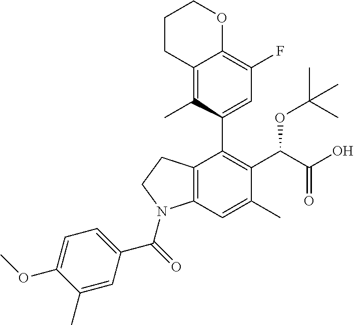

In a manner similar to that described in Example 45, the title compound was prepared after purification by reverse-phase HPLC. ¹H NMR (400 MHz, CDCl₃) δ 9.60 (br, 1H), 7.67 (s, 1H), 7.45-7.33 (m, 2H), 6.85 (d, J=8.4 Hz, 1H), 6.71 (d, J=11.2 Hz, 1H), 5.04 (s, 1H), 4.32-4.19 (m, 2H), 4.01 (t, J=8.3 Hz, 2H), 3.88 (s, 3H), 2.78-2.33 (m, 7H), 2.24 (s, 3H), 2.14-2.05 (m, 2H), 1.94 (s, 3H), 1.13 (s, 9H). LCMS (ES+) (m/z): 576.3 (M+1).

### Example 103: (S)-2-(tert-Butoxy)-2-((R)-1-(4-chloropicolinoyl)-4-(8-fluoro-5-methylchroman-6-yl)-6-methylindolin-5-yl)acetic acid

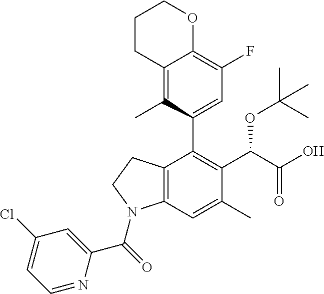

In a manner similar to that described in Example 45, the title compound was prepared after purification by reverse-phase HPLC. ¹H NMR (400 MHz, CDCl₃) δ 9.64 (br, 1H), 8.51 (d, J=5.2 Hz, 1H), 8.18 (s, 1H), 7.90 (s, 1H), 7.39 (d, J=4.0 Hz, 1H), 6.72 (d, J=11.2 Hz, 1H), 5.07 (s, 1H), 4.36-4.13 (m, 4H), 2.79-2.38 (m, 7H), 2.16-2.07 (m, 2H), 1.93 (s, 3H), 1.14 (s, 9H). LCMS (ES+) (m/z): 567.2/569.2 (M/M+2).

### Example 104: (S)-2-((R)-1-(4-Acetamidobenzoyl)-4-(8-fluoro-5-methylchroman-6-yl)-6-methylindolin-5-yl)-2-(tert-butoxy)acetic acid

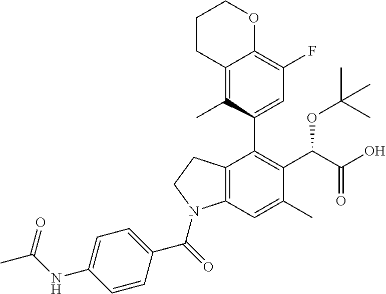

In a manner similar to that described in Example 45, the title compound was prepared after purification by reverse-phase HPLC. ¹H NMR (400 MHz, CDCl₃) δ 9.58 (br, 1H), 7.88 (s, 1H), 7.65-7.40 (m, 5H), 6.70 (d, J=11.3 Hz, 1H), 5.06 (s, 1H), 4.29-4.20 (m, 2H), 4.00 (t, J=8.0 Hz, 2H), 2.73-2.32 (m, 7H), 2.21 (s, 3H), 2.15-2.07 (m, 2H), 1.93 (s, 3H), 1.13 (s, 9H). LCMS (ES+) (m/z): 589.3 (M+1).

### Example 105: (2S)-2-(tert-Butoxy)-2-((4R)-4-(8-fluoro-5-methylchroman-6-yl)-6-methyl-1-(1,2,3,4-tetrahydronaphthalene-1-carbonyl)indolin-5-yl)acetic acid

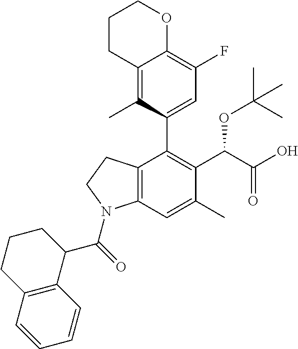

In a manner similar to that described in Example 45, the title compound was prepared after purification by reverse-phase HPLC. ¹H NMR (400 MHz, CDCl₃) δ 9.65 (br, 1H), 8.19 (s, 1H), 7.19-7.08 (m, 3H), 7.04-6.98 (m, 1H), 6.73 (dd, J=11.3, 7.0 Hz, 1H), 5.05 (s, 1H), 4.36-4.02 (m, 5H), 3.00-2.50 (m, 7H), 2.40 (s, 3H), 2.17-1.92 (m, 8H), 1.13 (d, J=1.9 Hz, 9H). LCMS (ES+) (m/z): 586.3 (M+1).

### Example 106: (S)-2-(tert-Butoxy)-2-((R)-1-(2,5-dimethoxybenzo yl)-4-(8-fluoro-5-methylchroman-6-yl)-6-methylindolin-5-yl)acetic acid

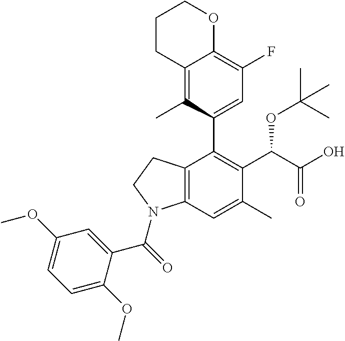

In a manner similar to that described in Example 45, the title compound was prepared after purification by reverse-phase HPLC. ¹H NMR (400 MHz, CDCl₃) δ 9.46 (br, 1H), 8.22 (s, 1H), 7.00-6.80 (m, 3H), 6.71 (d, J=11.4 Hz, 1H), 5.06 (s, 1H), 4.25 (t, J=5.0 Hz, 2H), 3.98-3.53 (m, 8H), 2.82-2.36 (m, 7H), 2.14-2.07 (m, 2H), 1.95 (s, 3H), 1.14 (s, 9H). LCMS (ES+) (m/z): 592.2 (M+1).

### Example 107: (S)-2-(tert-Butoxy)-2-((R)-4-(8-fluoro-5-methylchroman-6-yl)-1-(5-methoxy-2-methylbenzoyl)-6-methylindolin-5-yl)acetic acid

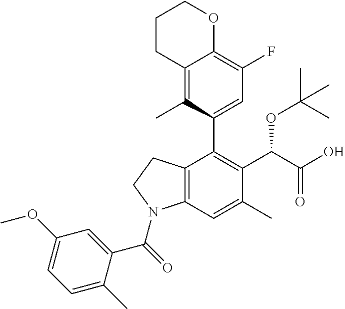

In a manner similar to that described in Example 45, the title compound was prepared after purification by reverse-phase HPLC. ¹H NMR (400 MHz, CDCl₃) δ 9.92 (br, 1H), 8.22 (s, 1H), 7.16 (d, J=8.3 Hz, 1H), 6.97-6.79 (m, 2H), 6.71 (d, J=11.2 Hz, 1H), 5.06 (s, 1H), 4.30-4.22 (m, 2H), 3.79 (s, 3H), 3.74-3.56 (m, 2H), 2.81-2.42 (m, 7H), 2.36-2.20 (m, 3H), 2.11 (s, 2H), 1.95 (s, 3H), 1.12 (d, J=16.7 Hz, 9H). LCMS (ES+) (m/z): 576.2 (M+1).

### Example 108: (S)-2-(tert-Butoxy)-2-((R)-4-(8-fluoro-5-methylchroman-6-yl)-6-methyl-1-(4-methylbenzoyl)indolin-5-yl)acetic acid

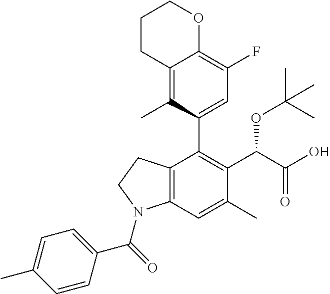

In a manner similar to that described in Example 45, the title compound was prepared after purification by reverse-phase HPLC. ¹H NMR (400 MHz, CDCl₃) δ 9.58 (br, 1H), 7.81 (s, 1H), 7.45 (d, J=8.1 Hz, 2H), 7.26-7.23 (m, 2H), 6.71 (d, J=11.3 Hz, 1H), 5.04 (s, 1H), 4.30-4.20 (m, 2H), 3.98 (t, J=7.9 Hz, 2H), 2.76-2.31 (m, 10H), 2.15-2.05 (m, 2H), 1.94 (s, 3H), 1.13 (s, 9H). LCMS (ES+) (m/z): 546.3 (M+1).

### Example 109: (S)-2-(tert-Butoxy)-2-((R)-1-(2,5-difluorobenzoyl)-4-(8-fluoro-5-methylchroman-6-yl)-6-methylindolin-5-yl)acetic acid

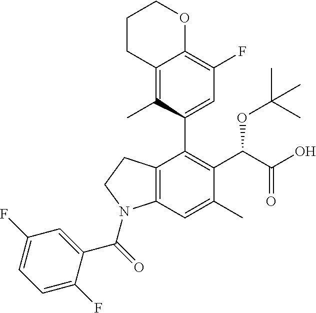

In a manner similar to that described in Example 45, the title compound was prepared after purification by reverse-phase HPLC. ¹H NMR (400 MHz, CDCl₃) δ 9.60 (br, 1H), 8.15 (s, 1H), 7.23-7.05 (m, 3H), 6.72 (d, J=11.2 Hz, 1H), 5.07 (s, 1H), 4.26 (t, J=5.1 Hz, 2H), 3.86 (t, J=8.1 Hz, 2H), 2.78-2.40 (m, 7H), 2.15-2.08 (m, 2H), 1.94 (s, 3H), 1.14 (s, 9H). LCMS (ES+) (m/z): 568.2 (M+1).

### Example 110: (S)-2-(tert-Butoxy)-2-((R)-1-(4-chloro-3-methylbenzoyl)-4-(8-fluoro-5-methylchroman-6-yl)-6-methylindolin-5-yl)acetic acid

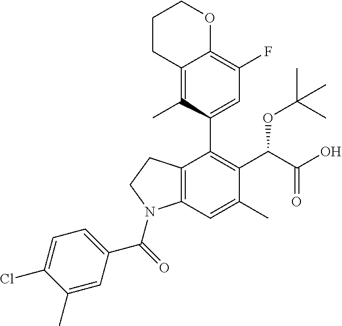

In a manner similar to that described in Example 45, the title compound was prepared after purification by reverse-phase HPLC. ¹H NMR (400 MHz, CDCl₃) δ 9.60 (br, 1H), 8.01 (s, 1H), 7.47-7.37 (m, 2H), 7.30 (dd, J=8.1, 2.0 Hz, 1H), 6.71 (d, J=11.2 Hz, 1H), 5.05 (s, 1H), 4.33-4.17 (m, 2H), 4.04-3.83 (m, 2H), 2.77-2.29 (m, 10H), 2.18-2.04 (m, 2H), 1.94 (s, 3H), 1.13 (s, 9H). LCMS (ES+) (m/z): 580.3/582.3 (M/M+2).

### Example 111: (S)-2-(tert-Butoxy)-2-((R)-1-(2,6-difluorobenzoyl)-4-(8-fluoro-5-methylchroman-6-yl)-6-methylindolin-5-yl)acetic acid

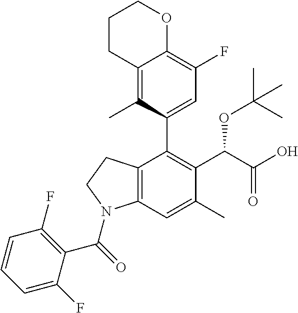

In a manner similar to that described in Example 76, the title compound was prepared after purification by reverse-phase HPLC. ¹H NMR (400 MHz, CDCl₃) δ 9.49 (br, 1H), 8.22 (s, 1H), 7.43-7.34 (m, 1H), 6.99 (t, J=8.1 Hz, 2H), 6.73 (d, J=11.3 Hz, 1H), 5.07 (s, 1H), 4.28-4.23 (m, 2H), 3.83 (t, J=8.4 Hz, 2H), 2.75-2.45 (m, 7H), 2.15-2.08 (m, 2H), 1.95 (s, 3H), 1.14 (s, 9H). LCMS (ES+) (m/z): 568.3 (M+1).

### Example 112: (S)-2-(tert-Butoxy)-2-((R)-4-(8-fluoro-5-methylchroman-6-yl)-6-methyl-1-(4-(piperidine-1-carbonyl)benzoyl)indolin-5-yl)acetic acid

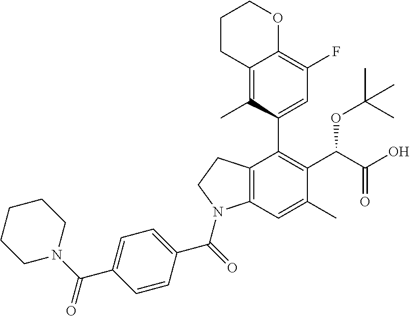

In a manner similar to that described in Example 45, the title compound was prepared after purification by reverse-phase HPLC. ¹H NMR (400 MHz, CDCl₃) δ 9.45 (br, 1H), 8.06 (s, 1H), 7.58 (d, J=8.1 Hz, 2H), 7.47 (d, J=8.0 Hz, 2H), 6.71 (d, J=11.4 Hz, 1H), 5.05 (s, 1H), 4.31-4.19 (m, 2H), 4.02-3.87 (m, 2H), 3.79-3.66 (m, 2H), 3.39-3.28 (m, 2H), 2.74-2.35 (m, 7H), 2.16-2.08 (m, 2H), 1.94 (s, 3H), 1.62-1.52 (m, 6H), 1.13 (s, 9H). LCMS (ES+) (m/z): 643.6 (M+1).

### Example 113: (S)-2-(tert-Butoxy)-2-((R)-1-(3,4-difluoro-2-methoxybenzoyl)-4-(8-fluoro-5-methylchroman-6-yl)-6-methylindolin-5-yl)acetic acid

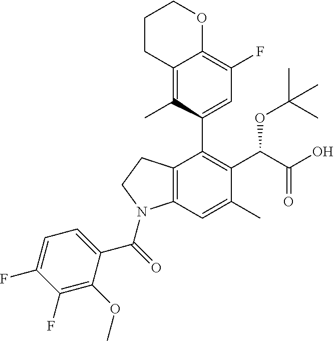

In a manner similar to that described in Example 45, the title compound was prepared after purification by reverse-phase HPLC. ¹H NMR (400 MHz, CDCl₃) δ 9.54 (br, 1H), 8.17 (s, 1H), 7.09-6.94 (m, 2H), 6.71 (d, J=11.3 Hz, 1H), 5.07 (s, 1H), 4.30-4.23 (m, 2H), 4.03 (d, J=1.6 Hz, 3H), 3.83-3.68 (m, 2H), 2.76-2.45 (m, 7H), 2.14-2.08 (m, 2H), 1.95 (s, 3H), 1.14 (s, 9H). LCMS (ES+) (m/z): 598.4 (M+1).

### Example 114: (S)-2-((R)-1-(Benzo[d][1,3]dioxole-4-carbonyl)-4-(8-fluoro-5-methylchroman-6-yl)-6-methylindolin-5-yl)-2-(tert-butoxy)acetic acid

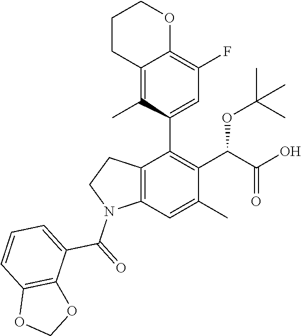

In a manner similar to that described in Example 45, the title compound was prepared after purification by reverse-phase HPLC. ¹H NMR (400 MHz, CDCl₃) δ 9.43 (br, 1H), 8.15 (s, 1H), 7.05-6.86 (m, 3H), 6.71 (d, J=11.2 Hz, 1H), 6.00 (s, 2H), 5.05 (s, 1H), 4.32-4.20 (m, 2H), 4.09-3.94 (m, 2H), 2.79-2.33 (m, 7H), 2.15-2.07 (m, 2H), 1.94 (s, 3H), 1.13 (s, 9H). LCMS (ES+) (m/z): 576.2 (M+1).

### Example 115: (S)-2-(tert-Butoxy)-2-((R)-1-(3-chloro-4-methylbenzoyl)-4-(8-fluoro-5-methylchroman-6-yl)-6-methylindolin-5-yl)acetic acid

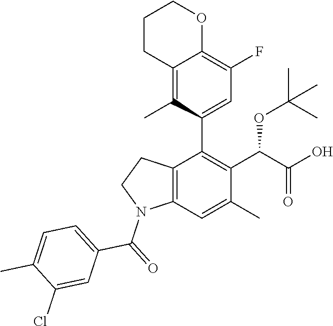

In a manner similar to that described in Example 45, the title compound was prepared after purification by reverse-phase HPLC. ¹H NMR (400 MHz, CDCl₃) δ 9.59 (br, 1H), 8.02 (s, 1H), 7.54 (d, J=1.4 Hz, 1H), 7.38-7.28 (m, 2H), 6.71 (d, J=11.4 Hz, 1H), 5.05 (s, 1H), 4.30-4.19 (m, 2H), 4.02-3.89 (m, 2H), 2.77-2.31 (m, 10H), 2.14-2.07 (m, 2H), 1.94 (s, 3H), 1.13 (s, 9H). LCMS (ES+) (m/z): 580.2/582.2 (M/M+2).

### Example 116: (S)-2-(tert-Butoxy)-2-((R)-4-(8-fluoro-5-methylchroman-6-yl)-6-methyl-1-(6-methylnicotinoyl)indolin-5-yl)acetic acid

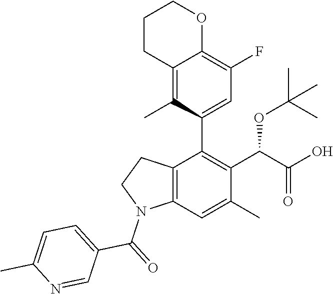

In a manner similar to that described in Example 45, the title compound was prepared after purification by reverse-phase HPLC. ¹H NMR (400 MHz, CDCl₃) δ 9.71 (br, 1H), 8.73 (d, J=1.7 Hz, 1H), 8.05 (s, 1H), 7.81 (dd, J=8.0, 2.1 Hz, 1H), 7.30-7.26 (m, 1H), 6.71 (d, J=11.3 Hz, 1H), 5.05 (s, 1H), 4.32-4.19 (m, 2H), 4.12-3.86 (m, 2H), 2.85-2.25 (m, 10H), 2.15-2.05 (m, 2H), 1.94 (s, 3H), 1.13 (s, 9H). LCMS (ES+) (m/z): 547.4 (M+1).

### Example 117: (S)-2-(tert-Butoxy)-2-((R)-1-(3-chloro-5-methylchroman-6-yl)-6-methylindolin-5-yl)acetic acid

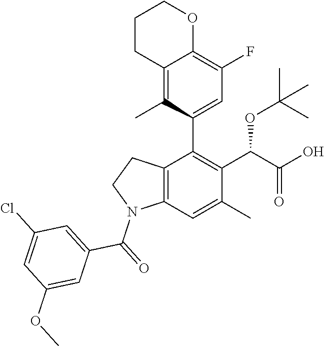

In a manner similar to that described in Example 45, the title compound was prepared after purification by reverse-phase HPLC. ¹H NMR (400 MHz, CDCl₃) δ 9.60 (br, 1H), 8.03 (s, 1H), 7.11-7.05 (m, 1H), 7.00 (s, 1H), 6.96-6.90 (m, 1H), 6.71 (d, J=11.3 Hz, 1H), 5.05 (s, 1H), 4.30-4.21 (m, 2H), 4.01-3.87 (m, 2H), 3.83 (s, 3H), 2.73-2.34 (m, 7H), 2.16-2.06 (m, 2H), 1.94 (s, 3H), 1.13 (s, 9H). LCMS (ES+) (m/z): 596.4/598.4 (M/M+2).

### Example 118: (S)-2-(tert-Butoxy)-2-((R)-1-(2-fluoro-3-methylbenzoyl)-4-(8-fluoro-5-methylchroman-6-yl)-6-methylindolin-5-yl)acetic acid

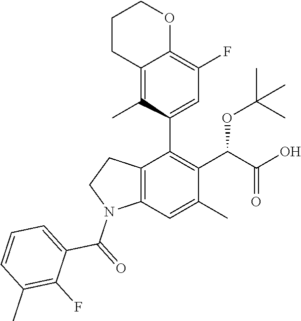

In a manner similar to that described in Example 45, the title compound was prepared after purification by reverse-phase HPLC. ¹H NMR (400 MHz, CDCl₃) δ 9.57 (s, 1H), 8.19 (s, 1H), 7.26-7.22 (m, 2H), 7.16-7.07 (m, J=7.5 Hz, 1H), 6.72 (d, J=11.4 Hz, 1H), 5.06 (s, 1H), 4.30-4.20 (m, 2H), 3.83 (t, J=8.2 Hz, 2H), 2.80-2.39 (m, 7H), 2.32 (s, 3H), 2.11 (d, J=3.1 Hz, 2H), 1.94 (s, 3H), 1.12 (d, J=17.8 Hz, 9H). LCMS (ES+) (m/z): 564.4 (M+1).

### Example 119: (S)-2-(tert-Butoxy)-2-((R)-1-(3-chloro-4-fluorobenzoyl)-4-(8-fluoro-5-methylchroman-6-yl)-6-methylindolin-5-yl)acetic acid

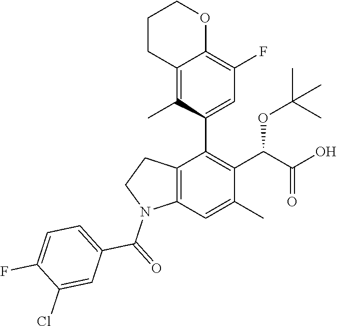

In a manner similar to that described in Example 45, the title compound was prepared after purification by reverse-phase HPLC. ¹H NMR (400 MHz, CDCl₃) δ 9.49 (br, 1H), 7.90 (s, 1H), 7.57 (dd, J=6.9, 2.0 Hz, 1H), 7.42-7.36 (m, 1H), 7.18-7.13 (m, 1H), 6.64 (d, J=11.3 Hz, 1H), 4.98 (s, 1H), 4.23-4.14 (m, 2H), 3.95-3.84 (m, 2H), 2.65-2.29 (m, 7H), 2.08-1.99 (m, 2H), 1.87 (s, 3H), 1.06 (s, 9H). LCMS (ES+) (m/z): 584.3/586.3 (M/M+2).

### Example 120: (S)-2-(tert-Butoxy)-2-((R)-4-(8-fluoro-5-methylchroman-6-yl)-6-methyl-1-pivaloylindolin-5-yl)acetic acid

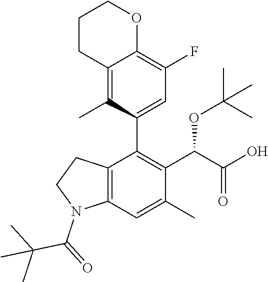

In a manner similar to that described in Example 45, the title compound was prepared after purification by reverse-phase HPLC. ¹H NMR (400 MHz, CDCl₃) δ 9.54 (br, 1H), 8.13 (s, 1H), 6.70 (d, J=11.4 Hz, 1H), 5.04 (s, 1H), 4.26 (t, J=5.2 Hz, 2H), 4.15 (t, J=8.2 Hz, 2H), 2.75-2.37 (m, 7H), 2.16-2.07 (m, 2H), 1.93 (s, 3H), 1.36 (s, 9H), 1.12 (s, 9H). LCMS (ES+) (m/z): 512.3 (M+1).

### Example 121: (S)-2-(tert-Butoxy)-2-((R)-1-(2,3-dimethylbenzoyl)-4-(8-fluoro-5-methylchroman-6-yl)-6-methylindolin-5-yl)acetic acid

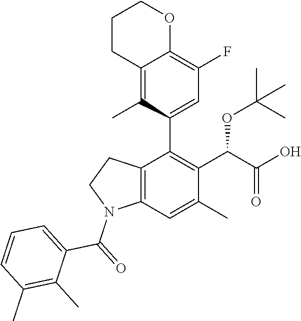

In a manner similar to that described in Example 45, the title compound was prepared after purification by reverse-phase HPLC. ¹H NMR (400 MHz, CDCl₃) δ 9.44 (br, 1H), 8.24 (s, 1H), 7.23-7.07 (m, 3H), 6.72 (d, J=11.2 Hz, 1H), 5.07 (s, 1H), 4.29-4.22 (m, 2H), 3.74-3.52 (m, 2H), 2.73-1.93 (m, 18H), 1.14 (s, 9H). LCMS (ES+) (m/z): 560.4 (M+1).

### Example 122: (S)-2-(tert-Butoxy)-2-((R)-1-(2,3-dihydro-1H-indene-5-carbonyl)-4-(8-fluoro-5-methylchroman-6-yl)-6-methylindolin-5-yl)acetic acid

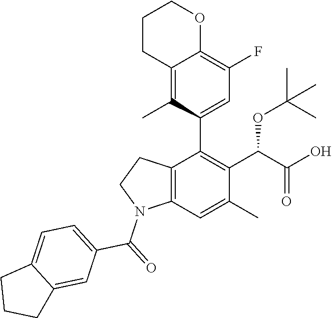

In a manner similar to that described in Example 45, the title compound was prepared after purification by reverse-phase HPLC. ¹H NMR (400 MHz, CDCl₃) δ 9.51 (br, 1H), 7.91 (s, 1H), 7.40 (s, 1H), 7.33-7.27 (m, 2H), 6.71 (d, J=11.3 Hz, 1H), 5.04 (s, 1H), 4.28-4.22 (m, 2H), 3.98 (t, J=8.3 Hz, 2H), 2.98-2.92 (m, 4H), 2.71-2.38 (m, 7H), 2.16-2.07 (m, 4H), 1.94 (s, 3H), 1.13 (s, 9H). LCMS (ES+) (m/z): 572.4 (M+1).

### Example 123: (S)-2-(tert-Butoxy)-2-((R)-1-(4-chloro-3-methoxybenzoyl)-4-(8-fluoro-5-methylchroman-6-yl)-6-methylindolin-5-yl)acetic acid

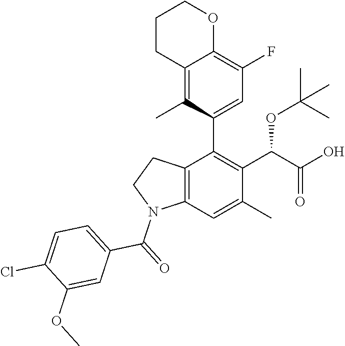

In a manner similar to that described in Example 45, the title compound was prepared after purification by reverse-phase HPLC. ¹H NMR (400 MHz, CDCl₃) δ 9.57 (br, 1H), 8.06 (s, 1H), 7.43 (d, J=8.0 Hz, 1H), 7.14 (d, J=1.7 Hz, 1H), 7.07 (dd, J=8.0, 1.8 Hz, 1H), 6.71 (d, J=11.1 Hz, 1H), 5.05 (s, 1H), 4.30-4.23 (m, 2H), 4.03-3.89 (m, 5H), 2.71-2.36 (m, 7H), 2.15-2.07 (m, 2H), 1.94 (s, 3H), 1.13 (s, 9H). LCMS (ES+) (m/z): 596.3/598.3 (M/M+2).

### Example 124: (S)-2-(tert-Butoxy)-2-((R)-1-(3-chloro-4-methoxybenzoyl)-4-(8-fluoro-5-methylchroman-6-yl)-6-methylindolin-5-yl)acetic acid

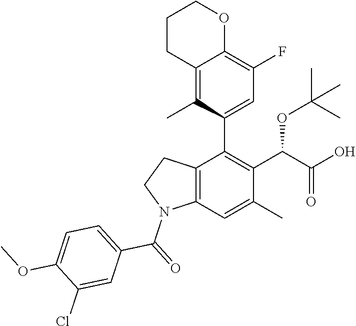

In a manner similar to that described in Example 45, the title compound was prepared after purification by reverse-phase HPLC. ¹H NMR (400 MHz, CDCl₃) δ 9.58 (br, 1H), 7.79 (s, 1H), 7.62 (d, J=2.1 Hz, 1H), 7.49 (dd, J=8.5, 2.1 Hz, 1H), 6.98 (d, J=8.6 Hz, 1H), 6.71 (d, J=11.3 Hz, 1H), 5.05 (s, 1H), 4.30-4.22 (m, 2H), 4.03-3.95 (m, 5H), 2.72-2.39 (m, 7H), 2.14-2.07 (m, 2H), 1.94 (s, 3H), 1.13 (s, 9H). LCMS (ES+) (m/z): 596.3/598.3 (M/M+2).

### Example 125: (S)-2-(tert-Butoxy)-2-((R)-4-(8-fluoro-5-methylchroman-6-yl)-6-methyl-1-(2-(trifluoromethyl)benzoyl)indolin-5-yl)acetic acid

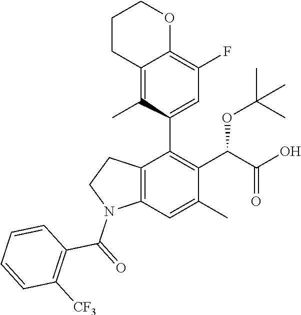

In a manner similar to that described in Example 45, the title compound was prepared after purification by reverse-phase HPLC. ¹H NMR (400 MHz, CDCl₃) δ 9.50 (br, 1H), 8.21 (s, 1H), 7.76-7.46 (m, 4H), 6.74 (s, 1H), 5.08 (s, 1H), 4.28-4.24 (m, 2H), 3.70-3.58 (m, 2H), 2.73-2.48 (m, 7H), 2.15-2.08 (m, 2H), 1.95 (s, 3H), 1.15 (s, 9H). LCMS (ES+) (m/z): 600.3 (M+1).

### Example 126: (S)-2-(tert-Butoxy)-2-((R)-4-(8-fluoro-5-methylchroman-6-yl)-6-methyl-1-(2,3,4-trimethoxybenzoyl)indolin-5-yl)acetic acid

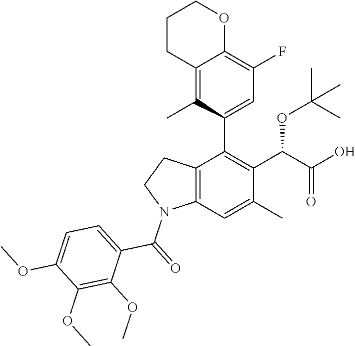

In a manner similar to that described in Example 45, the title compound was prepared after purification by reverse-phase HPLC. ¹H NMR (400 MHz, CDCl₃) δ 9.64 (br, 1H), 8.18 (s, 1H), 7.03 (d, J=8.5 Hz, 1H), 6.81-6.67 (m, 2H), 5.06 (s, 1H), 4.28-4.21 (m, 2H), 4.00-3.67 (m, 11H), 2.77-2.40 (m, 7H), 2.13-2.06 (m, 2H), 1.94 (s, 3H), 1.13 (s, 9H). LCMS (ES+) (m/z): 622.4 (M+1).

### Example 127: (S)-2-(tert-Butoxy)-2-((R)-1-(2,3-dihydrobenzofuran-5-carbonyl)-4-(8-fluoro-5-methylchroman-6-yl)-6-methylindolin-5-yl)acetic acid

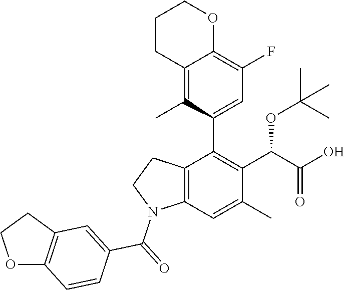

In a manner similar to that described in Example 45, the title compound was prepared after purification by reverse-phase HPLC. ¹H NMR (400 MHz, CDCl₃) δ 9.58 (br, 1H), 7.57 (s, 1H), 7.45 (s, 1H), 7.36 (d, J=8.2 Hz, 1H), 6.81 (d, J=8.2 Hz, 1H), 6.71 (d, J=11.4 Hz, 1H), 5.05 (s, 1H), 4.64 (t, J=8.8 Hz, 2H), 4.29-4.22 (m, 2H), 4.02 (t, J=8.3 Hz, 2H), 3.26 (t, J=8.7 Hz, 2H), 2.70-2.38 (m, 7H), 2.15-2.08 (m, 2H), 1.94 (s, 3H), 1.13 (s, 9H). LCMS (ES+) (m/z): 574.4 (M+1).

### Example 128: (S)-2-(tert-Butoxy)-2-((R)-1-(2-chloro-4-methoxybenzoyl)-4-(8-fluoro-5-methylchroman-6-yl)-6-methylindolin-5-yl)acetic acid

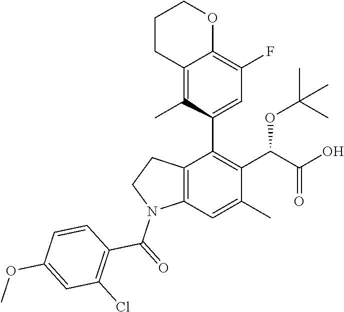

In a manner similar to that described in Example 45, the title compound was prepared after purification by reverse-phase HPLC. ¹H NMR (400 MHz, CDCl₃) δ 9.57 (br, 1H), 8.20 (s, 1H), 7.29 (d, J=8.5 Hz, 1H), 6.99-6.93 (m, 1H), 6.92-6.83 (m, 1H), 6.72 (d, J=11.0 Hz, 1H), 5.06 (s, 1H), 4.25 (t, J=5.2 Hz, 2H), 3.92-3.65 (m, 5H), 2.81-2.35 (m, 7H), 2.14-2.07 (m, 2H), 1.94 (s, 3H), 1.14 (s, 9H). LCMS (ES+) (m/z): 596.3/598.2 (M/M+2).

### Example 129: (S)-2-(tert-Butoxy)-2-((R)-4-(8-fluoro-5-methylchroman-6-yl)-1-(1H-indole-2-carbonyl)-6-methylindolin-5-yl)acetic acid

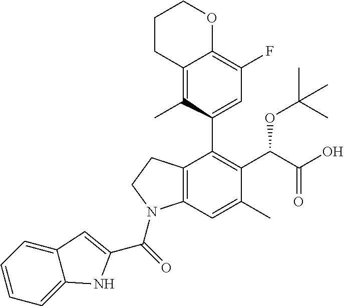

In a manner similar to that described in Example 45, the title compound was prepared after purification by reverse-phase HPLC. ¹H NMR (400 MHz, CDCl₃) δ 9.74 (br, 1H), 9.43 (s, 1H), 8.19 (s, 1H), 7.69 (d, J=8.3 Hz, 1H), 7.47 (d, J=8.3 Hz, 1H), 7.37-7.30 (m, 1H), 7.16 (t, J=7.2 Hz, 1H), 6.99 (d, J=1.4 Hz, 1H), 6.75 (d, J=11.2 Hz, 1H), 5.08 (s, 1H), 4.55-4.40 (m, 2H), 4.27 (t, J=5.2 Hz, 2H), 2.86-2.65 (m, 4H), 2.49 (s, 3H), 2.16-2.09 (m, 2H), 1.96 (s, 3H), 1.15 (s, 9H). LCMS (ES+) (m/z): 571.3 (M+1).

### Example 130: (S)-2-(tert-Butoxy)-2-((R)-1-(cyclohex-1-ene-1-carbonyl)-4-(8-fluoro-5-methylchroman-6-yl)-6-methylindolin-5-yl)acetic acid

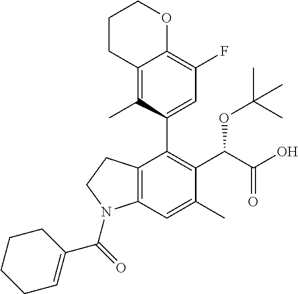

In a manner similar to that described in Example 45, the title compound was prepared after purification by reverse-phase HPLC. ¹H NMR (400 MHz, CDCl₃) δ 9.61 (br, 1H), 7.72 (s, 1H), 6.71 (d, J=11.3 Hz, 1H), 6.05 (s, 1H), 5.04 (s, 1H), 4.33-4.16 (m, 2H), 4.00 (t, J=8.4 Hz, 2H), 2.78-2.36 (m, 7H), 2.31 (s, 2H), 2.23-2.04 (m, 4H), 1.93 (s, 3H), 1.80-1.62 (m, 4H), 1.13 (s, 9H). LCMS (ES+) (m/z): 536.3 (M+1).

### Example 131: (S)-2-(tert-Butoxy)-2-((R)-4-(8-fluoro-5-methylchroman-6-yl)-1-(furan-2-carbonyl)-6-methylindolin-5-yl)acetic acid

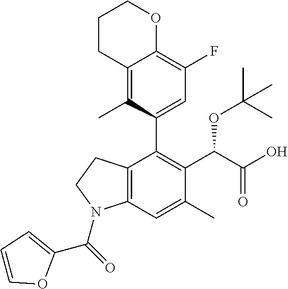

In a manner similar to that described in Example 45, the title compound was prepared after purification by reverse-phase HPLC. ¹H NMR (400 MHz, CDCl₃) δ 9.58 (br, 1H), 8.09 (s, 1H), 7.56 (d, J=0.9 Hz, 1H), 7.22 (d, J=3.5 Hz, 1H), 6.73 (d, J=11.4 Hz, 1H), 6.55 (dd, J=3.5, 1.7 Hz, 1H), 5.06 (s, 1H), 4.44-4.36 (m, 2H), 4.31-4.20 (m, 2H), 2.80-2.58 (m, 4H), 2.45 (s, 3H), 2.16-2.08 (m, 2H), 1.94 (s, 3H), 1.13 (s, 9H). LCMS (ES+) (m/z): 522.2 (M+1).

### Example 132: (S)-2-(tert-Butoxy)-2-((R)-1-(cyclopropanecarbonyl)-4-(8-fluoro-5-methylchroman-6-yl)-6-methylindolin-5-yl)acetic acid

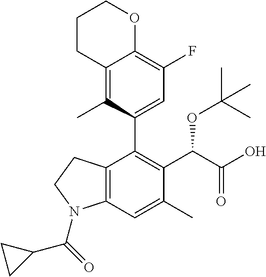

In a manner similar to that described in Example 45, the title compound was prepared after purification by reverse-phase HPLC. ¹H NMR (400 MHz, CDCl₃) δ 9.62 (br, 1H), 8.05 (s, 1H), 6.72 (d, J=11.3 Hz, 1H), 5.03 (s, 1H), 4.32-4.09 (m, 4H), 2.79-2.57 (m, 4H), 2.41 (s, 3H), 2.18-2.07 (m, 2H), 1.93 (s, 3H), 1.77-1.68 (m, 1H), 1.22-0.98 (m, 11H), 0.91-0.85 (m, 2H). LCMS (ES+) (m/z): 496.3 (M+1).

### Example 133: (S)-2-((R)-1-(3,5-Bis(trifluoromethyl)benzoyl)-4-(8-fluoro-5-methylchroman-6-yl)-6-methylindolin-5-yl)-2-(tert-butoxy)acetic acid

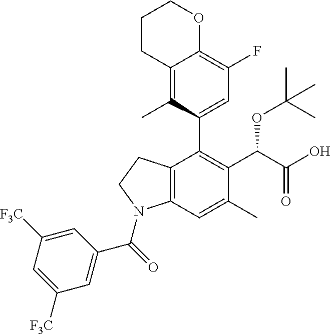

In a manner similar to that described in Example 45, the title compound was prepared after purification by reverse-phase HPLC. ¹H NMR (400 MHz, CDCl₃) δ 9.61 (br, 1H), 8.23-7.85 (m, 4H), 6.71 (d, J=11.1 Hz, 1H), 5.06 (s, 1H), 4.31-4.20 (m, 2H), 4.04-3.83 (m, 2H), 2.81-2.31 (m, 7H), 2.16-2.06 (m, 2H), 1.94 (s, 3H), 1.14 (s, 9H). LCMS (ES+) (m/z): 668.3 (M+1).

### Example 134: (2S)-2-(tert-Butoxy)-2-((4R)-4-(8-fluoro-5-methylchroman-6-yl)-6-methyl-1-(tetrahydrofuran-3-carbonyl)indolin-5-yl)acetic acid

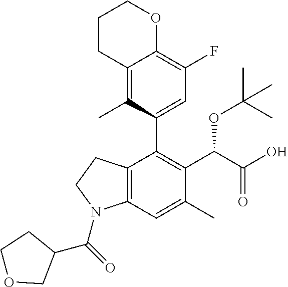

In a manner similar to that described in Example 45, the title compound was prepared after purification by reverse-phase HPLC. ¹H NMR (400 MHz, CDCl₃) δ 9.60 (br, 1H), 8.12 (s, 1H), 6.71 (d, J=11.1 Hz, 1H), 5.04 (s, 1H), 4.26 (t, J=5.2 Hz, 2H), 4.13-3.86 (m, 6H), 3.29-3.21 (m, 1H), 2.80-2.57 (m, 4H), 2.43 (s, 3H), 2.35-2.18 (m, 2H), 2.15-2.07 (m, 2H), 1.93 (s, 3H), 1.12 (s, 9H). LCMS (ES+) (m/z): 526.3 (M+1).

### Example 135: (S)-2-(tert-Butoxy)-2-((R)-1-(2-chloro-3-methylbenzoyl)-4-(8-fluoro-5-methylchroman-6-yl)-6-methylindolin-5-yl)acetic acid

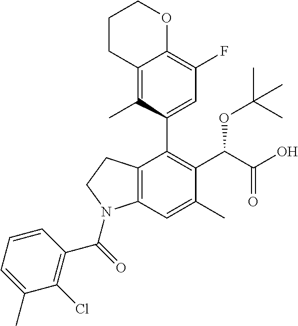

In a manner similar to that described in Example 45, the title compound was prepared after purification by reverse-phase HPLC. ¹H NMR (400 MHz, CDCl₃) δ 9.40 (br, 1H), 8.23 (s, 1H), 7.26-7.16 (m, 3H), 6.73 (d, J=10.5 Hz, 1H), 5.07 (s, 1H), 4.27-4.23 (m, 2H), 3.82-3.63 (m, 2H), 2.72-2.42 (m, 10H), 2.13-2.07 (m, 2H), 1.95 (s, 3H), 1.14 (s, 9H). LCMS (ES+) (m/z): 580.3/582.3 (M/M+2).

### Example 136: (S)-2-(tert-Butoxy)-2-((R)-1-(2-fluoro-4-(trifluoromethyl)benzoyl)-4-(8-fluoro-5-methylchroman-6-yl)-6-methylindolin-5-yl)acetic acid

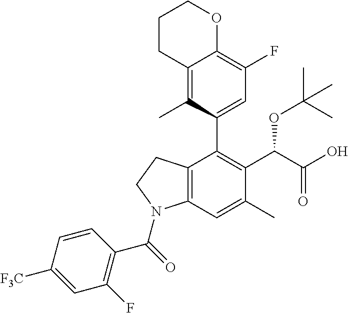

In a manner similar to that described in Example 45, the title compound was prepared after purification by reverse-phase HPLC. ¹H NMR (400 MHz, CDCl₃) δ 9.54 (br, 1H), 8.17 (s, 1H), 7.64-7.58 (m, 1H), 7.58-7.51 (m, 1H), 7.43 (d, J=9.1 Hz, 1H), 6.72 (d, J=11.6 Hz, 1H), 5.07 (s, 1H), 4.29-4.20 (m, 2H), 3.83 (t, J=8.0 Hz, 2H), 2.80-2.36 (m, 7H), 2.15-2.07 (m, 2H), 1.94 (s, 3H), 1.14 (s, 9H). LCMS (ES+) (m/z): 618.4 (M+1).

### Example 137: (S)-2-(tert-Butoxy)-2-((R)-1-(2-chloro-4-methylbenzoyl)-4-(8-fluoro-5-methylchroman-6-yl)-6-methylindolin-5-yl)acetic acid

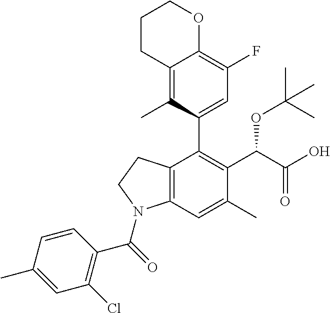

In a manner similar to that described in Example 45, the title compound was prepared after purification by reverse-phase HPLC. ¹H NMR (400 MHz, CDCl₃) δ 9.59 (br, 1H), 8.21 (s, 1H), 7.26-7.20 (m, 2H), 7.15 (d, J=7.8 Hz, 1H), 6.72 (d, J=10.9 Hz, 1H), 5.06 (s, 1H), 4.25 (t, J=5.1 Hz, 2H), 3.83-3.65 (m, 2H), 2.72-2.37 (m, 10H), 2.13-2.07 (m, 2H), 1.95 (s, 3H), 1.14 (s, 9H). LCMS (ES+) (m/z): 580.3/582.3 (M/M+2).

### Example 138: (S)-2-(tert-Butoxy)-2-((R)-1-(4-chloro-2-fluorobenzo yl)-4-(8-fluoro-5-methylchroman-6-yl)-6-methylindolin-5-yl)acetic acid

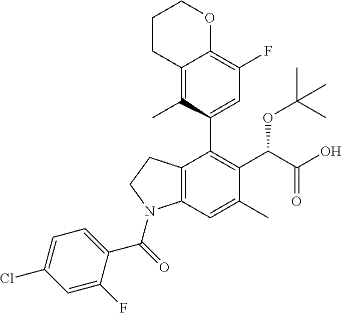

In a manner similar to that described in Example 45, the title compound was prepared after purification by reverse-phase HPLC. ¹H NMR (400 MHz, CDCl₃) δ 9.57 (br, 1H), 8.15 (s, 1H), 7.42 (t, J=7.7 Hz, 1H), 7.26-7.15 (m, 2H), 6.71 (d, J=11.1 Hz, 1H), 5.06 (s, 1H), 4.25 (t, J=5.2 Hz, 2H), 3.83 (t, J=7.8 Hz, 2H), 2.80-2.36 (m, 7H), 2.15-2.07 (m, 2H), 1.94 (s, 3H), 1.14 (s, 9H). LCMS (ES+) (m/z): 584.1/586.2 (M/M+2).

### Example 139: (S)-2-(tert-Butoxy)-2-((R)-1-(2-fluoro-4-methylbenzoyl)-4-(8-fluoro-5-methylchroman-6-yl)-6-methylindolin-5-yl)acetic acid

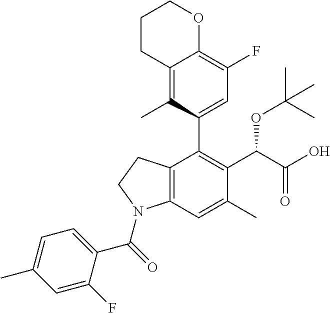

In a manner similar to that described in Example 45, the title compound was prepared after purification by reverse-phase HPLC. ¹H NMR (400 MHz, CDCl₃) δ 9.59 (br, 1H), 8.16 (s, 1H), 7.34 (t, J=7.5 Hz, 1H), 7.07-6.91 (m, 2H), 6.72 (d, J=11.3 Hz, 1H), 5.06 (s, 1H), 4.31-4.20 (m, 2H), 3.94-3.74 (m, 2H), 2.81-2.27 (m, 10H), 2.14-2.07 (m, 2H), 1.94 (s, 3H), 1.13 (s, 9H). LCMS (ES+) (m/z): 564.2 (M+1).

### Example 140: (S)-2-(tert-Butoxy)-2-((R)-1-(3-fluoro-4-(trifluoromethyl)benzoyl)-4-(8-fluoro-5-methylchroman-6-yl)-6-methylindolin-5-yl)acetic acid

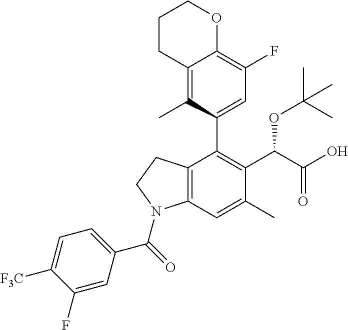

In a manner similar to that described in Example 45, the title compound was prepared after purification by reverse-phase HPLC. ¹H NMR (400 MHz, CDCl₃) δ 9.48 (br, 1H), 8.09 (s, 1H), 7.72 (t, J=7.4 Hz, 1H), 7.49-7.31 (m, 2H), 6.71 (d, J=11.3 Hz, 1H), 5.06 (s, 1H), 4.33-4.16 (m, 2H), 4.04-3.73 (m, 2H), 2.83-2.26 (m, 7H), 2.17-2.05 (m, 2H), 1.94 (s, 3H), 1.14 (s, 9H). LCMS (ES+) (m/z): 618.2 (M+1).

### Example 141: (S)-2-(tert-Butoxy)-2-((R)-1-(3-chloro-5-methylbenzo yl)-4-(8-fluoro-5-methylchroman-6-yl)-6-methylindolin-5-yl)acetic acid

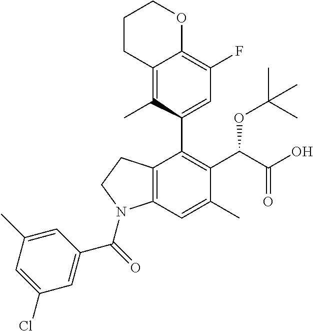

In a manner similar to that described in Example 45, the title compound was prepared after purification by reverse-phase HPLC. ¹H NMR (400 MHz, CDCl₃) δ 9.57 (br, 1H), 8.04 (s, 1H), 7.32-7.26 (m, 2H), 7.22 (s, 1H), 6.71 (d, J=11.2 Hz, 1H), 5.05 (s, 1H), 4.33-4.18 (m, 2H), 4.02-3.84 (m, 2H), 2.81-2.20 (m, 10H), 2.16-2.05 (m, 2H), 1.94 (s, 3H), 1.13 (s, 9H). LCMS (ES+) (m/z): 580.3/582.2 (M/M+2).

### Example 142: (S)-2-(tert-Butoxy)-2-((R)-1-(4-fluoro-3-methoxybenzoyl)-4-(8-fluoro-5-methylchroman-6-yl)-6-methylindolin-5-yl)acetic acid

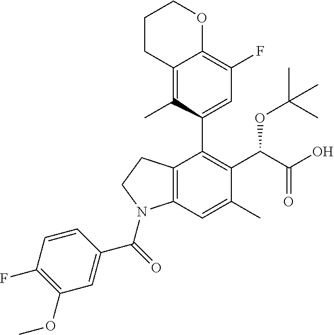

In a manner similar to that described in Example 45, the title compound was prepared after purification by reverse-phase HPLC. ¹H NMR (400 MHz, CDCl₃) δ 9.52 (br, 1H), 7.93 (s, 1H), 7.20 (dd, J=8.1, 1.7 Hz, 1H), 7.16-7.07 (m, 2H), 6.71 (d, J=11.4 Hz, 1H), 5.05 (s, 1H), 4.31-4.21 (m, 2H), 3.99 (t, J=7.9 Hz, 2H), 3.92 (s, 3H), 2.75-2.35 (m, 7H), 2.16-2.06 (m, 2H), 1.94 (s, 3H), 1.13 (s, 9H). LCMS (ES+) (m/z): 580.1 (M+1).

### Example 143: (S)-2-(tert-Butoxy)-2-((R)-1-(3-fluoro-5-(trifluoromethyl)benzoyl)-4-(8-fluoro-5-methylchroman-6-yl)-6-methylindolin-5-yl)acetic acid

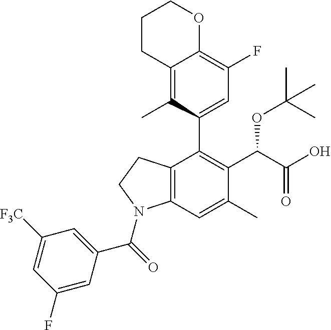

In a manner similar to that described in Example 45, the title compound was prepared after purification by reverse-phase HPLC. ¹H NMR (400 MHz, CDCl₃) δ 9.58 (br, 1H), 8.06 (s, 1H), 7.62 (s, 1H), 7.46 (d, J=8.1 Hz, 2H), 6.71 (d, J=11.2 Hz, 1H), 5.06 (s, 1H), 4.31-4.20 (m, 2H), 4.03-3.85 (m, 2H), 2.76-2.36 (m, 7H), 2.15-2.07 (m, 2H), 1.94 (s, 3H), 1.14 (s, 9H). LCMS (ES+) (m/z): 518.2 (M+1).

### Example 144: (S)-2-(tert-Butoxy)-2-((R)-1-(2-chloro-4-(trifluoromethyl)benzoyl)-4-(8-fluoro-5-methylchroman-6-yl)-6-methylindolin-5-yl)acetic acid

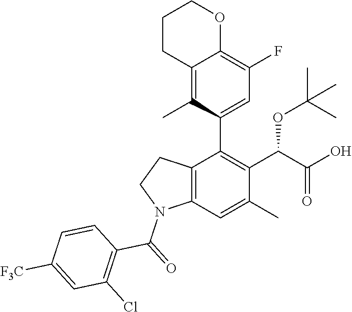

In a manner similar to that described in Example 45, the title compound was prepared after purification by reverse-phase HPLC. ¹H NMR (400 MHz, CDCl₃) δ 9.59 (br, 1H), 8.21 (s, 1H), 7.73 (s, 1H), 7.64 (d, J=8.0 Hz, 1H), 7.52 (d, J=7.9 Hz, 1H), 6.72 (d, J=11.2 Hz, 1H), 5.07 (s, 1H), 4.28-4.23 (m, 2H), 3.83-3.64 (m, 2H), 2.74-2.46 (m, 7H), 2.15-2.08 (m, 2H), 1.95 (s, 3H), 1.14 (s, 9H). LCMS (ES+) (m/z): 634.2/636.2 (M/M+2).

### Example 145: (S)-2-(tert-Butoxy)-2-((R)-1-(5-chloro-2-fluorobenzo yl)-4-(8-fluoro-5-methylchroman-6-yl)-6-methylindolin-5-yl)acetic acid

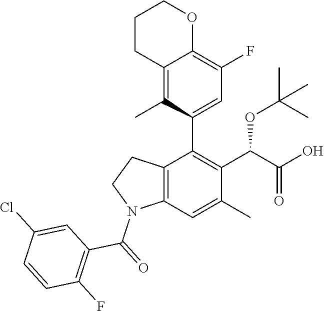

In a manner similar to that described in Example 45, the title compound was prepared after purification by reverse-phase HPLC. ¹H NMR (400 MHz, CDCl₃) δ 9.56 (br, 1H), 8.15 (s, 1H), 7.48-7.34 (m, 2H), 7.10 (t, J=8.8 Hz, 1H), 6.72 (d, J=11.2 Hz, 1H), 5.07 (s, 1H), 4.26 (t, J=5.2 Hz, 2H), 3.85 (t, J=7.7 Hz, 2H), 2.78-2.40 (m, 7H), 2.15-2.07 (m, 2H), 1.94 (s, 3H), 1.14 (s, 9H). LCMS (ES+) (m/z): 583.2/586.2 (M/M+2).

### Example 146: (S)-2-(tert-Butoxy)-2-((R)-4-(8-fluoro-5-methylchroman-6-yl)-6-methyl-1-(4-methyl-3-(trifluoromethyl)benzoyl)indolin-5-yl)acetic acid

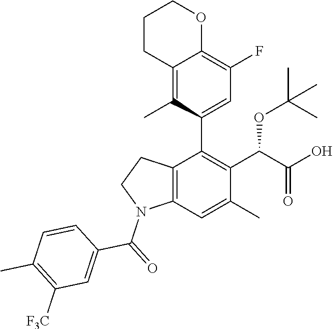

In a manner similar to that described in Example 45, the title compound was prepared after purification by reverse-phase HPLC. ¹H NMR (400 MHz, CDCl₃) δ 9.60 (br, 1H), 8.04 (s, 1H), 7.82 (s, 1H), 7.62 (d, J=7.8 Hz, 1H), 7.39 (d, J=7.9 Hz, 1H), 6.71 (d, J=11.4 Hz, 1H), 5.05 (s, 1H), 4.33-4.18 (m, 2H), 4.06-3.86 (m, 2H), 2.84-2.24 (m, 10H), 2.15-2.05 (m, 2H), 1.94 (s, 3H), 1.13 (s, 9H). LCMS (ES+) (m/z): 614.2 (M+1).

### Example 147: (S)-2-(tert-Butoxy)-2-((R)-1-(3-chloro-5-(trifluoromethyl)benzoyl)-4-(8-fluoro-5-methylchroman-6-yl)-6-methylindolin-5-yl)acetic acid

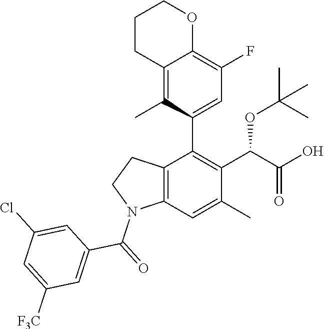

In a manner similar to that described in Example 45, the title compound was prepared after purification by reverse-phase HPLC. ¹H NMR (400 MHz, CDCl₃) δ 9.60 (br, 1H), 8.05 (s, 1H), 7.77-7.62 (m, 3H), 6.71 (d, J=11.3 Hz, 1H), 5.06 (s, 1H), 4.31-4.21 (m, 2H), 4.01-3.84 (m, 2H), 2.75-2.38 (m, 7H), 2.16-2.07 (m, 2H), 1.94 (s, 3H), 1.14 (s, 9H). LCMS (ES+) (m/z): 634.1/636.2 (M/M+2).

### Example 148: (S)-2-(tert-Butoxy)-2-((R)-1-(4-chloro-3-(trifluoromethyl)benzoyl)-4-(8-fluoro-5-methylchroman-6-yl)-6-methylindolin-5-yl)acetic acid

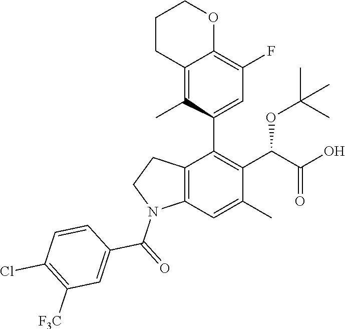

In a manner similar to that described in Example 45, the title compound was prepared after purification by reverse-phase HPLC. ¹H NMR (400 MHz, CDCl₃) δ 9.60 (br, 1H), 8.06 (s, 1H), 7.90 (d, J=1.6 Hz, 1H), 7.68 (dd, J=8.2, 1.7 Hz, 1H), 7.61 (d, J=8.2 Hz, 1H), 6.71 (d, J=11.2 Hz, 1H), 5.06 (s, 1H), 4.37-4.15 (m, 2H), 4.06-3.83 (m, 2H), 2.92-2.20 (m, 7H), 2.18-2.03 (m, 2H), 1.94 (s, 3H), 1.13 (s, 9H). LCMS (ES+) (m/z): 634.1/636.1 (M/M+2).

### Example 149: (S)-2-(tert-Butoxy)-2-((R)-1-(2-chloro-5-fluorobenzo yl)-4-(8-fluoro-5-methylchroman-6-yl)-6-methylindolin-5-yl)acetic acid

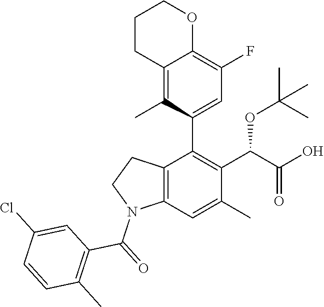

In a manner similar to that described in Example 45, the title compound was prepared after purification by reverse-phase HPLC. ¹H NMR (400 MHz, CDCl₃) δ 9.60 (br, 1H), 8.19 (s, 1H), 7.26-7.16 (m, 3H), 6.72 (d, J=11.3 Hz, 1H), 5.07 (s, 1H), 4.26 (t, J=5.1 Hz, 2H), 3.80-3.47 (m, 2H), 2.84-2.16 (m, 10H), 2.14-2.07 (m, 2H), 1.94 (s, 3H), 1.14 (s, 9H). LCMS (ES+) (m/z): 580.2/582.2 (M/M+2).

### Example 150: (S)-2-(tert-Butoxy)-2-((R)-1-(2-chloro-5-fluorobenzo yl)-4-(8-fluoro-5-methylchroman-6-yl)-6-methylindolin-5-yl)acetic acid

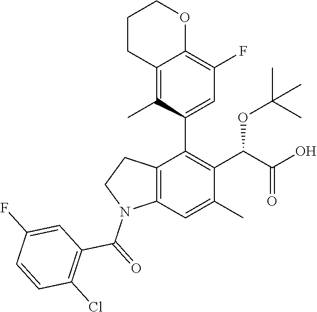

In a manner similar to that described in Example 45, the title compound was prepared after purification by reverse-phase HPLC. ¹H NMR (400 MHz, CDCl₃) δ 9.60 (br, 1H), 8.19 (s, 1H), 7.41 (dd, J=8.7, 4.6 Hz, 1H), 7.16-7.00 (m, 2H), 6.72 (d, J=11.4 Hz, 1H), 5.07 (s, 1H), 4.29-4.22 (m, 2H), 3.87-3.61 (m, 2H), 2.79-2.37 (m, 7H), 2.14-2.07 (m, 2H), 1.95 (s, 3H), 1.14 (s, 9H). LCMS (ES+) (m/z): 584.2/586.2 (M/M+2).

### Example 151: (2S)-2-(tert-Butoxy)-2-(4-(8-fluoro-5-methylchroman-6-yl)-1-(5-methoxynicotinoyl)-6-methylindolin-5-yl)acetic acid

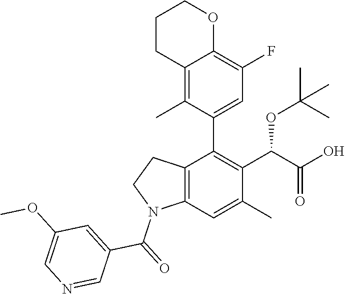 [SMILES: COc1cncc(C(=O)N2CCc3c2cc(C)c([C@H](OC(C)(C)C)C(=O)O)c3-c2cc(F)c3c(c2C)CCCO3)c1]

In a manner similar to that described in Example 45, the title compound was prepared after purification by reverse-phase HPLC. ¹H NMR (400 MHz, CDCl₃) δ 9.69 (br, 1H), 8.49-8.32 (m, 2H), 8.07 (s, 1H), 7.38 (dd, J=2.8, 1.7 Hz, 1H), 6.71 (d, J=11.2 Hz, 1H), 5.06 (s, 1H), 4.31-4.19 (m, 2H), 4.12-3.77 (m, 5H), 2.77-2.33 (m, 7H), 2.17-2.07 (m, 2H), 1.94 (s, 3H), 1.14 (s, 9H). LCMS (ES+) (m/z): 563.0 (M+1).

### Example 152: (S)-2-(tert-Butoxy)-2-((R)-4-(8-fluoro-5-methylchroman-6-yl)-1-(4-methoxy-2-(trifluoromethyl)benzoyl)-6-methylindolin-5-yl)acetic acid

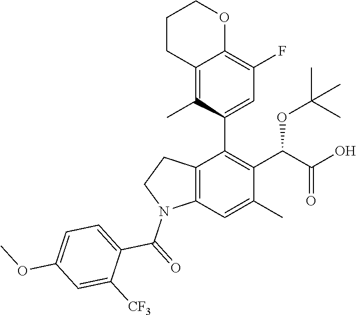

In a manner similar to that described in Example 45, the title compound was prepared after purification by reverse-phase HPLC. ¹H NMR (400 MHz, CDCl₃) δ 9.61 (br, 1H), 8.18 (s, 1H), 7.37-7.33 (m, 1H), 7.22-7.10 (m, 2H), 6.72 (d, J=9.9 Hz, 1H), 5.06 (s, 1H), 4.28-4.23 (m, 2H), 3.88 (s, 3H), 3.65 (t, J=8.4 Hz, 2H), 2.76-2.44 (m, 7H), 2.04-1.90 (m, 5H), 1.14 (s, 9H). LCMS (ES+) (m/z): 630.1 (M+1).

### Example 153: (S)-2-(tert-Butoxy)-2-((R)-4-(8-fluoro-5-methylchroman-6-yl)-6-methyl-1-(3-(methylcarbamoyl)benzoyl)indolin-5-yl)acetic acid

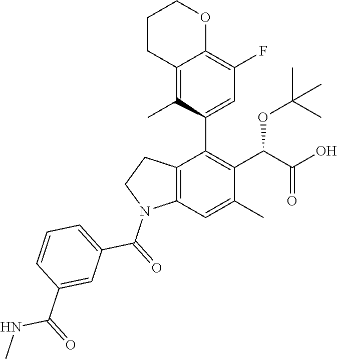

In a manner similar to that described in Example 45, the title compound was prepared after purification by reverse-phase HPLC. ¹H NMR (400 MHz, CDCl₃) δ 9.56 (br, 1H), 8.19-7.82 (m, 3H), 7.67 (d, J=7.7 Hz, 1H), 7.53 (t, J=7.7 Hz, 1H), 6.71 (d, J=11.3 Hz, 1H), 6.20 (d, J=4.7 Hz, 1H), 5.05 (s, 1H), 4.29-4.21 (m, 2H), 4.03-3.88 (m, 2H), 3.03 (d, J=4.9 Hz, 3H), 2.77-2.31 (m, 7H), 2.15-2.06 (m, 2H), 1.94 (s, 3H), 1.13 (s, 9H). LCMS (ES+) (m/z): 589.1 (M+1).

### Example 154: (S)-2-(tert-Butoxy)-2-((R)-1-(2-chloro-5-(trifluoromethyl)benzoyl)-4-(8-fluoro-5-methylchroman-6-yl)-6-methylindolin-5-yl)acetic acid

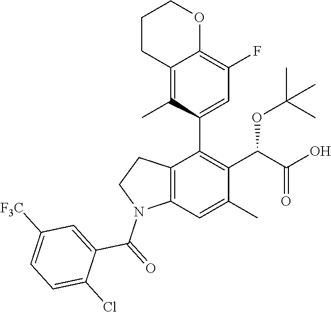

In a manner similar to that described in Example 45, the title compound was prepared after purification by reverse-phase HPLC. ¹H NMR (400 MHz, CDCl₃) δ 9.61 (br, 1H), 8.20 (s, 1H), 7.74-7.57 (m, 3H), 6.72 (d, J=11.7 Hz, 1H), 5.07 (s, 1H), 4.29-4.22 (m, 2H), 3.83-3.63 (m, 2H), 2.79-2.45 (m, 7H), 2.15-2.07 (m, 2H), 1.95 (s, 3H), 1.14 (s, 9H). LCMS (ES+) (m/z): 634.2/636.2 (M/M+2).

### Example 155: (S)-2-(tert-Butoxy)-2-((R)-1-(2-fluoro-5-(trifluoromethyl)benzoyl)-4-(8-fluoro-5-methylchroman-6-yl)-6-methylindolin-5-yl)acetic acid

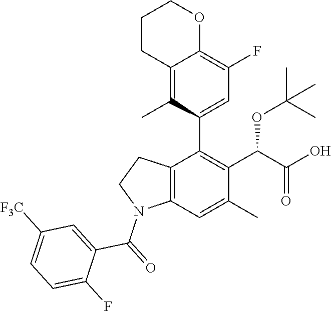

In a manner similar to that described in Example 45, the title compound was prepared after purification by reverse-phase HPLC. ¹H NMR (400 MHz, CDCl₃) δ 9.60 (br, 1H), 8.16 (s, 1H), 7.83-7.67 (m, 2H), 7.29 (d, J=8.7 Hz, 1H), 6.72 (d, J=11.3 Hz, 1H), 5.07 (s, 1H), 4.26 (t, J=5.2 Hz, 2H), 3.84 (t, J=8.2 Hz, 2H), 2.76-2.44 (m, 7H), 2.14-2.07 (m, 2H), 1.94 (s, 3H), 1.14 (s, 9H). LCMS (ES+) (m/z): 618.3 (M+1).

### Example 156: (S)-2-(tert-Butoxy)-2-((R)-1-(4-chloro-2-(trifluoromethyl)benzoyl)-4-(8-fluoro-5-methylchroman-6-yl)-6-methylindolin-5-yl)acetic acid

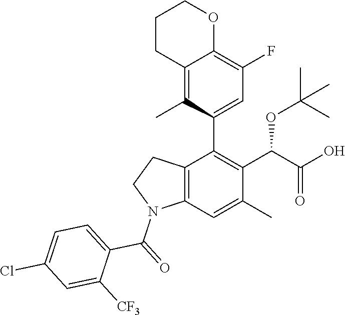

In a manner similar to that described in Example 45, the title compound was prepared after purification by reverse-phase HPLC. ¹H NMR (400 MHz, CDCl₃) δ 9.51 (br, 1H), 8.17 (s, 1H), 7.74 (d, J=1.8 Hz, 1H), 7.62 (dd, J=8.1, 1.9 Hz, 1H), 7.40 (d, J=8.2 Hz, 1H), 6.72 (d, J=10.5 Hz, 1H), 5.07 (s, 1H), 4.28-4.24 (m, 2H), 3.64 (t, J=8.3 Hz, 2H), 2.72-2.48 (m, 7H), 2.13-2.09 (m, 2H), 1.94 (s, 3H), 1.14 (s, 9H). LCMS (ES+) (m/z): 634.1/636.2 (M/M+2).

### Example 157: (S)-2-(tert-Butoxy)-2-((R)-4-(8-fluoro-5-methylchroman-6-yl)-1-(4-methoxy-3-(trifluoromethyl)benzoyl)-6-methylindolin-5-yl)acetic acid

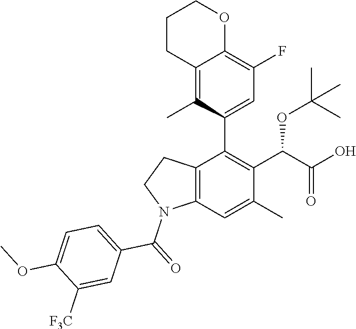

In a manner similar to that described in Example 45, the title compound was prepared after purification by reverse-phase HPLC. ¹H NMR (400 MHz, CDCl₃) δ 9.63 (br, 1H), 7.83 (d, J=1.7 Hz, 1H), 7.77 (dd, J=8.6, 2.0 Hz, 1H), 7.61 (s, 1H), 7.07 (d, J=8.6 Hz, 1H), 6.72 (d, J=11.3 Hz, 1H), 5.05 (s, 1H), 4.29-4.22 (m, 2H), 4.05-3.94 (m, 5H), 2.71-2.37 (m, 7H), 2.14-2.06 (m, 2H), 1.94 (s, 3H), 1.13 (s, 9H). LCMS (ES+) (m/z): 630.2 (M+1).

### Example 158: (S)-2-(tert-Butoxy)-2-((R)-1-(3-fluoro-5-methoxybenzoyl)-4-(8-fluoro-5-methylchroman-6-yl)-6-methylindolin-5-yl) acetic acid

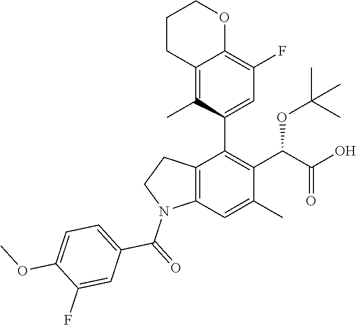

In a manner similar to that described in Example 45, the title compound was prepared after purification by reverse-phase HPLC. ¹H NMR (400 MHz, CDCl₃) δ 9.58 (br, 1H), 8.04 (s, 1H), 6.86 (d, J=1.9 Hz, 1H), 6.82 (dd, J=8.2, 2.2 Hz, 1H), 6.77-6.67 (m, 2H), 5.05 (s, 1H), 4.30-4.23 (m, 2H), 4.02-3.89 (m, 2H), 3.83 (s, 3H), 2.74-2.36 (m, 7H), 2.14-2.07 (m, 2H), 1.94 (s, 3H), 1.13 (s, 9H). LCMS (ES+) (m/z): 580.1 (M+1).

### Example 159: (S)-2-(tert-Butoxy)-2-((R)-4-(8-fluoro-5-methylchroman-6-yl)-1-(2-methoxy-4-methylbenzoyl)-6-methylindolin-5-yl)acetic acid

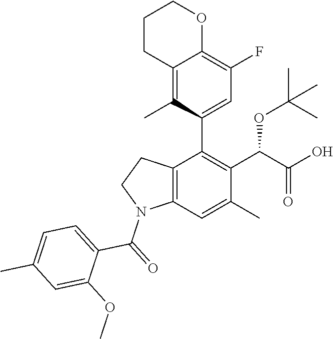

In a manner similar to that described in Example 45, the title compound was prepared after purification by reverse-phase HPLC. ¹H NMR (400 MHz, CDCl₃) δ 9.64 (br, 1H), 8.21 (s, 1H), 7.20 (d, J=7.5 Hz, 1H), 6.83 (d, J=7.4 Hz, 1H), 6.76 (s, 1H), 6.71 (d, J=11.3 Hz, 1H), 5.06 (s, 1H), 4.25 (t, J=5.1 Hz, 2H), 3.89-3.61 (m, 5H), 2.77-2.32 (m, 10H), 2.15-2.08 (m, 2H), 1.94 (s, 3H), 1.13 (s, 9H). LCMS (ES+) (m/z): 576.2 (M+1).

### Example 160: (S)-2-(tert-Butoxy)-2-((R)-1-(2-fluoro-3-(trifluoromethyl)benzoyl)-4-(8-fluoro-5-methylchroman-6-yl)-6-methylindolin-5-yl)acetic acid

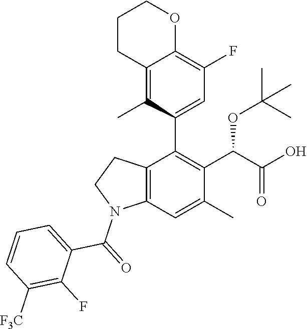

In a manner similar to that described in Example 45, the title compound was prepared after purification by reverse-phase HPLC. ¹H NMR (400 MHz, CDCl₃) δ 9.54 (br, 1H), 8.17 (s, 1H), 7.74-7.65 (m, 2H), 7.37 (t, J=7.7 Hz, 1H), 6.72 (d, J=11.4 Hz, 1H), 5.07 (s, 1H), 4.26 (t, J=5.1 Hz, 2H), 3.83 (t, J=8.0 Hz, 2H), 2.72-2.47 (m, 7H), 2.15-2.08 (m, 2H), 1.94 (s, 3H), 1.14 (s, 9H). LCMS (ES+) (m/z): 618.2 (M+1).

### Example 161: (S)-2-(tert-Butoxy)-2-((R)-1-(cyclobutanecarbonyl)-4-(8-fluoro-5-methylchroman-6-yl)-6-methylindolin-5-yl)acetic acid

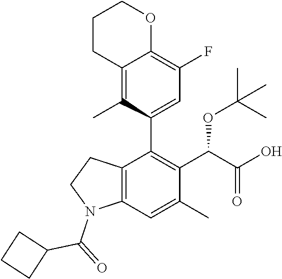

In a manner similar to that described in Example 45, the title compound was prepared after purification by reverse-phase HPLC. ¹H NMR (400 MHz, CDCl₃) δ 9.63 (br, 1H), 8.13 (s, 1H), 6.70 (d, J=11.4 Hz, 1H), 5.03 (s, 1H), 4.29-4.21 (m, 2H), 3.88 (t, J=8.5 Hz, 2H), 3.35-3.25 (m, 1H), 2.76-2.37 (m, 9H), 2.26-1.88 (m, 9H), 1.12 (s, 9H). LCMS (ES+) (m/z): 510.2 (M+1).

### Example 162: (S)-2-(tert-Butoxy)-2-((R)-4-(8-fluoro-5-methylchroman-6-yl)-1-(3-methoxybenzoyl)-6-methylindolin-5-yl)acetic acid

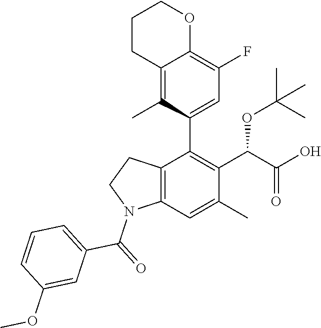

In a manner similar to that described in Example 45, the title compound was prepared after purification by reverse-phase HPLC. ¹H NMR (400 MHz, CDCl₃) δ 9.63 (br, 1H), 8.05 (s, 1H), 7.36 (t, J=7.9 Hz, 1H), 7.09 (dd, J=11.5, 5.0 Hz, 2H), 7.02 (d, J=8.2 Hz, 1H), 6.71 (d, J=11.3 Hz, 1H), 5.05 (s, 1H), 4.32-4.21 (m, 2H), 4.04-3.89 (m, 2H), 3.84 (s, 3H), 2.75-2.31 (m, 7H), 2.16-2.07 (m, 2H), 1.94 (s, 3H), 1.13 (s, 9H). LCMS (ES+) (m/z): 562.1 (M+1).

### Example 163: (S)-2-(tert-Butoxy)-2-((R)-4-(8-fluoro-5-methylchroman-6-yl)-1-(2-methoxy-4-(trifluoromethyl)benzoyl)-6-methylindolin-5-yl)acetic acid

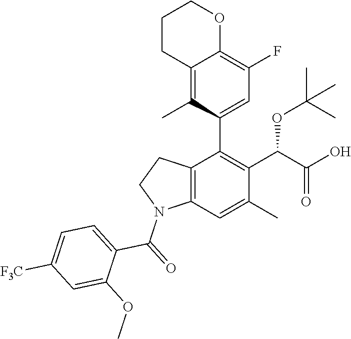

In a manner similar to that described in Example 45, the title compound was prepared after purification by reverse-phase HPLC. ¹H NMR (400 MHz, CDCl₃) δ 9.65 (br, 1H), 8.21 (s, 1H), 7.43 (d, J=7.8 Hz, 1H), 7.31 (d, J=7.6 Hz, 1H), 7.17 (s, 1H), 6.71 (d, J=11.2 Hz, 1H), 5.07 (s, 1H), 4.25 (t, J=5.2 Hz, 2H), 3.91 (s, 3H), 3.83-3.64 (m, 2H), 2.83-2.33 (m, 7H), 2.15-2.06 (m, 2H), 1.95 (s, 3H), 1.14 (s, 9H). LCMS (ES+) (m/z): 630.1 (M+1).

### Example 164: (S)-2-(tert-Butoxy)-2-((R)-4-(8-fluoro-5-methylchroman-6-yl)-6-methyl-1-(3-(piperidine-1-carbonyl)benzoyl)indolin-5-yl)acetic acid

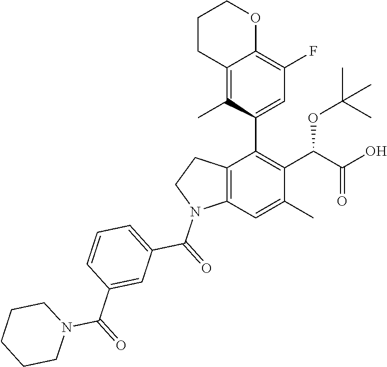

In a manner similar to that described in Example 45, the title compound was prepared after purification by reverse-phase HPLC. ¹H NMR (400 MHz, CDCl₃) δ 9.69 (br, 1H), 8.05 (s, 1H), 7.65-7.44 (m, 4H), 6.71 (d, J=11.4 Hz, 1H), 5.05 (s, 1H), 4.30-4.22 (m, 2H), 4.04-3.90 (m, 2H), 3.77-3.64 (m, 2H), 3.40-3.29 (m, 2H), 2.74-2.35 (m, 7H), 2.14-2.08 (m, 2H), 1.94 (s, 3H), 1.63-1.48 (m, 6H), 1.13 (s, 9H). LCMS (ES+) (m/z): 643.2 (M+1).

### Example 165: (S)-2-(tert-Butoxy)-2-((R)-1-(2-chloro-5-methylbenzoyl)-4-(8-fluoro-5-methylchroman-6-yl)-6-methylindolin-5-yl)acetic acid

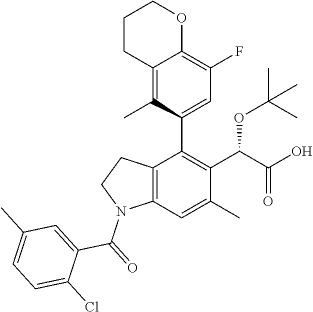

In a manner similar to that described in Example 45, the title compound was prepared after purification by reverse-phase HPLC. ¹H NMR (400 MHz, CDCl₃) δ 9.62 (br, 1H), 8.21 (s, 1H), 7.30 (d, J=8.1 Hz, 1H), 7.25-7.12 (m, 2H), 6.72 (d, J=10.3 Hz, 1H), 5.07 (s, 1H), 4.29-4.21 (m, 2H), 3.89-3.58 (m, 2H), 2.83-2.41 (m, 7H), 2.35 (s, 3H), 2.15-2.06 (m, 2H), 1.95 (s, 3H), 1.14 (s, 9H). LCMS (ES+) (m/z): 580.2/582.2 (M/M+2).

### Example 166: (S)-2-(tert-butoxy)-2-((R)-1-(chromane-8-carbonyl)-4-(8-fluoro-5-methylchroman-6-yl)-6-methylindolin-5-yl)acetic acid

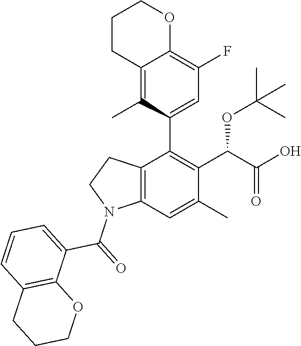

In a manner similar to that described in Example 45, the title compound was prepared after purification by reverse-phase HPLC. ¹H NMR (400 MHz, CDCl₃) δ 9.66 (br, 1H), 8.22 (s, 1H), 7.10 (dd, J=14.8, 7.6 Hz, 2H), 6.89 (d, J=7.2 Hz, 1H), 6.71 (d, J=11.4 Hz, 1H), 5.05 (s, 1H), 4.30-4.17 (m, 4H), 3.89-3.71 (m, 2H), 2.83-2.48 (m, 7H), 2.15-1.90 (m, 9H), 1.13 (s, 9H). LCMS (ES+) (m/z): 588.2 (M+1).

### Example 167: (S)-2-(tert-Butoxy)-2-((R)-1-(2-fluoro-5-methoxybenzoyl)-4-(8-fluoro-5-methylchroman-6-yl)-6-methylindolin-5-yl)acetic acid

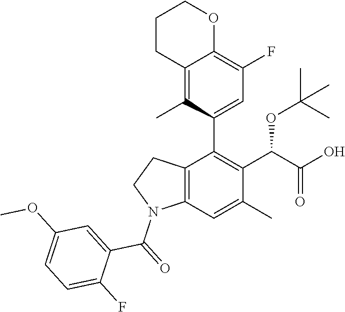

In a manner similar to that described in Example 45, the title compound was prepared after purification by reverse-phase HPLC. ¹H NMR (400 MHz, CDCl₃) δ 9.59 (br, 1H), 8.17 (s, 1H), 7.05 (t, J=9.3 Hz, 1H), 7.00-6.87 (m, 2H), 6.72 (d, J=11.2 Hz, 1H), 5.06 (s, 1H), 4.30-4.21 (m, 2H), 3.98-3.70 (m, 5H), 2.81-2.37 (m, 7H), 2.14-2.06 (m, 2H), 1.94 (s, 3H), 1.14 (s, 9H). LCMS (ES+) (m/z): 580.1 (M+1).

### Example 168: (S)-2-(tert-Butoxy)-2-((R)-4-(8-fluoro-5-methylchroman-6-yl)-1-(4-methoxy-2-methylbenzoyl)-6-methylindolin-5-yl)acetic acid

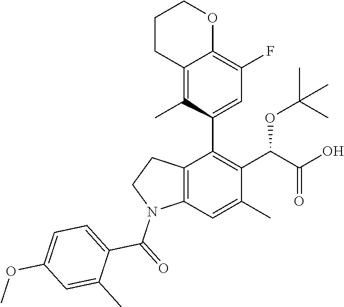

In a manner similar to that described in Example 45, the title compound was prepared after purification by reverse-phase HPLC. ¹H NMR (400 MHz, CDCl₃) δ 9.63 (br, 1H), 8.19 (s, 1H), 7.21 (d, J=8.0 Hz, 1H), 6.89-6.63 (m, 3H), 5.03 (s, 1H), 4.30-4.20 (m, 2H), 4.01-3.58 (m, 5H), 2.76-2.24 (m, 10H), 2.14-2.07 (m, 2H), 1.94 (s, 3H), 1.12 (s, 9H). LCMS (ES+) (m/z): 576.2 (M+1).

### Example 169: (S)-2-(tert-Butoxy)-2-((R)-1-(chromane-5-carbonyl)-4-(8-fluoro-5-methylchroman-6-yl)-6-methylindolin-5-yl)acetic acid

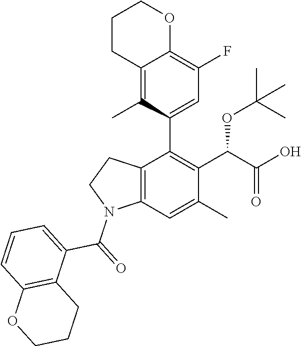

In a manner similar to that described in Example 45, the title compound was prepared after purification by reverse-phase HPLC. ¹H NMR (400 MHz, CDCl₃) δ 9.44 (br, 1H), 8.20 (s, 1H), 7.17-7.09 (m, 1H), 6.91-6.78 (m, 2H), 6.71 (d, J=11.3 Hz, 1H), 5.06 (s, 1H), 4.28-4.18 (m, 4H), 3.79-3.58 (m, 2H), 2.90-2.44 (m, 9H), 2.10-1.89 (m, 7H), 1.14 (s, 9H). LCMS (ES+) (m/z): 588.2 (M+1).

### Example 170: (S)-2-(tert-Butoxy)-2-((R)-4-(8-fluoro-5-methylchroman-6-yl)-1-(6-methoxynicotinoyl)-6-methylindolin-5-yl)acetic acid

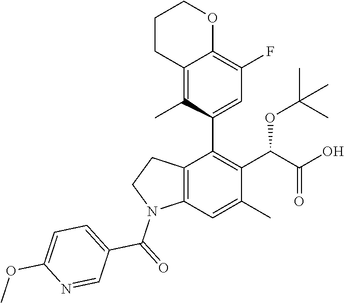

In a manner similar to that described in Example 45, the title compound was prepared after purification by reverse-phase HPLC. ¹H NMR (400 MHz, CDCl₃) δ 9.59 (br, 1H), 8.45 (d, J=2.3 Hz, 1H), 8.00-7.66 (m, 2H), 6.82 (d, J=8.6 Hz, 1H), 6.72 (d, J=11.3 Hz, 1H), 5.06 (s, 1H), 4.31-4.22 (m, 2H), 4.13-3.90 (m, 5H), 2.76-2.39 (m, 7H), 2.16-2.07 (m, 2H), 1.95 (s, 3H), 1.14 (s, 9H). LCMS (ES+) (m/z): 563.0 (M+1).

### Example 171: (S)-2-(tert-Butoxy)-2-((R)-4-(8-fluoro-5-methylchroman-6-yl)-1-(3-methoxyisonicotinoyl)-6-methylindolin-5-yl)acetic acid

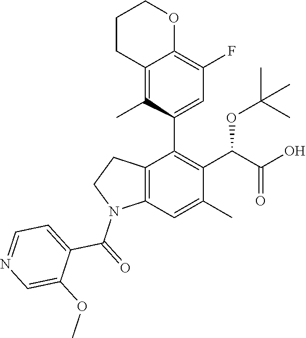

In a manner similar to that described in Example 45, the title compound was prepared after purification by reverse-phase HPLC. ¹H NMR (400 MHz, CDCl₃) δ 8.21 (d, J=5.1 Hz, 1H), 8.04 (s, 1H), 6.90 (dd, J=5.2, 1.3 Hz, 1H), 6.77 (s, 1H), 6.64 (d, J=11.3 Hz, 1H), 4.99 (s, 1H), 4.19 (t, J=5.1 Hz, 2H), 4.06-3.54 (m, 5H), 2.77-2.18 (m, 7H), 2.09-2.00 (m, 2H), 1.86 (s, 3H), 1.06 (s, 9H). (the acidic proton was not observed). LCMS (ES+) (m/z): 563.3 (M+1).

### Example 172: (S)-2-(tert-butoxy)-2-((R)-4-(8-fluoro-5-methylchroman-6-yl)-6-methyl-1-(6-(trifluoromethyl)nicotinoyl)indolin-5-yl)acetic acid

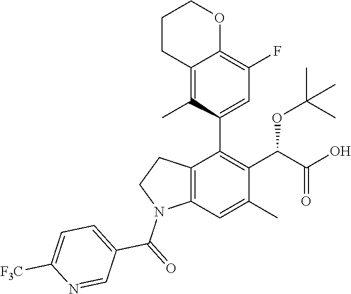

In a manner similar to that described in Example 45, the title compound was prepared after purification by reverse-phase HPLC. ¹H NMR (400 MHz, CDCl₃) δ 8.92 (d, J=1.5 Hz, 1H), 8.21-7.96 (m, 2H), 7.81 (d, J=8.0 Hz, 1H), 6.71 (d, J=11.3 Hz, 1H), 5.07 (s, 1H), 4.33-4.18 (m, 2H), 4.09-3.82 (m, 2H), 2.82-2.29 (m, 7H), 2.16-2.05 (m, 2H), 1.94 (s, 3H), 1.14 (s, 9H). (the acidic proton was not observed). LCMS (ES+) (m/z): 601.1 (M+1).

### Example 173: (S)-2-(tert-butoxy)-2-((R)-4-(8-fluoro-5-methylchroman-6-yl)-6-methyl-1-(2-methylnicotinoyl)indolin-5-yl)acetic acid

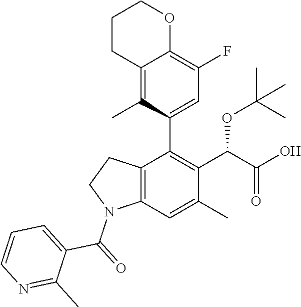

In a manner similar to that described in Example 45, the title compound was prepared after purification by reverse-phase HPLC. ¹H NMR (400 MHz, CDCl₃) δ 9.65 (br, 1H), 8.58 (d, J=4.2 Hz, 1H), 8.21 (s, 1H), 7.59 (d, J=7.9 Hz, 1H), 7.25-7.15 (m, 1H), 6.72 (d, J=11.1 Hz, 1H), 5.07 (s, 1H), 4.29-4.21 (m, 2H), 3.77-3.55 (m, 2H), 2.72-2.49 (m, 7H), 2.14-2.06 (m, 2H), 1.95 (s, 3H), 1.55 (s, 3H), 1.15 (s, 9H). LCMS (ES+) (m/z): 547.0 (M+1).

### Example 174: (S)-2-(tert-Butoxy)-2-((R)-1-(3,4-Difluoro-5-methoxybenzoyl)-4-(8-fluoro-5-methylchroman-6-yl)-6-methylindolin-5-yl)acetic acid

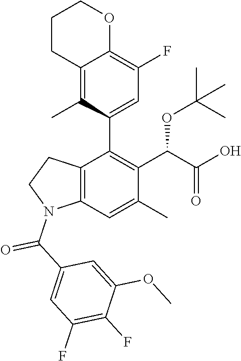

In a manner similar to that described in Example 3, (S)-2-(tert-butoxy)-2-((R)-1-(3,4-difluoro-5-methoxybenzoyl)-4-(8-fluoro-5-methylchroman-6-yl)-6-methylindolin-5-yl)acetic acid was prepared as a white solid in 6% yield, impure with ˜16% of the (P,S)-diastereomer. ¹H NMR (400 MHz, METHANOL-d₄) δ ppm 8.15-7.75 (m, 1H), 7.22-7.08 (m, 2H), 7.05-6.61 (m, 1H), 5.09-4.98 (m, 1H), 4.22 (t, J=4.9 Hz, 2H), 4.08-3.97 (m, 2H), 3.94 (s, 3H), 2.80-2.34 (m, 7H), 2.18-2.04 (m, 2H), 1.90 (s, 3H), 1.17-0.94 (m, 9H); LCMS (m/z) ES⁺=598.5 (M+1), ES⁻=596.4 (M−1).

### Example 175: (S)-2-(tert-Butoxy)-2-((R)-4-(8-fluoro-5-methylchroman-6-yl)-6-methyl-1-(2-oxo-2-phenylacetyl)indolin-5-yl)acetic acid

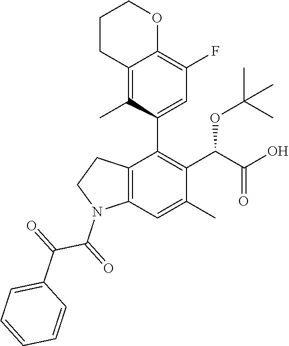

In a manner similar to that described in Example 3, (S)-2-(tert-butoxy)-2-((R)-4-(8-fluoro-5-methylchroman-6-yl)-6-methyl-1-(2-oxo-2-phenylacetyl)indolin-5-yl)acetic acid was prepared as a white solid in 7.6% yield, impure with ˜30% of the (P,S)-diastereomer. ¹H NMR (400 MHz, METHANOL-d₄) (mixture of atropisomers) 8 ppm 8.13-7.93 (m, 3H), 7.81-7.54 (m, 3H), 6.68 (d, J=11.0 Hz, 1H), 5.10-4.99 (m, 1H), 4.21 (t, J=5.0 Hz, 2H), 3.96 (t, J=8.2 Hz, 2H), 2.85-2.47 (m, 7H), 2.16-2.01 (m, 2H), 1.97-1.81 (m, 3H), 1.15-0.97 (m, 9H); LCMS (m/z) ES⁺=560.3 (M+1), ES⁻=558.3 (M−1).

### Example: 176: (S)-2-(tert-Butoxy)-2-((R)-4-(8-Fluoro-5-methylchroman-6-yl)-1-(3-methoxy-4-(trifluoromethyl)benzoyl)-6-methylindolin-5-yl)acetic acid

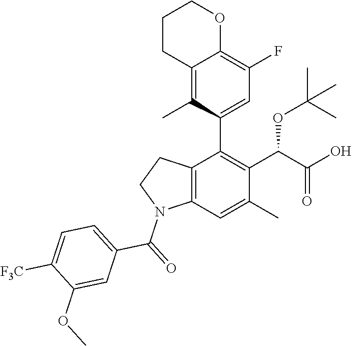

In a manner similar to that described in Example 3, (S)-2-(tert-butoxy)-2-((R)-4-(8-fluoro-5-methylchroman-6-yl)-1-(3-methoxy-4-(trifluoromethyl)benzoyl)-6-methylindolin-5-yl)acetic acid was prepared as a white solid in 17% yield, impure with ˜30% of the (P,S)-diastereomer. ¹H NMR (400 MHz, METHANOL-d₄) (mixture of atropisomers) 8 ppm 8.03 (br. s., 1H), 7.70 (d, J=7.7 Hz, 1H), 7.37 (s, 1H), 7.24 (d, J=7.7 Hz, 1H), 7.07-6.57 (m, 1H), 5.12-4.97 (m, 1H), 4.22 (t, J=4.9 Hz, 2H), 4.11-3.80 (m, 5H), 2.88-2.37 (m, 7H), 2.19-2.03 (m, 2H), 1.90 (s, 3H), 1.18-0.94 (m, 9H); LCMS (m/z) ES⁺=630.4 (M+1), ES−=628.5 (M−1).

### Example 177: (S)-2-(tert-Butoxy)-2-((R)-1-(2,3-dihydrobenzofuran-4-carbonyl)-4-(8-fluoro-5-methylchroman-6-yl)-6-methylindolin-5-yl)acetic acid

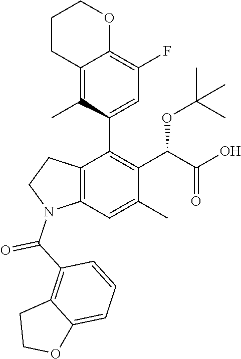

In a manner similar to that described in Example 3, (S)-2-(tert-butoxy)-2-((R)-1-(2,3-dihydrobenzofuran-4-carbonyl)-4-(8-fluoro-5-methylchroman-6-yl)-6-methylindolin-5-yl)acetic acid was prepared as a white solid in 16% yield, impure with ˜10% of the (P,S)-diastereomer. ¹H NMR (400 MHz, METHANOL-d₄) δ ppm 8.02 (br. s., 1H), 7.23 (br. s., 1H), 7.06-6.77 (m, 2H), 6.66 (d, J=11.4 Hz, 1H), 4.99 (br. s., 1H), 4.57 (t, J=8.2 Hz, 2H), 4.22 (t, J=4.9 Hz, 2H), 4.11-3.75 (m, 2H), 3.28-3.08 (m, 2H), 2.79-2.32 (m, 7H), 2.18-2.03 (m, 2H), 1.90 (s, 3H), 1.09 (s, 9H); LCMS (m/z) ES⁺=574.4 (M+1), ES⁻=572.4 (M−1).

### Example 178: (S)-2-(tert-Butoxy)-2-((R)-1-(2,3-Dihydrobenzofuran-6-carbonyl)-4-(8-fluoro-5-methylchroman-6-yl)-6-methylindolin-5-yl)acetic acid

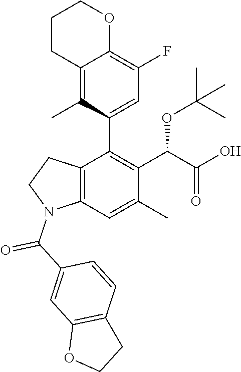

In a manner similar to that described in Example 3, (S)-2-(tert-butoxy)-2-((R)-1-(2,3-dihydrobenzofuran-6-carbonyl)-4-(8-fluoro-5-methylchroman-6-yl)-6-methylindolin-5-yl)acetic acid was prepared as a white solid in 9% yield, impure with ˜30% of the (P,S)-diastereomer. ¹H NMR (400 MHz, METHANOL-d₄) δ ppm 7.94 (br. s., 1H), 7.32 (d, J=7.5 Hz, 1H), 7.03 (d, J=7.3 Hz, 1H), 6.91 (s, 1H), 6.67 (d, J=11.4 Hz, 1H), 5.12-4.94 (m, 1H), 4.60 (t, J=8.6 Hz, 2H), 4.21 (t, J=5.0 Hz, 2H), 3.98 (br. s., 2H), 3.29-3.19 (m, 2H), 2.83-2.21 (m, 7H), 2.18-2.00 (m, 2H), 1.90 (s, 3H), 1.17-0.96 (m, 9H); LCMS (m/z) ES⁺=574.4 (M+1), ES⁻=572.3 (M−1).

### Example 179: (S)-2-(tert-Butoxy)-2-((R)-4-(8-Fluoro-5-methylchroman-6-yl)-6-methyl-1-(2-methylbenzo[d]oxazole-7-carbonyl)indolin-5-yl)acetic acid

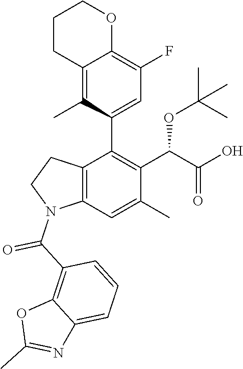

In a manner similar to that described in Example 3, (S)-2-(tert-butoxy)-2-((R)-4-(8-fluoro-5-methylchroman-6-yl)-6-methyl-1-(2-methylbenzo[d]oxazole-7-carbonyl)indolin-5-yl)acetic acid was prepared as a white solid in 27% yield, impure with ˜10% of the (P,S)-diastereomer. ¹H NMR (400 MHz, METHANOL-d₄) δ ppm 8.06 (br. s., 1H), 7.77 (br. s., 1H), 7.61-7.32 (m, 2H), 6.68 (d, J=11.4 Hz, 1H), 5.02 (br. s., 1H), 4.21 (br. s., 2H), 4.05-3.72 (m, 2H), 2.93-2.36 (m, 10H), 2.18-2.01 (m, 2H), 1.90 (s, 3H), 1.19-0.96 (m, 9H); LCMS (m/z) ES=587.4 (M+1), ES⁻=585.3 (M−1).

### Example 180: (S)-2-(tert-Butoxy)-2-((R)-4-(8-Fluoro-5-methylchroman-6-yl)-1-(2-methoxy-3-(trifluoromethyl)benzoyl)-6-methylindolin-5-yl)acetic acid

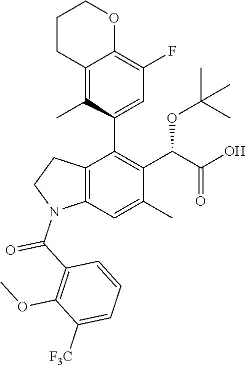

In a manner similar to that described in Example 3, (S)-2-(tert-butoxy)-2-((R)-4-(8-fluoro-5-methylchroman-6-yl)-1-(2-methoxy-3-(trifluoromethyl)benzoyl)-6-methylindolin-5-yl)acetic acid was prepared as a white solid in 14% yield. ¹H NMR (400 MHz, METHANOL-d₄) δ ppm 8.11 (s, 1H), 7.95-7.56 (m, 2H), 7.52-7.28 (m, 1H), 6.76-6.57 (m, 1H), 5.11-4.97 (m, 1H), 4.27-4.15 (m, 2H), 4.02-3.70 (m, 5H), 2.83-2.45 (m, 7H), 2.16-2.03 (m, 2H), 1.95-1.81 (m, 3H), 1.16-1.03 (m, 9H); LCMS (m/z) ES⁻=630.4 (M+1), ES⁻=628.3 (M−1).

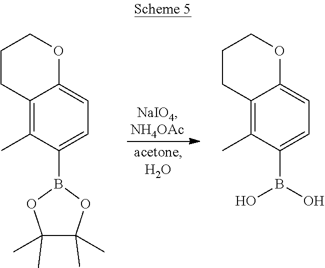 [SMILES: Cc1c(B2OC(C)(C)C(C)(C)O2)ccc2c1CCCO2 ; Cc1c(B(O)O)ccc2c1CCCO2]

### Example 181: (S)-2-(tert-Butoxy)-2-((R)-1-(3-fluoro-2-methoxybenzoyl)-6-methyl-4-(5-methylchroman-6-yl)indolin-5-yl)acetic acid

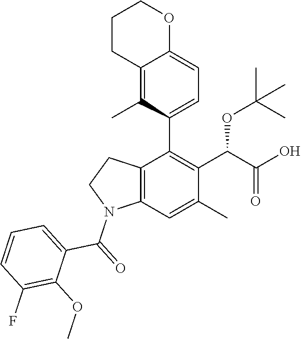

### Step 1: (5-Methylchroman-6-yl)boronic acid

A mixture of 4,4,5,5-tetramethyl-2-(5-methylchroman-6-yl)-1,3,2-dioxaborolane (200 mg, 0.73 mmol) (PCT Int. Appl., 2014053665, 10 Apr. 2014), AcONH₄ (225 mg, 2.9 mmol) and NaIO₄ (468 mg, 2.2 mmol) in acetone (2 mL) and water (1 mL) was stirred at 40° C. for 2 days. The resulting mixture was partitioned between water and EtOAc. The organic layer was washed with brine, dried over Na₂SO₄, filtered and concentrated under reduced pressure to give the crude product which was triturated with 10% EtOAc in PE to afford the title compound (69 mg, 49% yield) as a white solid. LCMS (ES+) (m/z): 193.3 (M+1).

### Step 2: (S)-2-(tert-Butoxy)-2-((R)-1-(3-fluoro-2-methoxybenzoyl)-6-methyl-4-(5-methylchroman-6-yl)indolin-5-yl)acetic acid

In a manner similar to that described in Example 45, the title compound was prepared after purification by reverse phase HPLC. ¹H NMR (400 MHz, CDCl₃) δ 9.57 (br, 1H), 8.19 (s, 1H), 7.18-7.06 (m, 3H), 6.83 (d, J=8.4 Hz, 1H), 6.69 (d, J=8.3 Hz, 1H), 5.10 (s, 1H), 4.17 (t, J=5.1 Hz, 2H), 3.99 (d, J=1.7 Hz, 3H), 3.83-3.64 (m, 2H), 2.73-2.43 (m, 7H), 2.10-1.97 (m, 5H), 1.12 (s, 9H). LCMS (ES+) (m/z): 562.3 (M+1).

### Example 182: (S)-2-(tert-Butoxy)-2-((R)-1-(2,3-dihydrobenzo[b][1,4]dioxine-5-carbonyl)-6-methyl-4-(5-methylchroman-6-yl)indolin-5-yl)acetic acid

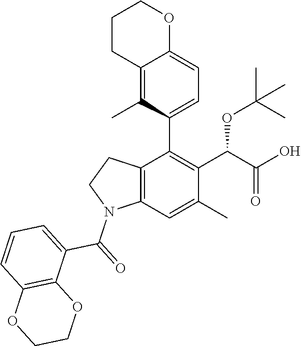

In a manner similar to that described in Example 181, the title compound was isolated after reverse-phase HPLC. ¹H NMR (400 MHz, CDCl₃) δ 9.71 (br, 1H), 8.21 (s, 1H), 7.03-6.87 (m, 3H), 6.84 (d, J=7.9 Hz, 1H), 6.69 (d, J=8.2 Hz, 1H), 5.10 (s, 1H), 4.38-4.06 (m, 6H), 3.91-3.73 (m, 2H), 2.80-2.38 (m, 7H), 2.13-1.98 (m, 5H), 1.12 (s, 9H). LCMS (ES+) (m/z): 572.2 (M+1).

### Example 183: (S)-2-(tert-Butoxy)-2-((R)-1-(4-chloro-2-methoxybenzoyl)-6-methyl-4-(5-methylchroman-6-yl)indolin-5-yl)acetic acid

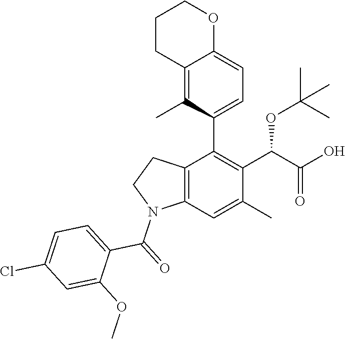

In a manner similar to that described in Example 181, the title compound was isolated after reverse-phase HPLC. ¹H NMR (400 MHz, CDCl₃) δ 9.61 (br, 1H), 8.18 (s, 1H), 7.26-7.23 (m, 1H), 7.01 (d, J=8.1 Hz, 1H), 6.95 (s, 1H), 6.83 (d, J=8.3 Hz, 1H), 6.68 (d, J=8.3 Hz, 1H), 5.10 (s, 1H), 4.17 (t, J=5.1 Hz, 2H), 3.91-3.63 (m, 5H), 2.79-2.38 (m, 7H), 2.12-1.96 (m, 5H), 1.12 (s, 9H). LCMS (ES+) (m/z): 578.2/580.1 (M/M+2).

### Example 184: (S)-2-(tert-Butoxy)-2-((R)-1-(3-methoxy-4-methylbenzo yl)-6-methyl-4-(5-methylchroman-6-yl)indolin-5-yl)acetic acid

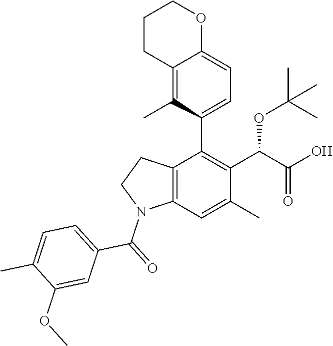

In a manner similar to that described in Example 181, the title compound was isolated after reverse-phase HPLC. ¹H NMR (400 MHz, CDCl₃) δ 9.51 (br, 1H), 7.92 (s, 1H), 7.17 (d, J=8.2 Hz, 1H), 7.07-6.97 (m, 2H), 6.83 (d, J=8.2 Hz, 1H), 6.69 (d, J=8.3 Hz, 1H), 5.08 (s, 1H), 4.17 (t, J=5.2 Hz, 2H), 3.99 (t, J=8.2 Hz, 2H), 3.85 (s, 3H), 2.72-2.23 (m, 10H), 2.15-1.94 (m, 5H), 1.11 (s, 9H). LCMS (ES+) (m/z): 558.2 (M+1).

### Example 185: (S)-2-(tert-Butoxy)-2-((R)-1-(2,4-dimethoxybenzo yl)-6-methyl-4-(5-methylchroman-6-yl)indolin-5-yl)acetic acid

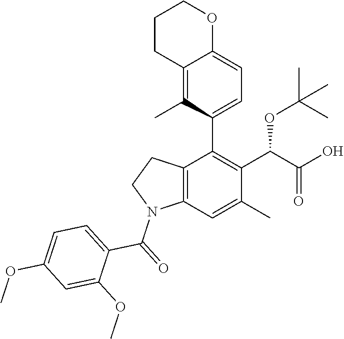

In a manner similar to that described in Example 181, the title compound was isolated after reverse-phase HPLC. ¹H NMR (400 MHz, CDCl₃) δ 9.64 (br, 1H), 8.18 (s, 1H), 7.32-7.26 (m, 1H), 6.83 (d, J=8.1 Hz, 1H), 6.68 (d, J=8.3 Hz, 1H), 6.54 (d, J=7.6 Hz, 1H), 6.49 (s, 1H), 5.09 (s, 1H), 4.17 (t, J=5.1 Hz, 2H), 3.92-3.67 (m, 8H), 2.74-2.42 (m, 7H), 2.09-1.97 (m, 5H), 1.11 (s, 9H). LCMS (ES+) (m/z): 574.3 (M+1).

### Example 186: (S)-2-(tert-Butoxy)-2-((R)-1-(3,4-dimethoxybenzo yl)-6-methyl-4-(5-methylchroman-6-yl)indolin-5-yl)acetic acid

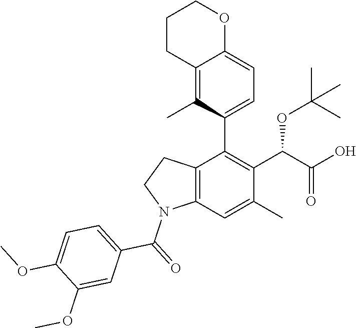

In a manner similar to that described in Example 181, the title compound was isolated after reverse-phase HPLC. ¹H NMR (400 MHz, CDCl₃) δ 9.60 (br, 1H), 7.52 (s, 1H), 7.21-7.12 (m, 2H), 6.90 (d, J=8.2 Hz, 1H), 6.84 (d, J=8.3 Hz, 1H), 6.69 (d, J=8.3 Hz, 1H), 5.08 (s, 1H), 4.17 (t, J=5.2 Hz, 2H), 4.02 (t, J=8.2 Hz, 2H), 3.97-3.85 (m, 6H), 2.74-2.33 (m, 7H), 2.13-1.96 (m, 5H), 1.11 (s, 9H). LCMS (ES+) (m/z): 574.2 (M+1).

### Example 187: (S)-2-(tert-Butoxy)-2-((R)-1-(3-fluoro-4-methoxybenzoyl)-6-methyl-4-(5-methylchroman-6-yl)indolin-5-yl)acetic acid

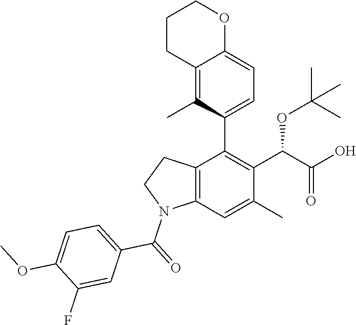

In a manner similar to that described in Example 181, the title compound was isolated after reverse-phase HPLC. ¹H NMR (400 MHz, CDCl₃) δ 9.61 (br, 1H), 7.68 (s, 1H), 7.39-7.29 (m, 2H), 7.01 (t, J=8.5 Hz, 1H), 6.83 (d, J=8.1 Hz, 1H), 6.69 (d, J=8.3 Hz, 1H), 5.08 (s, 1H), 4.17 (t, J=5.2 Hz, 2H), 3.99 (t, J=8.2 Hz, 2H), 3.95 (s, 3H), 2.74-2.30 (m, 7H), 2.14-1.91 (m, 5H), 1.11 (s, 9H). LCMS (ES+) (m/z): 562.2 (M+1).

### Example 188: (S)-2-(tert-Butoxy)-2-((R)-6-methyl-1-(2-methylbenzoyl)-4-(5-methylchroman-6-yl)indolin-5-yl)acetic acid

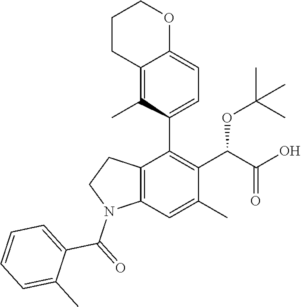

In a manner similar to that described in Example 181, the title compound was isolated after reverse-phase HPLC. ¹H NMR (400 MHz, CDCl₃) δ 9.61 (br, 1H), 8.21 (s, 1H), 7.52-7.26 (m, 4H), 6.84 (d, J=8.0 Hz, 1H), 6.69 (d, J=8.3 Hz, 1H), 5.10 (s, 1H), 4.17 (t, J=5.1 Hz, 2H), 3.77-3.51 (m, 2H), 2.78-2.22 (m, 10H), 2.10-1.96 (m, 5H), 1.12 (s, 9H). LCMS (ES+) (m/z): 528.2 (M+1).

### Example 189: (S)-2-(tert-Butoxy)-2-((R)-1-(3,5-difluorobenzoyl)-6-methyl-4-(5-methylchroman-6-yl)indolin-5-yl)acetic acid

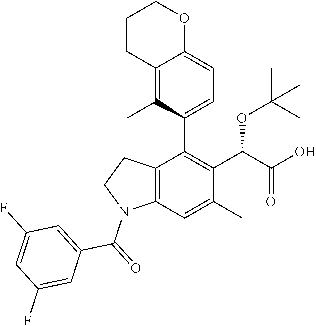

In a manner similar to that described in Example 181, the title compound was isolated after reverse-phase HPLC. ¹H NMR (400 MHz, CDCl₃) δ 9.60 (br, 1H), 8.05 (s, 1H), 7.07 (dd, J=7.2, 2.3 Hz, 2H), 6.93 (t, J=8.7 Hz, 1H), 6.83 (d, J=8.4 Hz, 1H), 6.70 (d, J=8.3 Hz, 1H), 5.09 (s, 1H), 4.18 (t, J=5.2 Hz, 2H), 4.01-3.86 (m, 2H), 2.78-2.35 (m, 7H), 2.11-1.93 (m, 5H), 1.12 (s, 9H). LCMS (ES+) (m/z): 550.2 (M+1).

### Example 190: (S)-2-(tert-Butoxy)-2-((R)-1-(4-methoxy-3-methylbenzo yl)-6-methyl-4-(5-methylchroman-6-yl)indolin-5-yl)acetic acid

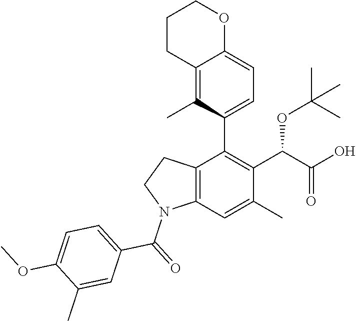

In a manner similar to that described in Example 181, the title compound was isolated after reverse-phase HPLC. ¹H NMR (400 MHz, CDCl₃) δ 9.63 (br, 1H), 7.61 (s, 1H), 7.43-7.37 (m, 2H), 6.89-6.81 (m, 2H), 6.69 (d, J=8.3 Hz, 1H), 5.08 (s, 1H), 4.17 (t, J=5.2 Hz, 2H), 4.00 (t, J=8.3 Hz, 2H), 3.88 (s, 3H), 2.70-2.37 (m, 7H), 2.24 (s, 3H), 2.12-2.04 (m, 2H), 1.98 (s, 3H), 1.11 (s, 9H). LCMS (ES+) (m/z): 558.2 (M+1).

### Example 191: (S)-2-(tert-Butoxy)-2-((R)-6-methyl-4-(5-methylchroman-6-yl)-1-(6-methylnicotinoyl)indolin-5-yl)acetic acid

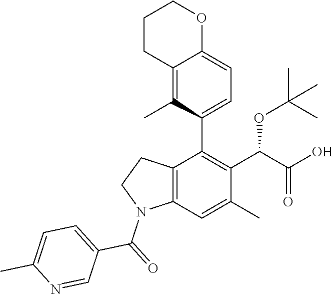

In a manner similar to that described in Example 181, the title compound was isolated after reverse-phase HPLC. ¹H NMR (400 MHz, CDCl₃) δ 9.67 (br, 1H), 8.72 (d, J=1.8 Hz, 1H), 8.03 (s, 1H), 7.81 (d, J=5.9 Hz, 1H), 7.30-7.27 (m, 1H), 6.83 (d, J=8.0 Hz, 1H), 6.70 (d, J=8.4 Hz, 1H), 5.09 (s, 1H), 4.17 (t, J=5.2 Hz, 2H), 4.06-3.93 (m, 2H), 2.80-2.29 (m, 10H), 2.08 (d, J=3.2 Hz, 2H), 1.98 (s, 3H), 1.11 (s, 9H). LCMS (ES+) (m/z): 529.2 (M+1).

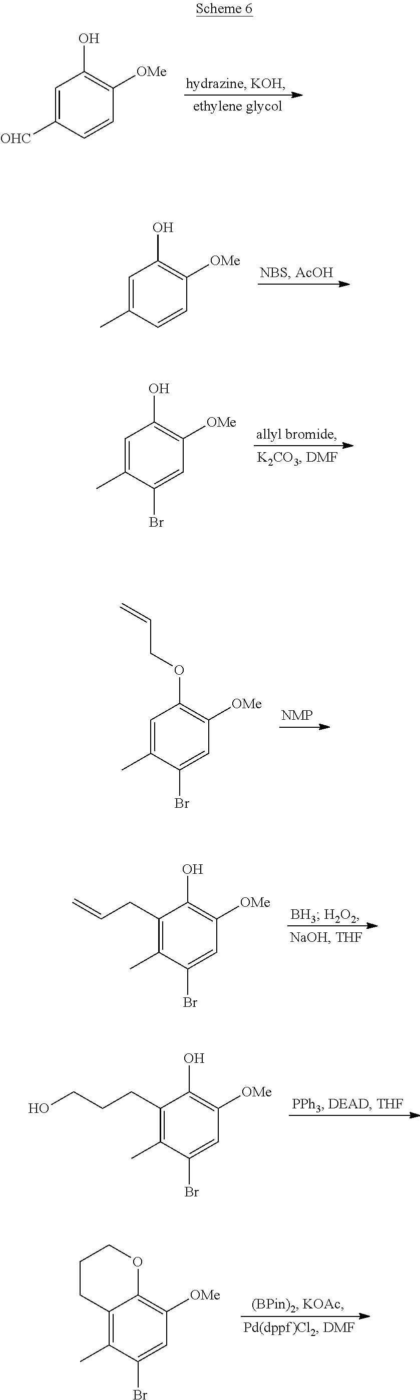

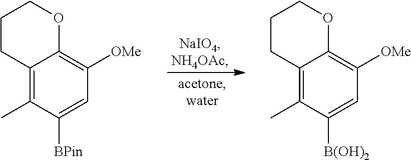

### Example 192: (S)-2-(tert-Butoxy)-2-((R)-1-(4-methoxy-3-methylbenzoyl)-4-(8-methoxy-5-methylchroman-6-yl)-6-methylindolin-5-yl)acetic acid

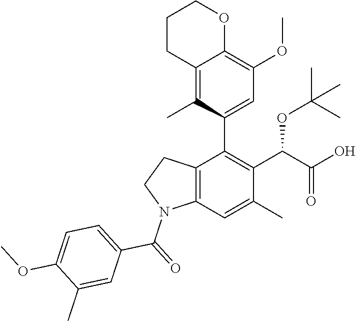

### Step 1: 2-Methoxy-5-methylphenol

To a suspension of 3-hydroxy-4-methoxybenzaldehyde (5 g, 33 mmol) and KOH (18.4 g, 328 mmol) in ethylene glycol (65 mL) was added hydrazine mono hydrate (9.7 g, 194 mmol). The reaction mixture was heated at 130° C. for 1 hr and then at 190° C. for 5 hr.

After cooled down to r.t., the resulting mixture was poured into a mixture of conc. HCl (42 mL) and ice water (150 mL) and extracted with ether. The organic layer was washed with brine, dried over Na₂SO₄, filtered and concentrated under reduced pressure to give the title compound (4.8 g, quant. yield) as a white solid. LCMS (ES+) (m/z): 139.0 (M+1). ¹H NMR (400 MHz, CDCl₃) δ 6.79-6.71 (m, 2H), 6.67-6.61 (m, 1H), 5.56 (s, 1H), 3.85 (s, 3H), 2.26 (s, 3H).

### Step 2: 4-Bromo-2-methoxy-5-methylphenol

To a solution of 2-methoxy-5-methylphenol (2.4 g, 17.8 mmol) in AcOH (100 mL) was added NBS (3.3 g, 18.5 mmol) portionwise over 20 min. After stirred at r.t. for 4 hr, the resulting mixture was concentrated under reduced pressure and the residue was diluted with water, neutralized with 6N NaOH and extracted with DCM. The organic layer was washed with brine, dried over Na₂SO₄, filtered and concentrated under reduced pressure to give the crude product which was purified by flash chromatography (silica gel, 0-40% EtOAc in PE) to afford the title compound (3.8 g, 99% yield) as a white solid. LCMS (ES−) (m/z): 214.9/216.9 (M−2/M). ¹H NMR (400 MHz, CDCl₃) δ 6.99 (s, 1H), 6.81 (s, 1H), 5.45 (s, 1H), 3.85 (s, 3H), 2.28 (s, 3H).

### Step 3: 1-Bromo-5-methoxy-2-methyl-4-(prop-2-yn-1-yloxy)benzene

To a mixture of 4-bromo-2-methoxy-5-methylphenol (3.8 g, 17.7 mmol) and K₂CO₃ (4.9 g, 35.5 mmol) in DMF (40 mL) was added 3-bromoprop-1-yne (2.9 g, 24.4 mmol). After stirred at r.t. for 2 hr, the resulting mixture was partitioned between H₂O and EtOAc. The layers were separated and the organic layer was washed with brine, dried over Na₂SO₄, filtered and concentrated under reduced pressure to give the crude product which was purified by flash chromatography (silica gel, 0-30% EtOAc in PE) to afford the title compound (3.2 g, 71% yield) as a yellow oil. LCMS (ES+) (m/z): 254.9/256.9 (M/M+2). ¹H NMR (400 MHz, CDCl₃) δ 7.04 (s, 1H), 6.90 (s, 1H), 4.73 (d, J=2.4 Hz, 2H), 3.84 (s, 3H), 2.51 (t, J=2.4 Hz, 1H), 2.33 (s, 3H).

### Step 4: 6-Bromo-8-methoxy-5-methyl-2H-chromene

A mixture of 1-bromo-5-methoxy-2-methyl-4-(prop-2-yn-1-yloxy)benzene (3.2 g, 12.5 mmol) in NMP (30 mL) was heated at 240° C. for 6 hr. After cooled down to r.t., the resulting mixture was poured into water and extracted with EtOAc. The organic layer was washed with brine, dried over Na₂SO₄, filtered and concentrated under reduced pressure to give the crude product which was purified by flash chromatography (silica gel, 0-30% EtOAc in PE) to afford the title compound (1.5 g, 47% yield) as a yellow oil. LCMS (ES+) (m/z): 254.9/256.9 (M/M+2). ¹H NMR (400 MHz, CDCl₃) δ 6.97 (s, 1H), 6.63 (dt, J=10.1, 1.8 Hz, 1H), 5.89 (dt, J=10.1, 3.8 Hz, 1H), 4.78 (dd, J=3.8, 1.8 Hz, 2H), 3.83 (s, 3H), 2.31 (s, 3H).

### Step 5: 2-(8-Methoxy-5-methyl-2H-chromen-6-yl)-4,4,5,5-tetramethyl-1,3,2-dioxaborolane

A mixture of 6-bromo-8-methoxy-5-methyl-2H-chromene (134 mg, 0.53 mmol), B₂Pin₂ (201 mg, 0.79 mmol), Pd(dppf)Cl₂.DCM (43 mg, 0.053 mmol) and KOAc (181 mg, 1.85 mmol) in DMF (1.5 mL) was stirred at 80° C. under N₂ atmosphere overnight. The resulting mixture was partitioned between water and EtOAc. The organic layer was washed with brine, dried over Na₂SO₄, filtered and concentrated under reduced pressure to give the crude product which was purified by flash chromatography (silica gel, 0-30% EtOAc in PE) to afford the title compound (110 mg, 71% yield) as a yellow oil. LCMS (ES+) (m/z): 303.5 (M+1). ¹H NMR (400 MHz, CDCl₃) δ 7.20 (s, 1H), 6.68 (dt, J=10.1, 1.7 Hz, 1H), 5.83 (dt, J=10.1, 3.7 Hz, 1H), 4.81 (dd, J=3.7, 1.8 Hz, 2H), 3.88 (s, 3H), 2.46 (s, 3H), 1.33 (s, 12H).

### Step 6: 2-(8-Methoxy-5-methylchroman-6-yl)-4,4,5,5-tetramethyl-1,3,2-dioxaborolane

A mixture of 2-(8-methoxy-5-methyl-2H-chromen-6-yl)-4,4,5,5-tetramethyl-1,3,2-dioxa borolane (2.0 g, 6.6 mmol) and 10% Pd/C (400 mg) in EtOAc (20 mL) was stirred at r.t. under H₂ atmosphere overnight. The resulting mixture filtered through a pad of Celite and the filtrate was concentrated under reduced pressure to give the title compound (2.0 g, 99% yield) as a white solid which was used in the next step without further purification. LCMS (ES+) (m/z): 305.2 (M+1). ¹H NMR (400 MHz, CDCl₃) δ 7.15 (s, 1H), 4.25-4.22 (m, 2H), 3.89 (s, 3H), 2.65 (t, J=6.6 Hz, 2H), 2.38 (s, 3H), 2.07-2.00 (m, 2H), 1.33 (s, 12H).

### Step 7: (8-Methoxy-5-methylchroman-6-yl)boronic acid

A mixture of 2-(8-methoxy-5-methylchroman-6-yl)-4,4,5,5-tetramethyl-1,3,2-dioxaborolane (1.6 g, 5.2 mmol), AcONH₄ (2.4 g, 31 mmol) and NaIO₄ (5 g, 23 mmol) in acetone (16 mL) and water (8 mL) was stirred at 40° C. for 2 days. The resulting mixture was partitioned between water and EtOAc. The organic layer was washed with brine, dried over Na₂SO₄, filtered and concentrated under reduced pressure to give the crude product which was triturated with 10% EtOAc in PE to afford the title compound (180 mg, 15% yield) as a white solid. LCMS (ES+) (m/z): 223.0 (M+1).

### Step 8: (S)-2-(tert-Butoxy)-2-((R)-1-(4-methoxy-3-methylbenzoyl)-4-(8-methoxy-5-methylchroman-6-yl)-6-methylindolin-5-yl)acetic acid

The title compound was prepared in a manner similar to that described in Example 45 after purification by reverse phase HPLC. ¹H NMR (400 MHz, CDCl₃) δ 9.46 (br, 1H), 7.58 (s, 1H), 7.44-7.35 (m, 2H), 6.85 (d, J=8.4 Hz, 1H), 6.46 (s, 1H), 5.13 (s, 1H), 4.26 (t, J=5.2 Hz, 2H), 4.01 (t, J=8.3 Hz, 2H), 3.88 (s, 3H), 3.79 (s, 3H), 2.74-2.32 (m, 7H), 2.24 (s, 3H), 2.12-2.03 (m, 2H), 1.93 (s, 3H), 1.12 (s, 9H). LCMS (ES+) (m/z): 588.2 (M+1).

### Example 193: (S)-2-(tert-Butoxy)-2-((R)-4-(8-methoxy-5-methylchroman-6-yl)-6-methyl-1-(6-methylnicotinoyl)indolin-5-yl)acetic acid

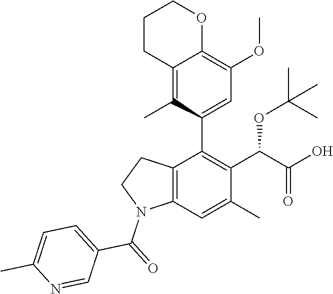

In a manner similar to that described in Example 192, the title compound was isolated after reverse-phase HPLC. ¹H NMR (400 MHz, CDCl₃) δ 9.45 (br, 1H), 8.72 (d, J=2.0 Hz, 1H), 8.25-7.89 (m, 2H), 7.79 (dd, J=8.0, 2.2 Hz, 1H), 6.45 (s, 1H), 5.14 (s, 1H), 4.26 (t, J=5.2 Hz, 2H), 4.08-3.93 (m, 2H), 3.79 (s, 3H), 2.87-2.19 (m, 10H), 2.14-2.02 (m, 2H), 1.92 (s, 3H), 1.13 (s, 9H). LCMS (ES+) (m/z): 559.0 (M+1).

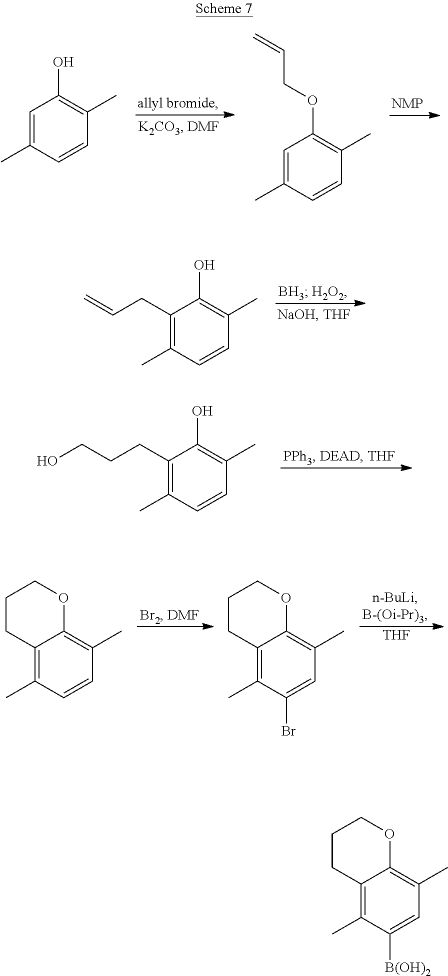

### Example 194: (S)-2-(tert-Butoxy)-2-((R)-4-(5,8-dimethylchroman-6-yl)-1-(4-methoxy-3-methylbenzoyl)-6-methylindolin-5-yl)acetic acid

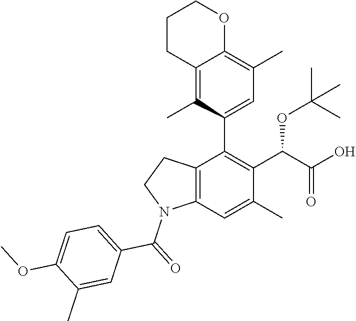

### Step 1: 1,4-Dimethyl-2-(prop-2-yn-1-yloxy)benzene

To a mixture of 2,5-dimethylphenol (5 g, 41 mmol) and K₂CO₃ (11 g, 81 mmol) in DMF (50 mL) was added 3-bromoprop-1-yne (6.8 g, 57 mmol). After stirred at r.t. for 3 hr, the resulting mixture was partitioned between H₂O and EtOAc. The layers were separated and the organic layer was washed with brine, dried over Na₂SO₄, filtered and concentrated under reduced pressure to give the crude product which was purified by flash chromatography (silica gel, 0-10% EtOAc in PE) to afford the title compound (6 g, 92% yield) as a yellow oil. ¹H NMR (400 MHz, CDCl₃) δ 7.03 (d, J=7.5 Hz, 1H), 6.79-6.70 (m, 2H), 4.70 (d, J=2.4 Hz, 2H), 2.51 (t, J=2.4 Hz, 1H), 2.34 (s, 3H), 2.21 (s, 3H).

### Step 2: 5,8-Dimethyl-2H-chromene

A mixture of 1,4-dimethyl-2-(prop-2-yn-1-yloxy)benzene (6.0 g, 37.5 mmol) in NMP (24 mL) was heated at 240° C. for 24 hr. After cooled down to r.t., the resulting mixture was poured into water and extracted with EtOAc. The organic layer was washed with brine, dried over Na₂SO₄, filtered and concentrated under reduced pressure to give the crude product which was purified by flash chromatography (silica gel, 0-10% EtOAc in PE) to afford the title compound (4.5 g, 75% yield) as a light yellow oil. ¹H NMR (400 MHz, CDCl₃) δ 6.87 (d, J=7.6 Hz, 1H), 6.69-6.57 (m, 2H), 5.83 (dt, J=9.9, 3.7 Hz, 1H), 4.75 (dd, J=3.7, 1.8 Hz, 2H), 2.26 (s, 3H), 2.14 (s, 3H).

### Step 3: 5,8-Dimethylchromane

A mixture of 5,8-dimethyl-2H-chromene (4.5 g, 28 mmol) and 10% Pd/C (900 mg) in MeOH (60 mL) was stirred at r.t. under H₂ atmosphere for 3 hr. The resulting mixture filtered through a pad of Celite and the filtrate was concentrated under reduced pressure to give the title compound (4.2 g, 93% yield) as a colorless oil which was used in the next step without further purification. ¹H NMR (400 MHz, CDCl₃) δ 6.86 (d, J=7.5 Hz, 1H), 6.63 (d, J=7.5 Hz, 1H), 4.19-4.13 (m, 2H), 2.63 (t, J=6.6 Hz, 2H), 2.17 (s, 3H), 2.15 (s, 3H), 2.06-1.93 (m, 2H).

### Step 4: 6-Bromo-5,8-dimethylchromane

To a solution of 5,8-dimethylchromane (2 g, 12.3 mmol) in DMF (20 mL) was added bromine (1.2 mL, 23.4 mmol) slowly. After stirred at r.t. for 3 hr, the resulting mixture was quenched with sat. Na₂S₂O₃ aq. solution and extracted with EtOAc. The organic layer was washed with brine, dried over Na₂SO₄, filtered and concentrated under reduced pressure to give the crude product which was purified by flash chromatography (silica gel, 0-10% EtOAc in PE) to afford the title compound (2.5 g, 83% yield) as a light yellow solid. ¹H NMR (400 MHz, CDCl₃) δ 7.16 (s, 1H), 4.16-4.10 (m, 2H), 2.67 (t, J=6.6 Hz, 2H), 2.25 (s, 3H), 2.11 (s, 3H), 2.05-1.96 (m, 2H).

### Step 5: (5,8-Dimethylchroman-6-yl)boronic acid

At −78° C., to a solution of 6-bromo-5,8-dimethylchromane (1 g, 4.2 mmol) in THF (10 mL) was added n-BuLi (2.5 M, 2.5 mL, 6.2 mmol). The reaction mixture was stirred at −78° C. for 5 min before the introduction of triisopropyl borate (1.6 g, 8.5 mmol). After warmed up to r.t., the resulting mixture was quenched with water and extracted with EtOAc. The organic layer was washed with brine, dried over Na₂SO₄, filtered and concentrated under reduced pressure to give the crude product which was triturated with 10% EtOAc in PE to afford the title compound (500 mg, 58% yield) as a white solid. LCMS (ES+) (m/z): 207.0 (M+1).

### Step 6: S)-2-(tert-Butoxy)-2-((R)-4-(5,8-dimethylchroman-6-yl)-1-(4-methoxy-3-methylbenzoyl)-6-methylindolin-5-yl)acetic acid

In a manner similar to that described in Example 45, the title compound was isolated after reverse-phase HPLC. ¹H NMR (400 MHz, CDCl₃) δ 9.57 (br, 1H), 7.62 (s, 1H), 7.44-7.35 (m, 2H), 6.85 (d, J=8.4 Hz, 1H), 6.70 (s, 1H), 5.08 (s, 1H), 4.19 (t, J=5.3 Hz, 2H), 4.00 (t, J=8.3 Hz, 2H), 3.88 (s, 3H), 2.72-2.48 (m, 4H), 2.38 (s, 3H), 2.24 (s, 3H), 2.14 (s, 3H), 2.11-2.03 (m, 2H), 1.94 (s, 3H), 1.11 (s, 9H). LCMS (ES+) (m/z): 572.4 (M+1).

### Example 195: (S)-2-(tert-Butoxy)-2-((R)-4-(5,8-dimethylchroman-6-yl)-6-methyl-1-(6-methylnicotinoyl)indolin-5-yl)acetic acid

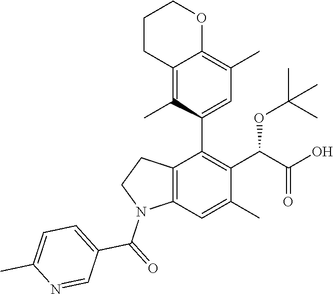

In a manner similar to that described in Example 194, the title compound was isolated after reverse-phase HPLC. ¹H NMR (400 MHz, CDCl₃) δ 8.73 (d, J=1.7 Hz, 1H), 8.03 (s, 1H), 7.81 (dd, J=8.0, 2.2 Hz, 1H), 7.30-7.26 (m, 1H), 6.70 (s, 1H), 5.09 (s, 1H), 4.19 (t, J=5.3 Hz, 2H), 4.05-3.92 (m, 2H), 2.75-2.36 (m, 10H), 2.14 (s, 3H), 2.08-2.01 (m, 2H), 1.94 (s, 3H), 1.12 (s, 9H). LCMS (ES+) (m/z): 543.4 (M+1).

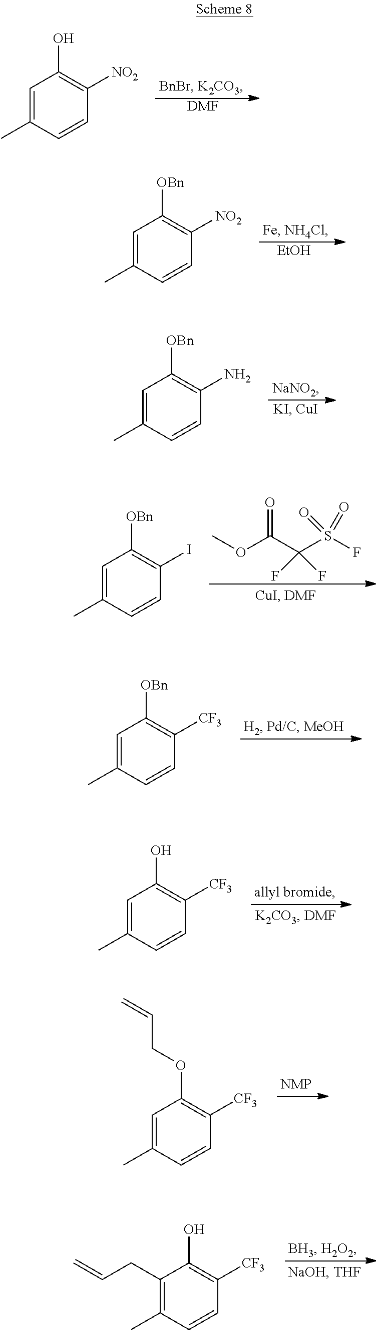

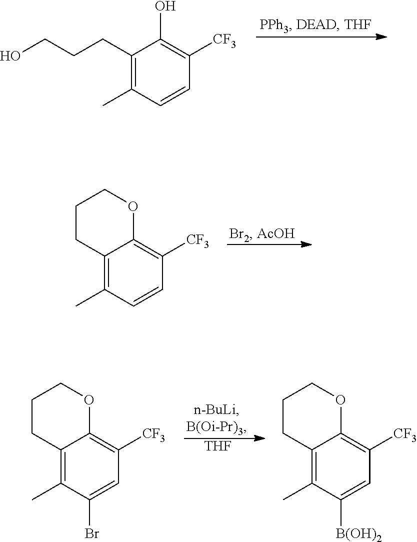 [SMILES: Cc1ccc(C(F)(F)F)c(O)c1CCCO ; "PPh3, DEAD, THF" ; Cc1ccc(C(F)(F)F)c2c1CCCO2 ; Cc1c(Br)cc(C(F)(F)F)c2c1CCCO2 ; Cc1c(B(O)O)cc(C(F)(F)F)c2c1CCCO2 ; "THF"]

### Example 196: (S)-2-(tert-Butoxy)-2-((R)-1-(4-methoxy-3-methylbenzo yl)-6-methyl-4-(5-methyl-8-(trifluoromethyl)chroman-6-yl)indolin-5-yl)acetic acid

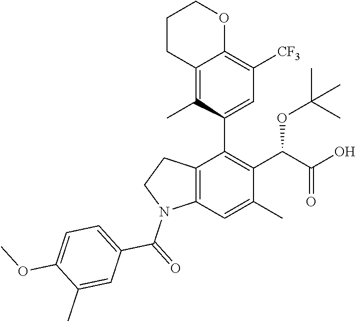

### Step 1: 2-(Benzyloxy)-4-methyl-1-nitrobenzene

To a suspension of 5-methyl-2-nitrophenol (15 g, 98 mmol) and K₂CO₃ (27 g, 19.6 mmol) in DMF (150 mL) was added benzyl bromide (23.4 g, 13.7 mmol). After stirred at r.t. for 3 hr, the resulting mixture was filtered and the filtrate was partitioned between water and EtOAc. The layers were separated and the organic layer was washed with brine, dried over Na₂SO₄, filtered and concentrated under reduced pressure to give the crude product which was triturated with petroleum ether to afford the title compound (22 g, 92% yield) as a white solid. LCMS (ES−) (m/z): 242.0 (M−1).

### Step 2: 2-(Benzyloxy)-4-methylaniline

A mixture of 2-(benzyloxy)-4-methyl-1-nitrobenzene (21 g, 86.4 mmol), iron powder (24 g, 429 mmol) and NH₄Cl (3 g, 56.1 mmol) in H₂O (500 mL) was refluxed for 1.5 hr under vigorous stirring. After cooled down to r.t., the resulting mixture was neutralized with 5% NaHCO₃ aq. solution to pH 7-8 and filtered. The filtrate was extracted with EtOAc and the organic layer was washed with brine, dried over Na₂SO₄, filtered and concentrated under reduced pressure to give the title compound (19 g, quant. yield) as a yellow solid which was used in the next step without further purification. LCMS (ES+) (m/z): 214.4 (M+1). ¹H NMR (400 MHz, CDCl₃) δ 7.80 (d, J=8.3 Hz, 1H), 7.47 (d, J=7.3 Hz, 2H), 7.43-7.36 (m, 2H), 7.35-7.30 (m, 1H), 6.92 (s, 1H), 6.83 (dd, J=8.3, 0.8 Hz, 1H), 5.21 (s, 2H), 2.39 (s, 3H). (the NH₂ was not observed)

### Step 3: 2-(Benzyloxy)-1-iodo-4-methylbenzene

At 0° C., to a suspension of 2-(benzyloxy)-4-methylaniline (19 g, 89 mmol) in H₂O (700 mL) and conc. H₂SO₄ (100 mL) was added a solution of NaNO₂ (6.8 g, 98 mmol) in H₂O (30 mL) dropwise. The reaction mixture was stirred at 0° C. for 1 hr before the addition of a solution of KI (19.2 g, 116 mmol) in H₂O (90 mL). After stirred at r.t. overnight, the resulting mixture was extracted with EtOAc and the organic layer was washed successively with 1N NaOH aq. solution, 1N Na₂S₂O₃ aq. solution, 1N HCl aq. solution, 1N NaHCO₃ aq. solution and brine, dried over Na₂SO₄, filtered and concentrated under reduced pressure to give the crude product which was purified by flash chromatography (silica gel, 0-20% EtOAc in PE) to afford the title compound (12.6 g, 44% yield) as a yellow solid. LCMS (ES−) (m/z): 323.0 (M−1). ¹H NMR (400 MHz, CDCl₃) δ 7.64 (d, J=7.9 Hz, 1H), 7.51 (d, J=7.2 Hz, 2H), 7.40 (t, J=7.4 Hz, 2H), 7.33 (d, J=7.3 Hz, 1H), 6.70 (d, J=1.0 Hz, 1H), 6.57 (d, J=7.9 Hz, 1H), 5.13 (s, 2H), 2.30 (s, 3H).

### Step 4: 2-(Benzyloxy)-4-methyl-1-(trifluoromethyl)benzene

A mixture of 2-(benzyloxy)-1-iodo-4-methylbenzene (12.6 g, 38.9 mmol), CuI (14.8 g, 77.7 mmol) and methyl 2,2-difluoro-2-(fluorosulfonyl)acetate (18.7 g, 97.4 mmol) in DMF (120 mL) was stirred at 90° C. under N₂ atmosphere overnight. The resulting mixture was filtered and the filtrate was partitioned between water and EtOAc. The layers were separated and the organic layer was washed with brine, dried over Na₂SO₄, filtered and concentrated under reduced pressure to give the crude product which was purified by flash chromatography (silica gel, 0-10% EtOAc in PE) to afford the title compound (5.5 g, 53% yield) as a yellow oil. LCMS (ES−) (m/z): 265.0 (M−1). ¹H NMR (400 MHz, CDCl₃) δ 7.48-7.42 (m, 3H), 7.41-7.37 (m, 2H), 7.32 (d, J=7.2 Hz, 1H), 6.86-6.79 (m, 2H), 5.16 (s, 2H), 2.37 (s, 3H).

### Step 5: 5-Methyl-2-(trifluoromethyl)phenol

A mixture of 2-(benzyloxy)-4-methyl-1-(trifluoromethyl)benzene (5.5 g, 20.7 mmol) and 10% Pd(OH)₂/C (2 g) in MeOH (55 mL) was stirred at 50° C. under H₂ atmosphere overnight. The resulting mixture filtered through a pad of Celite and the filtrate was concentrated under reduced pressure to give the title compound (3.6 g, quant. yield) as a yellow oil which was used in the next step without further purification. LCMS (ES−) (m/z): 175.0 (M−1). ¹H NMR (400 MHz, CDCl₃) δ 7.38 (d, J=8.0 Hz, 1H), 6.81 (d, J=8.0 Hz, 1H), 6.77 (s, 1H), 5.45 (s, 1H), 2.34 (s, 3H).

### Step 6: 4-Methyl-2-(prop-2-yn-1-yloxy)-1-(trifluoromethyl)benzene

To a mixture of 5-methyl-2-(trifluoromethyl)phenol (4 g, 22.7 mmol) and K₂CO₃ (6.3 g, 45.4 mmol) in DMF (40 mL) was added 3-bromoprop-1-yne (3.8 g, 31.8 mmol). After stirred at r.t. for 2 hr, the resulting mixture was partitioned between H₂O and EtOAc. The layers were separated and the organic layer was washed with brine, dried over Na₂SO₄, filtered and concentrated under reduced pressure to give the crude product which was purified by flash chromatography (silica gel, 0-20% EtOAc in PE) to afford the title compound (3.2 g, 65% yield) as a yellow oil. LCMS (ES−) (m/z): 213.1 (M−1). ¹H NMR (400 MHz, CDCl₃) δ 7.45 (d, J=7.9 Hz, 1H), 6.95 (s, 1H), 6.86 (d, J=7.9 Hz, 1H), 4.78 (d, J=2.4 Hz, 2H), 2.54 (t, J=2.4 Hz, 1H), 2.40 (s, 3H).

### Step 7: 5-Methyl-8-(trifluoromethyl)-2H-chromene

A mixture of 4-methyl-2-(prop-2-yn-1-yloxy)-1-(trifluoromethyl)benzene (3.1 g, 14.5 mmol) and [Bis(trifluoromethanesulfonyl)imidate](triphenylphosphine)gold(I) (54 mg, 0.07 mmol) in DCM (30 mL) was stirred at r.t. for 3 days. The resulting mixture was concentrated under reduced pressure to give the crude product which was purified by flash chromatography (silica gel, 0-10% EtOAc in PE) to afford the title compound (2.2 g, 71% yield) as a yellow oil. LCMS (ES+) (m/z): 215.1 (M+1). ¹H NMR (400 MHz, CDCl₃) δ 7.25 (d, J=6.9 Hz, 1H), 6.75 (d, J=8.0 Hz, 1H), 6.61 (dt, J=10.1, 1.8 Hz, 1H), 5.91 (dt, J=10.1, 3.7 Hz, 1H), 4.84 (dd, J=3.7, 1.8 Hz, 2H), 2.31 (s, 3H).

### Step 8: 5-Methyl-8-(trifluoromethyl)chromane

A mixture of 5-methyl-8-(trifluoromethyl)-2H-chromene (2.2 g, 10.3 mmol) and 10% Pd/C (1 g) in EtOAc (20 mL) was stirred at r.t. under H₂ atmosphere overnight. The resulting mixture filtered through a pad of Celite and the filtrate was concentrated under reduced pressure to give the title compound (2.1 g, 95% yield) as a white solid which was used in the next step without further purification. ¹H NMR (400 MHz, CDCl₃) δ 7.28 (d, J=7.9 Hz, 1H), 6.75 (d, J=7.9 Hz, 1H), 4.29-4.21 (m, 2H), 2.66 (t, J=6.6 Hz, 2H), 2.23 (s, 3H), 2.13-2.02 (m, 2H).

### Step 9: 6-bromo-5-methyl-8-(trifluoromethyl)chromane

To a solution of 5-methyl-8-(trifluoromethyl)chromane (2.1 g, 9.7 mmol) in DMF (20 mL) was added bromine (3.1 g, 19.4 mmol) portionwise. After stirred at r.t. for 2 hr, the resulting mixture was quenched with sat. Na₂SO₃ aq. solution and extracted with EtOAc.

The organic layer was washed with brine, dried over Na₂SO₄, filtered and concentrated under reduced pressure to give the crude product which was purified by flash chromatography (silica gel, 0-10% EtOAc in PE) to afford the title compound (2.8 g, 98% yield) as a white solid. ¹H NMR (400 MHz, CDCl₃) δ 7.58 (s, 1H), 4.24-4.16 (m, 2H), 2.73 (t, J=6.6 Hz, 2H), 2.33 (s, 3H), 2.11-2.04 (m, 2H). ¹⁹F NMR (376 MHz, CDCl₃) δ −62.46 (s, 1H).

### Step 10: (5-methyl-8-(trifluoromethyl)chroman-6-yl)boronic acid

At −78° C., to a solution of 6-bromo-5-methyl-8-(trifluoromethyl)chromane (1.3 g, 4.4 mmol) in THF (13 mL) was added n-BuLi (2.5 M, 3.5 mL, 8.8 mmol). The reaction mixture was stirred at −78° C. for 5 min before the introduction of triisopropyl borate (1.65 g, 8.8 mmol). After warmed up to r.t., the resulting mixture was quenched with sat. NaHCO₃ aq. solution and extracted with EtOAc. The organic layer was washed with brine, dried over Na₂SO₄, filtered and concentrated under reduced pressure to give the crude product which was triturated with 10% EtOAc in PE to afford the title compound (900 mg, 78% yield) as a white solid. LCMS (ES+) (m/z): 261.3 (M+1). ¹H NMR (400 MHz, DMSO) δ 7.98 (s, 1H), 4.22-4.16 (m, 2H), 2.70 (t, J=6.4 Hz, 2H), 2.59 (s, 3H), 2.04-1.98 (m, 2H). (the proton on B(OH)₂ was not observed).

### Step 11: (S)-2-(tert-Butoxy)-2-((R)-1-(4-methoxy-3-methylbenzoyl)-6-methyl-4-(5-methyl-8-(trifluoromethyl)chroman-6-yl)indolin-5-yl)acetic acid

In a manner similar to that described in Example 192, the title compound was isolated after reverse-phase HPLC. ¹H NMR (400 MHz, CDCl₃) δ 9.52 (br, 1H), 7.53 (s, 1H), 7.37-7.28 (m, 2H), 7.13 (s, 1H), 6.78 (d, J=8.3 Hz, 1H), 4.90 (s, 1H), 4.20 (t, J=5.4 Hz, 2H), 3.96 (t, J=8.4 Hz, 2H), 3.81 (s, 3H), 2.69-2.41 (m, 4H), 2.32 (s, 3H), 2.18 (s, 3H), 2.10-1.99 (m, 2H), 1.97 (s, 3H), 1.05 (s, 9H). LCMS (ES+) (m/z): 626.6 (M+1).

### Example 197: (S)-2-(tert-Butoxy)-2-((R)-6-methyl-4-(5-methyl-8-(trifluoromethyl)chroman-6-yl)-1-(6-methylnicotinoyl)indolin-5-yl)acetic acid

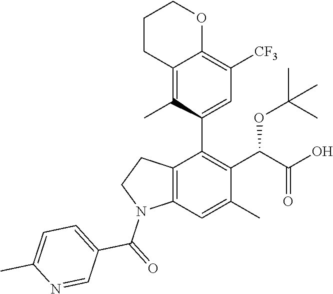

In a manner similar to that described in Example 196, the title compound was isolated after reverse-phase HPLC. ¹H NMR (400 MHz, CDCl₃) δ 9.65 (br, 1H), 8.66 (d, J=1.9 Hz, 1H), 7.98 (s, 1H), 7.74 (dd, J=8.0, 2.2 Hz, 1H), 7.23-7.20 (m, 1H), 7.13 (s, 1H), 4.91 (s, 1H), 4.21 (t, J=5.1 Hz, 2H), 4.01-3.87 (m, 2H), 2.69-2.27 (m, 10H), 2.09-2.01 (m, 2H), 1.97 (s, 3H), 1.05 (s, 9H). LCMS (ES+) (m/z): 597.6 (M+1).

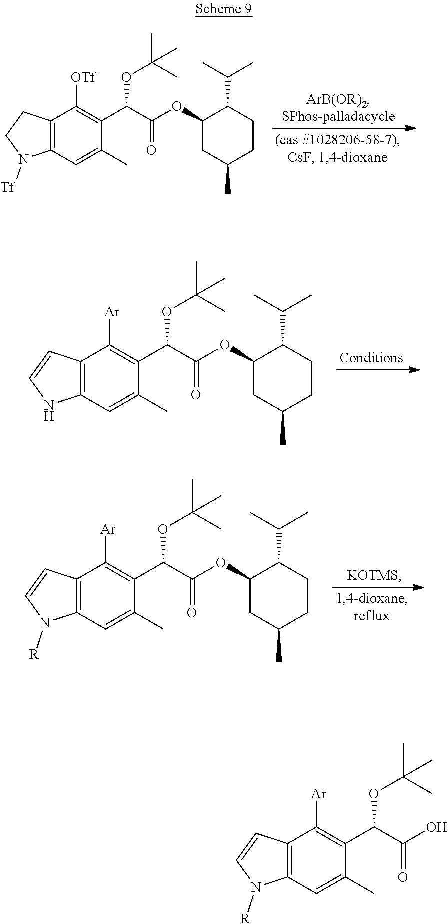

### Example 198: (S)-2-(tert-Butoxy)-2-((R)-1-(3,4-difluorobenzyl)-4-(8-fluoro-5-methylchroman-6-yl)-6-methyl-1H-indol-5-yl)acetic acid

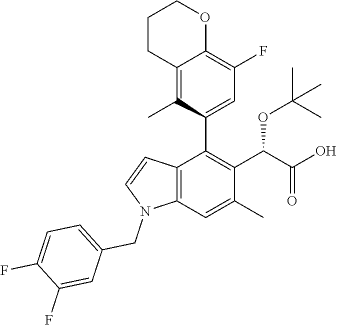

### Step 1: (1R,2S,5R)-2-Isopropyl-5-methylcyclohexyl (S)-2-(tert-butoxy)-2-((R)-4-(8-fluoro-5-methylchroman-6-yl)-6-methyl-1H-indol-5-yl)acetate

A mixture of (1R,2S,5R)-2-isopropyl-5-methylcyclohexyl (S)-2-(tert-butoxy)-2-(6-methyl-1-((trifluoromethyl)sulfonyl)-4-(((trifluoromethyl)sulfonyl)oxy)indolin-5-yl)acetate (1.2 g, 1.76 mmol), (8-fluoro-5-methylchroman-6-yl)boronic acid (0.92 g, 4.38 mmol), CsF (1.07 g, 7.04 mmol) and SPhos precatalyst (0.4 g, 0.53 mmol) in 1,4-dioxane (12 mL) was stirred at 130° C. in microwave apparatus under N₂ atmosphere for 40 min. The resulting mixture was filtered and concentrated to give the crude product which was purified by flash chromatography (silica gel, 0-10% EtOAc in PE) to afford the title compound (0.15 g, 15% yield) as a brown solid. LC-MS (ESI): m/z (M+Na)=586.4.

### Step 2: (1R,2S,5R)-2-Isopropyl-5-methylcyclohexyl (S)-2-(tert-butoxy)-2-((R)-1-(3,4-difluorobenzyl)-4-(8-fluoro-5-methylchroman-6-yl)-6-methyl-1H-indol-5-yl)acetate

To a solution of (1R,2S,5R)-2-isopropyl-5-methylcyclohexyl (S)-2-(tert-butoxy)-2-((R)-4-(8-fluoro-5-methylchroman-6-yl)-6-methyl-1H-indol-5-yl)acetate (56 mg, 0.1 mmol) in THF (1.0 mL) was added NaH (60%, 12 mg, 0.3 mmol). The resulting mixture was stirred at 0° C. for 30 min before the addition of 4-(bromomethyl)-1,2-difluoro benzene (41 mg, 0.2 mmol). After stirred at room temperature for 1 hour, then the reaction mixture was quenched with sat. NH₄Cl aq. solution and extracted with EtOAc. The layers were separated and the organic layer was washed with brine, dried over sodium sulfate, filtered, and concentrated under reduced pressure to give the crude product which was purified by flash chromatography (silica gel, 0-20% EtOAc in PE) to afford the title compound as a yellow oil (47 mg, 69% yield). LC-MS (ESI): m/z (M+Na)=712.3.

### Step C: (S)-2-(tert-Butoxy)-2-((R)-1-(3,4-difluorobenzyl)-4-(8-fluoro-5-methylchroman-6-yl)-6-methyl-1H-indol-5-yl)acetic acid

A mixture of (1R,2S,5R)-2-isopropyl-5-methylcyclohexyl (S)-2-(tert-butoxy)-2-((R)-1-(3,4-difluorobenzyl)-4-(8-fluoro-5-methylchroman-6-yl)-6-methyl-1H-indol-5-yl)acetate (47 mg, 0.068 mmol) and KOTMS (87 mg, 0.68 mmol) in dioxane (1 mL) was stirred at 110° C. overnight. The resulting mixture was quenched with 1N HCl and extracted with 15% i-PrOH in DCM. The organic layer was washed with brine, dried over sodium sulfate, filtered, and concentrated under reduced pressure to give the crude product which was purified by HPLC (C18, 0-100% MeCN in H2O with 0.1% formic acid) to afford the title compound (5 mg, 13% yield) as a white powder. LCMS (ES−) (m/z): 550.1 (M−1). 1H NMR (400 MHz, CDCl3) δ 9.81 (br, 1H), 7.15-7.03 (m, 2H), 6.99-6.84 (m, 4H), 5.93 (d, J=3.1 Hz, 1H), 5.26-5.19 (m, 3H), 4.31-4.27 (m, 2H), 2.76-2.69 (m, 2H), 2.51 (s, 3H), 2.17-2.10 (m, 2H), 1.91 (s, 3H), 1.12 (s, 9H).

### Example 199: (S)-2-(tert-Butoxy)-2-((R)-1-(4-fluoro-2-methylbenzyl)-4-(8-fluoro-5-methylchroman-6-yl)-6-methyl-1H-indol-5-yl)acetic acid

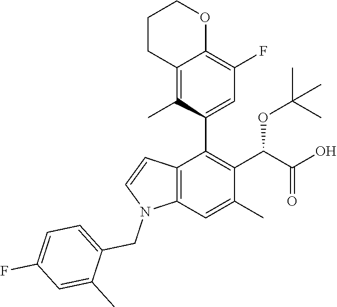

In a manner similar to that described in Example 198, the title compound was isolated after reverse-phase HPLC. ¹H NMR (400 MHz, CDCl₃) δ 9.74 (br, 1H), 7.08 (d, J=5.9 Hz, 1H), 6.93 (d, J=9.4 Hz, 1H), 6.90-6.73 (m, 4H), 5.90 (dd, J=7.5, 3.1 Hz, 1H), 5.45 (s, 1H), 5.18 (s, 2H), 4.33-4.23 (m, 2H), 2.75-2.65 (m, 2H), 2.56-2.48 (m, 3H), 2.30 (s, 3H), 2.17-2.10 (m, 2H), 1.87 (d, J=36.9 Hz, 3H), 1.09 (d, J=27.2 Hz, 9H). LCMS (ES+) (m/z): 548.2 (M+1).

### Example 200: (S)-2-(tert-Butoxy)-2-((R)-1-(3-Fluoro-2-methoxybenzyl)-4-(8-fluoro-5-methylchroman-6-yl)-6-methyl-1H-indol-5-yl)acetic acid

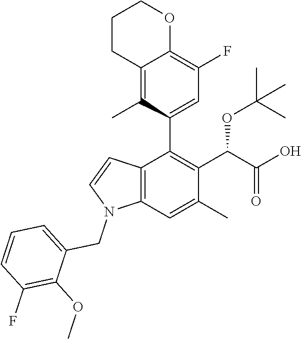

### Step 1: (S)-(1R,2S,5R)-2-Isopropyl-5-methylcyclohexyl 2-(tert-butoxy)-2-((R)-1-(3-fluoro-2-methoxybenzyl)-4-(8-fluoro-5-methylchroman-6-yl)-6-methyl-1H-indol-5-yl)acetate

A solution of (S)-(1R,2S,5R)-2-isopropyl-5-methylcyclohexyl 2-(tert-butoxy)-2-((R)-4-(8-fluoro-5-methylchroman-6-yl)-6-methyl-1H-indol-5-yl)acetate (50 mg, 0.089 mmol) in Acetonitrile (0.9 mL) was treated with cesium carbonate (116 mg, 0.355 mmol), 1-(bromomethyl)-3-fluoro-2-methoxybenzene (27.2 mg, 0.124 mmol), and stirred at r.t for 18 hours. The reaction was diluted with water and 1N HCl, extracted with EtOAc, washed with Brine, dried over sodium sulfate, filtered, and concentrated to give crude (S)-(1R,2S,5R)-2-isopropyl-5-methylcyclohexyl 2-(tert-butoxy)-2-((R)-1-(3-fluoro-2-methoxybenzyl)-4-(8-fluoro-5-methylchroman-6-yl)-6-methyl-1H-indol-5-yl)acetate (69 mg, 0.098 mmol, 111% yield) as yellow oil. LCMS (m/z) ES⁺=724.7 (M+23).

### Step 2: (S)-2-(tert-Butoxy)-2-((R)-1-(3-Fluoro-2-methoxybenzyl)-4-(8-fluoro-5-methylchroman-6-yl)-6-methyl-1H-indol-5-yl)acetic acid

A solution of crude (S)-(1R,2S,5R)-2-isopropyl-5-methylcyclohexyl 2-(tert-butoxy)-2-((R)-1-(3-fluoro-2-methoxybenzyl)-4-(8-fluoro-5-methylchroman-6-yl)-6-methyl-1H-indol-5-yl)acetate (69 mg, 0.098 mmol) in 1,4-Dioxane (1.5 mL) was treated with KOTMS (114 mg, 0.887 mmol) and stirred for 2 hours in pre-heated 150° C. oil bath. The reaction was cooled to r.t., diluted with ice/1 M HCl, extracted with EtOAc 2×, washed with 1N HCl, Brine, dried over sodium sulfate, filtered, and concentrated. Purification with reverse phase HPLC (30-100% MeCN-0.1% formic acid/H₂O—0.1% formic acid) afforded the title compound (20.2 mg, 0.033 mmol, 37.6% yield, as off-white solid impure with ˜20% (P,S)-diastereomer. ¹H NMR (400 MHz, METHANOL-d₄) (mixture of atropisomers) δ ppm 7.23-7.13 (m, 1H), 7.11-7.00 (m, 2H), 6.99-6.88 (m, 1H), 6.78-6.66 (m, 2H), 5.86-5.69 (m, 1H), 5.35 (s, 2H), 5.30-5.11 (m, 1H), 4.24 (t, J=5.0 Hz, 2H), 3.84 (s, 3H), 2.74 (t, J=6.4 Hz, 2H), 2.62-2.48 (m, 3H), 2.20-2.05 (m, 2H), 1.86-1.75 (m, 3H), 1.14-0.90 (m, 9H); LCMS (m/z) ES⁺=564.4 (M+1), ES⁻=562.2 (M−1).

### Example 201: (S)-2-(tert-Butoxy)-2-((R)-4-(8-Fluoro-5-methylchroman-6-yl)-6-methyl-1-(pyridin-2-ylmethyl)-1H-indol-5-yl)acetic acid

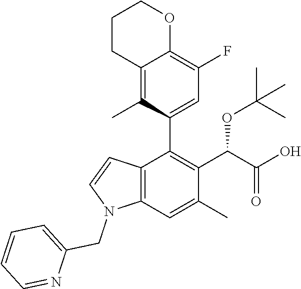

In a manner similar to that described in Example 200, the title compound was prepared as a white solid impure with 20% (P,S)-diastereomer. ¹H NMR (400 MHz, METHANOL-d₄) (mixture of atropisomers) 8 ppm 8.54 (d, J=4.6 Hz, 1H), 7.72 (t, J=7.7 Hz, 1H), 7.38-7.27 (m, 1H), 7.22-7.04 (m, 2H), 6.90 (d, J=7.7 Hz, 1H), 6.72 (d, J=11.5 Hz, 1H), 5.93-5.76 (m, 1H), 5.46 (s, 2H), 5.16 (s, 1H), 4.25 (t, J=5.0 Hz, 2H), 2.75 (t, J=6.4 Hz, 2H), 2.62-2.45 (m, 3H), 2.20-2.07 (m, 2H), 1.89-1.77 (m, 3H), 1.18-0.87 (m, 9H); LCMS (m/z) ES⁺=517.4 (M+1), ES⁻=515.3 (M−1).

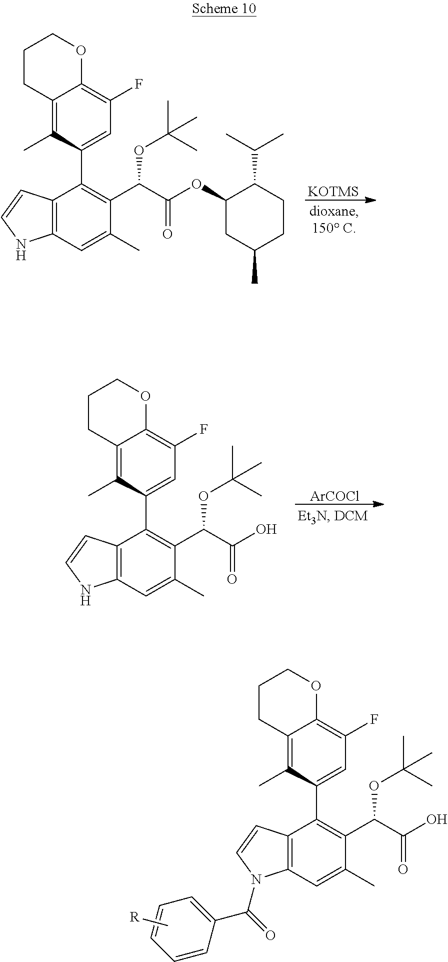

### Example 202: (S)-2-(tert-Butoxy)-2-((R)-4-(8-Fluoro-5-methylchroman-6-yl)-1-(4-methoxy-3-methylbenzoyl)-6-methyl-1H-indol-5-yl)acetic acid

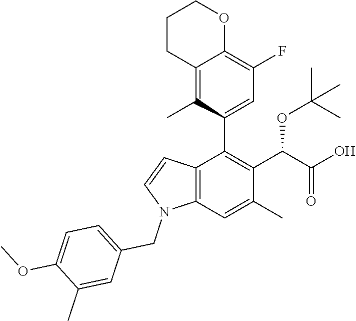

### Step 1: (S)-2-(tert-Butoxy)-2-((R)-4-(8-fluoro-5-methylchroman-6-yl)-6-methyl-1H-indol-5-yl)acetic acid

A solution of (S)-(1R,2S,5R)-2-isopropyl-5-methylcyclohexyl 2-(tert-butoxy)-2-((R)-4-(8-fluoro-5-methylchroman-6-yl)-6-methyl-1H-indol-5-yl)acetate (50 mg, 0.089 mmol) in 1,4-Dioxane (1.8 mL) was treated with KOTMS (114 mg, 0.887 mmol) and stirred for 5.5 hours in pre-heated 150° C. oil bath. The reaction was cooled to room temperature, diluted with ice/1 M HCl, extracted with EtOAc 2×, washed with 1N HCl, Brine, dried over sodium sulfate, filtered, and concentrated in vacuo. to give crude (S)-2-(tert-butoxy)-2-((R)-4-(8-fluoro-5-methylchroman-6-yl)-6-methyl-1H-indol-5-yl)acetic acid (54.2 mg, 0.127 mmol, 144% yield) as brown oil. LCMS (m/z) ES⁻=424.5 (M−1).

### Step 2: (S)-2-(tert-Butoxy)-2-((R)-4-(8-Fluoro-5-methylchroman-6-yl)-1-(4-methoxy-3-methylbenzoyl)-6-methyl-1H-indol-5-yl)acetic acid

A solution of crude (S)-2-(tert-butoxy)-2-((R)-4-(8-fluoro-5-methylchroman-6-yl)-6-methyl-1H-indol-5-yl)acetic acid (37.7 mg, 0.089 mmol) in Dichloromethane (DCM) (0.9 mL) was treated with Et3N (0.049 mL, 0.354 mmol), 4-methoxy-3-methylbenzoyl chloride (1 M in DCM) (0.106 mL, 0.106 mmol), and stirred at r.t. for 18 hours. The reaction was diluted with sat. NaHCO₃, extracted with DCM, washed with Brine, dried over Na2SO4, filtered, and concentrated. Purification with reverse phase HPLC (30-100% MeCN—0.1% formic acid/H2O—0.1% formic acid) afforded the title compound (15.2 mg, 0.025 mmol, 28.4% yield) as off-white solid impure with ˜5% (P,S)-diastereomer. ¹H NMR (400 MHz, METHANOL-d₄) δ ppm 7.60 (d, J=8.1 Hz, 1H), 7.41-7.23 (m, 2H), 7.12 (d, J=2.7 Hz, 1H), 6.93 (d, J=8.6 Hz, 1H), 6.77 (d, J=11.4 Hz, 1H), 5.77 (d, J=2.0 Hz, 1H), 5.43 (s, 1H), 4.38-4.11 (m, 2H), 3.89 (s, 3H), 2.78-2.43 (m, 5H), 2.22-1.93 (m, 5H), 1.67 (s, 3H), 1.26-0.98 (m, 9H); LCMS (m/z) ES⁻=572.3 (M−1).

### Example 203: (S)-2-(tert-Butoxy)-2-((R)-1-(3,4-difluorobenzoyl)-4-(8-fluoro-5-methylchroman-6-yl)-6-methyl-1H-indol-5-yl)acetic acid

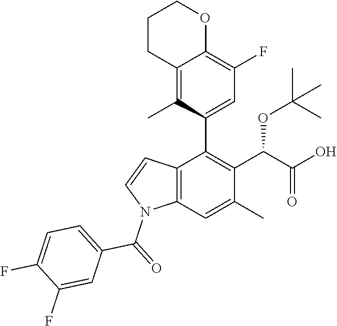

A solution of crude (S)-2-(tert-butoxy)-2-((R)-4-(8-fluoro-5-methylchroman-6-yl)-6-methyl-1H-indol-5-yl)acetic acid (11 mg, 0.027 mmol) in Dichloromethane (DCM) (0.5 mL) was treated with 3,4-difluorobenzoyl chloride (5.72 mg, 0.032 mmol), DMAP (5.6 mg, 0.046 mmol), Et3N (0.015 mL, 0.108 mmol), and stirred at r.t. for 1.5 hours. The reaction was treated with additional 3,4-difluorobenzoyl chloride (25 mg), et3N (30 uL), and stirred at r.t. for 3 hours. The mixture was diluted with sat. NaHCO₃, extracted with DCM, washed with Brine, dried over Na2SO4, filtered, and concentrated. Purification with reverse phase HPLC (20-100% MeCN-0.1% formic acid/H2O—0.1% formic acid) afforded the title compound (2.6 mg, 4.14 μmol, 15.32% yield) as white solid impure with ˜10% (P,S)-diastereomer. ¹H NMR (400 MHz, METHANOL-d₄) (mixture of atropisomers) 8 ppm 8.17 (s, 1H), 7.81-7.69 (m, 1H), 7.59 (br. s., 1H), 7.54-7.42 (m, 1H), 7.29-7.20 (m, 1H), 6.82-6.61 (m, 1H), 6.06-5.93 (m, 1H), 5.32-5.08 (m, 1H), 4.25 (t, J=5.0 Hz, 2H), 2.80-2.56 (m, 5H), 2.21-2.07 (m, 2H), 1.90-1.77 (m, 3H), 1.20-0.96 (m, 9H); LCMS (m/z) ES⁻=564.5 (M−1).

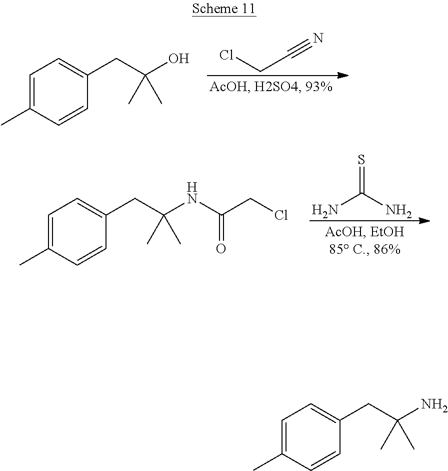

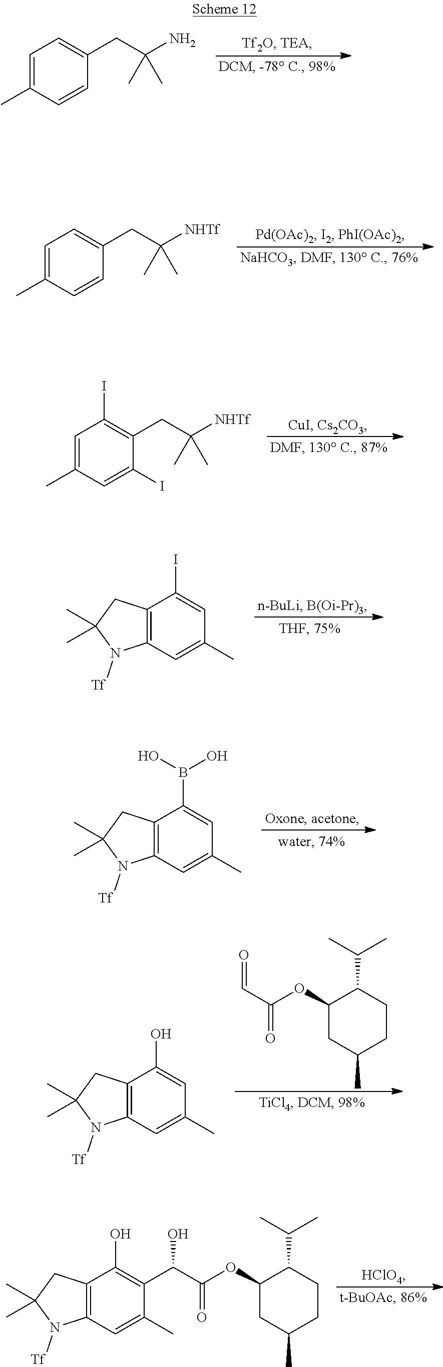

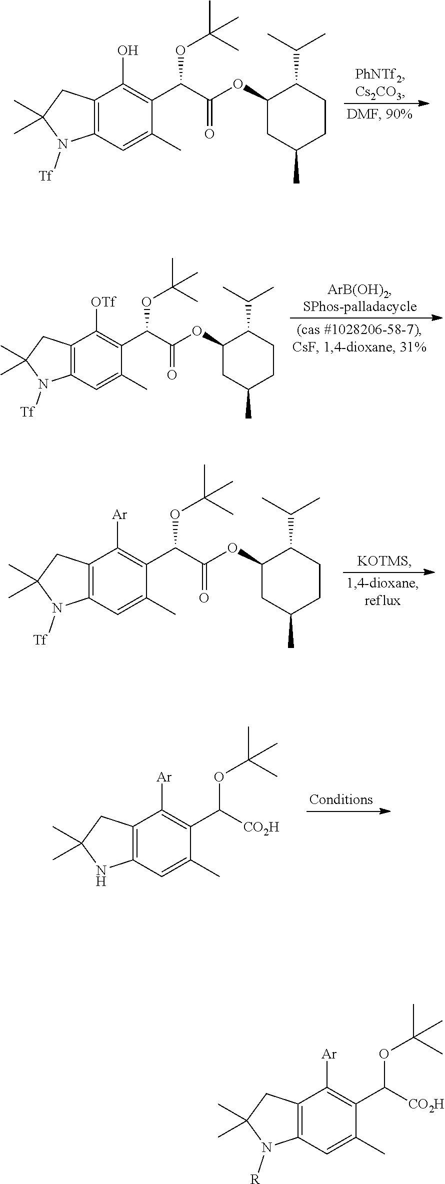

### Example 204: (S)-2-(tert-Butoxy)-2-((R)-4-(8-Fluoro-5-methylchroman-6-yl)-2,2,6-trimethyl-1-(6-methylnicotinoyl)indolin-5-yl)acetic acid

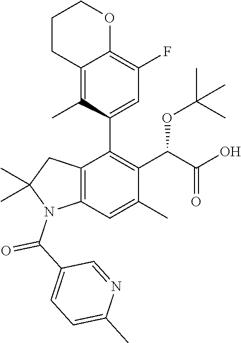

### Step 1: 2-Chloro-N-(2-methyl-1-(p-tolyl)propan-2-yl)acetamide

An ice cold mixture of 2-methyl-1-(p-tolyl)propan-2-ol (10 g, 60.9 mmol) and 2-chloroacetonitrile (11.56 mL, 183 mmol) in Acetic Acid (15 mL) was treated dropwise with H₂SO₄ (15 mL, 281 mmol) and stirred at room temperature for 5.5 hours. The reaction was poured into ice/water (240 mL), extracted with diethyl ether, washed with sat. NaHCO₃, Brine, dried over sodium sulfate, filtered, and concentrated in vacuo. to afford the title compound (13.6 g, 56.7 mmol, 93% yield) as off white solid. ¹H NMR (400 MHz, CHLOROFORM-d) δ ppm 7.17-7.08 (m, 2H), 7.08-6.99 (m, 2H), 6.25 (br. s., 1H), 3.96 (s, 2H), 2.99 (s, 2H), 2.34 (s, 3H), 1.38 (s, 6H); LCMS (m/z) ES⁺=240.2 (M+1).

### Step 2: 2-Methyl-1-(p-tolyl)propan-2-amine

A mixture of crude 2-chloro-N-(2-methyl-1-(p-tolyl)propan-2-yl)acetamide (13.6 g, 56.7 mmol, 93% yield) and thiourea (4.32 g, 56.7 mmol) in Ethanol (110 mL) was treated dropwise with acetic acid (22 mL, 384 mmol), and stirred at 85° C. for 4.5 hours. The reaction was treated with additional thiourea (872 mg), stirred at 85° C. for 1 hour, and then cooled to room temperature overnight. The mixture was diluted with water (600 mL), and then basified with 50% aq. NaOH. The mixture was extracted with hexanes, washed with Brine, dried over sodium sulfate, filtered, and concentrated in vacuo. to afford the title compound (8.51 g, 52.1 mmol, 86% yield) as yellow liquid. ¹H NMR (400 MHz, CHLOROFORM-d) δ ppm 7.19-7.00 (m, 4H), 2.63 (s, 2H), 2.34 (s, 3H), 1.42-1.19 (m, 2H), 1.12 (s, 6H); LCMS (m/z) ES⁺=164.1 (M+1).

### Step 3: 2,2,6-Trimethyl-1-((trifluoromethyl)sulfonyl)indolin-4-ol

In a manner similar to that described in Example 1, Step 1-5, the title compound was prepared as a yellow solid following purification with silica gel column chromatography (0-100% DCM/Hexanes) (1.3061 g, 4.22 mmol, 84% yield). ¹H NMR (400 MHz, CHLOROFORM-d) δ ppm 6.91 (s, 1H), 6.37 (s, 1H), 4.73 (s, 1H), 3.11-2.92 (m, 2H), 2.30 (s, 3H), 1.76 (s, 3H), 1.65 (s, 3H); LCMS (m/z) ES⁻=308.1 (M−1).

### Step 4: (S)-(1R,2S,5R)-2-Isopropyl-5-methylcyclohexyl 2-(tert-butoxy)-2-(4-hydroxy-2,2,6-trimethyl-1-((trifluoromethyl)sulfonyl)indolin-5-yl)acetate

In a manner similar to that described in Example 9, Step 1, the title compound was prepared as a as light yellow solid following purification with silica gel column chromatography (0-70% EtOAc/Hexanes) (1.1538 g, 1.997 mmol, 52.9% yield). ¹H NMR (400 MHz, CHLOROFORM-d) δ ppm 8.76-8.47 (m, 1H), 6.81 (s, 1H), 5.37-5.24 (m, 1H), 4.76-4.58 (m, 1H), 3.02 (br. s., 2H), 2.45-2.34 (m, 3H), 1.85-1.51 (m, 1 OH), 1.49-1.11 (m, 11H), 1.09-0.55 (m, 12H); LCMS (m/z) ES⁻=576.4 (M−1).

### Step 5: ((S)-2-(tert-Butoxy)-2-((R)-4-(8-fluoro-5-methylchroman-6-yl)-2,2,6-trimethylindolin-5-yl)acetic acid

In a manner similar to that described in Example 45, Steps 1-4 (replaced 1,4-dioxane in Step 3 with 1,2-dimethoxyethane), the title compound was prepared as brown foam. LCMS (m/z) ES⁺=456.4 (M+1).

### Step 6: (S)-2-(tert-Butoxy)-2-((R)-4-(8-Fluoro-5-methylchroman-6-yl)-2,2,6-trimethyl-1-(6-methylnicotinoyl)indolin-5-yl)acetic acid

A mixture of (S)-2-(tert-butoxy)-2-((R)-4-(8-Fluoro-5-methylchroman-6-yl)-2,2,6-trimethylindolin-5-yl)acetic acid (0.069 mmol) in Dichloromethane (DCM) (0.7 mL) was treated with Et3N (0.038 mL, 0.276 mmol), 6-methylnicotinoyl chloride (0.5M in DCM) (0.276 mL, 0.138 mmol), and stirred at r.t. for 18 hours. The reaction was diluted with sat. aq. NaHCO₃, extracted with DCM, washed with Brine, dried over Na2SO4, filtered, and concentrated. The residue was dissolved in Tetrahydrofuran (THF) (0.7 mL), treated with 2M LiOH (0.069 mL, 0.138 mmol), and stirred at r.t. for 2.5 hours. The reaction was diluted with 1N HCl, extracted with EtOAc, washed with Brine, dried over Na2SO4, filtered, and concentrated. Purification with reverse phase HPLC (30-100% MeCN—0.1% formic acid/H2O—0.1% formic acid) afforded title compound (4.8 mg, 7.93 μmol, 11.52% yield, (impure with ˜15-20% (P)(S) atropisomer)) as white solid. ¹H NMR (400 MHz, METHANOL-d₄) δ ppm 8.59 (s, 1H), 7.94 (d, J=8.1 Hz, 1H), 7.46 (d, J=7.9 Hz, 1H), 6.65 (d, J=11.4 Hz, 1H), 5.80 (s, 1H), 4.98-4.90 (m, 1H), 4.23 (t, J=4.9 Hz, 2H), 2.74 (t, J=6.2 Hz, 2H), 2.64 (s, 3H), 2.61-2.39 (m, 2H), 2.20-2.03 (m, 5H), 1.95-1.85 (m, 3H), 1.66-1.52 (m, 6H), 1.14-0.92 (m, 9H); LCMS (m/z) ES⁺=575.4 (M+1), ES⁻=573.4 (M−1).

### Example 205: (S)-2-(tert-Butoxy)-2-((R)-4-(8-Fluoro-5-methylchroman-6-yl)-1-(2-methoxy-5-methylbenzoyl)-2,2,6-trimethylindolin-5-yl)acetic acid

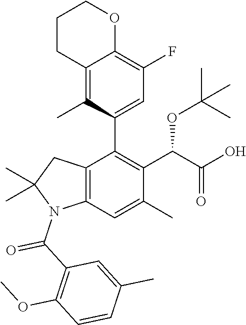

In a manner similar to that described in Example 204, (S)-2-(tert-butoxy)-2-((R)-4-(8-Fluoro-5-methylchroman-6-yl)-1-(2-methoxy-5-methylbenzoyl)-2,2,6-trimethylindolin-5-yl)acetic acid was prepared as a white solid in 49% yield (impure with ˜15-20% (P,S)-diastereomer). ¹H NMR (400 MHz, METHANOL-d₄) δ ppm 7.35 (d, J=8.2 Hz, 1H), 7.17 (br. s., 1H), 7.06-6.90 (m, 1H), 6.64 (dd, J=6.2, 11.0 Hz, 1H), 5.60 (br. s., 1H), 4.99-4.90 (m, 1H), 4.22 (t, J=4.7 Hz, 2H), 3.61 (d, J=12.6 Hz, 3H), 2.73 (br. s., 2H), 2.65-2.44 (m, 2H), 2.36 (br. s., 3H), 2.22-1.97 (m, 5H), 1.96-1.80 (m, 3H), 1.77-1.41 (m, 6H), 1.19-0.92 (m, 9H); LCMS (m/z) ES⁺=604.5 (M+1), ES⁻=602.4 (M−1).

### Example 206: (S)-2-(tert-Butoxy)-2-((R)-4-(8-fluoro-5-methylchroman-6-yl)-1-(4-methoxy-3-methylbenzoyl)-2,2,6-trimethylindolin-5-yl)acetic acid

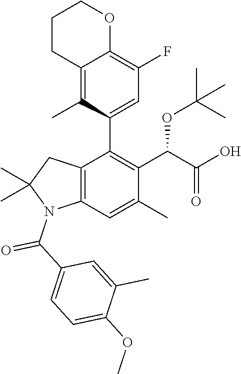

In a manner similar to that described in Example 204, (S)-2-(tert-butoxy)-2-((R)-4-(8-fluoro-5-methylchroman-6-yl)-1-(4-methoxy-3-methylbenzoyl)-2,2,6-trimethylindolin-5-yl)acetic acid was prepared as a white solid in 9% yield (impure with ˜20% (P,S)-diastereomer). ¹H NMR (400 MHz, METHANOL-d₄) δ ppm 7.50-7.28 (m, 2H), 7.08-6.93 (m, 1H), 6.66 (d, J=11.2 Hz, 1H), 5.80 (s, 1H), 4.98-4.91 (m, 1H), 4.32-4.14 (m, 2H), 3.92 (s, 3H), 2.74 (t, J=5.6 Hz, 2H), 2.63-2.32 (m, 2H), 2.23 (s, 3H), 2.18-2.00 (m, 5H), 1.91 (s, 3H), 1.66-1.51 (m, 6H), 1.13-0.91 (m, 9H); LCMS (m/z) ES⁻=602.6 (M−1).

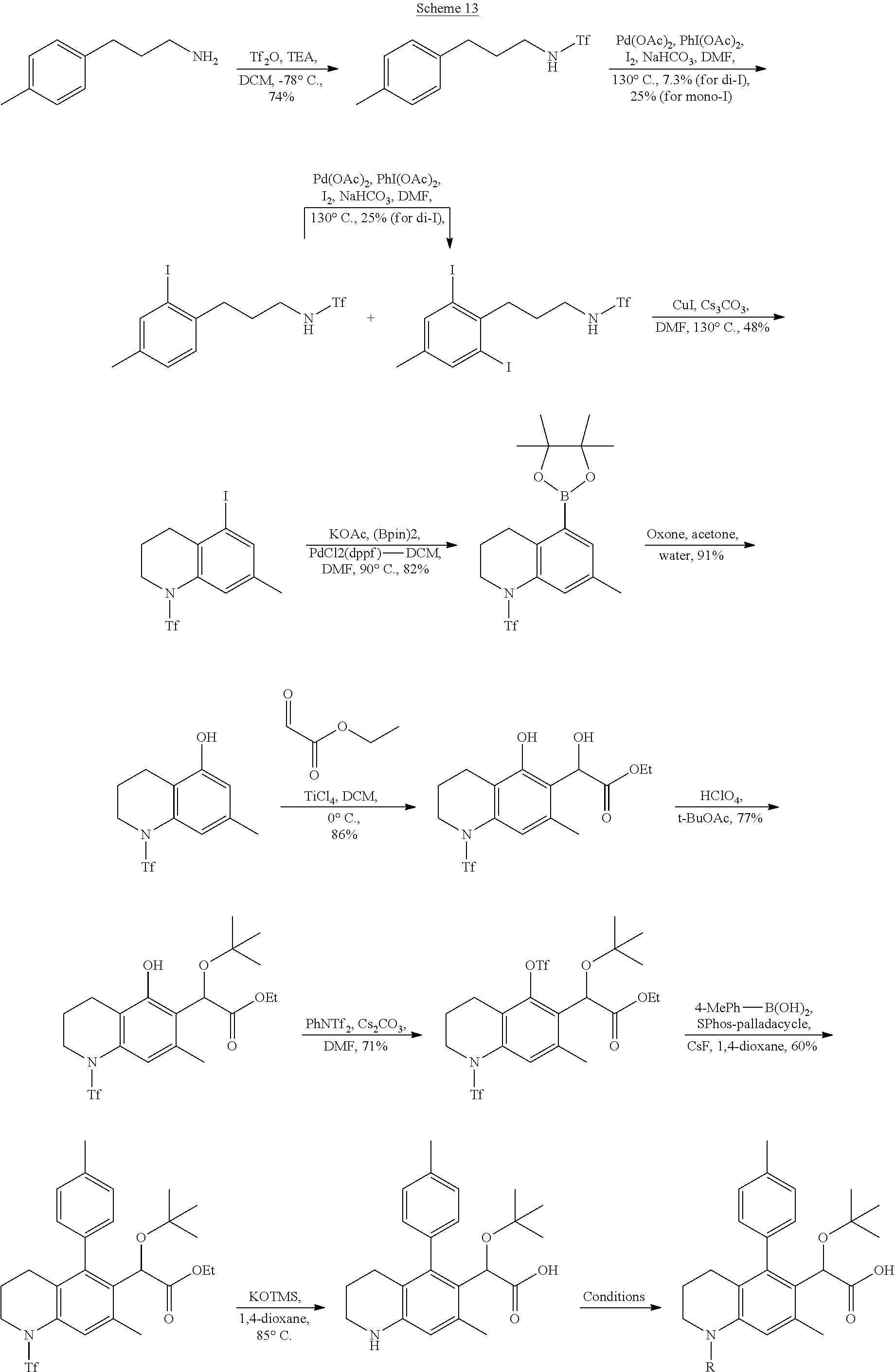

### Example 207: 2-(tert-Butoxy)-2-(7-methyl-1-(2-oxo-2-phenylethyl)-5-(p-tolyl)-1,2,3,4-tetrahydroquinolin-6-yl)acetic acid

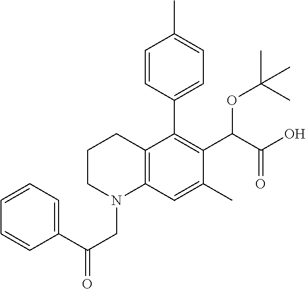 [SMILES: Cc1ccc(-c2c3c(cc(C)c2C(OC(C)(C)C)C(=O)O)N(CC(=O)c2ccccc2)CCC3)cc1]

### Step 1: 1,1,1-Trifluoro-N-(3-(p-tolyl)propyl)methanesulfonamide

3-(p-Tolyl)propan-1-amine (8.8 g, 59 mmol) in dichloromethane (90 mL) was cooled to −78° C. before triethylamine (9.04 mL, 64.9 mmol) was added. Trifluoromethanesulfonic anhydride (1M in dichloromethane) (64.9 mL, 64.9 mmol) was added dropwise and the mixture stirred for 1 hour at −78° C. The mixture was poured into ice water and extracted 2 times with dichloromethane. The combined organic layers were washed with brine, dried over sodium sulfate, and concentrated. The residue was purified by silica chromatography eluting with gradient of 50% to 100% of dichloromethane in hexanes. Fractions were concentrated to give 1,1,1-trifluoro-N-(3-(p-tolyl)propyl)methanesulfonamide (12.2 g, 43.5 mmol, 73.8% yield) as a clear liquid. ¹H NMR (400 MHz, DMSO-d₆) δ ppm 9.4 (s, 1H), 7.0-7.2 (m, 4H), 3.1 (t, J=6.96 Hz, 2H), 2.6 (t, J=7.69 Hz, 2H), 2.3 (s, 3H), 1.8 (q, J=7.33 Hz, 2H). LCMS (ES−)(m/z): 280.24 (M−H).

### Step 2: 1,1,1-Trifluoro-N-(3-(2-iodo-4-methylphenyl)propyl)methanesulfonamide and N-(3-(2,6-Diiodo-4-methylphenyl)propyl)-1,1,1-trifluoromethanesulfonamide

1,1,1-Trifluoro-N-(3-(p-tolyl)propyl)methanesulfonamide (5.80 g, 20.6 mmol), diacetoxypalladium (0.926 g, 4.12 mmol), iodobenzene diacetate (16.6 g, 51.5 mmol), sodium hydrogen carbonate (2.60 g, 30.9 mmol), and iodine (13.1 g, 51.5 mmol) in N,N-dimethylformamide (DMF) (100 mL) were heated at 130° C. overnight. The mixture was allowed to cool to room temperature and was diluted with brine. The mixture was extracted 3 times with ethyl acetate. The combined organic layers were washed 2 times with saturated aqueous sodium thiosulfate, washed with 5% aqueous lithium chloride, dried over sodium sulfate and concentrated. The dark residue was filtered through a silica plug with dichloromethane. Fractions were concentrated. The residue was purified by reverse phase medium-pressure chromatography eluting with a gradient of 30% to 100% (acetonitrile/water/0.1% formic acid). One set of fractions was concentrated to give 1,1,1-trifluoro-N-(3-(2-iodo-4-methylphenyl)propyl)methanesulfonamide (2.1 g, 5.16 mmol, 25.01% yield). ¹H NMR (400 MHz, CHLOROFORM-d) δ ppm 7.7 (s, 1H), 7.0-7.2 (m, 2H), 4.9 (br. s., 1H), 3.4 (q, J=6.59 Hz, 2H), 2.7-2.9 (m, 2H), 2.3 (s, 3H), 1.9 (quin, J=7.33 Hz, 2H). LCMS (ES−)(m/z): 406.09 (M−H).

A second set of fractions was concentrated to give N-(3-(2,6-diiodo-4-methylphenyl)propyl)-1,1,1-trifluoromethanesulfonamide (800 mg, 1.50 mmol, 7.28% yield). ¹H NMR (400 MHz, CHLOROFORM-d) δ ppm 7.6-7.8 (m, 2H), 4.9 (br. s., 1H), 3.5 (q, J=6.53 Hz, 2H), 3.0-3.2 (m, 2H), 2.2-2.3 (m, 3H), 1.8-1.9 (m, 2H). LCMS (ES−)(m/z): 532.18 (M−H).

### Step 3: 1,1,1-Trifluoro-N-(3-(2-iodo-4-methylphenyl)propyl)methanesulfonamide and N-(3-(2,6-Diiodo-4-methylphenyl)propyl)-1,1,1-trifluoromethanesulfonamide

1,1,1-Trifluoro-N-(3-(2-iodo-4-methylphenyl)propyl)methanesulfonamide (1.84 g, 4.52 mmol), palladium(II) acetate (0.152 g, 0.678 mmol), iodobenzene diacetate (2.91 g, 9.04 mmol), sodium bicarbonate (0.380 g, 4.52 mmol), and iodine (2.29 g, 9.04 mmol) were added sequentially to N,N-dimethylformamide (DMF) (30 mL). The reaction vessel was placed in an oil bath that was pre-heated to 130° C. and the mixture heated overnight. The mixture was allowed to cool to room temperature and diluted with brine. The mixture was extracted 3 times with 2-methyltetrahydrofuran. The combined organic layers were washed 2 times with saturated aqueous sodium thiosulfate, washed with 5% aqueous lithium chloride, dried over sodium sulfate and concentrated. The dark residue was filtered through a silica plug with dichloromethane. Fractions containing both monoiodide and diiodide were concentrated. The residue was purified by reverse phase ISCO chromatography (30% to 100% acetonitrile/water/0.1% formic acid). Fractions of the later eluting peak were concentrated to give N-(3-(2,6-diiodo-4-methylphenyl)propyl)-1,1,1-trifluoromethane sulfonamide (600 mg, 1.13 mmol, 24.9% yield). ¹H NMR (400 MHz, CHLOROFORM-d) δ ppm 7.7 (s, 2H), 4.9 (br. s., 1H), 3.5 (q, J=6.59 Hz, 2H), 3.0-3.2 (m, 2H), 2.1-2.4 (m, 3H), 1.8-2.0 (m, 2H). LCMS (ES−)(m/z): 532.01 (M−H).

Fractions of the earlier eluting peak were concentrated to give 1,1,1-trifluoro-N-(3-(2-iodo-4-methylphenyl)propyl)methanesulfonamide (260 mg, 0.639 mmol, 14.1% yield). ¹H NMR (400 MHz, CHLOROFORM-d) δ ppm 7.7 (s, 1H), 6.9-7.2 (m, 2H), 4.9 (br. s., 1H), 3.4 (q, J=6.53 Hz, 2H), 2.8 (t, J=7.69 Hz, 2H), 2.2-2.4 (m, 3H), 1.9 (quin, J=7.33 Hz, 2H). LCMS (ES−)(m/z): 406.10 (M−H).

### Step 4: 5-iodo-7-methyl-1-((trifluoromethyl)sulfonyl)-1,2,3,4-tetrahydroquinoline

N-(3-(2,6-Diiodo-4-methylphenyl)propyl)-1,1,1-trifluoromethanesulfonamide (3.43 g, 6.43 mmol), copper(I) iodide (0.613 g, 3.22 mmol), and cesium carbonate (2.31 g, 7.08 mmol) in N,N-dimethylformamide (DMF) (50 mL) were heated to 130° C. by immersing the reaction vessel in a pre-heated oil bath. The mixture was stirred and heated overnight.

The mixture was allowed to cool to room temperature, diluted with dichloromethane, and solids filtered off. The filtrate was concentrated and the residue purified by silica chromatography eluting with 25% dichloromethane in hexanes until desired product eluted, then ramped to 100% dichloromethane. Fractions were concentrated to give 5-iodo-7-methyl-1-((trifluoromethyl)sulfonyl)-1,2,3,4-tetrahydroquinoline (1.25 g, 3.09 mmol, 47.9% yield). ¹H NMR (400 MHz, CHLOROFORM-d) δ ppm 7.6 (s, 1H), 7.3 (s, 1H), 3.8 (t, J=5.68 Hz, 2H), 2.8 (t, J=6.96 Hz, 2H), 2.3-2.4 (m, 3H), 2.1-2.2 (m, 2H).

### Step 5: 7-Methyl-5-(4,4,5,5-tetramethyl-1,3,2-dioxaborolan-2-yl)-1-((trifluoromethyl)sulfonyl)-1,2,3,4-tetrahydroquinoline

5-Iodo-7-methyl-1-((trifluoromethyl)sulfonyl)-1,2,3,4-tetrahydroquinoline (1.2 g, 3.0 mmol), potassium acetate (0.872 g, 8.89 mmol), and bis(pinacolato)diboron (1.13 g, 4.44 mmol) in N,N-dimethylformamide (DMF) (30 mL) were degassed with nitrogen for 10 minutes. PdCl₂(dppf)-CH₂Cl₂adduct (0.242 g, 0.296 mmol) was added and the mixture heated at 90° C. for 4 hours. The mixture was allowed to cool to room temperature and diluted with brine. The mixture was extracted 2 times with ethyl acetate. The combined organic layers were washed with brine, dried over sodium sulfate, and concentrated. The residue was purified by silica chromatography eluting with a gradient of 0% to 100% dichloromethane in hexanes. Fractions were concentrated to give 7-methyl-5-(4,4,5,5-tetramethyl-1,3,2-dioxaborolan-2-yl)-1-((trifluoromethyl)sulfonyl)-1,2,3,4-tetrahydroquinoline (980 mg, 2.42 mmol, 82% yield) as a white solid. ¹H NMR (400 MHz, CHLOROFORM-d) δ ppm 7.5 (s, 1H), 7.4 (s, 1H), 3.8 (t, J=6.32 Hz, 2H), 3.1 (t, J=6.68 Hz, 2H), 2.3 (s, 3H), 2.1 (t, J=6.50 Hz, 2H), 1.4 (s, 12H).

### Step 6: 7-Methyl-1-((trifluoromethyl)sulfonyl)-1,2,3,4-tetrahydroquinolin-5-ol

To a solution of 7-methyl-5-(4,4,5,5-tetramethyl-1,3,2-dioxaborolan-2-yl)-1-((trifluoromethyl)sulfonyl)-1,2,3,4-tetrahydroquinoline (950 mg, 2.34 mmol) in acetone (16 mL) at 0° C. was added oxone (1730 mg, 2.81 mmol) in water (8.00 mL) dropwise. The mixture was allowed to warm to room temperature and stirred for 2 hours. The mixture was poured into an iced water/sodium bicarbonate solution and extracted 2 times with ethyl acetate. The combined organic layers were washed with brine, dried over sodium sulfate, and concentrated. The residue was purified by silica chromatography eluting with a gradient of 0% to 30% ethyl acetate in hexanes. Fractions were concentrated to give an oil. NMR showed that 0.36 eq. of ethyl acetate was present. The product was dried under high vacuum for 3 hours and the oil partially crystallized to give 7-methyl-1-((trifluoromethyl)sulfonyl)-1,2,3,4-tetrahydroquinolin-5-ol (628 mg, 2.13 mmol, 91% yield).

¹H NMR (400 MHz, CHLOROFORM-d) δ ppm 7.0 (s, 1H), 6.5 (s, 1H), 4.8 (s, 1H), 3.7-3.9 (m, 2H), 2.8 (t, J=6.96 Hz, 2H), 2.3 (s, 3H), 2.1-2.2 (m, 2H). LCMS (ES+) (m/z): 296.24 (M+H).

### Step 7: Ethyl 2-hydroxy-2-(5-hydroxy-7-methyl-1-((trifluoromethyl)sulfonyl)-1,2,3,4-tetrahydroquinolin-6-yl)acetate

Titanium tetrachloride (1M in dichloromethane) (2.302 mL, 2.302 mmol) was added dropwise over about 8 minutes to a 0° C. solution of 7-methyl-1-((trifluoromethyl)sulfonyl)-1,2,3,4-tetrahydroquinolin-5-ol (618 mg, 2.09 mmol) in dichloromethane (DCM) (20 mL).

The solution became deep red with addition of the titanium tetrachloride. The mixture was stirred an additional 5 minutes after addition was complete. A solution of ethyl glyoxylate solution (50% in toluene) (0.568 mL, 2.72 mmol) in 10 mL of dichloromethane was added dropwise and the mixture stirred an additional 15 minutes. The mixture was poured carefully into iced 1N hydrochloric acid. The mixture was extracted 3 times with 2-methyltetrahydrofuran. The combined organic layers were washed with brine, dried over sodium sulfate, and concentrated. The residue was purified by silica chromatography eluting with a gradient of 0% to 10% ethyl acetate in dichloromethane. Fractions were concentrated to give ethyl 2-hydroxy-2-(5-hydroxy-7-methyl-1-((trifluoromethyl)sulfonyl)-1,2,3,4-tetrahydroquinolin-6-yl)acetate (717 mg, 1.80 mmol, 86% yield). ¹H NMR (400 MHz, CHLOROFORM-d) δ ppm 8.0 (s, 1H), 7.0 (s, 1H), 5.5 (s, 1H), 4.3 (dq, J=10.67, 7.19 Hz, 1H), 4.1-4.3 (m, 1H), 3.8 (t, J=5.31 Hz, 2H), 3.5 (s, 1H), 2.6-3.0 (m, 2H), 2.4 (s, 3H), 1.9-2.2 (m, 2H), 1.1-1.4 (m, 3H). LCMS (ES+) (m/z): 398.25 (M+H).

### Step 8: Ethyl 2-(tert-butoxy)-2-(5-hydroxy-7-methyl-1-((trifluoromethyl)sulfonyl)-1,2,3,4-tetrahydroquinolin-6-yl)acetate

Ethyl 2-hydroxy-2-(5-hydroxy-7-methyl-1-((trifluoromethyl)sulfonyl)-1,2,3,4-tetrahydroquinolin-6-yl)acetate (717 mg, 1.80 mmol) was dissolved in tert-butyl acetate (20 mL, 148 mmol) before perchloric acid (0.310 mL, 3.61 mmol) was added dropwise. The mixture was stirred for 15 minutes, quenched with saturated sodium bicarbonate, and extracted 2 times with 2-methyltetrahydrofuran. The combined organic layers were washed with brine, dried over sodium sulfate, and concentrated. The residue was purified by silica chromatography eluting with a gradient of 0% to 20% ethyl acetate in hexanes.

Fractions were concentrated to give ethyl 2-(tert-butoxy)-2-(5-hydroxy-7-methyl-1-((trifluoromethyl)sulfonyl)-1,2,3,4-tetrahydroquinolin-6-yl)acetate (633 mg, 1.40 mmol, 77% yield) as a clear oil. ¹H NMR (400 MHz, CHLOROFORM-d) δ ppm 8.6 (s, 1H), 6.9 (s, 1H), 5.3 (s, 1H), 4.0-4.3 (m, 2H), 3.7-3.9 (m, 2H), 2.8 (t, J=6.96 Hz, 2H), 2.4 (s, 3H), 2.1 (t, J=6.32 Hz, 2H), 1.3 (s, 9H), 1.2 (t, J=7.14 Hz, 3H). LCMS (ES+) (m/z): 454.32 (M+H).

### Step 9: Ethyl 2-(tert-butoxy)-2-(7-methyl-1-((trifluoromethyl)sulfonyl)-5-(((trifluoromethyl)sulfonyl)oxy)-1,2,3,4-tetrahydroquinolin-6-yl)acetate

1,1,1-Trifluoro-N-phenyl-N-((trifluoromethyl)sulfonyl)methanesulfonamide (598 mg, 1.68 mmol) was added to a stirring mixture of ethyl 2-(tert-butoxy)-2-(5-hydroxy-7-methyl-1-((trifluoromethyl)sulfonyl)-1,2,3,4-tetrahydroquinolin-6-yl)acetate (633 mg, 1.40 mmol) and cesium carbonate (910 mg, 2.79 mmol) in N,N-dimethylformamide (DMF) (10 mL) at room temperature. The mixture was stirred for 1 hour, quenched with 5% lithium chloride, and extracted 3 times with ethyl acetate. The combined organic layers were washed with 5% lithium chloride, dried over sodium sulfate, and concentrated. The residue was purified by silica chromatography eluting with a gradient of 0% to 20% ethyl acetate in hexanes. Fractions were concentrated to give ethyl 2-(tert-butoxy)-2-(7-methyl-1-((trifluoromethyl)sulfonyl)-5-(((trifluoromethyl)sulfonyl)oxy)-1,2,3,4-tetrahydroquinolin-6-yl)acetate (579 mg, 0.989 mmol, 70.8% yield) as a clear oil. ¹H NMR (400 MHz, CHLOROFORM-d) δ ppm 7.4 (s, 1H), 5.5 (s, 1H), 4.1-4.3 (m, 2H), 3.9-4.1 (m, 1H), 3.6-3.8 (m, 1H), 2.9 (d, J=8.97 Hz, 2H), 2.4 (s, 3H), 2.0-2.3 (m, 2H), 1.1-1.3 (m, 12H). LCMS (ES+) (m/z): 608.32 (M+Na). LCMS (ES−)(m/z): 584.55 (M−H).

### Step 10: Ethyl 2-(tert-butoxy)-2-(7-methyl-5-(p-tolyl)-1-((trifluoromethyl)sulfonyl)-1,2,3,4-tetrahydroquinolin-6-yl)acetate and Ethyl 2-(tert-butoxy)-2-(7-methyl-5-(p-tolyl)-1,2,3,4-tetrahydroquinolin-6-yl)acetate

Ethyl 2-(tert-butoxy)-2-(7-methyl-1-((trifluoromethyl)sulfonyl)-5-(((trifluoromethyl)sulfonyl)oxy)-1,2,3,4-tetrahydroquinolin-6-yl)acetate (150 mg, 0.256 mmol), p-tolylboronic acid (87 mg, 0.64 mmol), cesium fluoride (156 mg, 1.03 mmol), and SPhos-Pd (58.5 mg, 0.0770 mmol) in 1,2-dimethoxyethane (DME) (4 mL) were purged with nitrogen before being heated at 130° C. in a microwave reactor for 30 minutes. After cooling, the reaction mixture filtered through a cotton plug. The filtrate was concentrated and the residue purified by silica chromatography eluting with a gradient of 0% to 40% gradient of ethyl acetate in hexanes. One set of fractions was concentrated to give ethyl 2-(tert-butoxy)-2-(7-methyl-5-(p-tolyl)-1-((trifluoromethyl)sulfonyl)-1,2,3,4-tetrahydroquinolin-6-yl)acetate (81 mg, 0.154 mmol, 59.9% yield). ¹H NMR (400 MHz, CHLOROFORM-d) δ ppm 7.2-7.3 (m, 3H), 7.2-7.2 (m, 1H), 7.0 (d, J=7.69 Hz, 1H), 4.9 (s, 1H), 4.1 (dd, J=12.18, 7.05 Hz, 2H), 3.7-3.9 (m, 2H), 2.4-2.5 (m, 7H), 2.3 (s, 1H), 2.0 (s, 2H), 1.2-1.3 (m, 3H), 1.0 (s, 9H). LCMS (ES+) (m/z): 550.34 (M+Na).

A separate set of fractions was concentrated to give ethyl 2-(tert-butoxy)-2-(7-methyl-5-(p-tolyl)-1,2,3,4-tetrahydroquinolin-6-yl)acetate (11 mg, 0.028 mmol, 11% yield).

¹H NMR (400 MHz, CHLOROFORM-d) δ ppm 7.2 (s, 2H), 7.2 (d, J=7.87 Hz, 1H), 7.1 (d, J=7.51 Hz, 1H), 6.3 (s, 1H), 4.8 (s, 1H), 4.0-4.2 (m, 2H), 3.2-3.3 (m, 2H), 2.4 (s, 3H), 2.3-2.4 (m, 5H), 2.0-2.2 (m, 1H), 1.7-1.9 (m, 2H), 1.2-1.3 (m, 3H), 1.0 (s, 9H). LCMS (ES+) (m/z): 396.37 (M+H).

### Step 11: 2-(tert-Butoxy)-2-(7-methyl-5-(p-tolyl)-1,2,3,4-tetrahydroquinolin-6-yl)acetic acid

Ethyl 2-(tert-butoxy)-2-(7-methyl-5-(p-tolyl)-1-((trifluoromethyl)sulfonyl)-1,2,3,4-tetrahydroquinolin-6-yl)acetate (100 mg, 0.190 mmol) and potassium trimethylsilanolate (135 mg, 0.948 mmol) were heated at 85° C. under nitrogen for 5 hours. The mixture was allowed to cool to room temperature and quenched with 1N hydrochloric acid (5 drops at a time until pH=2-3), diluted with water (2 mL), and extracted 3 times with ethyl acetate. The combined organic layers were dried over sodium sulfate and concentrated to give 2-(tert-butoxy)-2-(7-methyl-5-(p-tolyl)-1,2,3,4-tetrahydroquinolin-6-yl)acetic acid (76 mg, 0.19 mmol, 98% yield). ¹H NMR (400 MHz, CHLOROFORM-d) δ ppm 7.4 (d, J=7.14 Hz, 1H), 7.2-7.3 (m, 2H), 7.1 (d, J=7.33 Hz, 1H), 6.3 (s, 1H), 5.0 (s, 1H), 3.7 (s, 1H), 3.1-3.4 (m, 2H), 2.4 (s, 4H), 2.3 (s, 3H), 2.1 (dt, J=16.48, 5.13 Hz, 2H), 1.8-1.9 (m, 1H), 1.7-1.8 (m, 1H), 0.9-1.1 (m, 9H). LCMS (ES+) (m/z): 368.44 (M+H).

### Step 12: 2-(tert-Butoxy)-2-(7-methyl-1-(2-oxo-2-phenylethyl)-5-(p-tolyl)-1,2,3,4-tetrahydroquinolin-6-yl)acetic acid

2-(tert-Butoxy)-2-(7-methyl-5-(p-tolyl)-1,2,3,4-tetrahydroquinolin-6-yl)acetic acid (14 mg, 0.034 mmol) was dissolved in ethanol (0.25 mL) before potassium carbonate (11.9 mg, 0.086 mmol) and 2-bromo-1-phenylethanone (6.8 mg, 0.034 mmol) were added. The mixture was stirred at room temperature for 3 hours. The mixture was filtered through a cotton plug that was washed with DMF and the filtrate purified by reverse phase HPLC (acetonitrile/water/0.1% formic acid). Fractions were concentrated. Acetonitrile was added and concentrated to remove residual water and give 2-(tert-butoxy)-2-(7-methyl-1-(2-oxo-2-phenylethyl)-5-(p-tolyl)-1,2,3,4-tetrahydroquinolin-6-yl)acetic acid (10 mg, 0.020 mmol, 58% yield) as a clear residue. ¹H NMR (400 MHz, CHLOROFORM-d) δ ppm 7.9 (d, J=7.87 Hz, 2H), 7.6 (d, J=7.33 Hz, 1H), 7.4-7.5 (m, 2H), 7.2-7.3 (m, 2H), 7.0-7.2 (m, 2H), 6.3 (s, 1H), 5.1-5.4 (m, 2H), 5.1 (s, 1H), 3.2 (d, J=4.58 Hz, 2H), 2.4 (s, 3H), 2.3-2.4 (m, 4H), 2.1 (d, J=16.85 Hz, 1H), 1.7-1.9 (m, 3H), 0.9-1.0 (m, 9H). LCMS (ES+) (m/z): 486.38 (M+H).

### Example 208: 2-(1-((Benzyloxy)carbonyl)-7-methyl-5-(p-tolyl)-1,2,3,4-tetrahydroquinolin-6-yl)-2-(tert-butoxy)acetic acid

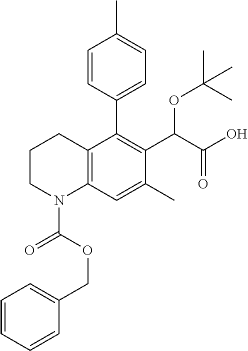 [SMILES: Cc1ccc(-c2c3c(cc(C)c2C(OC(C)(C)C)C(=O)O)N(C(=O)OCc2ccccc2)CCC3)cc1]

2-(tert-Butoxy)-2-(7-methyl-5-(p-tolyl)-1,2,3,4-tetrahydroquinolin-6-yl)acetic acid (14 mg, 0.034 mmol) was dissolved in dichloromethane (DCM) (0.25 mL) before pyridine (8.32 μl, 0.103 mmol) and benzyl chloroformate (4.9 μl, 0.034 mmol) were added. The mixture was stirred at room temperature for 3 hours and concentrated. The residue was purified by reverse phase HPLC (acetonitrile/water/0.1% formic acid). Fractions were concentrated and the residue azeotroped with acetonitrile to remove residual water. Concentration gave 2-(1-((benzyloxy)carbonyl)-7-methyl-5-(p-tolyl)-1,2,3,4-tetrahydroquinolin-6-yl)-2-(tert-butoxy)acetic acid (3.5 mg, 6.91 μmol, 20% yield) as a clear residue. ¹H NMR (400 MHz, CHLOROFORM-d) δ ppm 7.5 (br. s., 1H), 7.3-7.4 (m, 6H), 7.2-7.3 (m, 2H), 7.1 (d, J=7.33 Hz, 1H), 5.3 (s, 2H), 5.1 (s, 1H), 3.8 (br. s., 1H), 3.7 (br. s., 1H), 2.3-2.5 (m, 7H), 2.1-2.3 (m, 1H), 1.7-1.9 (m, 2H), 1.0 (s, 9H). LCMS (ES+) (m/z): 502.39 (M+H).

### Example 209: 2-(tert-Butoxy)-2-(1-(3-(methoxycarbonyl)benzyl)-7-methyl-5-(p-tolyl)-1,2,3,4-tetrahydroquinolin-6-yl)acetic acid

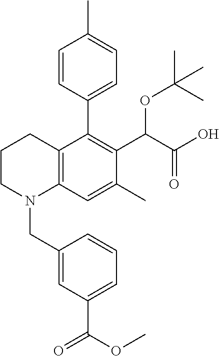 [SMILES: COC(=O)c1cccc(CN2CCCc3c2cc(C)c(C(OC(C)(C)C)C(=O)O)c3-c2ccc(C)cc2)c1]

2-(tert-Butoxy)-2-(7-methyl-5-(p-tolyl)-1,2,3,4-tetrahydroquinolin-6-yl)acetic acid (14 mg, 0.034 mmol) was dissolved in ethanol (0.25 mL) before potassium carbonate (11.9 mg, 0.0860 mmol) and methyl 3-(bromomethyl)benzoate (7.85 mg, 0.0340 mmol) were added. The mixture was stirred at room temperature for 3 hours. The mixture was filtered through a cotton plug that was subsequently washed with DMF and the filtrate injected onto a reverse phase HPLC system eluting with acetonitrile/water/0.1% formic acid. Fractions were concentrated. Acetonitrile was added and evaporated to remove residual water and give 2-(tert-butoxy)-2-(1-(3-(methoxycarbonyl)benzyl)-7-methyl-5-(p-tolyl)-1,2,3,4-tetrahydroquinolin-6-yl)acetic acid (7.6 mg, 0.014 mmol, 40% yield). ¹H NMR (400 MHz, CHLOROFORM-d) δ ppm 9.4-10.1 (m, 1H), 7.9-8.0 (m, 2H), 7.5-7.5 (m, 1H), 7.4-7.5 (m, 2H), 7.2-7.3 (m, 1H), 7.2 (s, 1H), 7.1 (d, J=7.14 Hz, 1H), 6.3 (s, 1H), 5.0 (s, 1H), 4.4-4.6 (m, 2H), 3.8-4.0 (m, 3H), 3.1-3.4 (m, 2H), 2.4-2.6 (m, 1H), 2.4 (s, 3H), 2.2 (s, 3H), 2.1-2.2 (m, 1H), 1.7-1.9 (m, 2H), 0.9-1.0 (m, 9H). LCMS (ES+) (m/z): 516.45 (M+H).

### Example 210: 2-(tert-Butoxy)-2-(7-methyl-1-(propylcarbamoyl)-5-(p-tolyl)-1,2,3,4-tetrahydroquinolin-6-yl)acetic acid

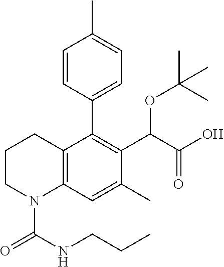 [SMILES: CCCNC(=O)N1CCCc2c1cc(C)c(C(OC(C)(C)C)C(=O)O)c2-c1ccc(C)cc1]

1-Isocyanatopropane (6.25 μl, 0.0650 mmol) was added to a solution of 2-(tert-butoxy)-2-(7-methyl-5-(p-tolyl)-1,2,3,4-tetrahydroquinolin-6-yl)acetic acid (12 mg, 0.033 mmol) and triethylamine (0.018 mL, 0.131 mmol) in 1,2-dichloroethane (DCE) (0.25 mL). The mixture was stirred at room temperature overnight and then concentrated. The residue was purified by reverse phase HPLC (acetonitrile/water/0.1% formic acid). Fractions were concentrated and the residue dried under vacuum to give 2-(tert-butoxy)-2-(7-methyl-1-(propylcarbamoyl)-5-(p-tolyl)-1,2,3,4-tetrahydroquinolin-6-yl)acetic acid (4.7 mg, 9.5 μmol, 29% yield) as a white powder. ¹H NMR (400 MHz, CHLOROFORM-d) b ppm 8.0-8.1 (m, 1H), 7.4 (d, J=7.51 Hz, 1H), 7.2-7.3 (m, 1H), 7.2 (d, J=7.69 Hz, 1H), 7.1 (s, 1H), 7.1 (d, J=7.51 Hz, 1H), 5.2 (t, J=5.04 Hz, 1H), 5.1 (s, 1H), 3.7-3.8 (m, 1H), 3.6 (dd, J=7.51, 5.13 Hz, 1H), 3.3 (q, J=6.59 Hz, 2H), 2.4 (s, 3H), 2.4 (s, 3H), 2.1-2.2 (m, 1H), 2.0 (s, 1H), 1.8 (dd, J=12.45, 6.23 Hz, 1H), 1.7 (dd, J=13.28, 7.23 Hz, 1H), 1.5-1.6 (m, 2H), 1.0 (s, 9H), 0.9 (t, J=7.33 Hz, 3H). LCMS (ES+) (m/z): 453.42 (M+H).

### Example 211: 2-(tert-Butoxy)-2-(7-methyl-1-phenyl-5-(p-tolyl)-1,2,3,4-tetrahydroquinolin-6-yl)acetic acid

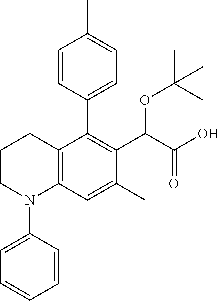 [SMILES: Cc1ccc(-c2c3c(cc(C)c2C(OC(C)(C)C)C(=O)O)N(c2ccccc2)CCC3)cc1]

Ethyl 2-(tert-butoxy)-2-(7-methyl-5-(p-tolyl)-1,2,3,4-tetrahydroquinolin-6-yl)acetate (11 mg, 0.028 mmol), cesium carbonate (23 mg, 0.071 mmol), bromobenzene (3.8 μl, 0.036 mmol), BINAP (2.77 mg, 4.45 μmol), and tris(dibenzylideneacetone)dipalladium(0) (2.55 mg, 2.78 μmol) in toluene (1 mL) were heated at 90° C. under nitrogen for 16 hours. The mixture was concentrated and the residue was dissolved in 1,4-dioxane (1 mL). Potassium trimethylsilanolate (21.4 mg, 0.167 mmol) was added and the mixture heated at 90° C. for 2 hours. The mixture was allowed to cool to room temperature, quenched with 1N hydrochloric acid, and extracted 3 times with ethyl acetate. The combined organic layers were concentrated and the residue purified by reverse phase HPLC (acetonitrile/water/0.1% formic acid). Fractions were concentrated to give 2-(tert-butoxy)-2-(7-methyl-1-phenyl-5-(p-tolyl)-1,2,3,4-tetrahydroquinolin-6-yl)acetic acid (3.0 mg, 6.6 μmol, 24% yield). ¹H NMR (400 MHz, CHLOROFORM-d) δ ppm 9.5-10.1 (m, 1H), 7.4 (d, J=6.96 Hz, 1H), 7.3-7.4 (m, 2H), 7.2-7.3 (m, 3H), 7.2 (d, J=7.87 Hz, 1H), 7.1-7.2 (m, 2H), 6.6 (s, 1H), 5.0 (s, 1H), 3.4-3.7 (m, 2H), 2.5-2.6 (m, 1H), 2.4 (s, 3H), 2.1-2.3 (m, 4H), 1.8-2.0 (m, 2H), 1.0 (s, 9H). LCMS (ES+) (m/z): 444.35 (M+H).

### Example 212: 2-(tert-Butoxy)-2-(1-(3,4-difluorobenzyl)-7-methyl-5-(p-tolyl)-1,2,3,4-tetrahydroquinolin-6-yl)acetic acid

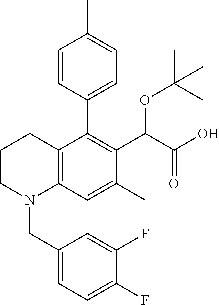 [SMILES: Cc1ccc(-c2c3c(cc(C)c2C(OC(C)(C)C)C(=O)O)N(Cc2ccc(F)c(F)c2)CCC3)cc1]

2-(tert-Butoxy)-2-(7-methyl-5-(p-tolyl)-1,2,3,4-tetrahydroquinolin-6-yl)acetic acid (10 mg, 0.024 mmol) was dissolved in ethanol (0.25 mL) before potassium carbonate (8.5 mg, 0.061 mmol) and 4-(bromomethyl)-1,2-difluorobenzene (3.5 μl, 0.027 mmol) were added. The mixture was stirred at room temperature overnight, allowed to cool to room temperature, and was filtered through a cotton plug. The filtrate was diluted with DMF and injected onto a reverse phase HPLC system (acetonitrile/water/0.1% formic acid). Fractions were concentrated to give 2-(tert-butoxy)-2-(1-(3,4-difluorobenzyl)-7-methyl-5-(p-tolyl)-1,2,3,4-tetrahydroquinolin-6-yl)acetic acid (5.4 mg, 11 μmol, 43% yield). ¹H NMR (400 MHz, CHLOROFORM-d) δ ppm 9.3-10.2 (m, 1H), 7.4 (d, J=7.14 Hz, 1H), 7.2-7.3 (m, 2H), 7.1-7.2 (m, 3H), 7.0 (br. s., 1H), 6.3 (s, 1H), 5.0 (s, 1H), 4.4 (d, J=3.66 Hz, 2H), 3.1-3.4 (m, 2H), 2.3-2.5 (m, 4H), 2.2-2.3 (m, 4H), 1.7-2.0 (m, 2H), 1.0 (s, 9H). LCMS (ES+) (m/z): 494.4 (M+H).

### Example 213: 2-(tert-Butoxy)-2-(7-methyl-1-(piperidin-1-ylsulfonyl)-5-(p-tolyl)-1,2,3,4-tetrahydroquinolin-6-yl)acetic acid

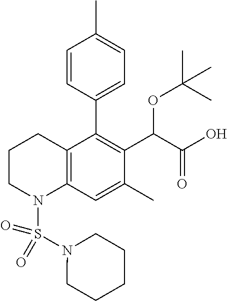 [SMILES: Cc1ccc(-c2c3c(cc(C)c2C(OC(C)(C)C)C(=O)O)N(S(=O)(=O)N2CCCCC2)CCC3)cc1]

Ethyl 2-(tert-butoxy)-2-(7-methyl-5-(p-tolyl)-1-((trifluoromethyl)sulfonyl)-1,2,3,4-tetrahydroquinolin-6-yl)acetate (13 mg, 0.025 mmol) and potassium trimethylsilanolate (19.0 mg, 0.148 mmol) were heated at 80° C. under nitrogen for 5 hours. The mixture was allowed to cool to room temperature, quenched with 1N hydrochloric acid, and extracted 3 times with 2-methyltetrahydrofuran. The combined organic layers were dried over sodium sulfate and concentrated to give 2-(tert-butoxy)-2-(7-methyl-5-(p-tolyl)-1,2,3,4-tetrahydroquinolin-6-yl)acetic acid. The residue was dissolved in dichloromethane (DCM) (1.000 mL) before pyridine (2.0 μl, 0.025 mmol) was added followed by piperidine-1-sulfonyl chloride (4.2 μl, 0.030 mmol). After stirring for several hours, the mixture was concentrated and the residue purified by reverse phase HPLC (acetonitrile/water/0.1% formic acid). Fractions were concentrated. Excess water was azeotroped with acetonitrile and concentrated to give 2-(tert-butoxy)-2-(7-methyl-1-(piperidin-1-ylsulfonyl)-5-(p-tolyl)-1,2,3,4-tetrahydroquinolin-6-yl)acetic acid (2.2 mg, 4.2 μmol, 17% yield). ¹H NMR (400 MHz, CHLOROFORM-d) δ ppm 7.4 (s, 1H), 7.4 (d, J=6.59 Hz, 1H), 7.2-7.3 (m, 2H), 7.1 (d, J=7.14 Hz, 1H), 5.0 (s, 1H), 3.5-3.8 (m, 2H), 3.2 (t, J=4.85 Hz, 4H), 2.3-2.5 (m, 6H), 2.1-2.3 (m, 1H), 1.9-2.1 (m, 2H), 1.7-1.8 (m, 2H), 1.6 (d, J=3.11 Hz, 6H), 1.0 (s, 9H). LCMS (ES+) (m/z): 515.5 (M+H).

### Example 214: 2-(tert-Butoxy)-2-(1-(3-fluorobenzoyl)-7-methyl-5-(p-tolyl)-1,2,3,4-tetrahydroquinolin-6-yl)acetic acid

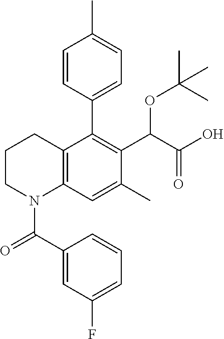 [SMILES: Cc1ccc(-c2c3c(cc(C)c2C(OC(C)(C)C)C(=O)O)N(C(=O)c2cccc(F)c2)CCC3)cc1]

Ethyl 2-(tert-butoxy)-2-(7-methyl-5-(p-tolyl)-1-((trifluoromethyl)sulfonyl)-1,2,3,4-tetrahydroquinolin-6-yl)acetate (27 mg, 0.051 mmol) and potassium trimethylsilanolate (65.7 mg, 0.512 mmol) in 1,4-dioxane (0.5 mL) were heated at 100° C. overnight under nitrogen. The mixture was allowed to cool to room temperature, cooled to 0° C., and acidified to pH=˜2 with 1N hydrochloric acid. The mixture was extracted 3 times with 2-methyltetrahydrofuran. The combined organic layers were washed with brine, dried over sodium sulfate, and concentrated. The residue was dissolved in ethyl acetate (0.500 mL) before 3-fluorobenzoic acid (14.34 mg, 0.102 mmol), triethylamine (0.071 mL, 0.512 mmol), and 1-propanephosphonic acid cyclic anhydride, 50 wt. % solution in ethyl acetate (0.183 mL, 0.307 mmol) were added. The mixture was stirred for 2 hours, quenched with saturated sodium bicarbonate, and extracted 2 times with 2-methyltetrahydrofuran. The combined organic layers were washed with 10% citric acid, washed with brine, dried over sodium sulfate, and concentrated. The residue was purified by reverse phase ISCO chromatography (acetonitrile/water/0.1% formic acid). Fractions were concentrated to give 2-(tert-butoxy)-2-(1-(3-fluorobenzoyl)-7-methyl-5-(p-tolyl)-1,2,3,4-tetrahydroquinolin-6-yl)acetic acid (1.0 mg, 2.0 μmol, 3.9% yield). ¹H NMR (400 MHz, CHLOROFORM-d) b ppm 7.4-7.5 (m, 1H), 7.3 (s, 4H), 7.1-7.2 (m, 2H), 7.0-7.1 (m, 2H), 6.4-6.7 (m, 1H), 5.1 (s, 1H), 3.9-4.2 (m, 1H), 3.5-3.7 (m, 1H), 2.4 (s, 6H), 2.1 (s, 4H), 1.0 (s, 9H). LCMS (ES+) (m/z): 490.4 (M+H).

TABLE 1

| Example | IC50 (μM) |
|---|---|
| 1 | 0.517 |
| 2 | 0.330 |
| 3 | 0.012 |
| 4 | 7.524 |
| 5 | 0.343 |
| 6 | 0.369 |
| 7 | 3.007 |
| 8 | 0.015 |
| 9 | 0.011 |
| 10 | 0.012 |
| 11 | 0.004 |
| 12 | 0.004 |
| 13 | 0.011 |
| 14 | 0.007 |
| 15 | 0.117 |
| 16 | 0.012 |
| 17 | 0.011 |
| 18 | 0.011 |
| 19 | 0.005 |
| 20 | 0.066 |
| 21 | 0.009 |
| 22 | 0.011 |
| 23 | 0.095 |
| 24 | 0.047 |
| 25 | 0.043 |
| 26 | 0.123 |
| 27 | 0.007 |
| 28 | 11.42 |
| 29 | 0.017 |
| 30 | 0.008 |
| 31 | 0.012 |
| 32 | 0.011 |
| 33 | 0.013 |
| 34 | 0.069 |
| 35 | 9.800 |
| 36 | 0.065 |
| 37 | 0.080 |
| 38 | 0.018 |
| 39 | 0.027 |
| 40 | 0.283 |
| 41 | 0.017 |
| 42 | 0.005 |
| 43 | 0.010 |
| 44 | 0.006 |
| 45 | 0.001 |
| 46 | 0.004 |
| 47 | 0.004 |
| 48 | 0.004 |
| 49 | 0.002 |
| 50 | 0.005 |
| 51 | 0.017 |
| 52 | 0.002 |
| 53 | 0.003 |
| 54 | 0.012 |
| 55 | 0.006 |
| 56 | 0.002 |
| 57 | 0.002 |
| 58 | 0.041 |
| 59 | 0.006 |
| 60 | 0.006 |
| 61 | 0.017 |
| 62 | 0.006 |
| 63 | 0.004 |
| 64 | 0.005 |
| 65 | 0.378 |
| 66 | 0.006 |
| 67 | 0.005 |
| 68 | 0.013 |
| 69 | 0.005 |
| 70 | 0.005 |
| 71 | 0.004 |
| 72 | 0.007 |
| 73 | 0.006 |
| 74 | 0.005 |
| 75 | 0.0066148 |
| 76 | 0.015 |
| 77 | 0.005 |
| 78 | 0.310 |
| 79 | 0.055 |
| 80 | 0.004 |
| 81 | 0.014 |
| 82 | 0.012 |
| 83 | 0.007 |
| 84 | 0.041 |
| 85 | 0.006 |
| 86 | 0.005 |
| 87 | 0.005 |
| 88 | 0.005 |
| 89 | 0.003 |
| 90 | 0.005 |
| 91 | 0.156 |
| 92 | 0.006 |
| 93 | 0.019 |
| 94 | 0.004 |
| 95 | 0.142 |
| 96 | 0.004 |
| 97 | 0.006 |
| 98 | 0.002 |
| 99 | 0.004 |
| 100 | 0.005 |
| 101 | 0.005 |
| 102 | 0.002 |
| 103 | 0.016 |
| 104 | 0.323 |
| 105 | 0.021 |
| 106 | 0.011 |
| 107 | 0.005 |
| 108 | 0.004 |
| 109 | 0.011 |
| 110 | 0.004 |
| 111 | 0.043 |
| 112 | 0.295 |
| 113 | 0.002 |
| 114 | 0.005 |
| 115 | 0.005 |
| 116 | 0.011 |
| 117 | 0.016 |
| 118 | 0.005 |
| 119 | 0.003 |
| 120 | 0.091 |
| 121 | 0.007 |
| 122 | 0.017 |
| 123 | 0.003 |
| 124 | 0.003 |
| 125 | 0.005 |
| 126 | 0.005 |
| 127 | 0.002 |
| 128 | 0.005 |
| 129 | 0.022 |
| 130 | 0.004 |
| 131 | 0.11 |
| 132 | 0.414 |
| 133 | 0.067 |
| 134 | 0.453 |
| 135 | 0.004 |
| 136 | 0.041 |
| 137 | 0.004 |
| 138 | 0.005 |
| 139 | 0.006 |
| 140 | 0.014 |
| 141 | 0.006 |
| 142 | 0.002 |
| 143 | 0.014 |
| 144 | 0.026 |
| 145 | 0.014 |
| 146 | 0.005 |
| 147 | 0.017 |
| 148 | 0.008 |
| 149 | 0.005 |
| 150 | 0.008 |
| 151 | 0.017 |
| 152 | 0.013 |
| 153 | 0.125 |
| 154 | 0.052 |
| 155 | 0.072 |
| 156 | 0.005 |
| 157 | 0.006 |
| 158 | 0.003 |
| 159 | 0.004 |
| 160 | 0.014 |
| 161 | 0.081 |
| 162 | 0.007 |
| 163 | 0.009 |
| 164 | 0.176 |
| 165 | 0.006 |
| 166 | 0.004 |
| 167 | 0.007 |
| 168 | 0.013 |
| 169 | 0.002 |
| 170 | 0.014 |
| 171 | 0.014 |
| 172 | 0.006 |
| 173 | 0.013 |
| 174 | 0.006 |
| 175 | 0.090 |
| 176 | 0.005 |
| 177 | 0.002 |
| 178 | 0.004 |
| 179 | 0.012 |
| 180 | 0.005 |
| 181 | 0.002 |
| 182 | 0.005 |
| 183 | 0.004 |
| 184 | 0.004 |
| 185 | 0.009 |
| 186 | 0.006 |
| 187 | 0.006 |
| 188 | 0.005 |
| 189 | 0.006 |
| 190 | 0.124 |
| 191 | 0.868 |
| 192 | 0.005 |
| 193 | 0.009 |
| 194 | 0.043 |
| 195 | 0.121 |
| 196 | 0.359 |
| 197 | 0.712 |
| 198 | 0.150 |
| 199 | 0.042 |
| 200 | 0.034 |
| 201 | 0.080 |
| 202 | 0.093 |
| 203 | 0.016 |
| 204 | 0.019 |
| 205 | 0.020 |
| 206 | 0.013 |
| 207 | 3.50 |
| 208 | 1.12 |
| 209 | 1.34 |
| 210 | 4.29 |
| 211 | 10.39 |
| 212 | 1.03 |
| 213 | 3.41 |
| 214 | 3.56 |

1. A compound of Formula I: 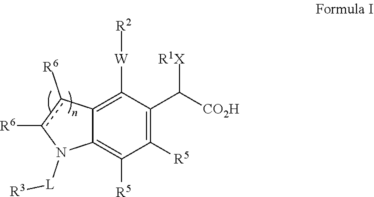
or a pharmaceutically acceptable salt thereof wherein:
the dashed line between the carbons to which the R⁶ groups are bonded is meant to indicate that the bond can be either a single bond or a double bond;
n is 1 or 2 with the proviso that when n is 2 the dashed line must be a single bond;
X is O or CH₂;
R¹ is C₁₋₆alkyl wherein said alkyl may contain cycloalkyl portions;
W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂(CO)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉ heteroaryl, wherein each R² group is optionally substituted by one to four substituents selected from halo, C₁₋₆alkyl, C₁₋₆heteroalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
L is a bond, —CH₂(CO)—, C₁₋₃alkylene, —SO₂—, —SO₂NH—, —C(O)—, —C(O)NH—, —C(O)NHC₁₋₂alkyl-, —C(O)OCH₂—, —C(O)O—, —C(O)C(O)—, or —C(O)C₁₋₂alkyl-;
R³ is H, CN, C₁₋₆alkyl, C₅₋₁₄aryl, C₅₋₁₄aryl, C₃₋₇cycloalkyl, C₃₋₇cycloalkyl, C₃₋₇spirocycloalkyl, C₃₋₇cycloalkenyl, C₃₋₉heterocycle, C₅₋₉heteroaryl, or tetrahydronaphthyl, and wherein R³ is optionally substituted by one to four substituents selected from halo, oxo, C₁₋₆alkyl, C₃₋₇cycloalkyl, C₁₋₃fluoroalkyl, —OC₁₋₆alkyl, —C(O)C₁₋₃alkyl, —C(O)N(H)C₁₋₃alkyl, —NHC(O)C₁₋₃ alkyl, —C(O)NHR⁴, C₅₋₁₄aryl, C₁₋₆heteroalkyl, —B(OH)₂, C₃₋₉heterocycle, C₅₋₉heteroaryl, —C(O)OC₁₋₆alkyl, or the following divalent substituents may be bonded to adjacent atoms of R³ to form a fused ring, —C₂₋₅alkylene-, —OC₁₋₃alkyleneO-, —OC₁₋₄alkylene-, or —N═C(CH₃)O—;
each R⁵ is independently H, C₁₋₃alkyl, C₃₋₆cycloalkyl, CH₂F, CHF₂, or CF₃;
each R⁶ is independently H, C₁₋₃alkyl, C₅₋₁₄aryl, C₃₋₉heterocycle, C₅₋₉heteroaryl, —C(O)NR⁴, or —C(O)NHR⁴, or an R⁶ may represent a gem dimethyl, or two R⁶ groups may together comprise 2-4 carbon atoms and join together to form a fused ring system wherein the ring formed by the two R⁶ groups can be cycloalkyl, or heterocycle, aryl, or heteroaryl;
and wherein each heterocycle, heteroaryl, heteroalkyl, and heteroalkylene comprises one to three heteroatoms selected from S, N, B, or O.

2. A compound or salt according to claim 1 wherein n is 1.

3. A compound or salt according to claim 2 wherein the dashed line represents a single bond.

4. A compound or salt according to claim 1 wherein W is a bond.

5. A compound or salt according to claim 1, wherein R¹ is t-butyl.

6. A compound or salt according to claim 1 wherein X is O.

7. A compound or salt according to claim 1 wherein R² is phenyl optionally substituted by one to four substituents selected from halo, C₁₋₆alkyl, C₁₋₆heteroalkyl, or C₁₋₆alkylene or C₁₋₆hetereoalklylene wherein said C₁₋₆alkylene or C₁₋₆hetereoalklylene is bonded to adjacent carbon atoms on said phenyl to form a fused ring and wherein each heteroalkyl and heteroalkylene comprises one to two heteroatoms selected from S, N, or O.

8. A compound or salt according to claim 7 wherein R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

9. A compound or salt according to claim 1 wherein L is CH₂, —C(O)—, a bond, —C(O)C(O)—, —C(O)NH—, —C(O)O—, —C(O)CH₂—, SO₂, —C(O)CH₂CH₂—, —CH₂C(O)—, or —C(O)CH₂—.

10. A compound or salt according to claim 9 wherein L is —C(O)—.

11. A compound or salt according to claim 1 wherein R³ is C₂₋₆alkyl, C₅₋₆cycloalkenyl, C₅₋₆aryl, C₃₋₆cycloalkyl, C₅₋₆heterocycle containing 1 oxygen atom or 1 nitrogen atom, C₅₋₆heteroaryl containing 1-3 heteroatoms selected from N, S, and O, wherein R³ is optionally substituted by one to three substituents selected from F, Cl, C₁₋₃alkyl, OC₁₋₃alkyl, C₁₋₃fluoroalkyl, NHC(O)C₁₋₃alkyl, C(O)NHC₁₋₃alkyl, C(O)OC₁₋₃alkyl, or the following divalent substituents may be bonded to adjacent atoms of R³ to form a fused ring, —C₂₋₅alkylene-, —OC₁₋₃alkyleneO-, —OC₁₋₄alkylene-, or —N═C(CH₃)O—.

12. A compound or salt according to claim 11 wherein R³ is phenyl optionally substituted by one to three substituents selected from F, Cl, C₁₋₃alkyl, OC₁₋₃alkyl, C₁₋₃fluoroalkyl, NHC(O)C₁₋₃alkyl, C(O)NHC₁₋₃alkyl, C(O)OC₁₋₃alkyl, or the following divalent substituents may be bonded to adjacent atoms of R³ to form a fused ring, —C₂₋₅alkylene-, —OC₁₋₃alkyleneO-, —OC₁₋₄alkylene-, or —N═C(CH₃)O—.

13. A compound or salt according to claim 1 wherein one R⁵ is methyl and the other is H.

14. A compound or salt according to claim 1 wherein each R⁶ is H.

15. A compound or salt according to claim 1 wherein the stereochemistry on the carbon to 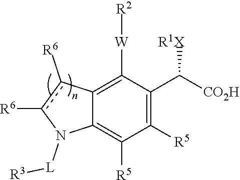
which XR¹ is bound is as shown above.

16. A compound of Formula II 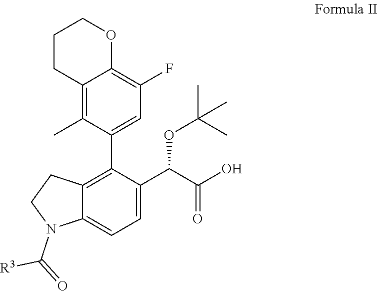
or a pharmaceutically acceptable salt thereof wherein R³ is phenyl optionally substituted by one to three substituents selected from F, Cl, C₁₋₃alkyl, OC₁₋₃alkyl, C₁₋₃fluoroalkyl, NHC(O)C₁₋₃alkyl, C(O)NHC₁₋₃alkyl, C(O)OC₁₋₃alkyl, or the following divalent substituents may be bonded to adjacent atoms of R³ to form a fused ring, —C₂₋₅alkylene-, —OC₁₋₃alkyleneO-, —OC₁₋₄alkylene-, or —N═C(CH₃)O—.

17. (canceled)

18. A pharmaceutical compositions comprising a compound or salt according to claim 1.

19. A method for treating or preventing a viral infection in a patient mediated at least in part by a virus in the retrovirus family of viruses, comprising administering to said patient a composition according to claim 18.

20. The method of claim 19 wherein the viral infection is mediated by the HIV virus.

21. The method of claim 20 further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

22-24. (canceled)
